(19) United States
(12) Patent Application Publication
(10) Pub. No.: US 2015/0331314 A1
(43) Pub. Date: Nov. 19, 2015

(54) PATTERN FORMING METHOD, COMPOUND USED THEREIN, ACTINIC RAY-SENSITIVE OR RADIATION-SENSITIVE RESIN COMPOSITION, RESIST FILM, MANUFACTURING METHOD OF ELECTRONIC DEVICE, AND ELECTRONIC DEVICE

(71) Applicant: FUJIFILM CORPORATION, Tokyo (JP)

(72) Inventors: Shuhei YAMAGUCHI, Haibara-gun (JP); Takamitsu TOMIGA, Haibara-gun (JP); Fumihiro YOSHINO, Haibara-gun (JP); Hajime FURUTANI, Haibara-gun (JP); Michihiro SHIRAKAWA, Haibara-gun (JP); Mitsuhiro FUJITA, Ashigarakami-gun (JP)

(73) Assignee: FUJIFILM CORPORATION, Tokyo (JP)

(21) Appl. No.: 14/811,926

(22) Filed: Jul. 29, 2015

Related U.S. Application Data
(63) Continuation of application No. PCT/JP2014/052177, filed on Jan. 24, 2014.
(60) Provisional application No. 61/758,973, filed on Jan. 31, 2013.

(30) Foreign Application Priority Data
Jan. 31, 2013 (JP) 2013-017949

(51) Int. Cl. G03F 7/004; C07D 409/02; G03F 7/32; C07C 311/48; G03F 7/038; G03F 7/20

(52) U.S. Cl. CPC G03F 7/0045; G03F 7/038; G03F 7/20; G03F 7/325; C07C 311/48; C07D 409/02; C07C 2101/16

(57) ABSTRACT

There is provided an actinic ray-sensitive or radiation-sensitive resin composition comprising: (A) a resin having a group capable of decomposing by an action of an acid to produce a polar group, (C1) a compound containing a group capable of generating a first acidic functional group upon irradiation with an actinic ray or radiation and a group capable of generating a second acidic functional group different from the first acidic functional group upon irradiation with an actinic ray or radiation, and (C2) at least one compound containing two or more groups selected from the group consisting of the groups capable of generating the structures represented by the specific formulae upon irradiation with an actinic ray or radiation.

### CROSS REFERENCE TO RELATED APPLICATION

This is a continuation of International Application No. PCT/JP2014/052177 filed on Jan. 24, 2014, and claims priority from Japanese Patent Application No. 2013-017949 filed on Jan. 31, 2013, U.S. Provisional Application No. 61/758,973 filed on Jan. 31, 2013, the entire disclosures of which are incorporated therein by reference.

### TECHNICAL FIELD

The present invention relates to a pattern forming method, a compound used therein, an actinic ray-sensitive or radiation-sensitive resin composition, a resist film, a manufacturing method of an electronic device, and an electronic device. More specifically, the present invention relates to a pattern forming method suitable for lithography in the process of producing a semiconductor such as IC or the production of a liquid crystal device or a circuit board such as thermal head and further in other photo-fabrication processes, a compound used in the pattern forming method, an actinic ray-sensitive or radiation-sensitive resin composition, a resist film, a manufacturing method of an electronic device, and an electronic device. Above all, the present invention relates to a pattern forming method suitable for exposure by an ArF exposure apparatus, an ArF immersion-type projection exposure apparatus and an EUV exposure apparatus each using a light source that emits a far ultraviolet ray having a wavelength of 300 nm or less, a compound used in the pattern forming method, an actinic ray-sensitive or radiation-sensitive resin composition, a resist film, a manufacturing method of an electronic device, and an electronic device.

### BACKGROUND ART

Since the advent of a resist for KrF excimer laser (248 nm), a pattern forming method utilizing chemical amplification is used so as to compensate for sensitivity reduction due to light absorption. For example, in the positive chemical amplification method, first, a photoacid generator contained in the exposed area decomposes upon irradiation with light to generate an acid and in the course of baking or the like after exposure (PEB: Post Exposure Bake), an alkali-insoluble group contained in the photosensitive composition is changed into an alkali-soluble group by the catalytic action of the acid generated. Thereafter, development is performed using, for example, an alkali solution, whereby the exposed area is removed and a desired pattern is obtained.

As for the alkali developer used in the method above, various developers have been proposed. For example, as the alkali developer, an aqueous alkali developer of 2.38 mass % TMAH (aqueous tetramethylammonium hydroxide solution) is being used for general purposes.

In the positive chemical amplification method, from the standpoint of improving the resolution, dry etching resistance, pattern forming performance and the like, attempts have been made to provide a group capable of decomposing by the action of an acid to a polymer main chain through a polycyclic hydrocarbon group as a spacer (for example, Japanese Patent No. 3,390,702, JP-A-2008-58538 (the term “JP-A” as used herein means an “unexamined published Japanese patent application”), JP-A-2010-254639, JP-A-2010-256873, and JP-A-2000-122295).

Also, in the positive chemical amplification method, from the standpoint of enhancing the exposure latitude and suppressing the line edge roughness, it is known to use a photoacid generator containing a specific sulfonylimide structure or sufonylmethide structure (JP-A-2011-37825).

Miniaturization of a semiconductor device has promoted progress in shortening the wavelength of the exposure light source and increasing the numerical aperture (higher NA) of a projection lens, and an exposure machine using an ArF excimer laser having a wavelength of 193 nm as the light source has been so far developed. As a technique to more increase the resolution, a method of filling the space between the projection lens and the sample with a high refractive-index liquid (hereinafter, sometimes referred to as an “immersion liquid”) (that is, an immersion method) has been proposed (see, JP-A-2011-76057). Furthermore, EUV lithography of performing exposure to ultraviolet light with a shorter wavelength (13.5 nm) has also been proposed.

However, it is actually very difficult to find out an appropriate combination of a resist composition, a developer, a rinsing solution and the like, which are necessary to form a pattern having overall excellent performance.

In recent years, a pattern forming method using an organic solvent-containing developer has also been developed (see, for example, JP-A-2008-292975, JP-A-2010-197619). For example, JP-A-2008-292975, JP-A-2010-197619 disclose a pattern forming method including a step of coating a substrate with a resist composition capable of increasing the solubility for an alkali developer and decreasing the solubility for an organic solvent-containing developer upon irradiation with an actinic ray or radiation, an exposure step, and a step of performing development by using an organic solvent-containing developer. According to this method, a high-definition fine pattern can be stably formed.

### SUMMARY OF INVENTION

However, in the pattern forming method above, more improvements are required in terms of roughness performance, local pattern dimension uniformity, exposure latitude and reduction in the film loss during development.

In WO2012/053527A1 and JP-A-2012-123208, it is attempted to improve these matters by the addition of a compound capable of generating an acid different from a fluoroalkylsulfonic acid that is usually employed in an ArF resist. However, the improvement is insufficient particularly in terms of local pattern dimension uniformity and film loss during development.

An object of the present invention is to provide a pattern forming method ensuring that the roughness performance such as line width roughness, the local pattern dimension uniformity and the exposure latitude are excellent and reduction in the film thickness of the pattern part formed by development, so-called film loss, can be suppressed, a compound used therein, an actinic ray-sensitive or radiation-sensitive resin composition, a resist film, a manufacturing method of an electronic device, and an electronic device.

The present invention includes the following configurations, and the above-described object of the present invention can be attained by these configurations.

[1] An actinic ray-sensitive or radiation-sensitive resin composition comprising:

(A) a resin having a group capable of decomposing by an action of an acid to produce a polar group,

(C1) a compound containing a group capable of generating a first acidic functional group upon irradiation with an actinic ray or radiation and a group capable of generating a second acidic functional group different from the first acidic functional group upon irradiation with an actinic ray or radiation, and

(C2) at least one compound containing two or more groups selected from the group consisting of a group capable of generating a structure represented by the following formula (a) upon irradiation with an actinic ray or radiation, a group capable of generating a structure represented by the following formula (b) upon irradiation with an actinic ray or radiation, a group capable of generating a structure represented by the following formula (c) upon irradiation with an actinic ray or radiation, and a group capable of generating a structure represented by the following formula (d) upon irradiation with an actinic ray or radiation:

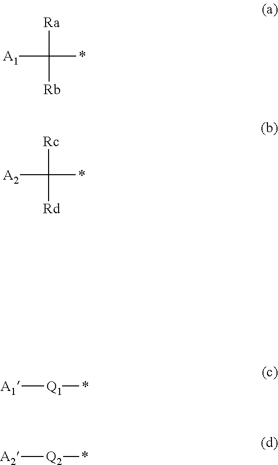

wherein in formulae (a), (b), (c) and (d),

A₁, A₂, A₁′ and A₂′ represent the same acidic functional group,

each of Ra, Rb, Rc and Rd independently represents a hydrogen atom or a substituent,

each of Q₁ and Q₂ represents a cyclic group,

provided that the structure represented by formula (a) is different from the structure represented by formula (b) and the structure represented by formula (c) is different from the structure represented by formula (d), and

* represents a bond.

[2] The actinic ray-sensitive or radiation-sensitive resin composition as described in [1],

wherein the compound (C1) is a compound capable of generating, as the first acidic functional group and the second acidic functional group, different groups from each other selected from the group consisting of groups represented by the following formulae (Ca-1) to (Ca-19), upon irradiation with an actinic ray or radiation:

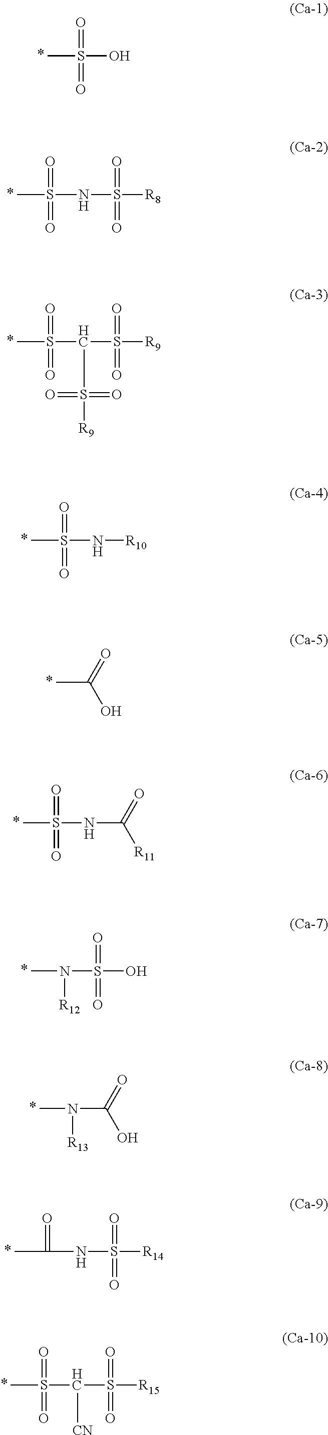

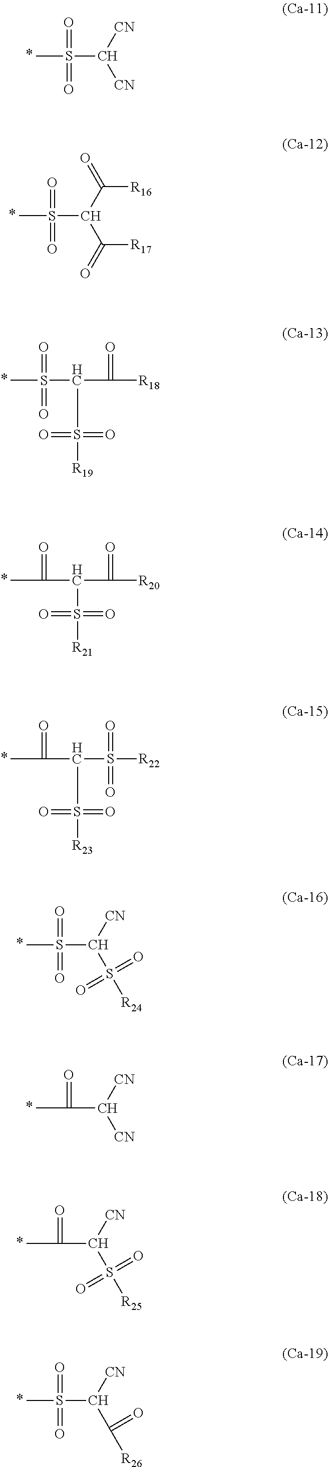

wherein in formulae (Ca-2) to (Ca-4), (Ca-6) to (Ca-10), (Ca-12) to (Ca-16), (Ca-18) and (Ca-19),

each of R₈, R₉, R₁₁ and R₁₄ to R₂₆ independently represents an alkyl group, a cycloalkyl group or an aryl group,

R₁₀ represents a hydrogen atom, an alkyl group, a cycloalkyl group or an aryl group, and

each of R₁₂ and R₁₃ independently represents a hydrogen atom, an alkyl group, an aryl group, or a single bond, alkylene group or arylene group capable of bonding to any one atom in the molecule to form a ring.

[3] The actinic ray-sensitive or radiation-sensitive resin composition as described in [2],

wherein the compound (C1) is a compound capable of generating a group selected from the group consisting of groups represented by the following formulae (Cb-1) to (Cb-4), upon irradiation with an actinic ray or radiation:

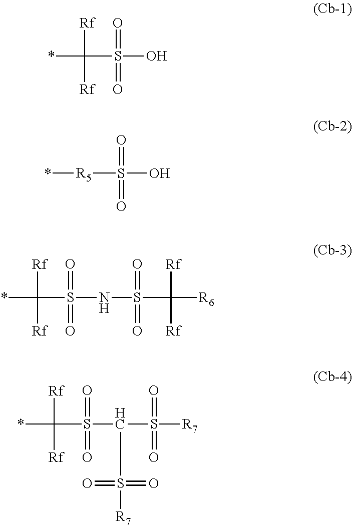

wherein in formulae (Cb-1) to (Cb-4),

each Rf independently represents a fluorine atom or an alkyl group substituted with at least one fluorine atom,

R₅ represents an arylene group containing a fluorine atom or an alkyl group substituted with at least one fluorine atom,

R₆ represents a hydrogen atom, a fluorine atom or an alkyl group,

each R₇ independently represents an alkyl group, a cycloalkyl group or an aryl group, and

* represents a bond.

[4] The actinic ray-sensitive or radiation-sensitive resin composition as described in [1],

wherein the compound (C2) is a compound capable of generating, as A₁ in formula (a), A₂ in formula (b), A₁′ in formula (c) and A₂′ in formula (d), the same groups as each other selected from the group consisting of groups represented by the following formulae (Ca-1) to (Ca-19), upon irradiation with an actinic ray or radiation:

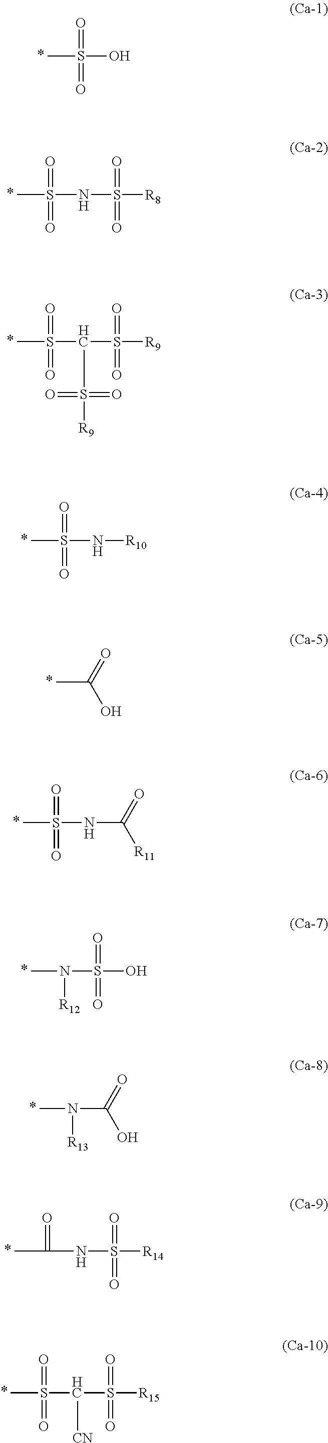

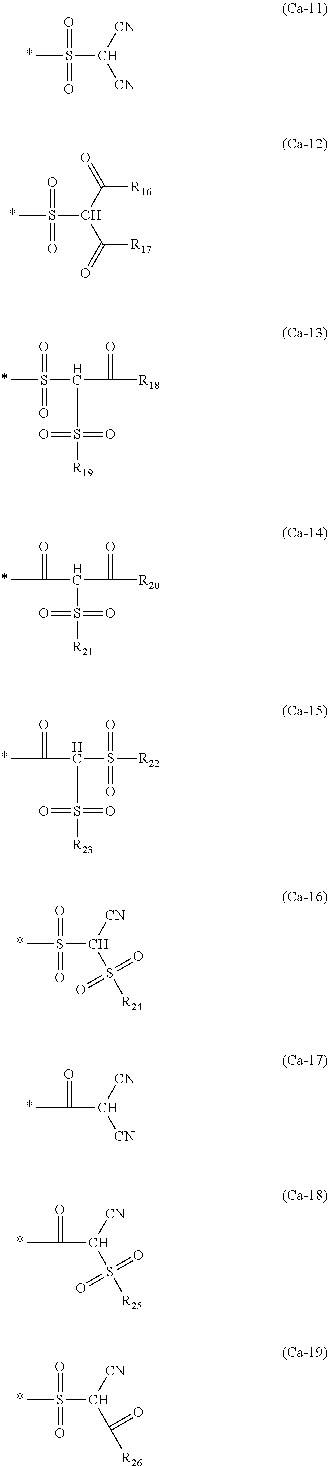

wherein in formulae (Ca-2) to (Ca-4), (Ca-6) to (Ca-10), (Ca-12) to (Ca-16), (Ca-18) and (Ca-19),

each of R₈, R₉, R₁₁, and R₁₄ to R₂₆ independently represents an alkyl group, a cycloalkyl group or an aryl group,

R₁₀ represents a hydrogen atom, an alkyl group, a cycloalkyl group or an aryl group, and

each of R₁₂ and R₁₃ independently represents a hydrogen atom, an alkyl group, an aryl group, or a single bond, alkylene group or arylene group capable of bonding to any one atom in the molecule to form a ring.

[5] The actinic ray-sensitive or radiation-sensitive resin composition as described in [4],

wherein the compound (C2) is a compound capable of generating a group selected from the group consisting of groups represented by the following formulae (Cb-1) to (Cb-4), upon irradiation with an actinic ray or radiation:

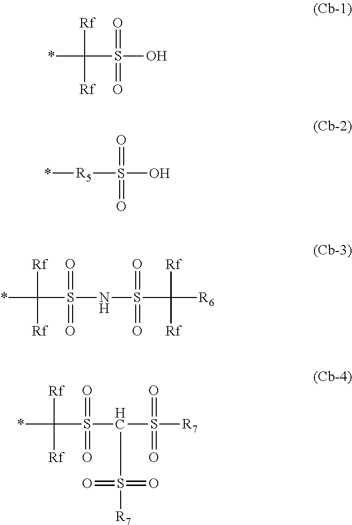

wherein in formulae (Cb-1) to (Cb-4),

each Rf independently represents a fluorine atom or an alkyl group substituted with at least one fluorine atom,

R₅ represents an arylene group containing a fluorine atom or an alkyl group substituted with at least one fluorine atom,

R₆ represents a hydrogen atom, a fluorine atom or an alkyl group,

each R₇ independently represents an alkyl group, a cycloalkyl group or an aryl group, and

* represents a bond.

[6] The actinic ray-sensitive or radiation-sensitive resin composition as described in [1],

wherein the compound (C2) is a compound containing a group capable of generating a structure represented by formula (a) upon irradiation with an actinic ray or radiation and a group capable of generating a structure represented by formula (b) upon irradiation with an actinic ray or radiation,

at least either one of Ra and Rb in formula (a) represents a fluorine atom or an alkyl fluoride group, and

each of Rc and Rd in formula (b) independently represents a hydrogen atom or an alkyl group not substituted with a fluorine atom.

[7] The actinic ray-sensitive or radiation-sensitive resin composition as described in any one of [1] to [6], further comprising:

(B) a compound capable of generating an acid upon irradiation with an actinic ray or radiation, which is different from the compounds (C1) and (C2).

[8] The actinic ray-sensitive or radiation-sensitive resin composition as described in [7],

wherein the compound (B) is a compound capable of generating an organic acid represented by the following formula (V) or (VI) upon irradiation with an actinic ray or radiation:

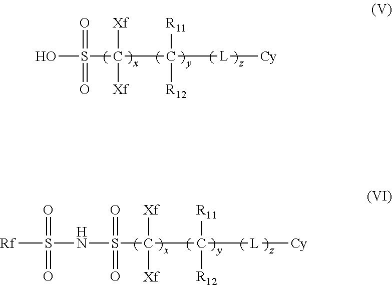

wherein in formulae (V) and (VI),

each Xf independently represents a fluorine atom or an alkyl group substituted with at least one fluorine atom,

each L independently represents a divalent linking group,

each of R₁₁ and R₁₂ independently represents a hydrogen atom, a fluorine atom or an alkyl group,

Cy represents a cyclic organic group,

Rf represents a fluorine atom-containing group,

x represents an integer of 1 to 20,

y represents an integer of 0 to 10, and

z represents an integer of 0 to 10.

[9] The actinic ray-sensitive or radiation-sensitive resin composition as described in any one of [1] to [8],

wherein the resin (A) contains (AI) a repeating unit capable of decomposing by an action of an acid to produce a carboxyl group.

[10] The actinic ray-sensitive or radiation-sensitive resin composition as described in [9],

wherein the content of the repeating unit (AI) is 50 mol % or more based on all repeating units in the resin (A).

[11] The actinic ray-sensitive or radiation-sensitive resin composition as described in any one of [1] to [10], further comprising:

(D) a hydrophobic resin different from the resin (A).

[12]A pattern forming method comprising:

(i) a step of forming a film by using the actinic ray-sensitive or radiation-sensitive resin composition described in any one of [1] to [11],

(ii) a step of exposing the film, and

(iii) a step of developing the exposed film by using a developer to form a pattern.

[13] The pattern forming method as described in [12],

wherein the step (iii) is a step of developing the exposed film by using an organic solvent-containing developer to form a negative pattern.

[14] The pattern forming method as described in [12],

wherein the exposure in the step (ii) is immersion exposure.

[15] The pattern forming method as described in [13] or [14],

wherein the developer is a developer containing at least one kind of an organic solvent selected from the group consisting of a ketone-based solvent, an ester-based solvent, an alcohol-based solvent, an amide-based solvent and an ether-based solvent.

[16]A resist film formed of the actinic ray-sensitive or radiation-sensitive resin composition described in any one of [1] to [11].
[17]A method for manufacturing an electronic device, comprising the pattern forming method described in any one of [12] to [15].
[18] An electronic device manufactured by the manufacturing method of an electronic device described in [17].
[19] A compound represented by the following formula (C-1) or (C-2):

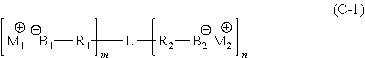

wherein in formula (C-1),

each of M₁ and M₂ represents an organic counter cation structure,

B₁ represents an acid anion moiety of a first acidic functional group,

B₂ represents an acid anion moiety of a second acidic functional group different from the first acidic functional group,

each of R₁ and R₂ independently represents a single bond, an alkylene group, a cycloalkylene group or an arylene group,

L represents an (m+n)-valent linking group,

each of m and n represents an integer, and m≧n;

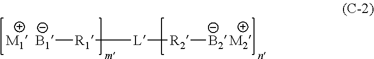

wherein in formula (C-2),

M₁′, M₂′, R₁′, R₂′, L′, m′ and n′ have the same meanings as M₁, M₂, R₁, R₂, L, m and n, respectively, in formula (C-1), m′≧n′, and

B₁′ and B₂′ represent different kinds of acid anion structures selected from the group consisting of an acid anion structure of a structure represented by the following formula (a), an acid anion structure of a structure represented by the following formula (b), an acid anion structure of a structure represented by the following formula (c), and an acid anion structure of a structure represented by the following formula (d):

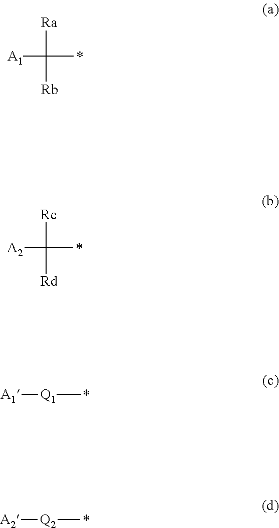

wherein in formulae (a), (b), (c) and (d),

A₁, A₂, A₁′ and A₂′ represent the same acidic functional group,

each of Ra, Rb, Rc and Rd independently represents a hydrogen atom or a substituent,

each of Q₁ and Q₂ represents a cyclic group,

provided that the structure represented by formula (a) is different from the structure represented by formula (b) and the structure represented by formula (c) is different from the structure represented by formula (d), and

* represents a bond.

According to the present invention, a pattern forming method ensuring that the roughness performance such as line width roughness, the local pattern dimension uniformity and the exposure latitude are excellent and reduction in the film thickness of the pattern part formed by development, so-called film loss, can be suppressed, a compound used therein, an actinic ray-sensitive or radiation-sensitive resin composition, a resist film, a manufacturing method of an electronic device, and an electronic device can be provided.

### BRIEF DESCRIPTION OF THE DRAWINGS

FIG. 1 is a view showing the ¹H-NMR chart of Compound (C-1) synthesized in Examples.

FIG. 2 is a view showing the ¹⁹F-NMR chart of Compound (C-1) synthesized in Examples.

### DESCRIPTION OF EMBODIMENTS

The mode for carrying out the present invention is described below.

In the description of the present invention, when a group (atomic group) is denoted without specifying whether substituted or unsubstituted, the group encompasses both a group having no substituent and a group having a substituent. For example, “an alkyl group” encompasses not only an alkyl group having no substituent (unsubstituted alkyl group) but also an alkyl group having a substituent (substituted alkyl group).

In the description of the present invention, the “actinic ray” or “radiation” means, for example, a bright line spectrum of mercury lamp, a far ultraviolet ray typified by excimer laser, an extreme-ultraviolet ray (EUV light), an X-ray or an electron beam (EB). Also, in the present invention, the “light” means an actinic ray or radiation.

Furthermore, in the description of the present invention, unless otherwise indicated, the “exposure” encompasses not only exposure to a mercury lamp, a far ultraviolet ray typified by excimer laser, an extreme ultraviolet ray, an X-ray, EUV light or the like but also lithography with a particle beam such as electron beam and ion beam.

The actinic ray-sensitive or radiation-sensitive resin composition of the present invention comprises (A) a resin having a group capable of decomposing by an action of an acid to produce a polar group, (C1) a compound containing a group capable of generating a first acidic functional group upon irradiation with an actinic ray or radiation and a group capable of generating a second acidic functional group different from the first acidic functional group upon irradiation with an actinic ray or radiation, and (C2) at least one compound containing two or more groups selected from the group consisting of a group capable of generating a structure represented by the following formula (a) upon irradiation with an actinic ray or radiation, a group capable of generating a structure represented by the following formula (b) upon irradiation with an actinic ray or radiation, a group capable of generating a structure represented by the following formula (c) upon irradiation with an actinic ray or radiation, and a group capable of generating a structure represented by the following formula (d) upon irradiation with an actinic ray or radiation:

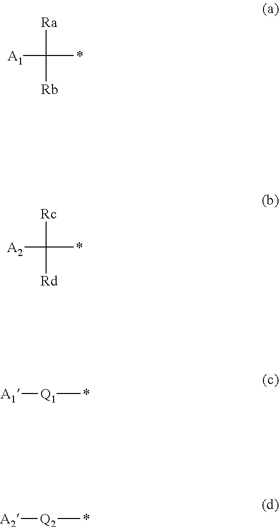

In formulae (a), (b), (c) and (d),

A₁, A₂, A₁′ and A₂′ represent the same acidic functional group,

each of Ra, Rb, Rc and Rd independently represents a hydrogen atom or a substituent,

each of Q₁ and Q₂ represents a cyclic group,

provided that the structure represented by formula (a) is different from the structure represented by formula (b) and the structure represented by formula (c) is different from the structure represented by formula (d), and

* represents a bond.

Thanks to this configuration, a pattern forming method ensuring that the roughness performance such as line width roughness, the local pattern dimension uniformity and the exposure latitude are excellent and reduction in the film thickness of the pattern part formed by development, so-called film loss, can be suppressed, a compound used therein, an actinic ray-sensitive or radiation-sensitive resin composition, a resist film, a manufacturing method of an electronic device, and an electronic device can be provided.

The reason therefor is not clearly known but is presumed as follows.

The compound (C1) or (C2) contained in the actinic ray-sensitive or radiation-sensitive resin composition of the present invention has two or more sites generating an acid upon exposure and when the compound (C1) or (C2) is decomposed by the exposure, the dissolution rate of the exposed area for the developer is significantly reduced. Also, since the compound (C1) or (C2) has two or more sites generating an acid as described above, an acid group is strongly hydrogen-bonded and the diffusibility of an acid itself is kept from excessively increasing. This is considered to contribute to the fact that the roughness performance such as line width roughness, the local pattern dimension uniformity and the exposure latitude are excellent and reduction in the film thickness of the pattern part formed by development, so-called film loss, can be suppressed.

The actinic ray-sensitive or radiation-sensitive resin composition according to the present invention is described below.

The actinic ray-sensitive or radiation-sensitive resin composition according to the present invention is preferably used for negative development (development where the solubility for developer is decreased upon exposure, as a result, the exposed area remains as a pattern and the unexposed area is removed) particularly in the case of forming a pattern having an ultrafine width in a resist film. That is, the actinic ray-sensitive or radiation-sensitive resin composition according to the present invention can be an actinic ray-sensitive or radiation-sensitive resin composition for organic solvent development, which is used for development using an organic solvent-containing developer. The term “for organic solvent development” as used herein means usage where the composition is subjected to at least a step of performing development by using an organic solvent-containing developer.

The actinic ray-sensitive or radiation-sensitive resin composition of the present invention is typically a resist composition and is preferably a negative or positive resist composition, more preferably a negative resist composition (that is, a resist composition for organic solvent development), because high effects can be obtained. Also, the composition according to the present invention is typically a chemical amplification resist composition.

### [1](A) Resin Having a Group Capable of Decomposing by an Action of an Acid to Produce a Polar Group

The resin (A) contained in the actinic ray-sensitive or radiation-sensitive resin composition of the present invention is, for example, a resin having a group capable of decomposing by an action of an acid to produce a polar group (hereinafter, sometimes referred to as “acid-decomposable group”), on either one or both of the main chain and the side chain of the resin (hereinafter, sometimes referred to as “acid-decomposable resin” or “resin (A)”).

Here, the resin (A) is a resin capable of increasing the polarity by an action of an acid to decrease the solubility for an organic solvent-containing developer. Also, the resin (A) is at the same time a resin capable of increasing the polarity by an action of an acid to increase the solubility for an alkali developer.

The acid-decomposable group preferably has a structure where a polar group is protected by a group capable of leaving by an action of an acid.

The polar group is not particularly limited as long as it is a group capable of being sparingly solubilized or insolubilized in an organic solvent-containing developer, but examples thereof include an acidic group (a group capable of dissociating in an aqueous 2.38 mass % tetramethylammonium hydroxide solution which has been conventionally used as the developer for a resist) such as phenolic hydroxyl group, carboxyl group, fluorinated alcohol group (preferably a hexafluoroisopropanol group), sulfonic acid group, sulfonamide group, sulfonylimide group, (alkylsulfonyl)(alkylcarbonyl)methylene group, (alkylsulfonyl)(alkylcarbonyl)imide group, bis(alkylcarbonyl)methylene group, bis(alkylcarbonyl)imide group, bis(alkylsulfonyl)methylene group, bis(alkylsulfonyl)imide group, tris(alkylcarbonyl)methylene group and tris(alkylsulfonyl)methylene group, and an alcoholic hydroxyl group.

The alcoholic hydroxyl group is a hydroxyl group bonded to a hydrocarbon group and indicates a hydroxyl group except for a hydroxyl group directly bonded on an aromatic ring (phenolic hydroxyl group), and the hydroxyl group excludes an aliphatic alcohol substituted with an electron-withdrawing group such as fluorine atom on the α-position (for example, a fluorinated alcohol group (e.g., hexafluoroisopropanol group)). The alcoholic hydroxyl group is preferably a hydroxyl group having a pKa of 12 to 20.

Preferred polar groups include a carboxyl group, a fluorinated alcohol group (preferably a hexafluoroisopropanol group), and a sulfonic acid group.

The group preferred as the acid-decomposable group is a group where a hydrogen atom of the group above is substituted for by a group capable of leaving by the action of an acid.

The group capable of leaving by the action of an acid includes, for example, —C(R₃₆)(R₃₇)(R₃₈), —C(R₃₆)(R₃₇)(OR₃₉), and —C(R₀₁)(R₀₂)(OR₃₉).

In the formulae, each of R₃₆ to R₃₉ independently represents an alkyl group, a cycloalkyl group, an aryl group, an aralkyl group or an alkenyl group. R₃₆ and R₃₇ may combine with each other to form a ring.

Each of R₀₁ and R₀₂ independently represents a hydrogen atom, an alkyl group, a cycloalkyl group, an aryl group, an aralkyl group or an alkenyl group.

The alkyl group of R₃₆ to R₃₉, R₀₁, and R₀₂ is preferably an alkyl group having a carbon number of 1 to 8, and examples thereof include a methyl group, an ethyl group, a propyl group, an n-butyl group, a sec-butyl group, a hexyl group, and an octyl group.

The cycloalkyl group of R₃₆ to R₃₉, R₀₁ and R₀₂ may be monocyclic or polycyclic and is preferably a cycloalkyl group having a carbon number of 3 to 20.

The aryl group of R₃₆ to R₃₉, R₀₁ and R₀₂ is preferably an aryl group having a carbon number of 6 to 10, and examples thereof include a phenyl group, a naphthyl group, and an anthryl group.

The aralkyl group of R₃₆ to R₃₉, R₀₁ and R₀₂ is preferably an aralkyl group having a carbon number of 7 to 12, and examples thereof include a benzyl group, a phenethyl group, and a naphthylmethyl group.

The alkenyl group of R₃₆ to R₃₉, R₀₁ and R₀₂ is preferably an alkenyl group having a carbon number of 2 to 8, and examples thereof include a vinyl group, an allyl group, a butenyl group and a cyclohexenyl group.

The ring formed by combining R₃₆ and R₃₇ is preferably a cycloalkyl group (monocyclic or polycyclic). The cycloalkyl group is preferably a monocyclic cycloalkyl group such as cyclopentyl group and cyclohexyl group, or a polycyclic cycloalkyl group such as norbornyl group, tetracyclodecanyl group, tetracyclododecanyl group and adamantyl group, more preferably a monocyclic cycloalkyl group having a carbon number of 5 to 6, still more preferably a monocyclic cycloalkyl group having a carbon number of 5.

The acid-decomposable group is preferably a cumyl ester group, an enol ester group, an acetal ester group, a tertiary alkyl ester group or the like, more preferably a tertiary alkyl ester group.

The resin (A) preferably contains a repeating unit having an acid-decomposable group.

Also, the resin (A) preferably contains, as the repeating unit having an acid-decomposable group, (AI) a repeating unit capable of decomposing by the action of an acid to produce a carboxyl group (hereinafter, sometimes referred to as “repeating unit (AI)”), more preferably a repeating unit represented by the following formula (aI). The repeating unit represented by formula (aI) generates a carboxyl group as a polar group by the action of an acid, and a high hydrogen bonding interaction is exhibited among a plurality of carboxyl groups, so that the glass transition temperature (Tg) of the resin (A) can be more enhanced. In turn, even when a film is deposited in the periphery of a resist pattern by CVD method (particularly, high-temperature CVD method), high rectangularity in the cross-sectional profile of the resist pattern is less likely to be impaired by heat during film growth, as a result, an increase in the process cost can be more suppressed.

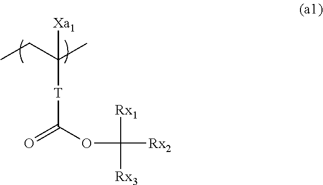

In formula (aI),

Xa₁ represents a hydrogen atom, an alkyl group, a cyano group or a halogen atom,

T represents a single bond or a divalent linking group,

each of Rx₁ to Rx₃ independently represents an alkyl group or a cycloalkyl group, and

two members out of Rx₁ to Rx₃ may combine to form a ring structure.

Examples of the divalent linking group of T include an alkylene group, a —COO-Rt- group, a —O-Rt- group, and a phenylene group. In the formulae, Rt represents an alkylene group or a cycloalkylene group.

T is preferably a single bond or a —COO-Rt- group. Rt is preferably an alkylene group having a carbon number of 1 to 5, more preferably a —CH₂— group, —(CH₂)₂— group or a —(CH₂)₃— group. T is more preferably a single bond.

The alkyl group of Xa₁ may have a substituent, and the substituent includes, for example, a hydroxyl group and a halogen atom (preferably fluorine atom).

The alkyl group of Xa₁ is preferably an alkyl group having a carbon number of 1 to 4, and examples thereof include a methyl group, an ethyl group, a propyl group, a hydroxymethyl group and a trifluoromethyl group, with a methyl group being preferred.

Xa₁ is preferably a hydrogen atom or a methyl group.

The alkyl group of Rx₁, Rx₂ and Rx₃ may be linear or branched and is preferably an alkyl group having a carbon number of 1 to 4, such as methyl group, ethyl group, n-propyl group, isopropyl group, n-butyl group, isobutyl group and tert-butyl group.

The cycloalkyl group of Rx₁, Rx₂ and Rx₃ is preferably a monocyclic cycloalkyl group such as cyclopentyl group and cyclohexyl group, or a polycyclic cycloalkyl group such as norbornyl group, tetracyclodecanyl group, tetracyclododecanyl group and adamantyl group.

The ring structure formed by combining two members out of Rx₁, Rx₂ and Rx₃ is preferably a monocyclic cycloalkane ring such as cyclopentyl ring and cyclohexyl ring, or a polycyclic cycloalkane ring such as norbornane ring, tetracyclodecane ring, tetracyclododecane ring and adamantane ring, more preferably a monocyclic cycloalkane ring having a carbon number of 5 or 6.

Each of Rx₁, Rx₂ and Rx₃ is independently preferably an alkyl group, more preferably a linear or branched alkyl group having a carbon number of 1 to 4.

Each of the groups above may have a substituent, and the substituent includes, for example, an alkyl group (having a carbon number of 1 to 4), a cycloalkyl group (having a carbon number of 3 to 8), a halogen atom, an alkoxy group (having a carbon number of 1 to 4), a carboxyl group, and an alkoxycarbonyl group (having a carbon number of 2 to 6). The carbon number is preferably 8 or less. Above all, from the standpoint of more enhancing the dissolution contrast for an organic solvent-containing developer between before and after acid decomposition, the substituent is preferably a group free from a heteroatom such as oxygen atom, nitrogen atom and sulfur atom (for example, preferably not an alkyl group substituted with a hydroxyl group), more preferably a group composed of only a hydrogen atom and a carbon atom, still more preferably a linear or branched alkyl group or a cycloalkyl group.

Specific examples of the repeating unit represented by formula (aI) are illustrated below, but the present invention is not limited thereto.

In specific examples, Rx represents a hydrogen atom, CH₃, CF₃ or CH₂OH. Each of Rxa and Rxb represents an alkyl group having a carbon number 1 to 4. Xaᵢ represents a hydrogen atom, CH₃, CF₃ or CH₂OH. Z represents a substituent and when a plurality of Z are present, each Z may be the same as or different from every other Z. p represents 0 or a positive integer. Specific examples and preferred examples of Z are the same as specific examples and preferred examples of the substituent which may be substituted on each of the groups such as Rx₁ to Rx₃.

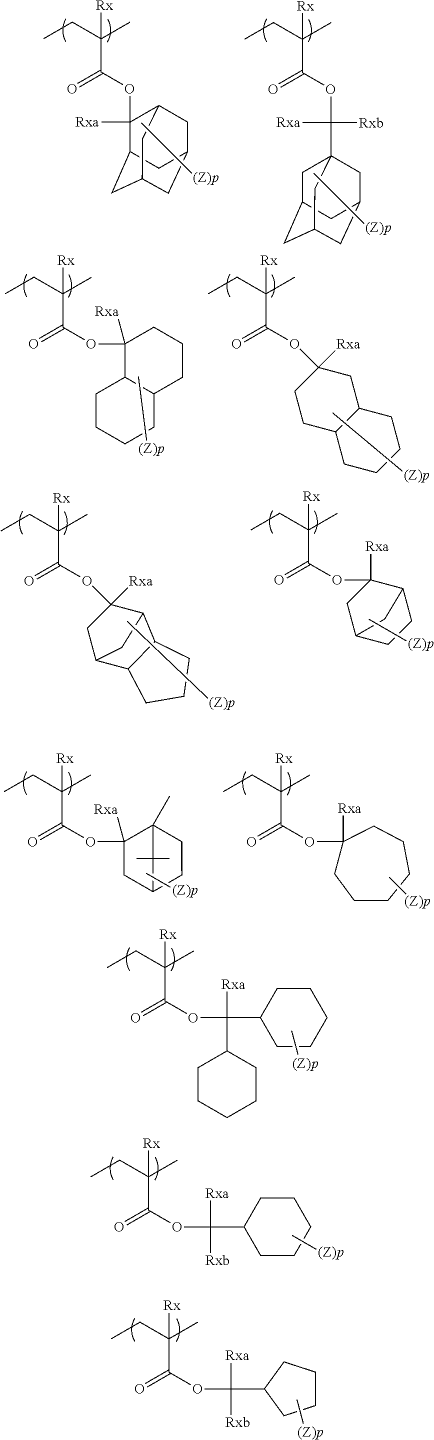

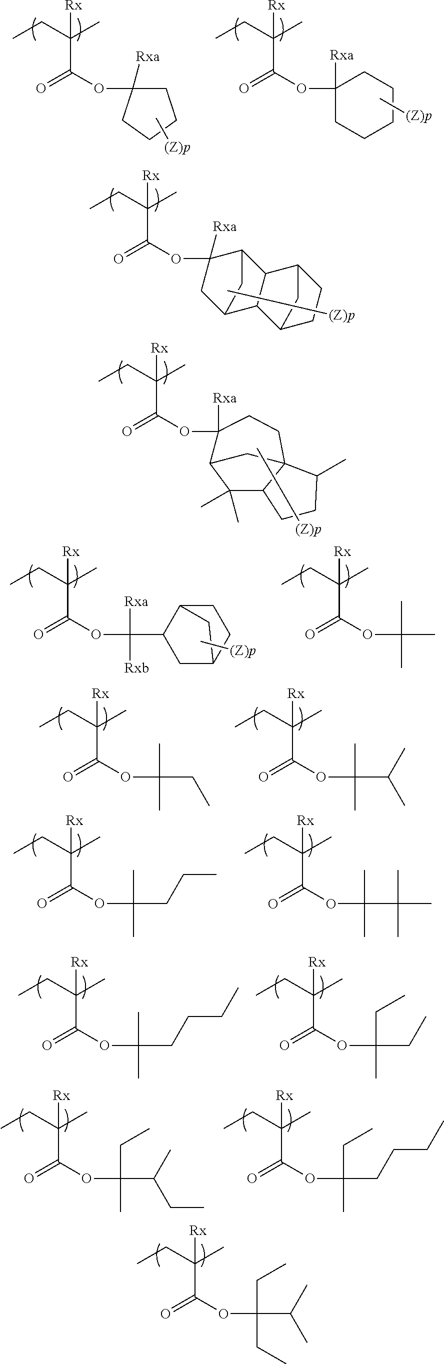

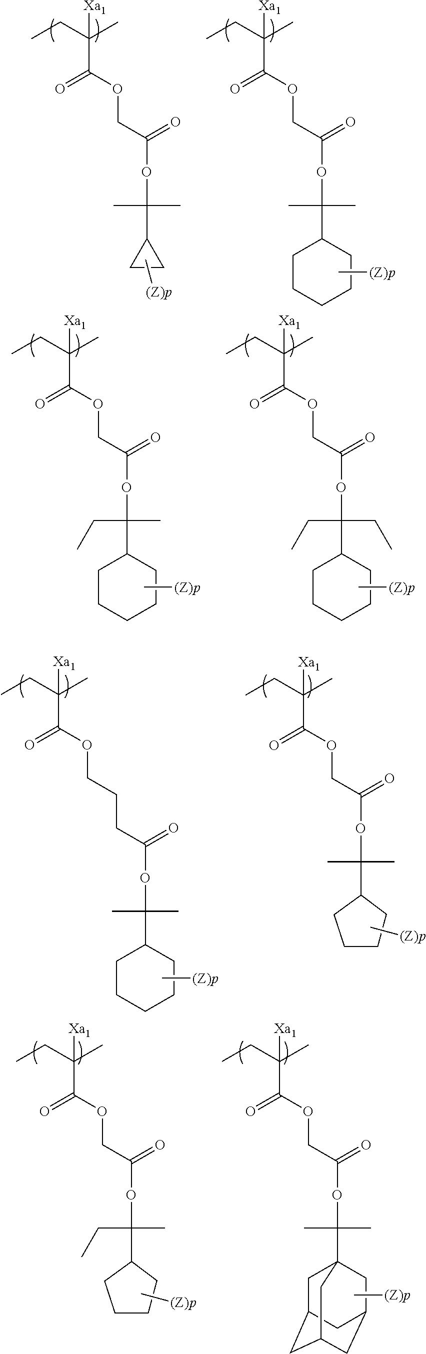

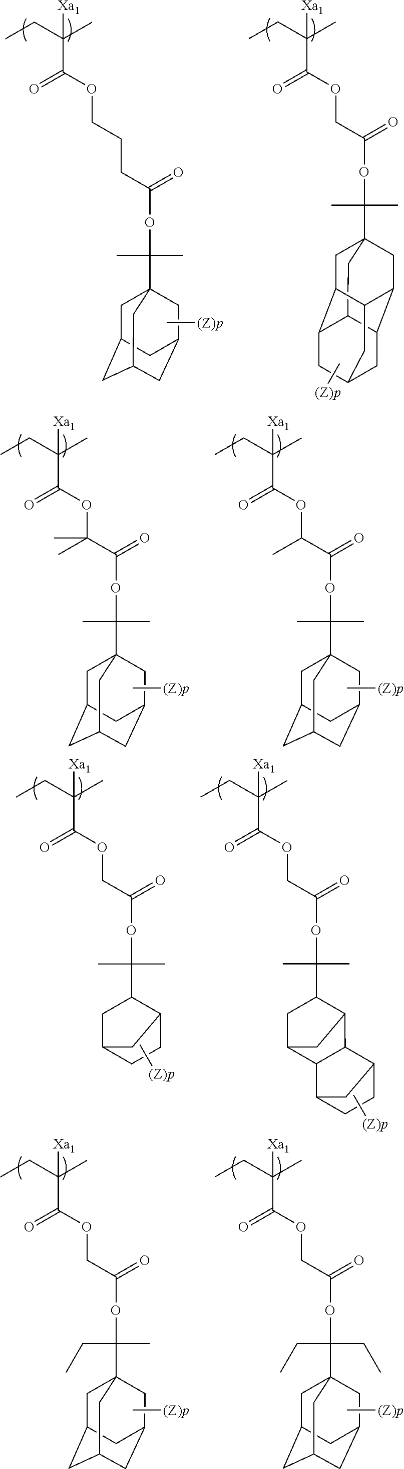

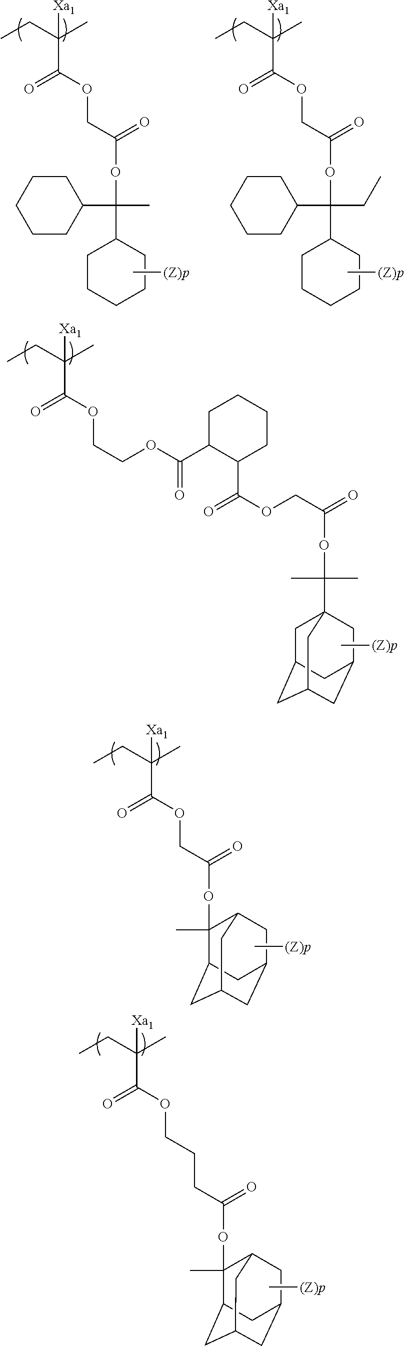

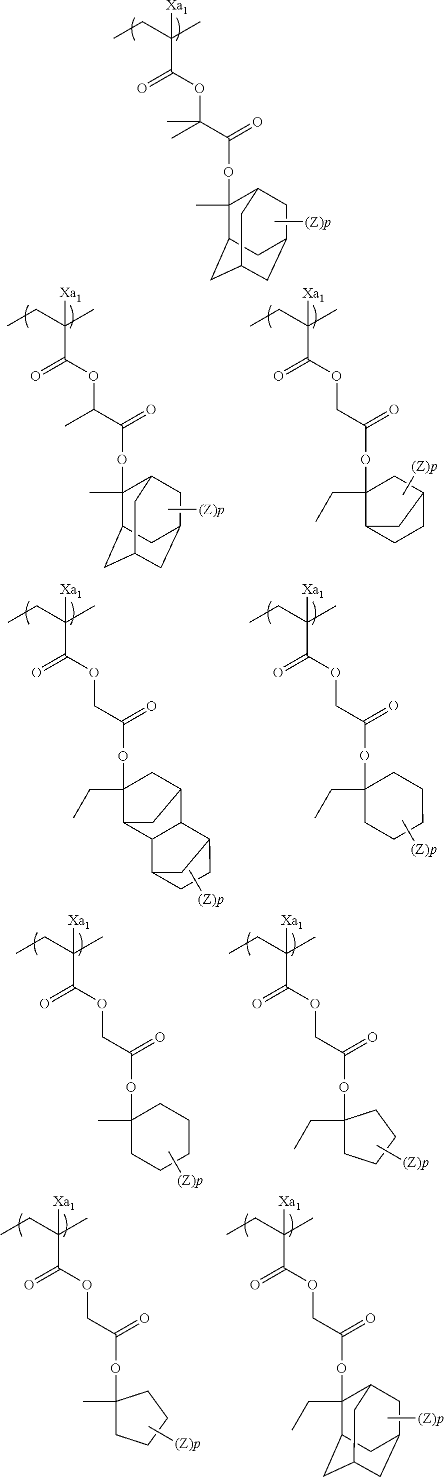

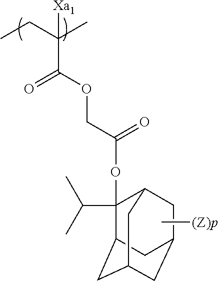

In specific examples below, Xa represents a hydrogen atom, an alkyl group, a cyano group or a halogen atom.

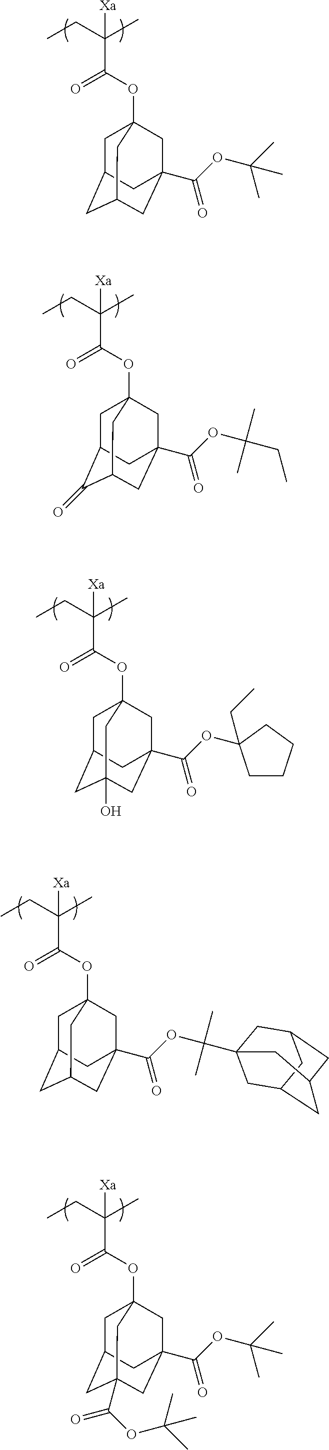

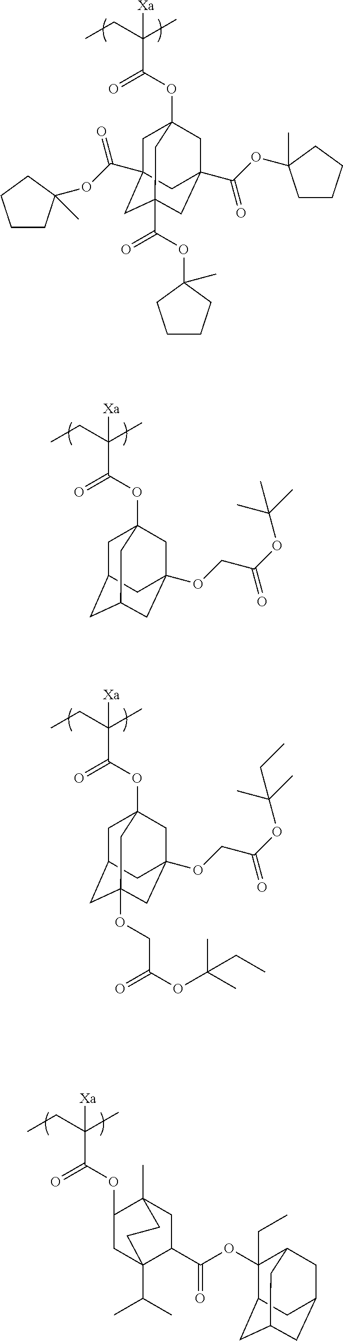

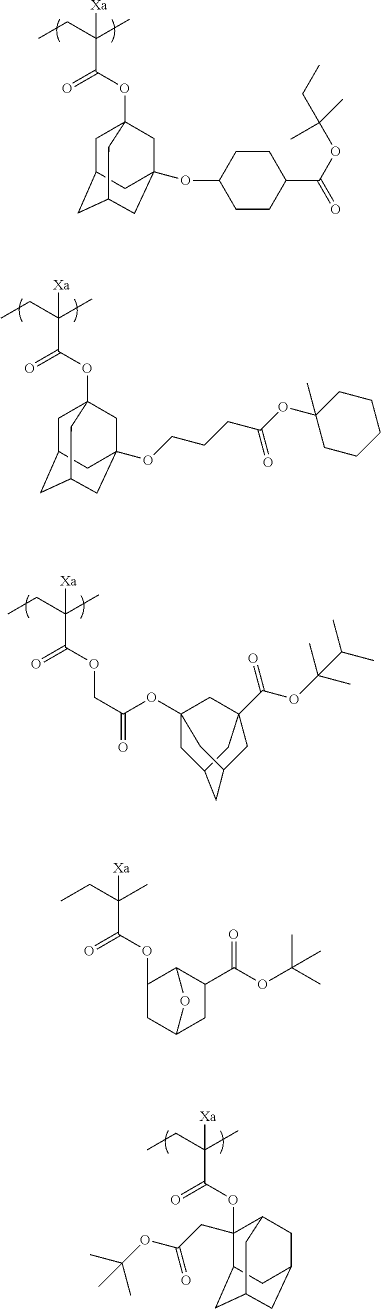

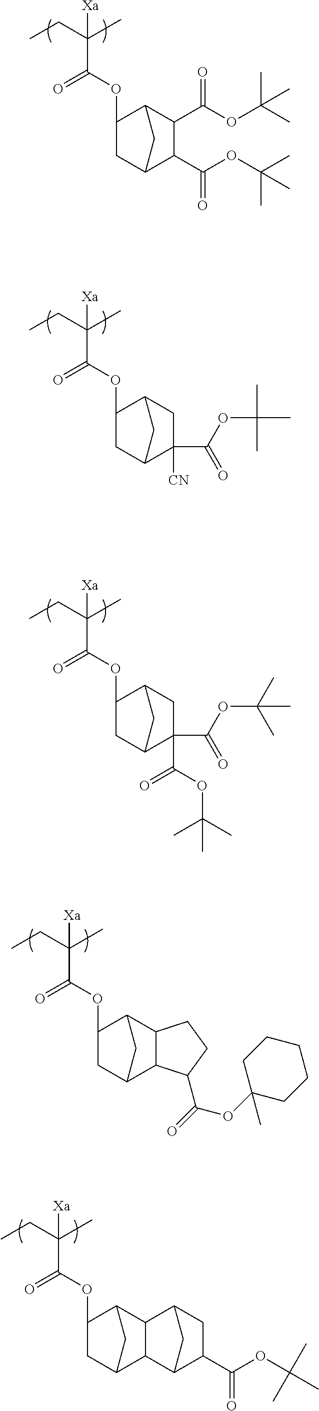

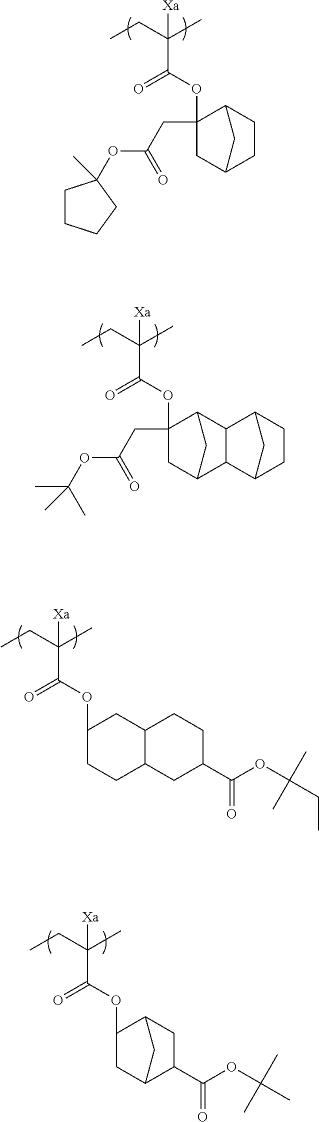

Also, the resin (A) may contain, as the repeating unit having an acid-decomposable group, a repeating unit illustrated below, which is a repeating unit capable of decomposing by the action of an acid to produce an alcoholic hydroxyl group.

In specific examples below, Xa₁ represents a hydrogen atom, CH₃, CF₃ or CH₂OH.

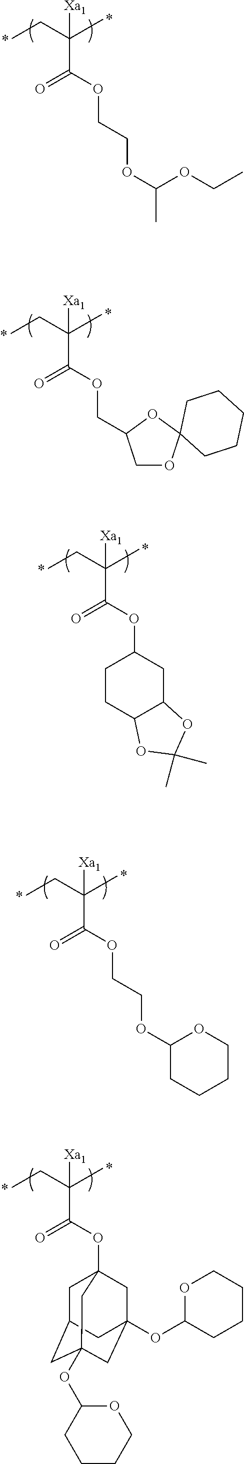

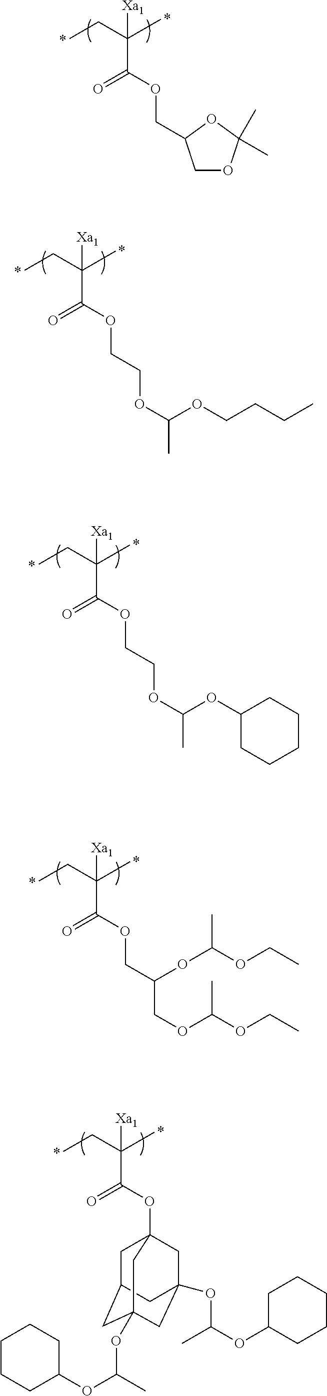

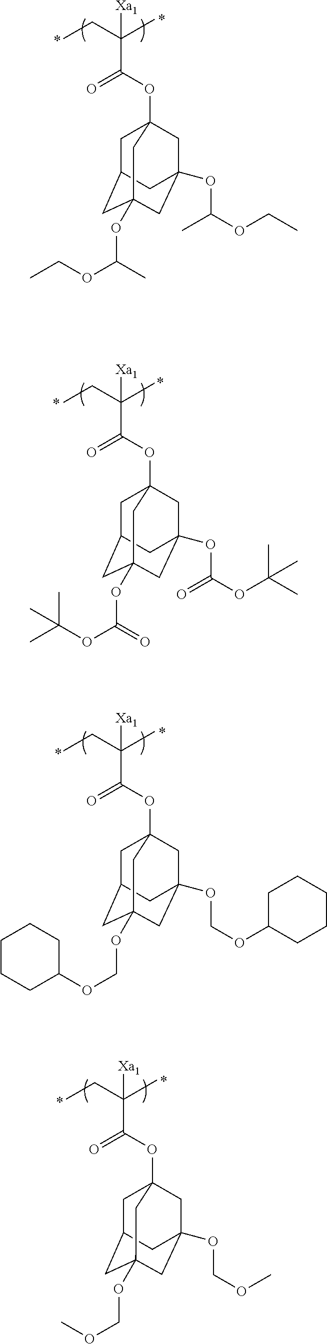

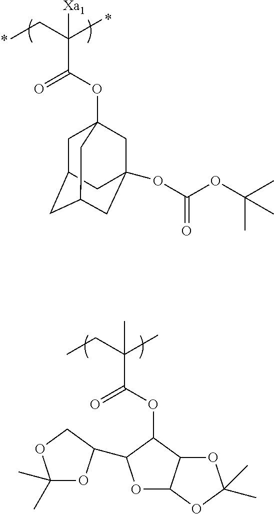

As for the repeating unit having an acid-decomposable group, one kind may be used, or two or more kinds may be used in combination.

In the case of containing two or more kinds of repeating units in combination, the possible combination includes, for example, the following combinations and a combination of a repeating unit represented by formula (aI) and a repeating unit capable of decomposing by the action of an acid to produce an alcoholic hydroxyl group.

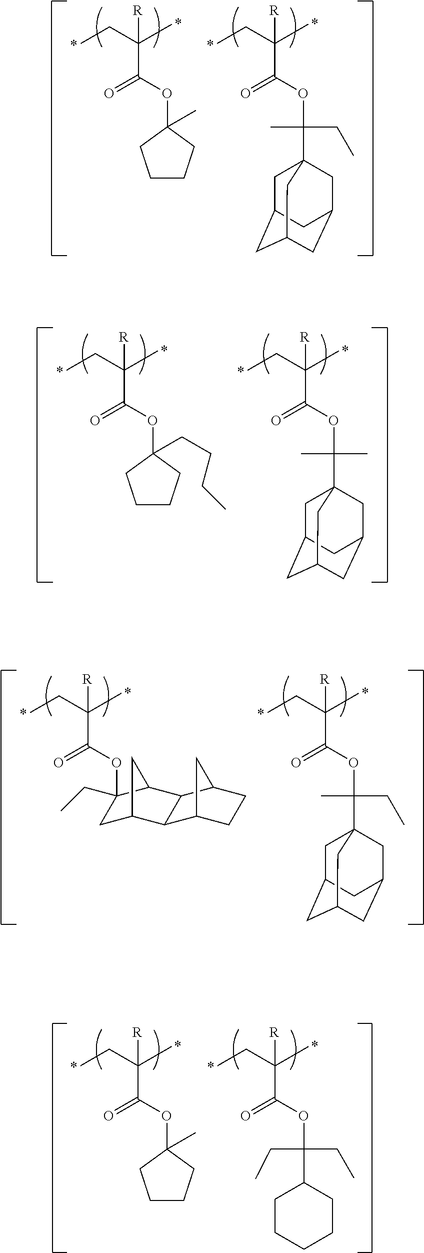

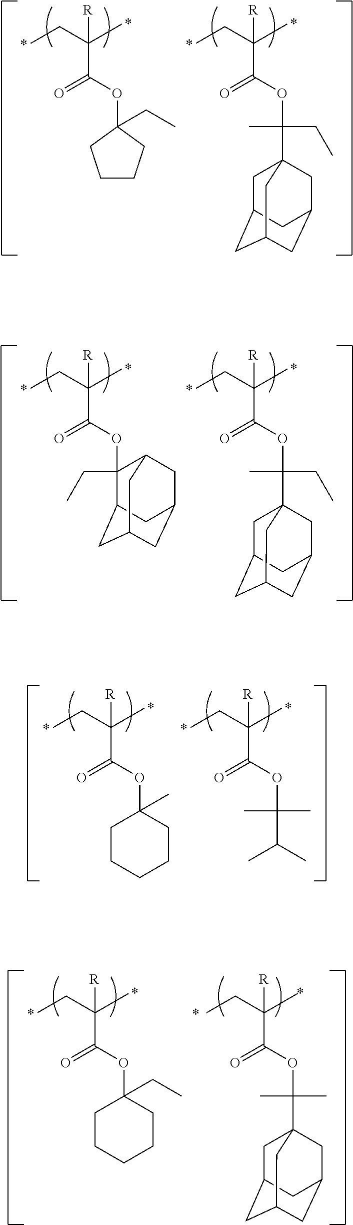

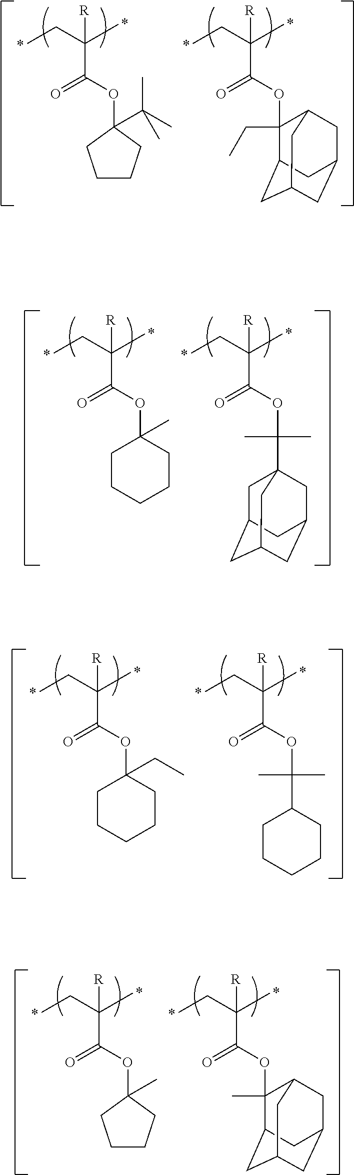

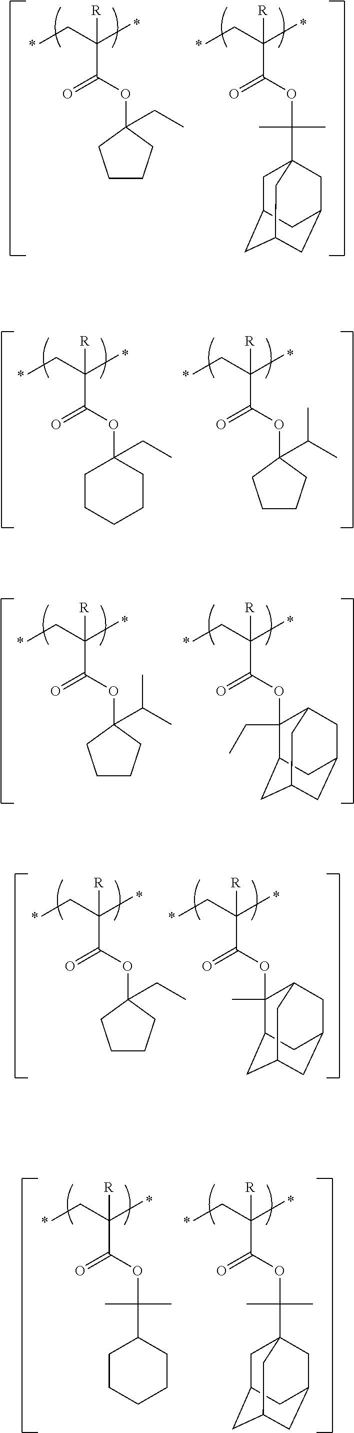

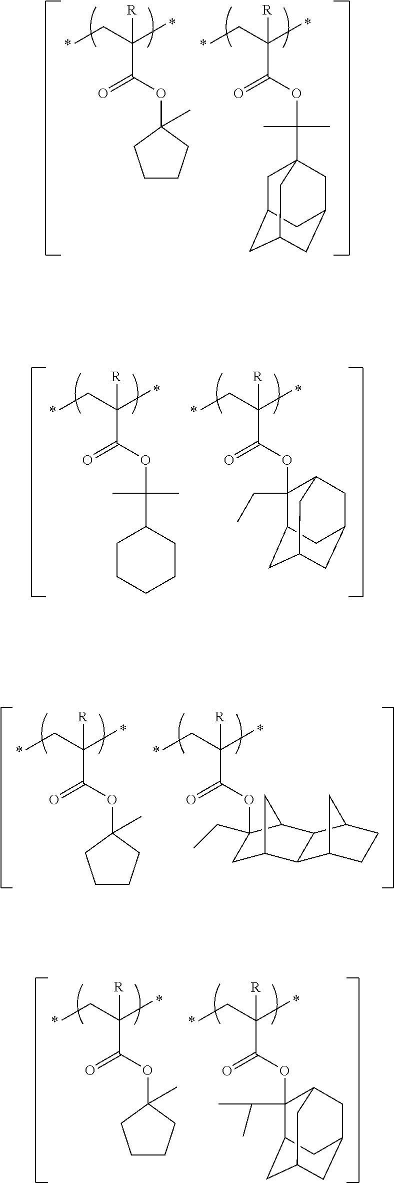

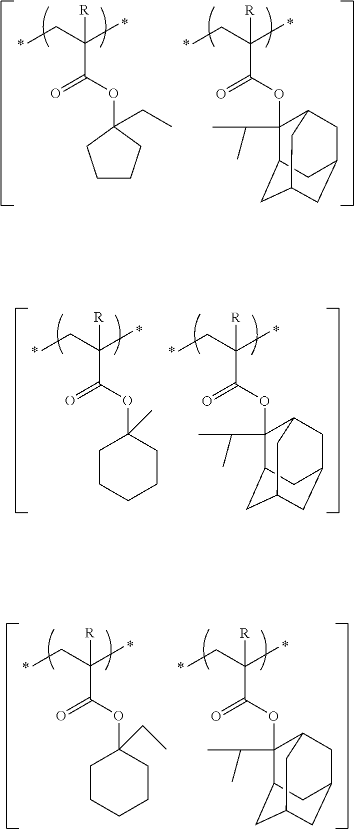

The content of the acid-decomposable group-containing repeating unit contained in the resin (A) (in the case where a plurality of acid-decomposable group-containing repeating units are present, the total thereof) is preferably 15 mol % or more, more preferably 20 mol % or more, still more preferably 25 mol % or more, yet still more preferably 40 mol % or more, based on all repeating units in the resin (A). Above all, it is preferred that the resin (A) contains the repeating unit (AI) and the content of the repeating unit (AI) is 50 mol % or more based on all repeating units in the reins (A).

When the content of the acid-decomposable group-containing repeating unit is 50 mol % or more based on all repeating units in the resin (A), the glass transition temperature (Tg) of the resin (A) can be made high without fail and in turn, the effect capable of suppressing an increase in the production cost can be obtained more reliably.

Also, the content of the repeating unit having an acid-decomposable group is preferably 80 mol % or less, more preferably 70 mol % or less, still more preferably 65 mol % or less, based on all repeating units in the resin (A).

The resin (A) may contain a repeating unit having a lactone structure or a sultone structure.

As the lactone structure or sultone structure, any structure may be used as long as it has a lactone structure or a sultone structure, but the structure is preferably a 5- to 7-membered ring lactone structure or a 5- to 7-membered ring sultone structure, more preferably a 5- to 7-membered ring lactone structure to which another ring structure is fused in the form of forming a bicyclo or spiro structure, or a 5- to 7-membered ring sultone structure to which another ring structure is fused in the form of forming a bicyclo or spiro structure. The resin more preferably contains a repeating unit having a lactone structure represented by any one of the following formulae (LC1-1) to (LC1-21) or a sultone structure represented by any one of the following formulae (SL1-1) to (SL1-3). The lactone structure or sultone structure may be bonded directly to the main chain. Preferred lactone structures are (LC1-1), (LC1-4), (LC1-5), (LC1-6), (LC1-13), (LC1-14) and (LC1-17), with the lactone structure of (LC1-4) being more preferred. By using such a specific lactone structure, LER and development defect are improved.

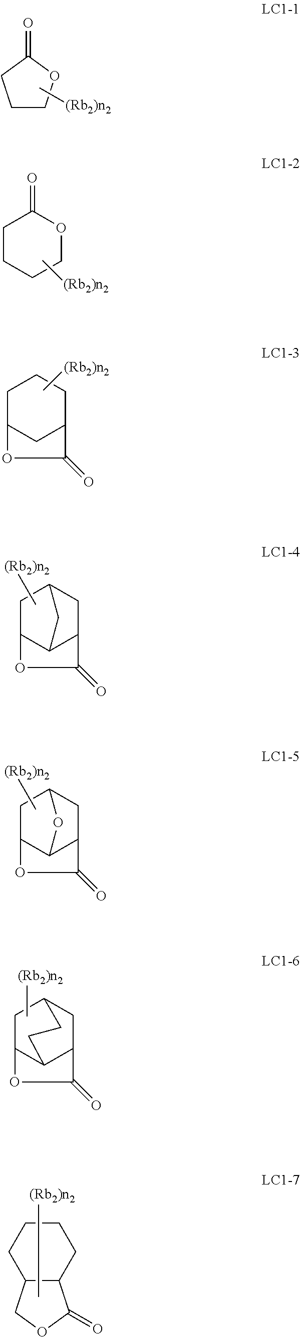

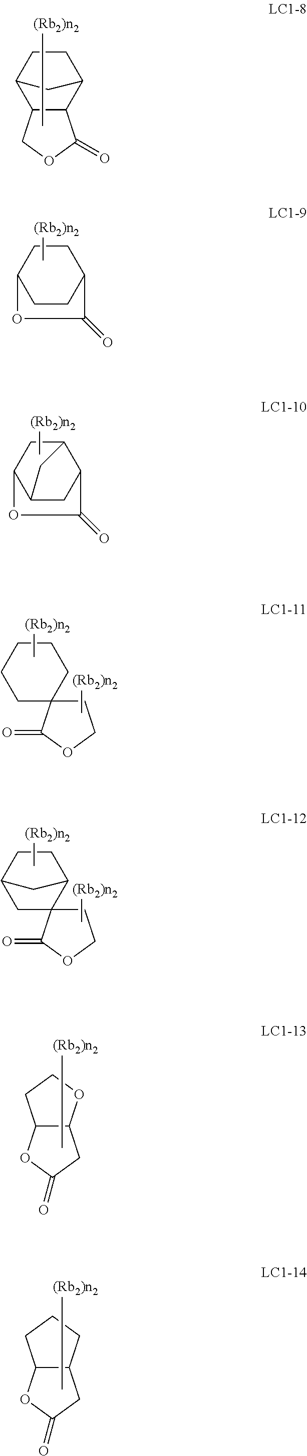

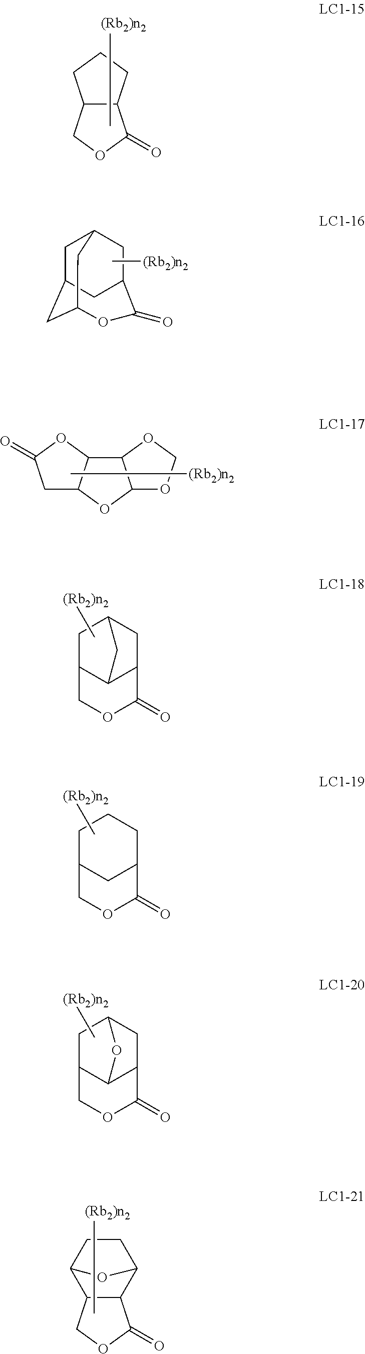

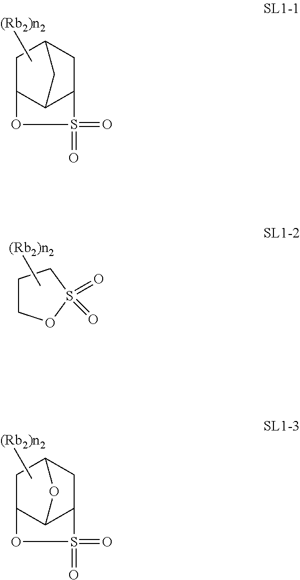

The lactone structure moiety or sultone structure moiety may or may not have a substituent (Rb₂). Preferred examples of the substituent (Rb₂) include an alkyl group having a carbon number of 1 to 8, a cycloalkyl group having a carbon number of 4 to 7, an alkoxy group having a carbon number of 1 to 8, an alkoxycarbonyl group having a carbon number of 2 to 8, a carboxyl group, a halogen atom, a hydroxyl group, a cyano group, and an acid-decomposable group. Among these, an alkyl group having a carbon number of 1 to 4, a cyano group and an acid-decomposable group are more preferred. n₂ represents an integer of 0 to 4. When n₂ is an integer of 2 or more, each substituent (Rb₂) may be the same as or different from every other substituent (Rb₂), and also, the plurality of substituents (Rb₂) may combine with each other to form a ring.

The repeating unit having a lactone or sultone structure usually has an optical isomer, and any optical isomer may be used. One optical isomer may be used alone, or a plurality of optical isomers may be mixed and used. In the case of mainly using one optical isomer, the optical purity (ee) thereof is preferably 90% or more, more preferably 95% or more.

The repeating unit having a lactone or sultone structure is preferably a repeating unit represented by the following formula (III):

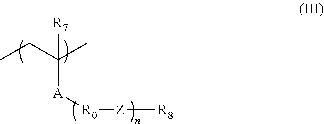

In formula (III),

A represents an ester bond (a group represented by —COO—) or an amide bond (a group represented by —CONH—),

R₀ represents, when a plurality of R₀ are present, each independently represents, an alkylene group, a cycloalkylene group or a combination thereof,

Z represents, when a plurality of Z are present, each independently represents, a single bond, an ether bond, an ester bond, an amide bond, a urethane bond

(a group represented by

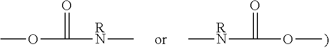

or a urea bond
(a group represented by

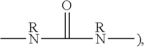

wherein each R independently represents a hydrogen atom, an alkyl group, a cycloalkyl group or an aryl group,

R₈ represents a monovalent organic group having a lactone structure or a sultone structure,

n is the repetition number of the structure represented by —R₀—Z— and represents an integer of 0 to 5, preferably 0 or 1, more preferably 0, and when n is 0, —R₀—Z— is not present and a single bond is formed, and

R₇ represents a hydrogen atom, a halogen atom or an alkyl group.

The alkylene group and cycloalkylene group of R₀ may have a substituent.

Z is preferably an ether bond or an ester bond, more preferably an ester bond.

The alkyl group of R₇ is preferably an alkyl group having a carbon number of 1 to 4, more preferably a methyl group or an ethyl group, still more preferably a methyl group.

The alkyl group in the alkylene group and cycloalkylene group of R₀ and in R₇ may be substituted, and examples of the substituent include a halogen atom such as fluorine atom, chlorine atom and bromine atom, a mercapto group, a hydroxyl group, an alkoxy group such as methoxy group, ethoxy group, isopropoxy group, tert-butoxy group and benzyloxy group, and an acyloxy group such as acetyloxy group and propionyloxy group.

R₇ is preferably a hydrogen atom, a methyl group, a trifluoromethyl group or a hydroxymethyl group.

The chain alkylene group in R₀ is preferably a chain alkylene group having a carbon number of 1 to 10, more preferably having a carbon number of 1 to 5, and examples thereof include a methylene group, an ethylene group and a propylene group. The cycloalkylene group is preferably a cycloalkylene group having a carbon number of 3 to 20, and examples thereof include a cyclohexylene group, a cyclopentylene group, a norbornylene group and an adamantylene group. For bringing out the effects of the present invention, a chain alkylene group is more preferred, and a methylene group is still more preferred.

The monovalent organic group having a lactone or sultone structure represented by R₈ is not limited as long as it has a lactone or sultone structure. Specific examples thereof include those having a lactone or sultone structure represented by any one of formulae (LC1-1) to (LC1-21) and (SL1-1) to (SL1-3), and among these, the structure represented by (LC1-4) is preferred. In (LC1-1) to (LC1-21), n₂ is preferably 2 or less.

R₈ is preferably a monovalent organic group having an unsubstituted lactone or sultone structure, or a monovalent organic group having a lactone or sultone structure containing a methyl group, a cyano group or an alkoxycarbonyl group as a substituent, more preferably a monovalent organic group having a lactone structure containing a cyano group as a substituent (cyanolactone).

Specific examples of the repeating unit containing a group having a lactone or sultone structure are illustrated below, but the present invention is not limited thereto.

(In the formulae, Rx represents H, CH₃, CH₂OH or CF₃.)

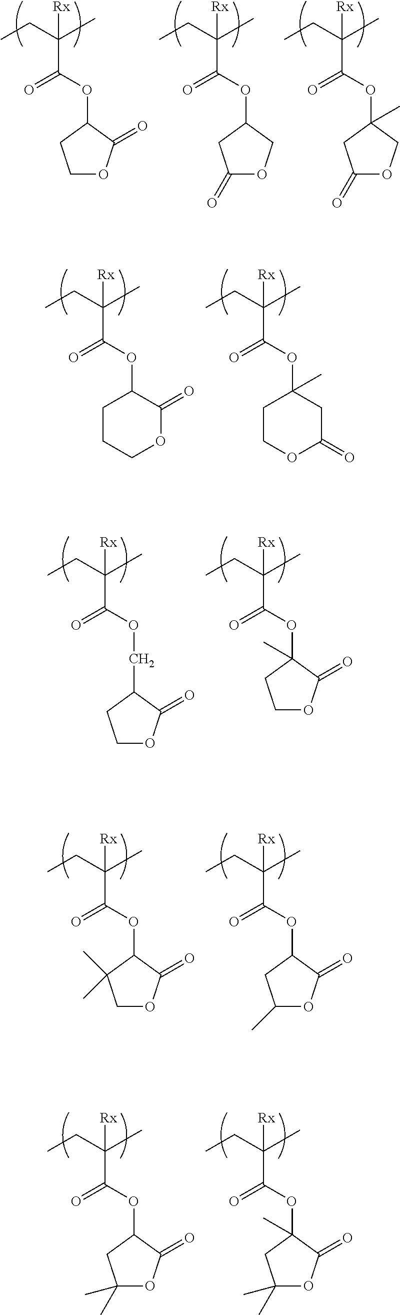

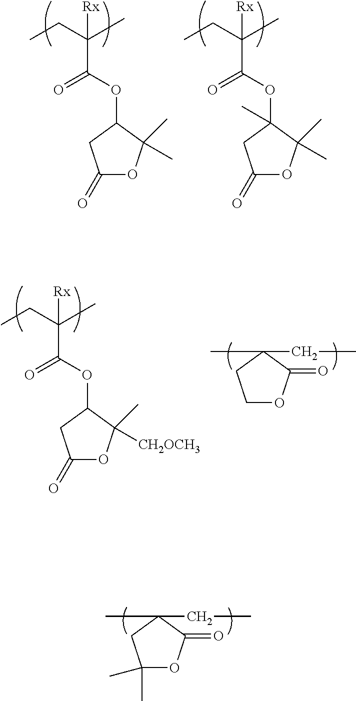

(In the formulae, Rx represents H, CH₃, CH₂OH or CF₃.)

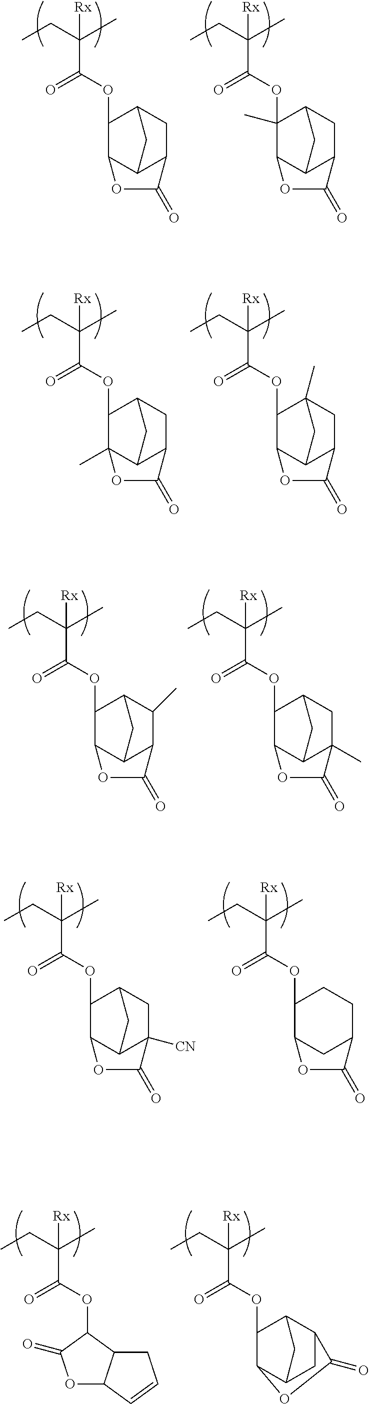

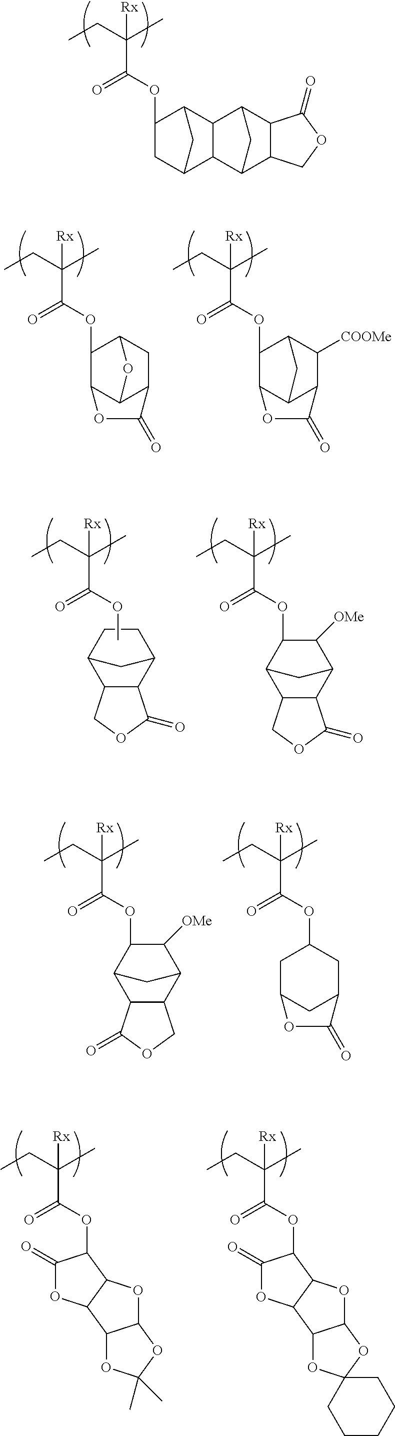

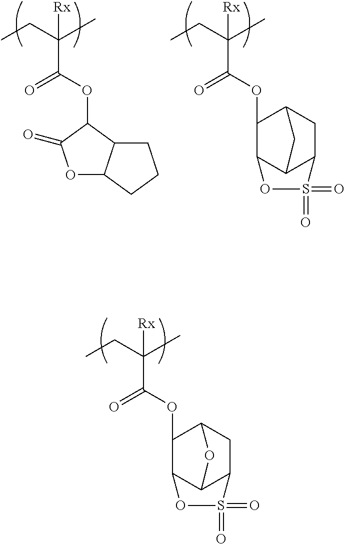

(In the formulae, Rx represents H, CH₃, CH₂OH or CF₃.)

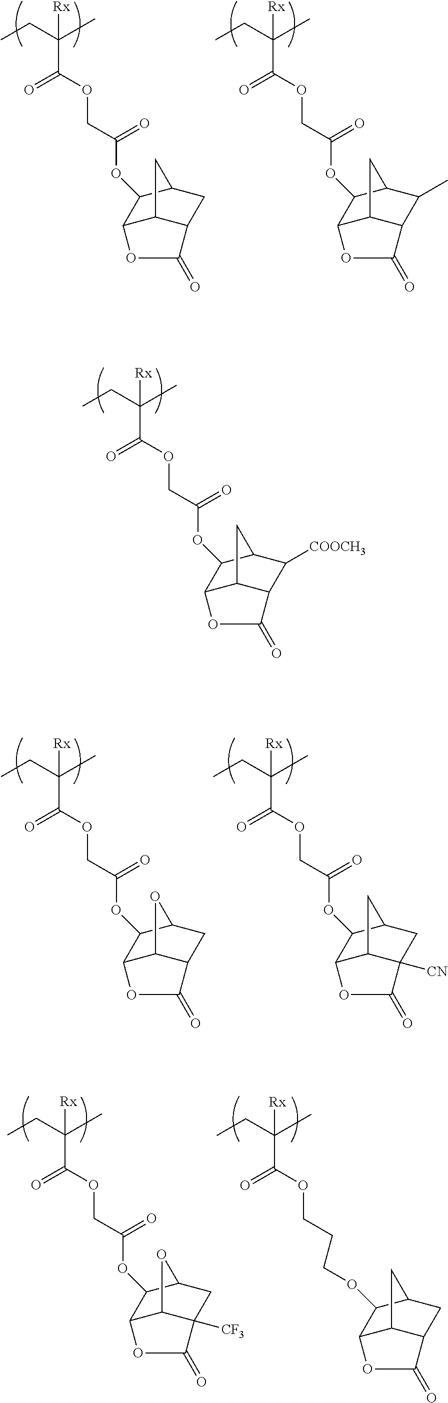

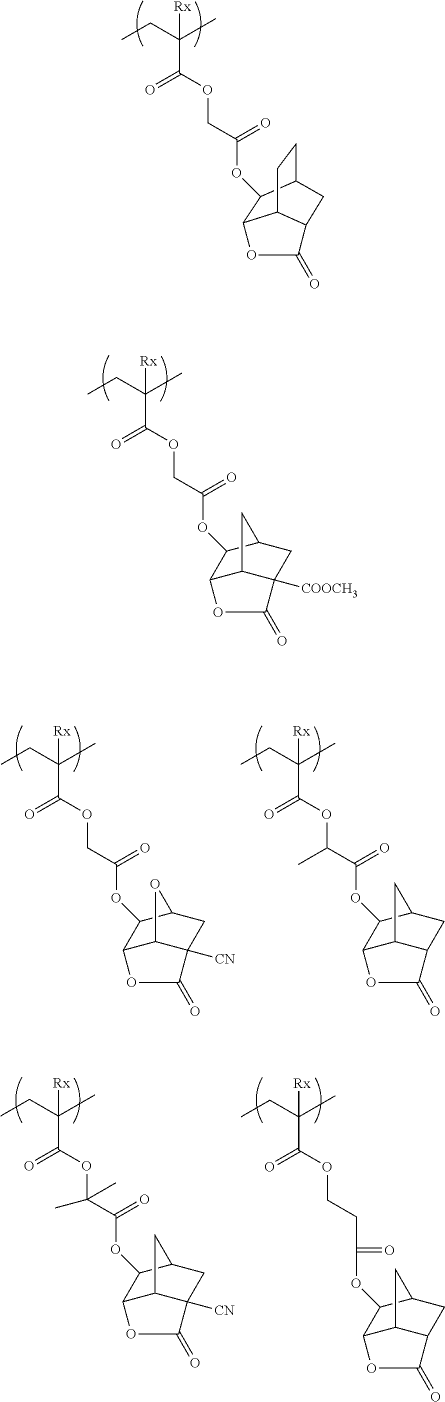

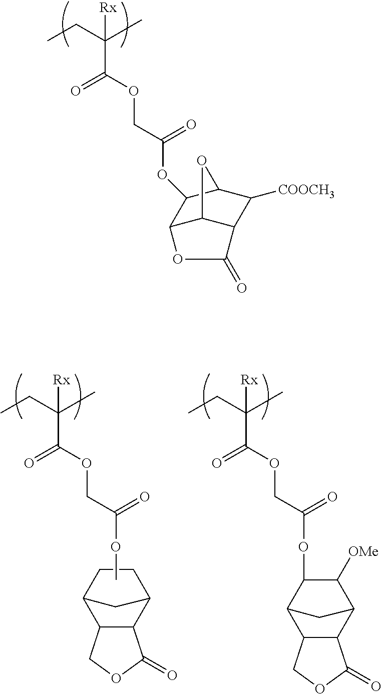

In order to increase the effects of the present invention, two or more kinds of repeating units having a lactone or sultone structure may be used in combination.

In the case where the resin (A) contains a repeating unit having a lactone or sultone structure, the content of the repeating unit having a lactone or sultone structure is preferably from 5 to 60 mol %, more preferably from 5 to 55 mol %, still more preferably from 10 to 50 mol %, based on all repeating units in the resin (A).

Also, the resin (A) may contain a repeating unit having a cyclic carbonic acid ester structure.

The repeating unit having a cyclic carbonic acid ester structure is preferably a repeating unit represented by the following formula (A-1):

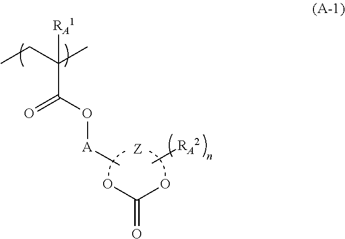

In formula (A-1),

R_{A}¹ represents a hydrogen atom or an alkyl group,

R_{A}² represents, when n is 2 or more, each independently represents, a substituent,

A represents a single bond or a divalent linking group,

Z represents an atomic group necessary for forming a monocyclic or polycyclic structure together with the group represented by —O—C(═O)—O— in the formula, and

n represents an integer of 0 or more.

Formula (A-1) is described in detail below.

The alkyl group represented by R_{A}¹ may have a substituent such as fluorine atom. R_{A}¹ preferably represents a hydrogen atom, a methyl group or a trifluoromethyl group, more preferably represents a methyl group.

The substituent represented by R_{A}² is, for example, an alkyl group, a cycloalkyl group, a hydroxyl group, an alkoxy group, an amino group or an alkoxycarbonyl group and is preferably an alkyl group having a carbon number of 1 to 5, and examples thereof include a linear alkyl group having a carbon number of 1 to 5 and a branched alkyl group having a carbon number of 3 to 5. The alkyl may have a substituent such as hydroxyl group.

n represents the number of substituents and is an integer of 0 or more. n is preferably from 0 to 4, more preferably 0.

The divalent linking group represented by A includes, for example, an alkylene group, a cycloalkylene group, an ester bond, an amide bond, an ether bond, a urethane bond, a urea bond, and a combination thereof. The alkylene group is preferably an alkylene group having a carbon number of 1 to 10, more preferably an alkylene group having a carbon number of 1 to 5.

In one embodiment of the present invention, A is preferably a single bond or an alkylene group.

The monocyclic ring containing —O—C(═O)—O— represented by Z includes, for example, a 5- to 7-membered ring where in the cyclic carbonic acid ester represented by the following formula (a), n_{A} is from 2 to 4, and is preferably a 5- or 6-membered ring (n_{A} is 2 or 3), more preferably a 5-membered ring (n_{A} is 2).

The polycyclic ring containing —O—C(═O)—O— represented by Z includes, for example, a structure where the cyclic carbonic acid ester represented by the following formula (a) forms a condensed ring together with one other ring structure or two or more other ring structures, and a structure where a spiro ring is formed. The “other ring structure” capable of forming a condensed ring or a spiro ring may be an alicyclic hydrocarbon group or an aromatic hydrocarbon group or may be a heterocyclic ring.

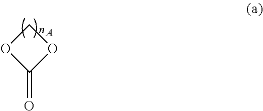

In the resin (A), one of repeating units represented by formula (A-1) may be contained alone, or two or more thereof may be contained.

In the resin (A), the content percentage of the repeating unit having a cyclic carbonic acid ester structure (preferably the repeating unit represented by formula (A-1)) is preferably from 3 to 80 mol %, more preferably from 3 to 60 mol %, still more preferably from 3 to 30 mol %, and most preferably from 10 to 15 mol %, based on all repeating units constituting the resin (A). With such a content percentage, the developability, low defect rate, low LWR, low PEB temperature dependency, profile and the like of the resist can be improved.

Specific examples of the repeating unit represented by formula (A-1) are illustrated below, but the present invention is not limited thereto.

In specific examples, R_{A}¹ has the same meaning as R_{A}¹ in formula (A-1).

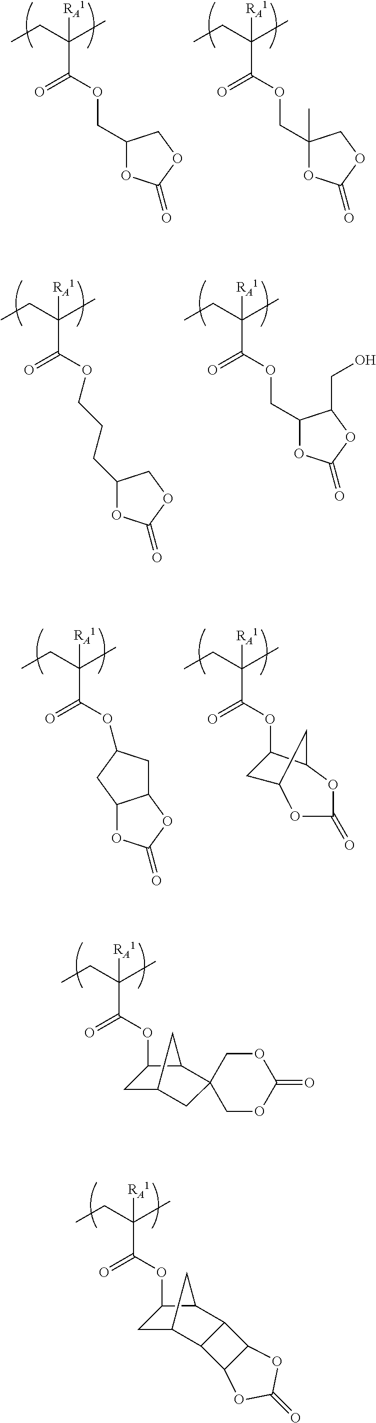

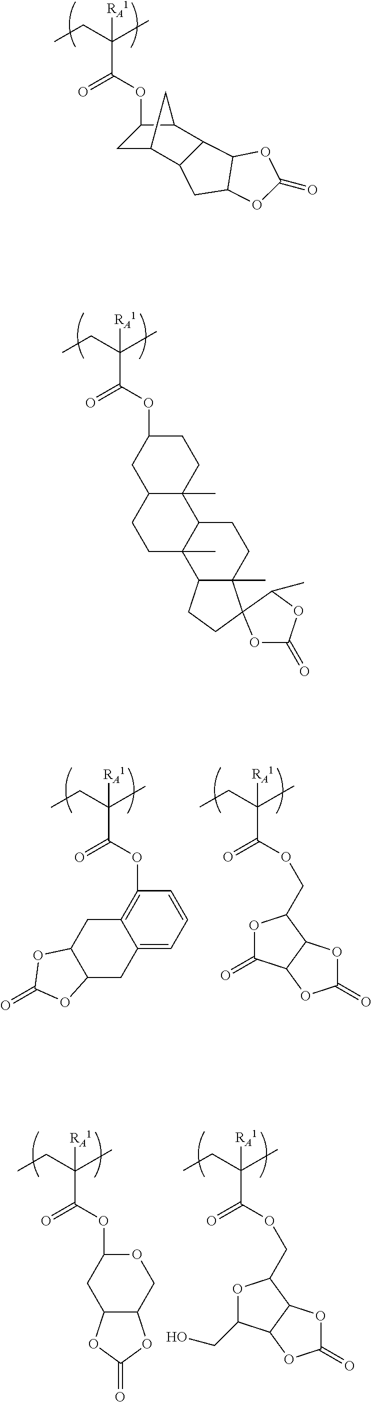

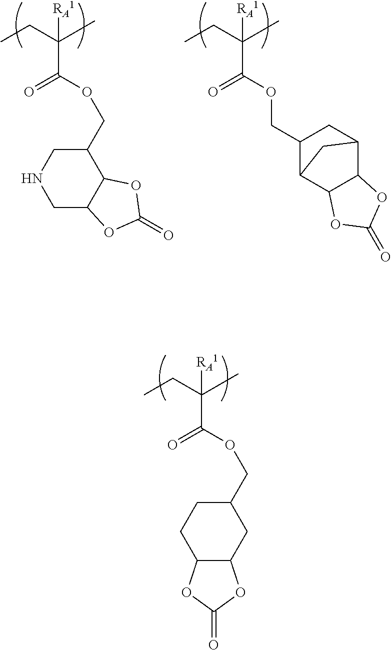

The resin (A) may contain a repeating unit having a hydroxyl group, a cyano group or a carbonyl group. Thanks to this repeating unit, the adherence to substrate and affinity for developer are enhanced. The repeating unit having a hydroxyl group, a cyano group or a carbonyl group is preferably a repeating unit having an alicyclic hydrocarbon structure substituted with a hydroxyl group, a cyano group or a carbonyl group and preferably has no acid-decomposable group.

Also, the repeating unit having an alicyclic hydrocarbon structure substituted with a hydroxyl group, a cyano group or a carbonyl group is preferably different from the repeating unit having an acid-decomposable group (that is, preferably a repeating unit stable to acid).

The alicyclic hydrocarbon structure in the alicyclic hydrocarbon structure substituted with a hydroxyl group, a cyano group or a carbonyl group is preferably an adamantyl group, a diadamantyl group or a norbornane group.

The repeating is more preferably a repeating unit represented by any one of the following formulae (AIIa) to (AIIc):

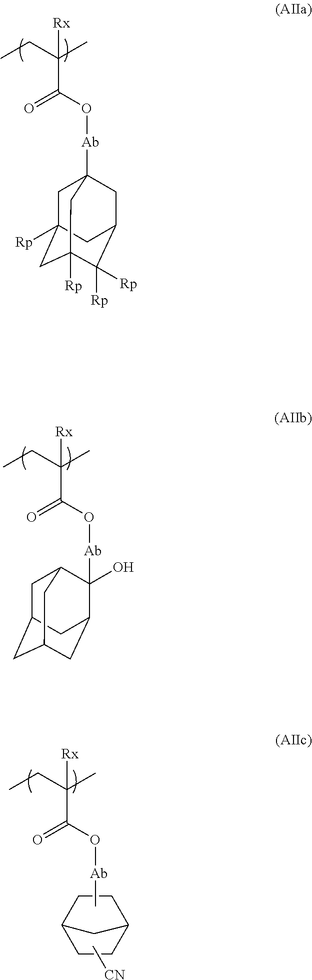

In the formulae, Rx represents a hydrogen atom, a methyl group, a hydroxymethyl group or a trifluoromethyl group.

Ab represents a single bond or a divalent linking group.

The divalent linking group represented by Ab includes, for example, an alkylene group, a cycloalkylene group, an ester bond, an amide bond, an ether bond, a urethane bond, a urea bond, and a combination thereof. The alkylene group is preferably an alkylene group having a carbon number of 1 to 10, more preferably an alkylene group having a carbon number of 1 to 5, and examples thereof include a methylene group, an ethylene group, and a propylene group.

In one embodiment of the present invention, Ab is preferably a single bond or an alkylene group.

Rp represents a hydrogen atom, a hydroxyl group or a hydroxyalkyl group. Each Rp may be the same as or different from every other Rp, but out of a plurality of Rp, at least one represents a hydroxyl group or a hydroxyalkyl group.

The resin (A) may or may not contain a repeating unit having a hydroxyl group, a cyano group or a carbonyl group, but in the case where the resin (A) contains a repeating unit having a hydroxyl group, a cyano group or a carbonyl group, the content of the repeating unit having a hydroxyl group, a cyano group or a carbonyl group is preferably from 1 to 40 mol %, more preferably from 3 to 30 mol %, still more preferably from 5 to 25 mol %, based on all repeating units in the resin (A).

Specific examples of the repeating unit having a hydroxyl group, a cyano group or a carbonyl group are illustrated below, but the present invention is not limited thereto.

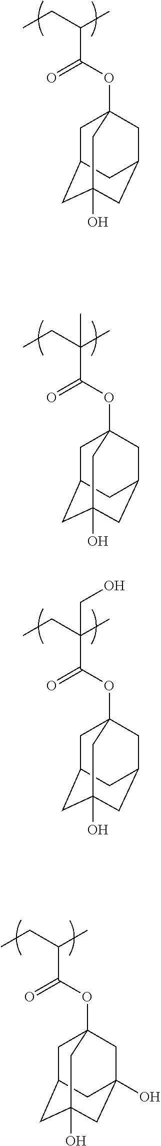

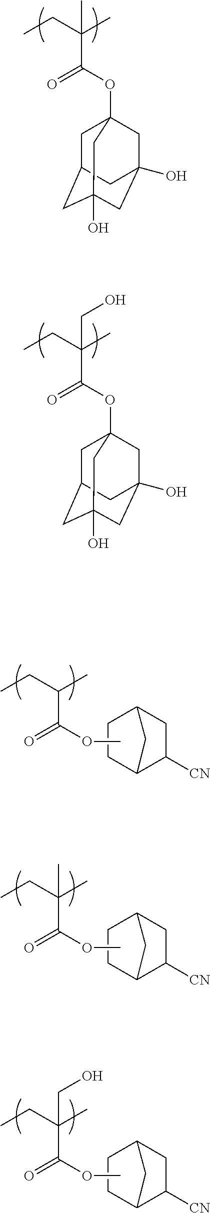

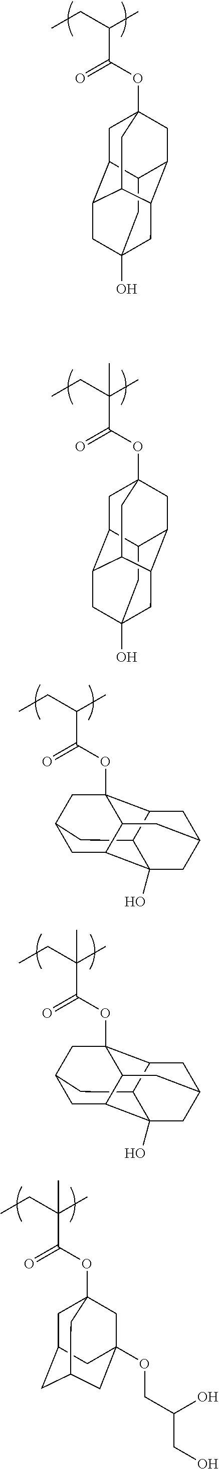

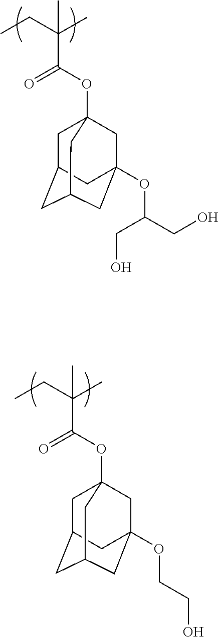

The repeating unit is more preferably a repeating unit represented by the following formula (AIIIa) or (AIIIb):

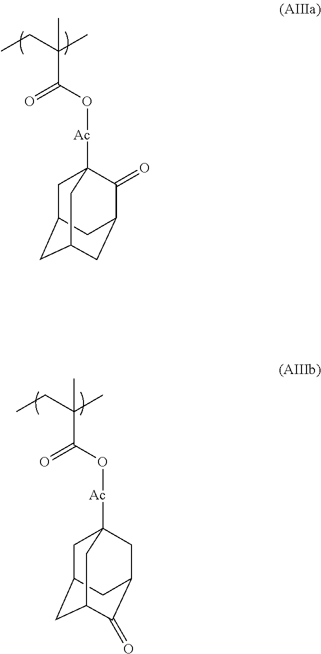

In formulae (AIIIa) and (AIIIb), Ac represents a single bond or a divalent linking group, and the preferred range thereof is the same as that of Ab in the repeating unit represented by any one of formulae (AIIa) to (AIIc).

Specific examples of the repeating unit represented by formula (AIIIa) or (AIIIb) are illustrated below, but the present invention is not limited thereto.

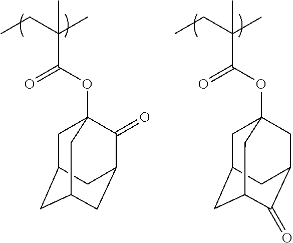

In addition, for example, monomers and corresponding repeating units described in paragraph [0011] et seq. of International Publication No. 2011/122336 may also be appropriately used.

The resin (A) may contain a repeating unit having an acid group. The acid group includes a carboxyl group, a sulfonamide group, a sulfonylimide group, a bissulfonylimide group, a naphthol structure, and an aliphatic alcohol group substituted with an electron-withdrawing group on the α-position (for example, a hexafluoroisopropanol group), and it is more preferred to contain a repeating unit having a carboxyl group. By virtue of containing a repeating unit having an acid group, the resolution increases in the usage of forming contact holes. As for the repeating unit having an acid group, all of a repeating unit where an acid group is directly bonded to the main chain of the resin, such as repeating unit by an acrylic acid or a methacrylic acid, a repeating unit where an acid group is bonded to the main chain of the resin through a linking group, and a repeating unit where an acid group is introduced into the polymer chain terminal by using an acid group-containing polymerization initiator or chain transfer agent at the polymerization, are preferred. The linking group may have a monocyclic or polycyclic cyclohydrocarbon structure. In particular, a repeating unit by an acrylic acid or a methacrylic acid is preferred.

The resin (A) may or may not contain a repeating unit having an acid group, but in the case of containing a repeating unit having an acid group, the content thereof is preferably 25 mol % or less, more preferably 20 mol % or less, based on all repeating units in the resin (A). In the case where the resin (A) contains a repeating unit having an acid group, the content of the acid group-containing repeating unit in the resin (A) is usually 1 mol % or more.

Specific examples of the repeating unit having an acid group are illustrated below, but the present invention is not limited thereto.

In specific examples, Rx represents H, CH₃, CH₂OH or CF₃.

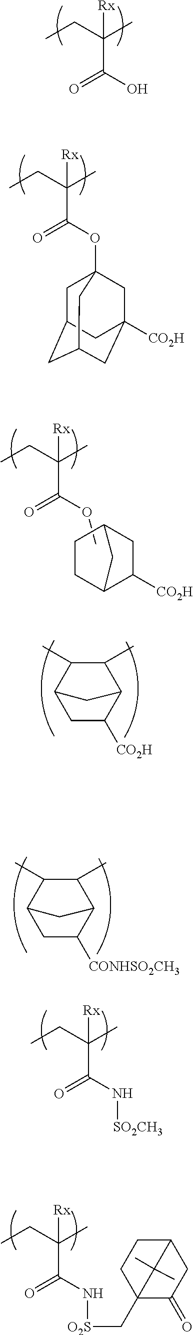

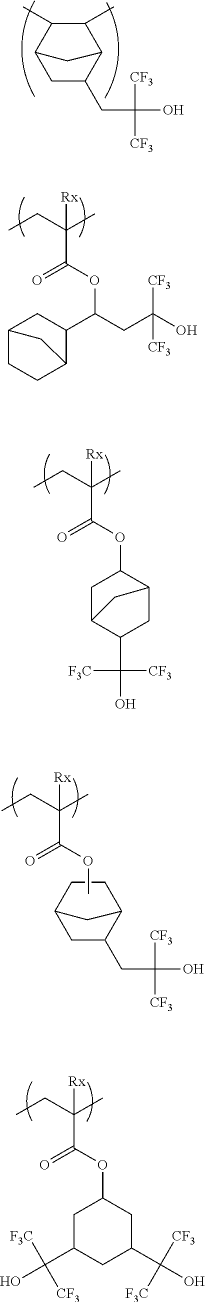

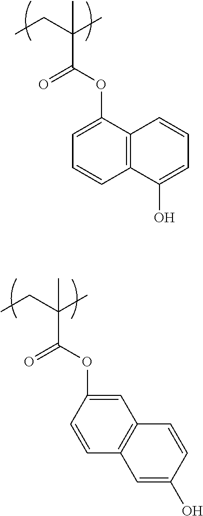

The resin (A) for use in the present invention may further contain a repeating unit having an alicyclic hydrocarbon structure free from a polar group (for example, the above-described acid group, a hydroxyl group or a cyano group) and not exhibiting acid decomposability. Thanks to this repeating unit, elution of a low molecular component from the resist film to the immersion liquid can be reduced at the immersion exposure and in addition, the solubility of the resin at the development using an organic solvent-containing developer can be appropriately adjusted. Such a repeating unit includes a repeating unit represented by formula (IV):

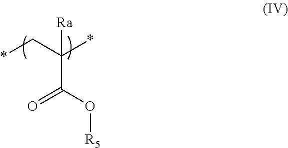

In formula (IV), R₅ represents a hydrocarbon group containing at least one cyclic structure and having no polar group.

Ra represents a hydrogen atom, an alkyl group or a —CH₂—O—Ra₂ group, wherein Ra₂ represents a hydrogen atom, an alkyl group or an acyl group. Ra is preferably a hydrogen atom, a methyl group, a hydroxymethyl group or a trifluoromethyl group, more preferably a hydrogen atom or a methyl group.

The cyclic structure contained in R₅ includes a monocyclic hydrocarbon group and a polycyclic hydrocarbon group. The monocyclic hydrocarbon group includes, for example, a cycloalkyl group having a carbon number of 3 to 12, such as cyclopentyl group, cyclohexyl group, cycloheptyl group and cyclooctyl group, and a cycloalkenyl group having a carbon number of 3 to 12, such as cyclohexenyl group. The monocyclic hydrocarbon group is preferably a monocyclic hydrocarbon group having a carbon number of 3 to 7, more preferably a cyclopentyl group or a cyclohexyl group.

The polycyclic hydrocarbon group includes a ring-assembly hydrocarbon group and a crosslinked cyclic hydrocarbon group. Examples of the ring-assembly hydrocarbon group include a bicyclohexyl group and a perhydronaphthalenyl group. Examples of the crosslinked cyclic hydrocarbon ring include a bicyclic hydrocarbon ring such as pinane ring, bornane ring, norpinane ring, norbornane ring and bicyclooctane ring (e.g., bicyclo[2.2.2]octane ring, bicyclo[3.2.1]octane ring), a tricyclic hydrocarbon ring such as homobledane ring, adamantane ring, tricyclo[5.2.1.0^{2,6}]decane ring and tricyclo[4.3.1.1^{2,5}]undecane ring, and a tetracyclic hydrocarbon ring such as tetracyclo[4.4.0.1^{2,5}.1^{7,10}]dodecane ring and perhydro-1,4-methano-5,8-methanonaphthalene ring. The crosslinked cyclic hydrocarbon ring also includes a condensed cyclic hydrocarbon ring, for example, a condensed ring formed by fusing a plurality of 5- to 8-membered cycloalkane rings, such as perhydronaphthalene (decalin) ring, perhydroanthracene ring, perhydrophenathrene ring, perhydroacenaphthene ring, perhydrofluorene ring, perhydroindene ring and perhydrophenalene ring.

Preferred examples of the crosslinked cyclic hydrocarbon ring include a norbornyl group, an adamantyl group, a bicyclooctanyl group, and a tricyclo[5,2,1,0^{2,6}]decanyl group. Among these crosslinked cyclic hydrocarbon rings, a norbomyl group and an adamantyl group are more preferred.

Such an alicyclic hydrocarbon group may have a substituent, and preferred examples of the substituent include a halogen atom, an alkyl group, a hydroxyl group with a hydrogen atom being substituted for, and an amino group with a hydrogen atom being substituted for. The halogen atom is preferably bromine atom, chlorine atom or fluorine atom, and the alkyl group is preferably a methyl group, an ethyl group, an n-butyl group or a tert-butyl group. This alkyl group may further have a substituent, and the substituent which may be further substituted on the alkyl group includes a halogen atom, an alkyl group, a hydroxyl group with a hydrogen atom being substituted for, and an amino group with a hydrogen atom being substituted for.

The substituent for the hydrogen atom includes, for example, an alkyl group, a cycloalkyl group, an aralkyl group, a substituted methyl group, a substituted ethyl group, an alkoxycarbonyl group, and an aralkyloxycarbonyl group. The alkyl group is preferably an alkyl group having a carbon number of 1 to 4; the substituted methyl group is preferably a methoxymethyl group, a methoxythiomethyl group, a benzyloxymethyl group, a tert-butoxymethyl group or a 2-methoxyethoxymethyl group; the substituted ethyl group is preferably a 1-ethoxyethyl group or a 1-methyl-1-methoxyethyl group; the acyl group is preferably an aliphatic acyl group having a carbon number of 1 to 6, such as formyl group, acetyl group, propionyl group, butyryl group, isobutyryl group, valeryl group and pivaloyl group; and the alkoxycarbonyl group includes, for example, an alkoxycarbonyl group having a carbon number of 1 to 4.

The resin (A) may or may not contain a repeating unit having an alicyclic hydrocarbon structure free from a polar group and not exhibiting acid decomposability, but in the case of containing this repeating unit, the content thereof is preferably from 1 to 50 mol %, more preferably from 5 to 50 mol %, further more preferably from 5 to 30 mol %, based on all repeating units in the resin (A).

Specific examples of the repeating unit having an alicyclic hydrocarbon structure free from a polar group and not exhibiting acid decomposability are illustrated below, but the present invention is not limited thereto. In the formulae, Ra represents H, CH₃, CH₂OH or CF₃.

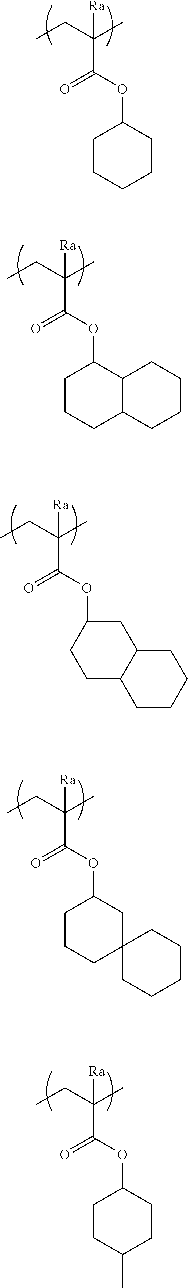

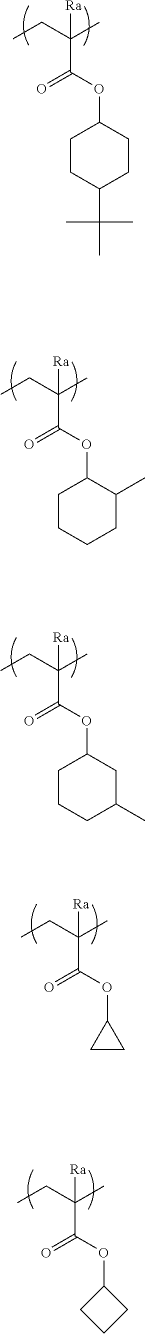

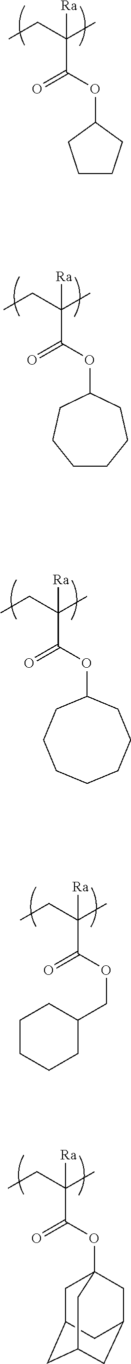

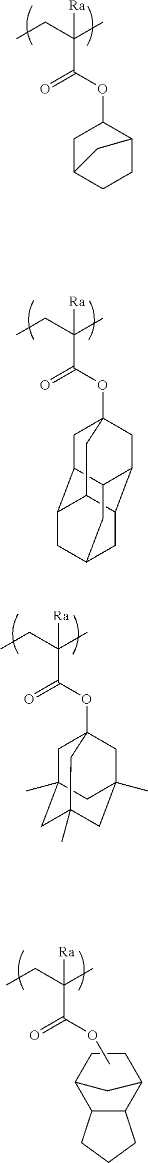

In the present invention, the resins suitably used for exposure particularly to an ArF excimer laser are set forth also in Examples, but, for example, the following resins are suitably used as well.

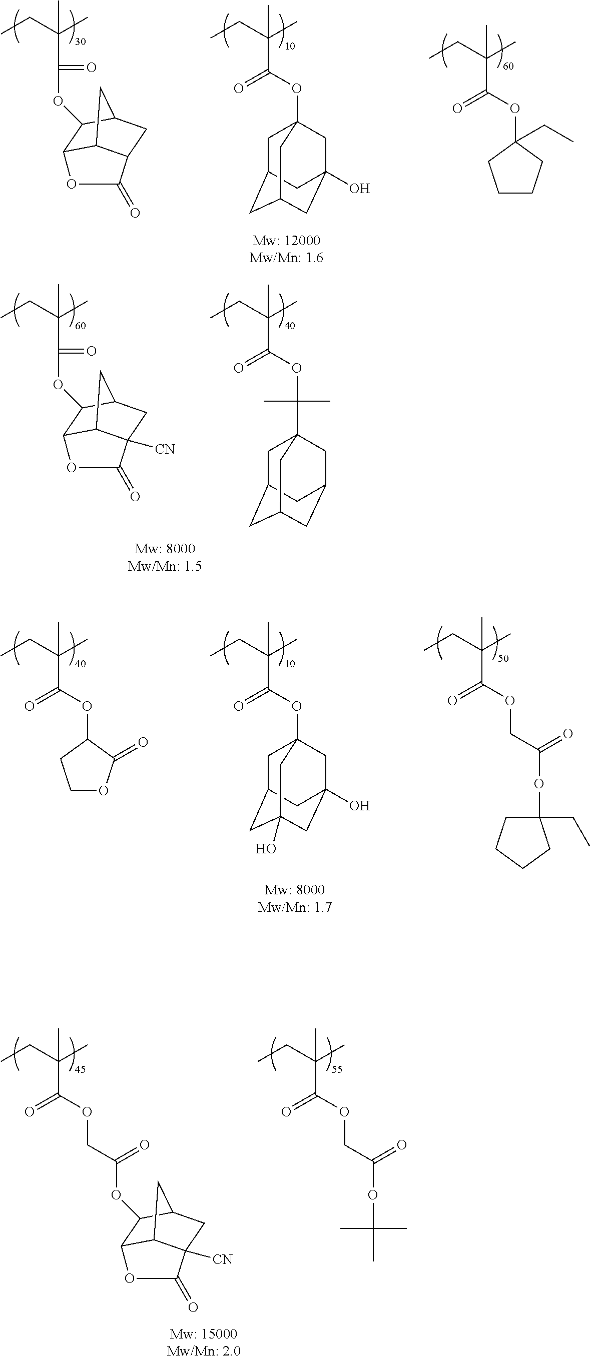

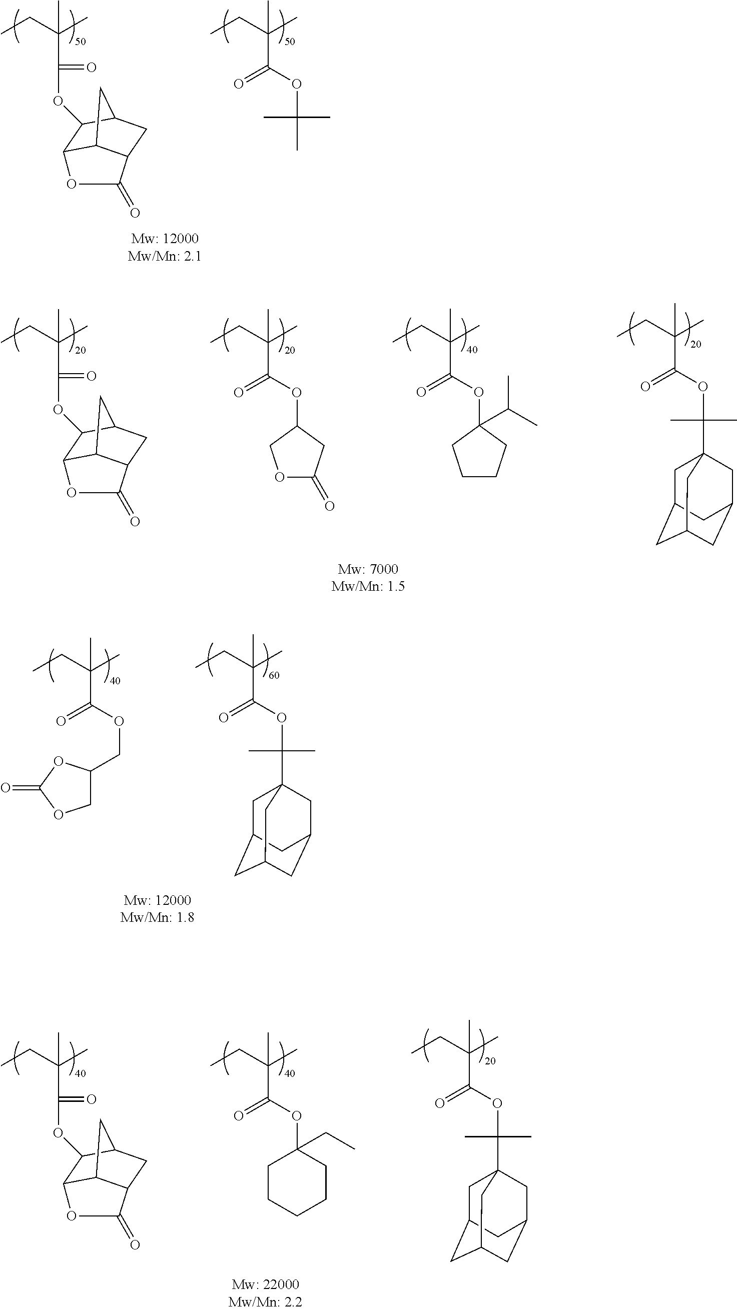

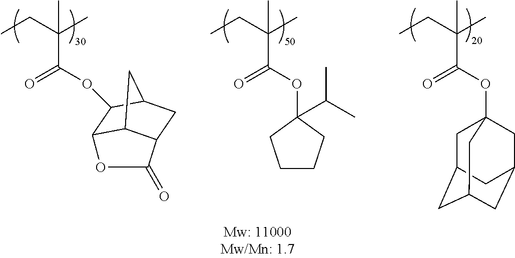

In the case of irradiating the composition of the present invention with KrF excimer laser light, electron beam, X-ray or high-energy beam at a wavelength of 50 nm or less (e.g., EUV), the resin (A) preferably contains an aromatic ring-containing unit typified by a hydroxystyrene repeating unit. More preferably, the resin (A) is a copolymer of a hydroxystyrene and a hydroxystyrene protected by a group capable of leaving by the action of an acid, or a copolymer of a hydroxystyrene and a tertiary alkyl (meth)acrylate.

Specifically, such a resin includes a resin containing a repeating unit represented by the following formula (A):

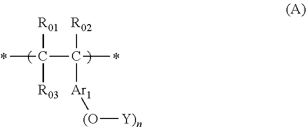

In the formula, each of R₀₁, R₀₂ and R₀₃ independently represents, for example, a hydrogen atom, an alkyl group, a cycloalkyl group, a halogen atom, a cyano group or an alkoxycarbonyl group, and Ar₁ represents, for example, an aromatic ring group. Incidentally, R₀₃ and Ar₁ may be an alkylene group and these two members may combine to form a 5- or 6-membered ring together with the —C—C— chain.

Each of n Ys independently represents a hydrogen atom or a group capable of leaving by the action of an acid, provided that at least one Y represents a group capable of leaving by the action of an acid.

n represents an integer of 1 to 4 and is preferably 1 or 2, more preferably 1.

The alkyl group as R₀₁ to R₀₃ is, for example, an alkyl group having a carbon number of 20 or less and is preferably a methyl group, an ethyl group, a propyl group, an isopropyl group, an n-butyl group, a sec-butyl group, a hexyl group, a 2-ethylhexyl group, an octyl group or a dodecyl group. The alkyl group is more preferably an alkyl group having a carbon number of 8 or less. These alkyl groups may have a substituent.

As the alkyl group contained in the alkoxycarbonyl group, the same as those for the alkyl group in R₀₁ to R₀₃ are preferred.

The cycloalkyl group may be a monocyclic cycloalkyl group or a polycyclic cycloalkyl group and is preferably a monocyclic cycloalkyl group having a carbon number of 3 to 8, such as cyclopropyl group, cyclopentyl group and cyclohexyl group. These cycloalkyl groups may have a substituent.

The halogen atom includes a fluorine atom, a chlorine atom, a bromine atom and an iodine atom and is preferably a fluorine atom.

In the case where R₀₃ represents an alkylene group, the alkylene group is preferably an alkylene group having a carbon number of 1 to 8, such as methylene group, ethylene group, propylene group, butylene group, hexylene group and octylene group.

The aromatic ring group as Art is preferably an aromatic ring group having a carbon number of 6 to 14, and examples thereof include a benzene group, a tolylene group and a naphthylene group. These aromatic ring groups may have a substituent.

The group Y capable of leaving by the action of an acid includes, for example, groups represented by —C(R₃₆)(R₃₇)(R₃₈), —C(═O)—O—C(R₃₆)(R₃₇)(R₃₈), —C(R₀₁)(R₀₂)(OR₃₉), —C(R₀₁)(R₀₂)—C((═O)—O—C(R₃₆)(R₃₇)(R₃₈) and —CH(R₃₆)(Ar).

In the formulae, each of R₃₆ to R₃₉ independently represents an alkyl group, a cycloalkyl group, an aryl group, an aralkyl group or an alkenyl group. R₃₆ and R₃₇ may combine with each other to form a ring structure.

Each of R₀₁ and R₀₂ independently represents a hydrogen atom, an alkyl group, a cycloalkyl group, an aryl group, an aralkyl group or an alkenyl group.

Ar represents an aryl group.

The alkyl group as R₃₆ to R₃₉, R₀₁ and R₀₂ is preferably an alkyl group having a carbon number of 1 to 8, and examples thereof include a methyl group, an ethyl group, a propyl group, an n-butyl group, a sec-butyl group, a hexyl group and an octyl group.

The cycloalkyl group as R₃₆ to R₃₉, R₀₁ and R₀₂ may be a monocyclic cycloalkyl group or a polycyclic cycloalkyl group. The monocyclic cycloalkyl group is preferably a cycloalkyl group having a carbon number of 3 to 8, and examples thereof include a cyclopropyl group, a cyclobutyl group, a cyclopentyl group, a cyclohexyl group and a cyclooctyl group. The polycyclic cycloalkyl group is preferably a cycloalkyl group having a carbon number of 6 to 20, and examples thereof include an adamantyl group, a norbornyl group, an isoboronyl group, a camphanyl group, a dicyclopentyl group, an α-pinel group, a tricyclodecanyl group, a tetracyclododecyl group and an androstanyl group. Incidentally, a part of carbon atoms in the cycloalkyl group may be substituted with a heteroatom such as oxygen atom.

The aryl group as R₃₆ to R₃₉, R₀₁, R₀₂ and Ar is preferably an aryl group having a carbon number of 6 to 10, and examples thereof include a phenyl group, a naphthyl group and an anthryl group.

The aralkyl group as R₃₆ to R₃₉, R₀₁ and R₀₂ is preferably an aralkyl group having a carbon number of 7 to 12, and preferred examples thereof include a benzyl group, a phenethyl group and a naphthylmethyl group.

The alkenyl group as R₃₆ to R₃₉, R₀₁ and R₀₂ is preferably an alkenyl group having a carbon number of 2 to 8, and examples thereof include a vinyl group, an allyl group, a butenyl group and a cyclohexenyl group.

The ring that can be formed by combining R₃₆ and R₃₇ with each other may be monocyclic or polycyclic. The monocyclic ring is preferably a cycloalkane structure having a carbon number of 3 to 8, and examples thereof include a cyclopropane structure, a cyclobutane structure, a cyclopentane structure, a cyclohexane structure, a cycloheptane structure and a cyclooctane structure. The polycyclic ring is preferably a cycloalkane structure having a carbon number of 6 to 20, and examples thereof include an adamantane structure, a norbornane structure, a dicyclopentane structure, a tricyclodecane structure and a tetracyclododecane structure. Incidentally, a part of carbon atoms in the ring structure may be substituted with a heteroatom such as oxygen atom.

Each of the groups above may have a substituent, and the substituent includes, for example, an alkyl group, a cycloalkyl group, an aryl group, an amino group, an amido group, a ureido group, a urethane group, a hydroxyl group, a carboxyl group, a halogen atom, an alkoxy group, a thioether group, an acyl group, an acyloxy group, an alkoxycarbonyl group, a cyano group and a nitro group. The carbon number of such a substituent is preferably 8 or less.

The group Y capable of leaving by the action of an acid is more preferably a structure represented by the following formula (B):

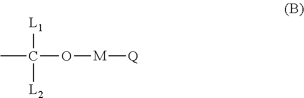

In the formula, each of L₁ and L₂ independently represents a hydrogen atom, an alkyl group, a cycloalkyl group, an aryl group or an aralkyl group.

M represents a single bond or a divalent linking group.

Q represents an alkyl group, a cycloalkyl group, a cyclic aliphatic group, an aromatic ring group, an amino group, an ammonium group, a mercapto group, a cyano group or an aldehyde group. The cyclic aliphatic group and the aromatic ring group may contain a heteroatom.

At least two members of Q, M and L₁ may combine with each other to form a 5- or 6-membered ring.

The alkyl group as L₁ and L is, for example, an alkyl group having a carbon number of 1 to 8 and specifically, includes a methyl group, an ethyl group, a propyl group, an n-butyl group, a sec-butyl group, a hexyl group and an octyl group.

The cycloalkyl group as L₁ and L₂ is, for example, a cycloalkyl group having a carbon number of 3 to 15 and specifically, includes a cyclopentyl group, a cyclohexyl group, a norbomyl group and an adamantyl group.

The aryl group as L₁ and L₂ is, for example, an aryl group having a carbon number of 6 to 15 and specifically, includes a phenyl group, a tolyl group, a naphthyl group and an anthryl group.

The aralkyl group as L₁ and L₂ is, for example, an aralkyl group having a carbon number of 6 to 20 and specifically, includes a benzyl group and a phenethyl group.

The divalent linking group as M includes, for example, an alkylene group (such as methylene group, ethylene group, propylene group, butylene group, hexylene group and octylene group), a cycloalkylene group (such as cyclopentylene group and cyclohexylene group), an alkenylene group (such as ethylene group, propenylene group and butenylene group), an arylene group (such as phenylene group, tolylene group and naphthylene group), —S—, —O—, —CO—, —SO₂—, —N(R₀)—, and a combination of two or more thereof. Here, R₀ is a hydrogen atom or an alkyl group. The alkyl group as R₀ is, for example, an alkyl group having a carbon number of 1 to 8 and specifically, includes a methyl group, an ethyl group, a propyl group, an n-butyl group, a sec-butyl group, a hexyl group and an octyl group.

The alkyl group and cycloalkyl group as Q are the same as respective groups of L₁ and L₂ described above.

The cyclic aliphatic group and aromatic ring group as Q include the above-described cycloalkyl group and aryl group of L₁ and L₂. These cycloalkyl group and aryl group are preferably a group having a carbon number of 3 to 15.

The heteroatom-containing cyclic aliphatic group or aromatic ring group as Q includes, for example, a group having a heterocyclic structure such as thiirane, cyclothiolane, thiophene, furan, pyrrole, benzothiophene, benzofuran, benzopyrrole, triazine, imidazole, benzimidazole, triazole, thiadiazole, thiazole and pyrrolidone, but the ring is not limited thereto as long as it is a ring composed of carbon and a heteroatom or a ring composed of only a heteroatom.

The ring structure which may be formed by combining at least two members of Q, M and L₁ with each other includes, for example, a 5- or 6-membered ring structure where a propylene group or a butylene group is formed by the members above. Incidentally, this 5- or 6-membered ring structure contains an oxygen atom.

In formula (2), each of the groups represented by L₁, L₂, M and Q may have a substituent, and the substituent includes, for example, an alkyl group, a cycloalkyl group, an aryl group, an amino group, an amido group, a ureido group, a urethane group, a hydroxyl group, a carboxyl group, a halogen atom, an alkoxy group, a thioether group, an acyl group, an acyloxy group, an alkoxycarbonyl group, a cyano group and a nitro group. The carbon number of such a substituent is preferably 8 or less.

The group represented by -(M-Q) is preferably a group having a carbon number of 1 to 20, more preferably a group having a carbon number of 1 to 10, still more preferably a group having a carbon number of 1 to 8.

Specific examples of the resin (A) suited to the case of using KrF excimer laser light, electron beam, X ray or EUV light for patterning are illustrated below, but the present invention is not limited thereto.

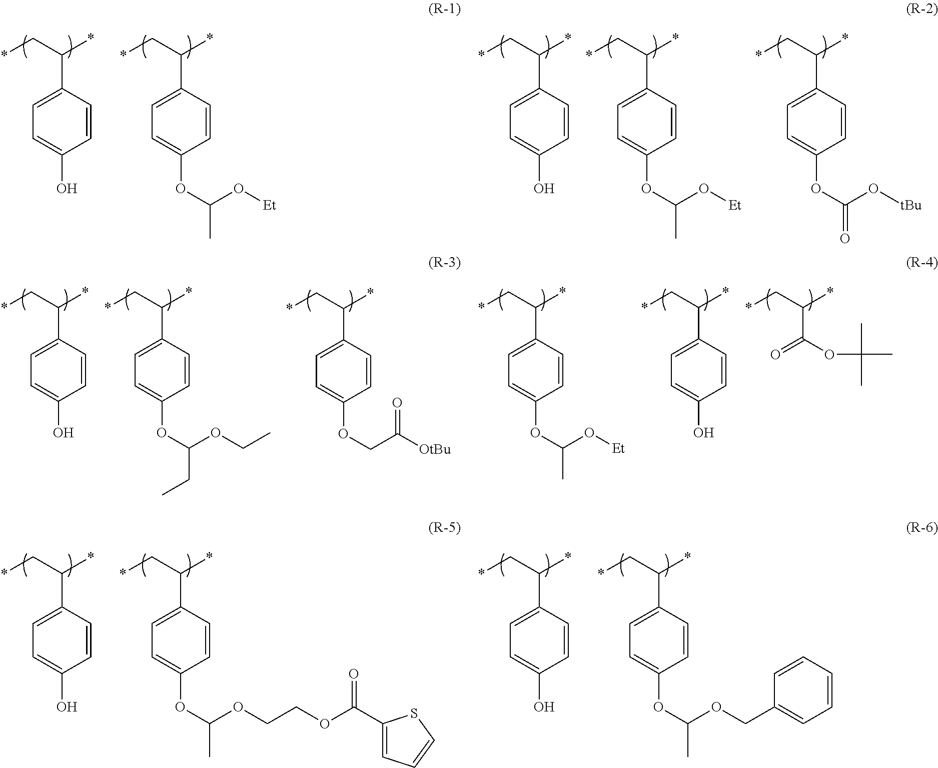

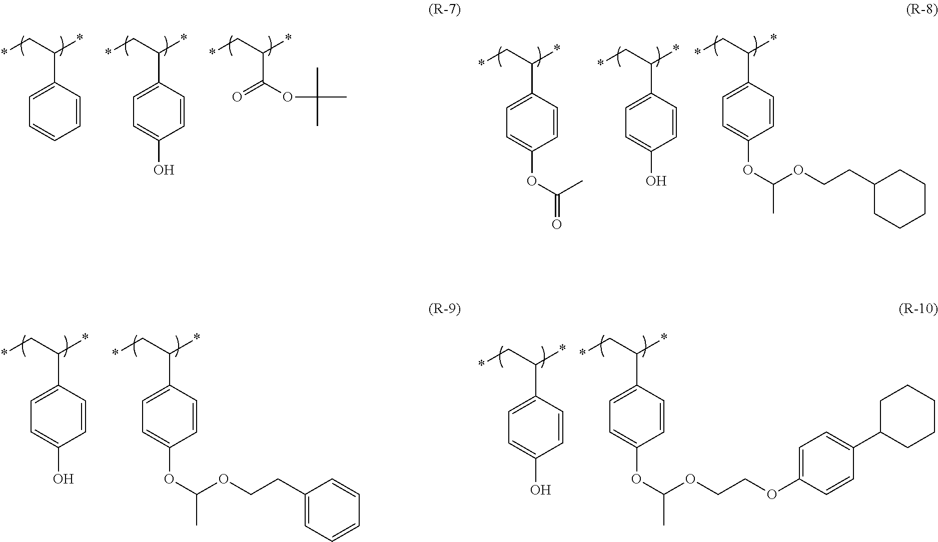

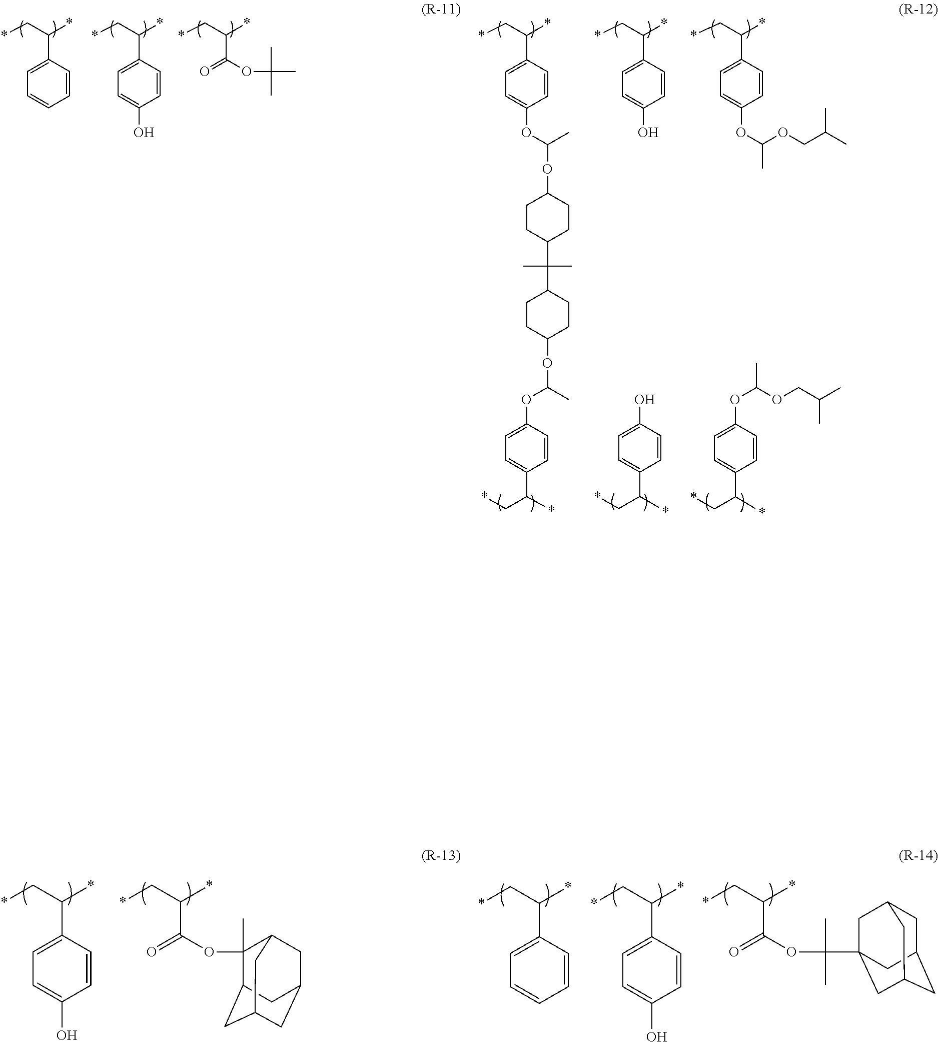

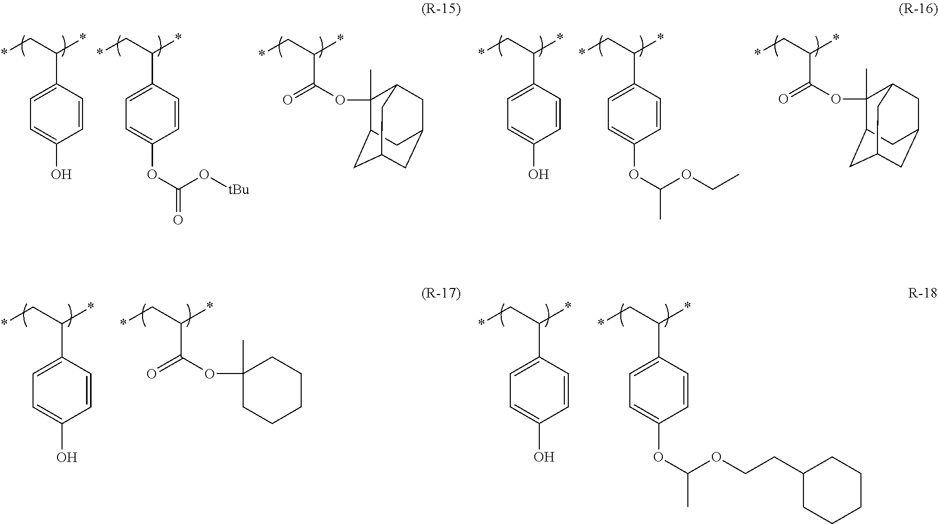

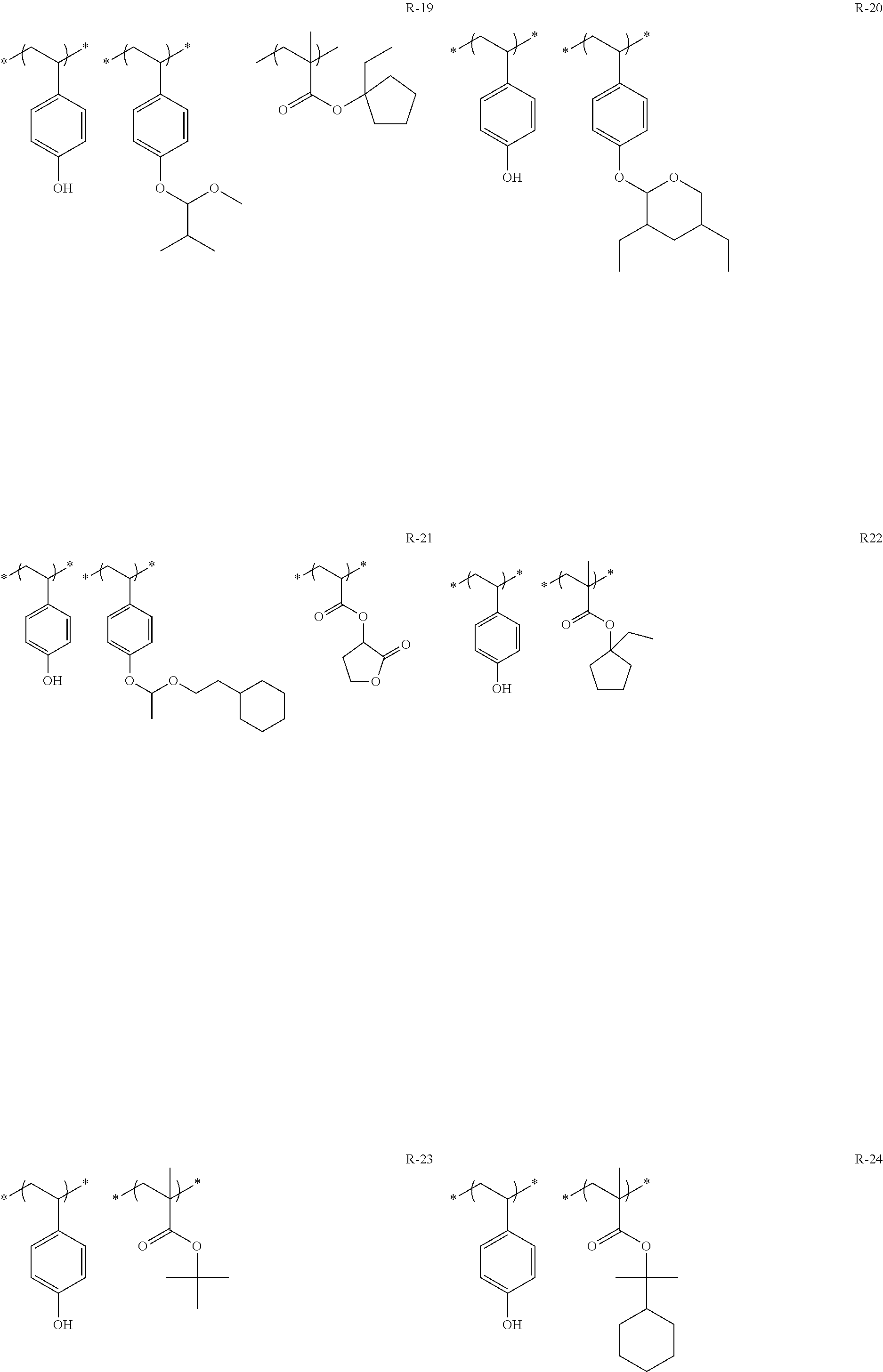

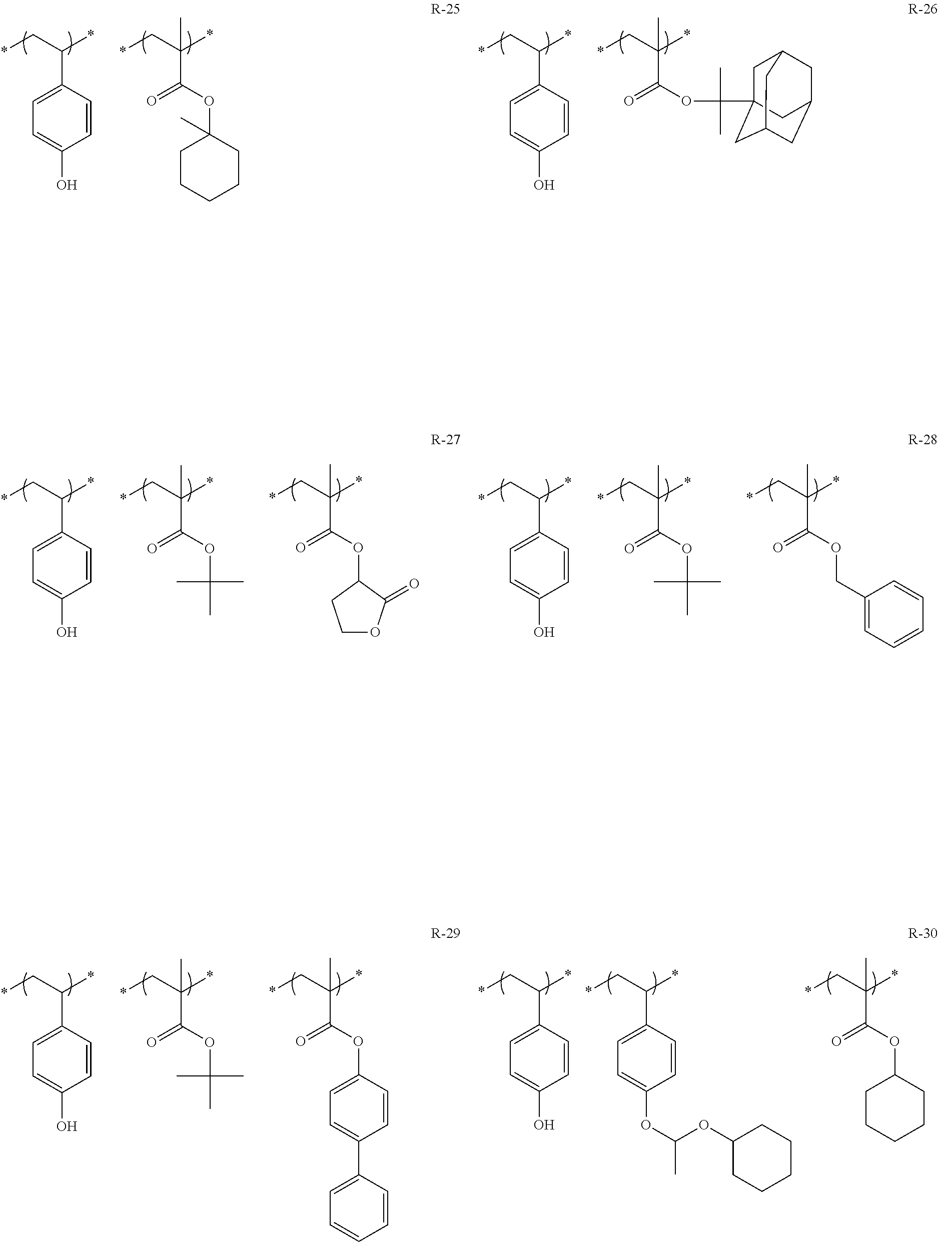

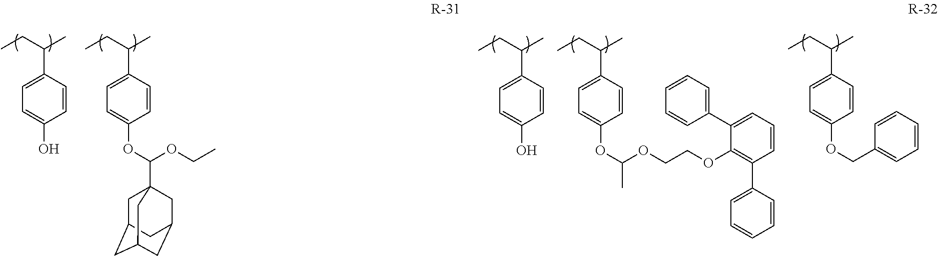

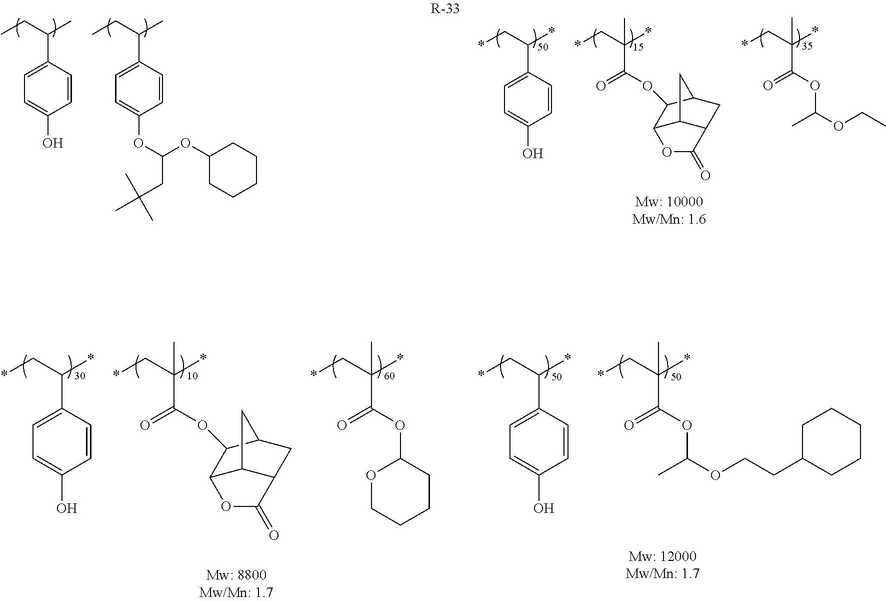

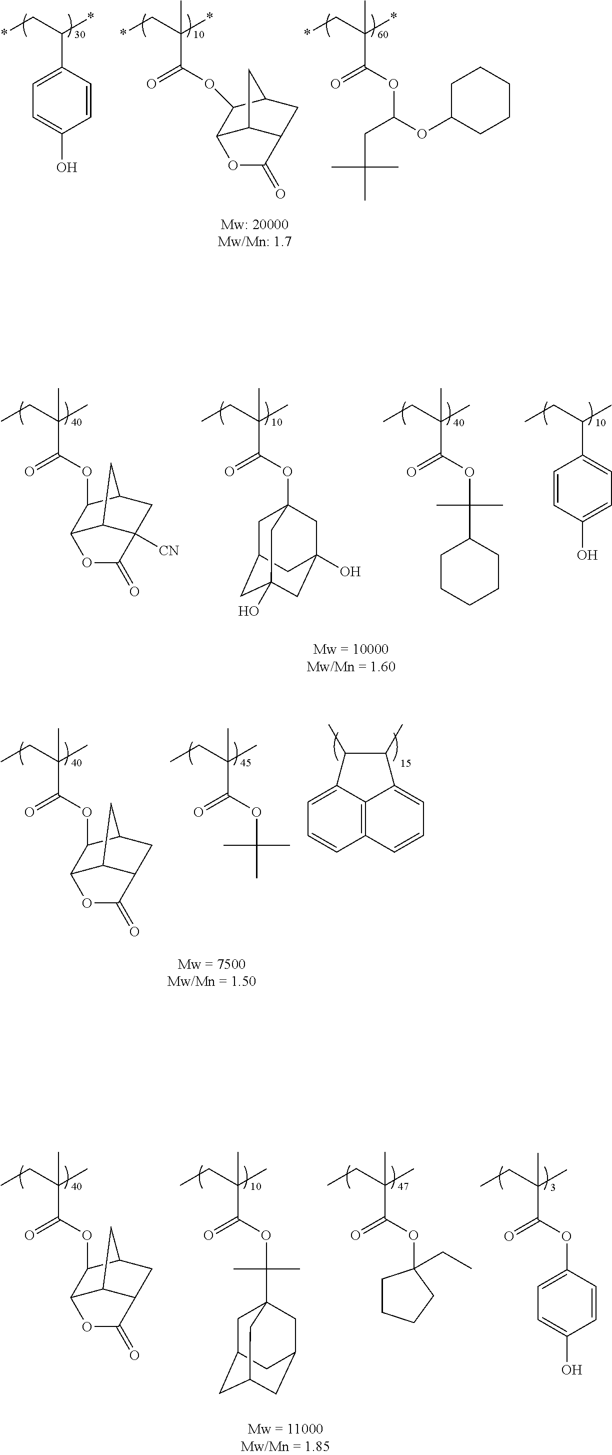

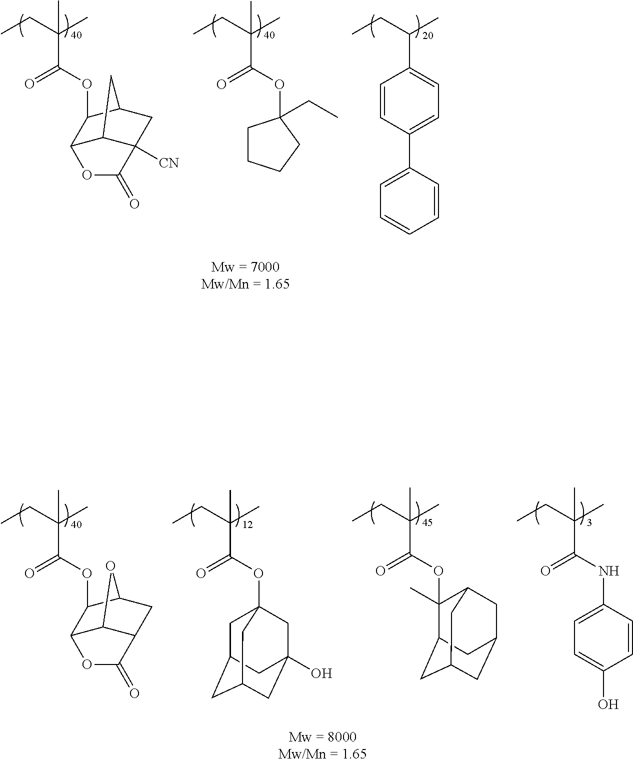

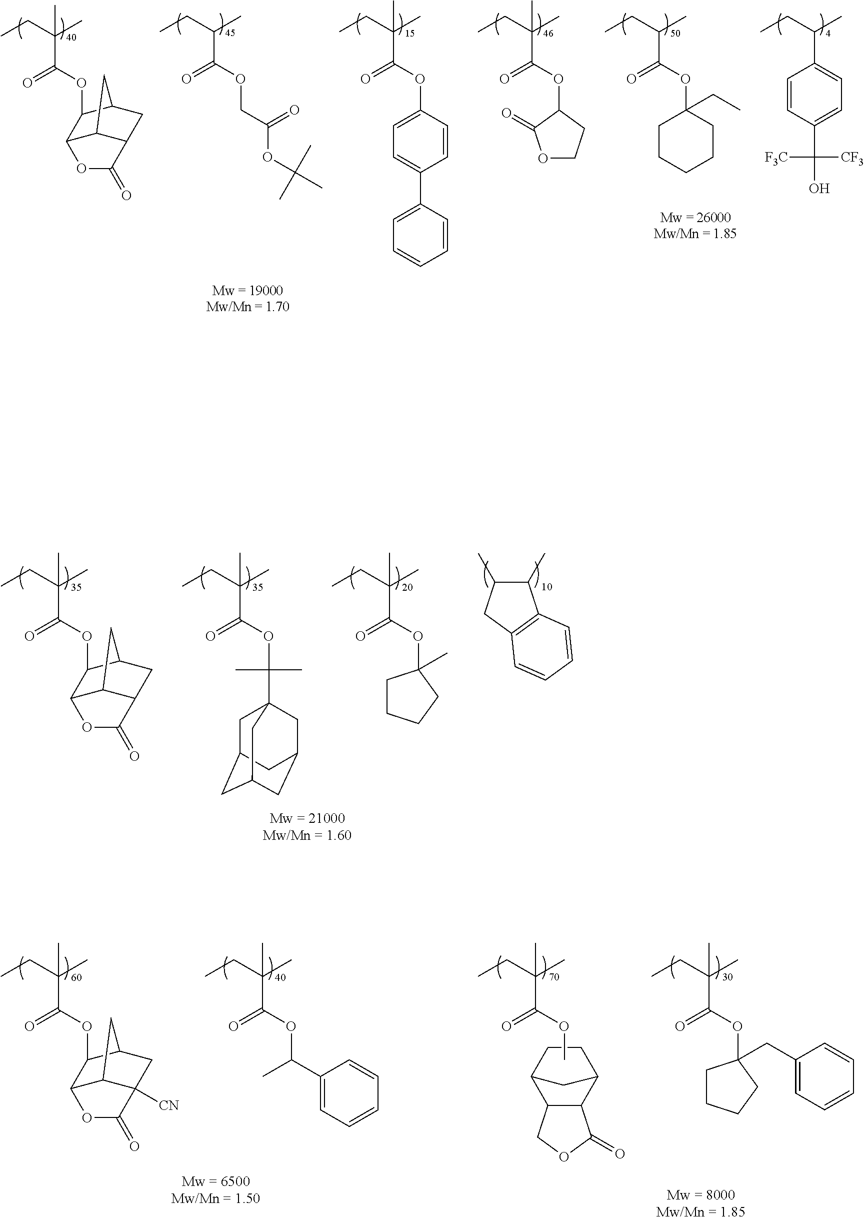

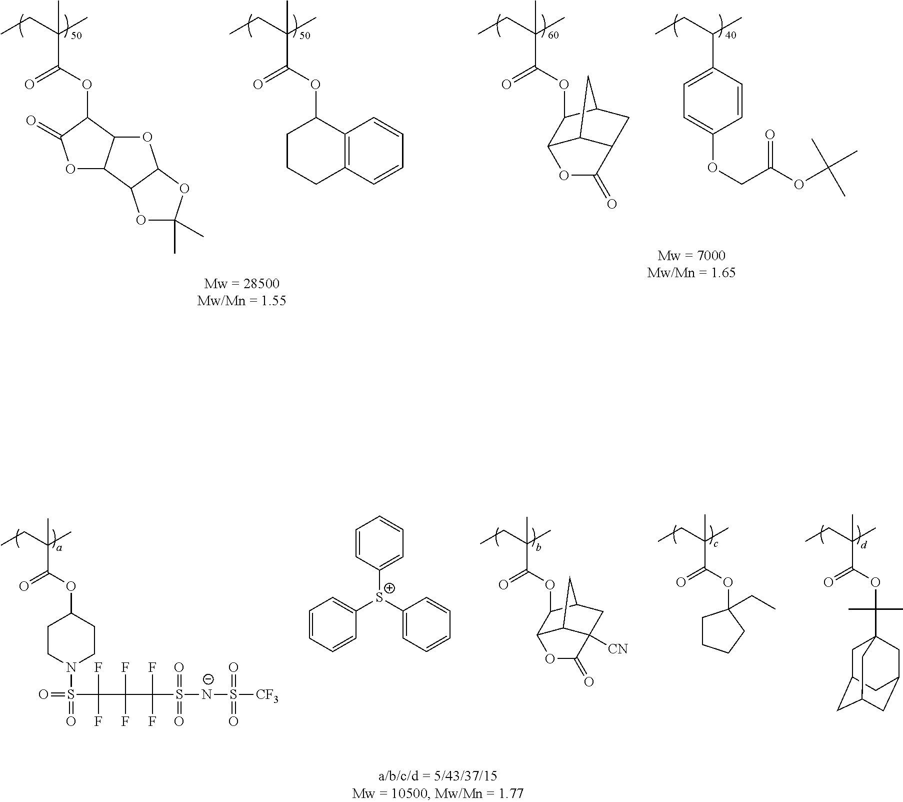

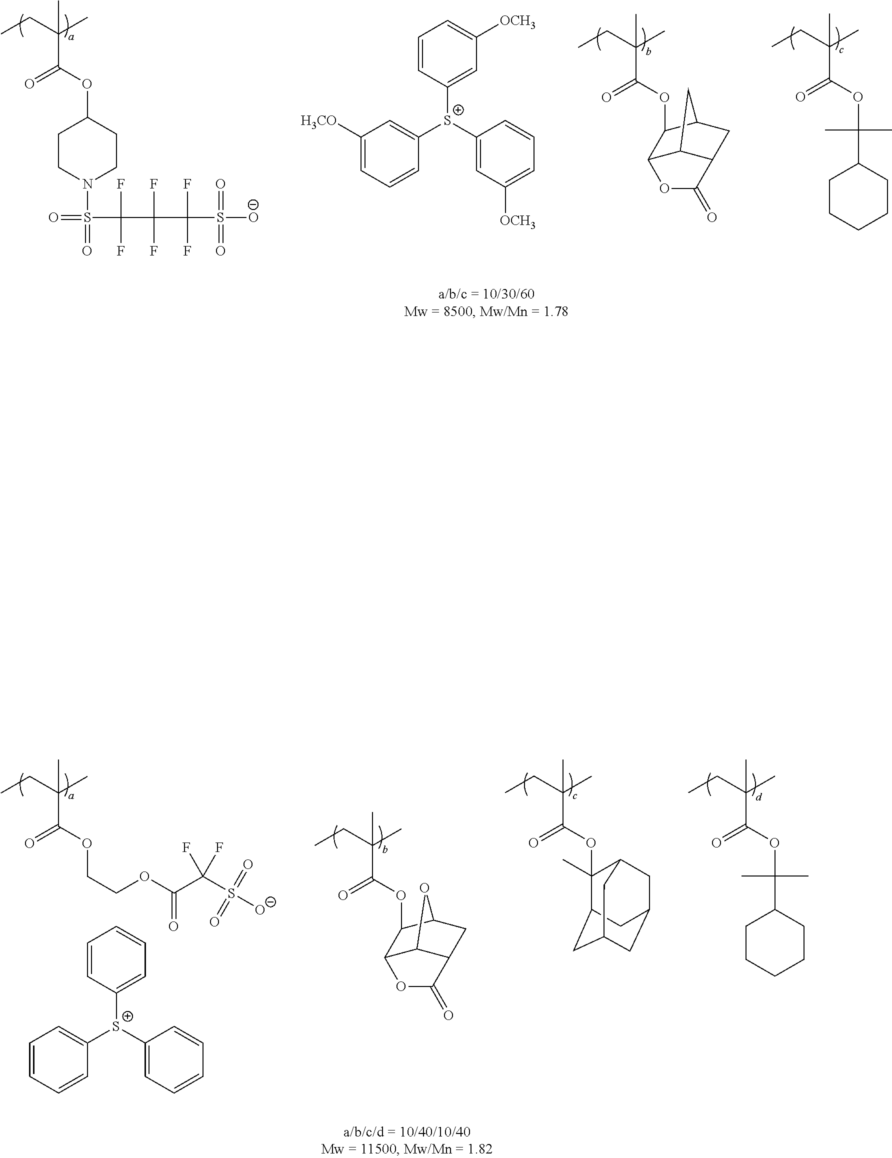

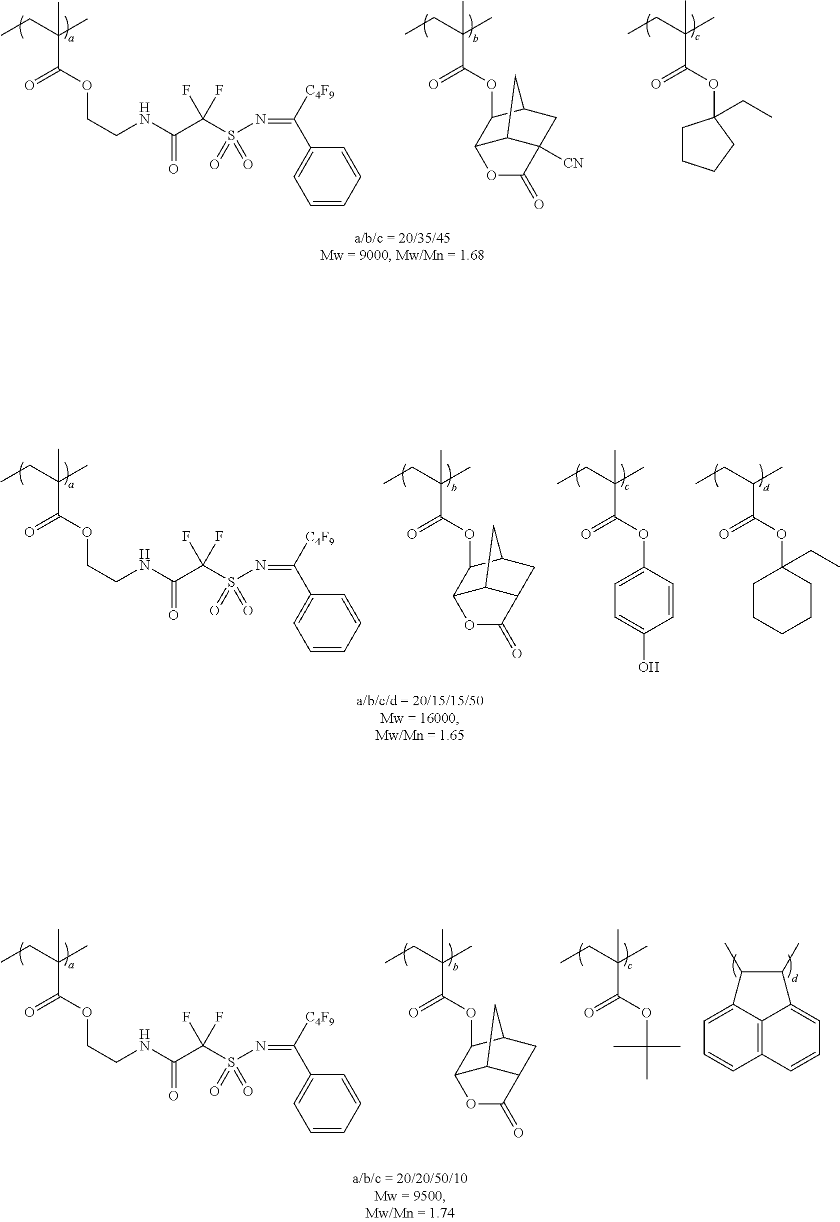

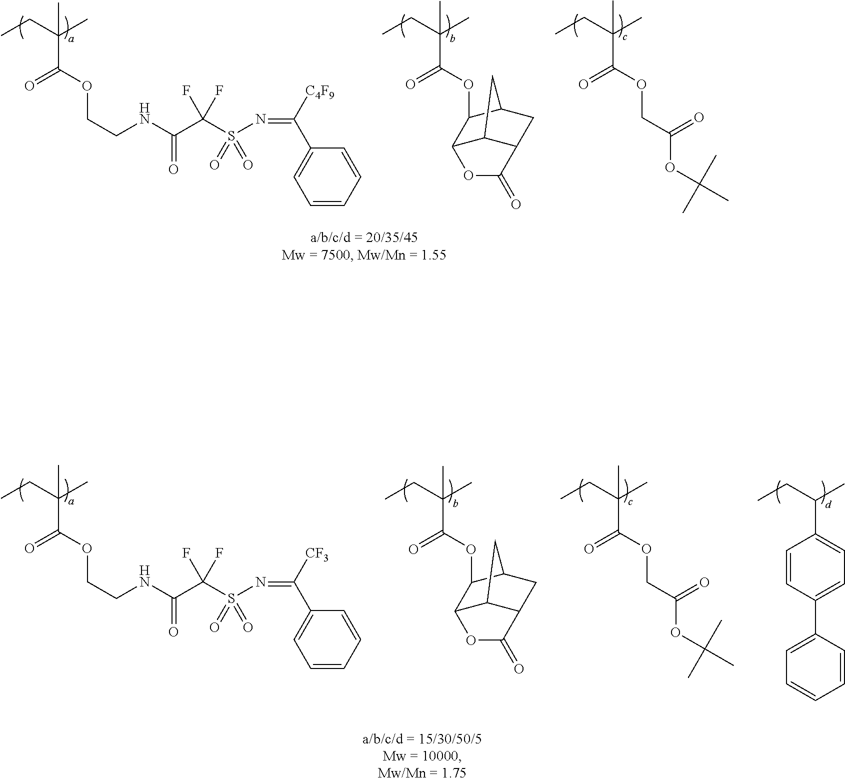

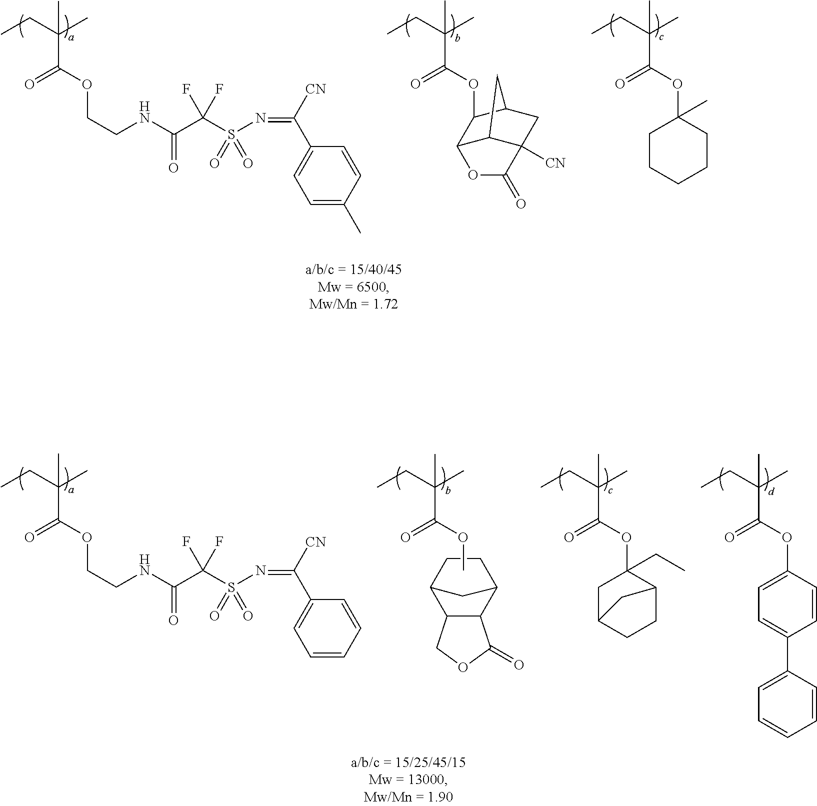

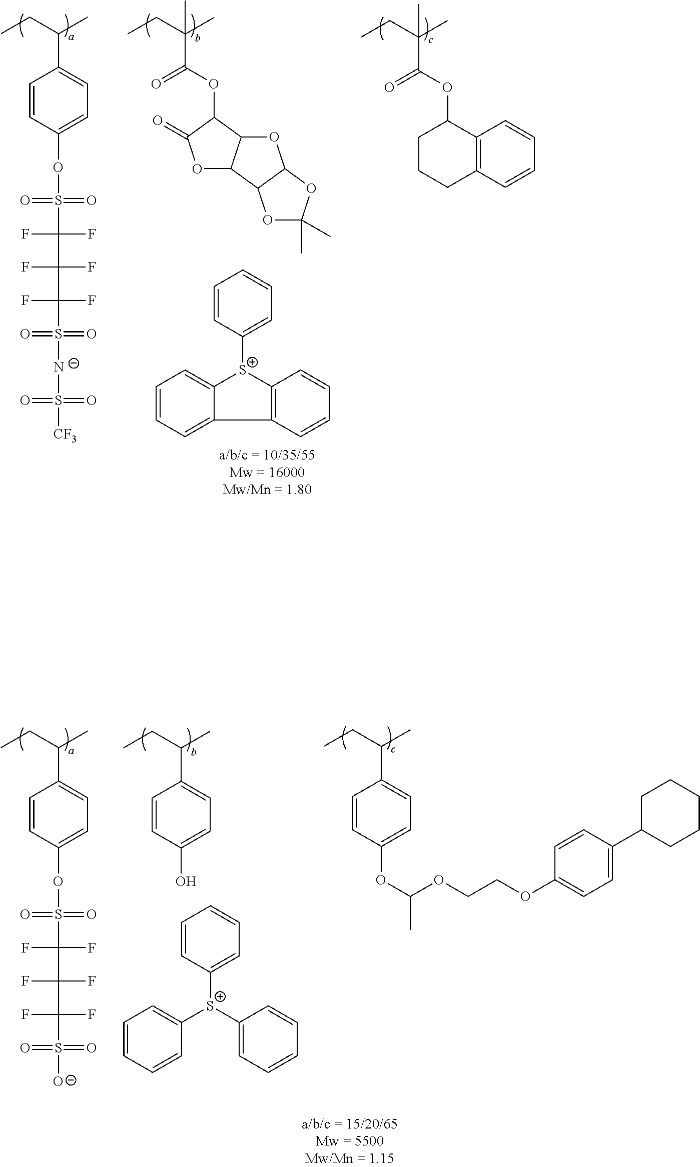

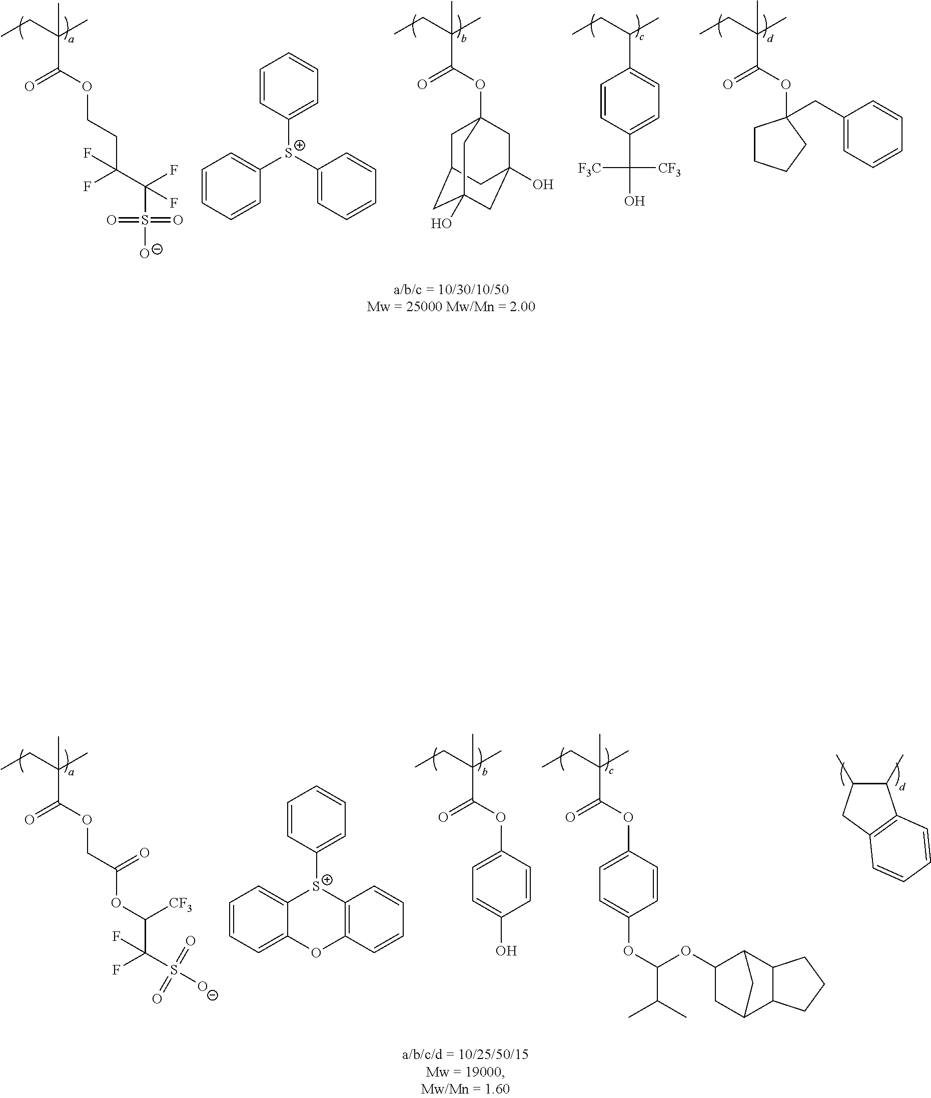

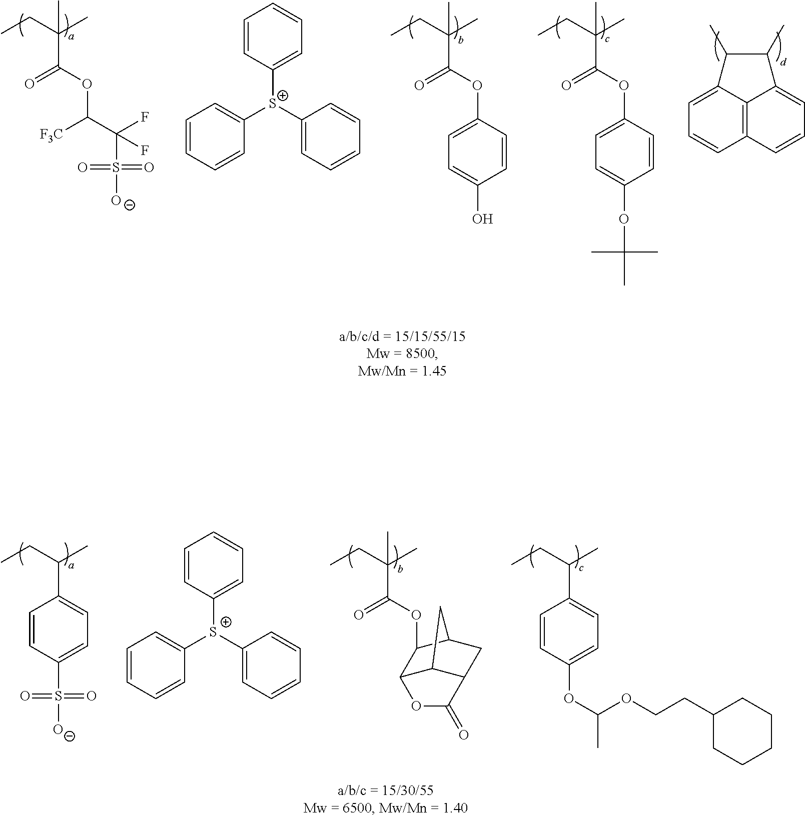

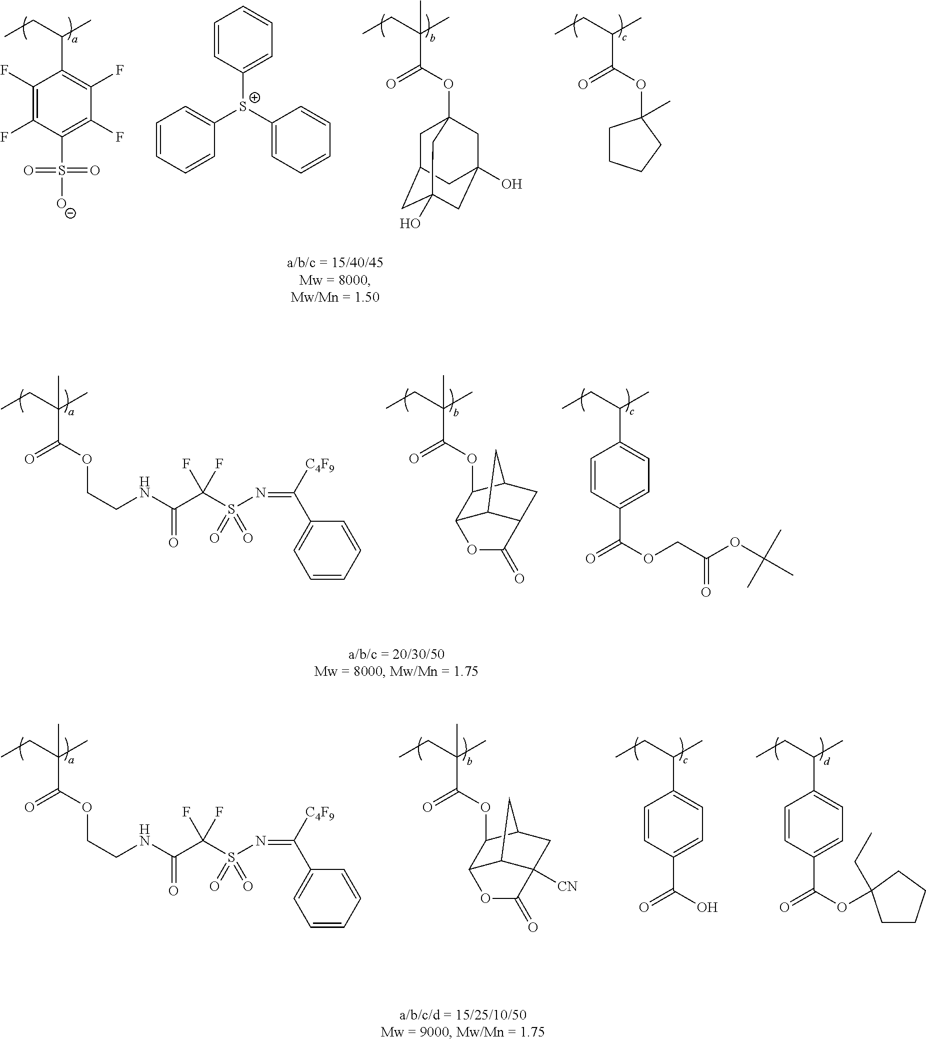

In these specific examples, “tBu” indicates a tert-butyl group.

The resin (A) for use in the composition of the present invention may contain, in addition to the above-described repeating structural units, various repeating structural units for the purpose of controlling dry etching resistance, suitability for standard developer, adherence to substrate, resist profile and properties generally required of an actinic ray-sensitive or radiation-sensitive resin composition, such as resolution, heat resistance and sensitivity.

Such a repeating structural unit includes repeating structural units corresponding to the monomers described below, but the present invention is not limited thereto.

Thanks to such a repeating structural unit, the performance required of the resin used in the composition of the present invention, particularly

(1) solubility for the coating solvent,
(2) film-forming property (glass transition temperature),
(3) alkali developability,
(4) film loss (selection of hydrophilic, hydrophobic or alkali-soluble group),
(5) adherence of unexposed area to substrate,
(6) dry etching resistance,
and the like, can be subtly controlled.

Such a monomer includes, for example, a compound having one addition-polymerizable unsaturated bond selected from acrylic acid esters, methacrylic acid esters, acrylamides, methacrylamides, allyl compounds, vinyl ethers and vinyl esters.

Other than these compounds, an addition-polymerizable unsaturated compound copolymerizable with the monomers corresponding to the above-described various repeating structural units may be copolymerized.

In the resin (A) for use in the composition of the present invention, the molar ratio of respective repeating structural units contained is appropriately set to control dry etching resistance of the actinic ray-sensitive or radiation-sensitive resin composition, suitability for standard developer, adherence to substrate, resist profile and performances generally required of an actinic ray-sensitive or radiation-sensitive resin composition, such as resolution, heat resistance and sensitivity.

In the case where the composition of the present invention is used for ArF exposure, in view of transparency to ArF light, the resin (A) for use in the composition of the present invention preferably has substantially no aromatic ring (specifically, the proportion of an aromatic group-containing repeating unit in the resin is preferably 5 mol % or less, more preferably 3 mol % or less, and ideally 0 mol %, that is, the resin does not have an aromatic group). The resin (A) preferably has a monocyclic or polycyclic alicyclic hydrocarbon structure.

The form of the resin (A) for use in the present invention may be any form of random type, block type, comb type and star type. The resin (A) can be synthesized, for example, by radical, cationic or anionic polymerization of unsaturated monomers corresponding to respective structures. The target resin can al be obtained by polymerizing unsaturated monomers corresponding to precursors of respective structures and then performing a polymer reaction.

In the case where the composition of the present invention contains the later-described resin (D), the resin (A) preferably contains no fluorine atom and no silicon atom in view of compatibility with the resin (D).

The resin (A) for use in the composition of the present invention is preferably a resin where all repeating units are composed of a (meth)acrylate-based repeating unit. In this case, all repeating units may be a methacrylate-based repeating unit, all repeating units may be an acrylate-based repeating unit, or all repeating units may be composed of a methacrylate-based repeating unit and an acrylate-based repeating unit, but the content of the acrylate-based repeating unit is preferably 50 mol % or less based on all repeating units.

The resin (A) for use in the present invention can be synthesized by a conventional method (for example, radical polymerization). The general synthesis method includes, for example, a batch polymerization method of dissolving monomer species and an initiator in a solvent and heating the solution, thereby effecting the polymerization, and a dropping polymerization method of adding dropwise a solution containing monomer species and an initiator to a heated solvent over 1 to 10 hours. A dropping polymerization method is preferred. The reaction solvent includes, for example, tetrahydrofuran, 1,4-dioxane, ethers such as diisopropyl ether, ketones such as methyl ethyl ketone and methyl isobutyl ketone, an ester solvent such as ethyl acetate, an amide solvent such as dimethylformamide and dimethylacetamide, and the later-described solvent capable of dissolving the composition of the present invention, such as propylene glycol monomethyl ether acetate, propylene glycol monomethyl ether and cyclohexanone. The polymerization is more preferably performed using the same solvent as the solvent used in the photosensitive composition of the present invention. By the use of the same solvent, production of particles during storage can be suppressed.

The polymerization reaction is preferably performed in an inert gas atmosphere such as nitrogen or argon. As for the polymerization initiator, the polymerization is started using a commercially available radical initiator (e.g., azo-based initiator, peroxide). The radical initiator is preferably an azo-based initiator, and an azo-based initiator having an ester group, a cyano group or a carboxyl group is preferred. Preferred initiators include azobisisobutyronitrile, azobisdimethylvaleronitrile, dimethyl 2,2′-azobis(2-methylpropionate), and the like. The initiator is added additionally or in parts as needed, and after the completion of reaction, the reaction solution is poured in a solvent to collect the desired polymer by powder, solid or other recovery methods. The concentration at the reaction is from 5 to 50 mass %, preferably from 10 to 30 mass %, and the reaction temperature is usually from 10 to 150° C., preferably from 30 to 120° C., more preferably from 60 to 100° C.

After the completion of reaction, the reaction solution is allowed to cool to room temperature and purified. The purification may be performed by a normal method, for example, a liquid-liquid extraction method of applying water washing or combining it with an appropriate solvent to remove residual monomers or oligomer components; a purification method in a solution sate, such as ultrafiltration of extracting and removing only polymers having a molecular weight not more than a specific value; a reprecipitation method of adding dropwise the resin solution in a poor solvent to solidify the resin in the poor solvent and thereby remove residual monomers and the like; and a purification method in a solid state, such as washing of a resin slurry with a poor solvent after separation of the slurry by filtration.

For example, the resin is precipitated as a solid by contacting the reaction solution with a solvent in which the resin is sparingly soluble or insoluble (poor solvent) and which is in a volumetric amount of 10 times or less, preferably from 10 to 5 times, the reaction solution.

The solvent used at the operation of precipitation or reprecipitation from the polymer solution (precipitation or reprecipitation solvent) may be sufficient if it is a poor solvent for the polymer, and the solvent which can be used may be appropriately selected from, for example, a hydrocarbon, a halogenated hydrocarbon, a nitro compound, an ether, a ketone, an ester, a carbonate, an alcohol, a carboxylic acid, water, and a mixed solvent containing such a solvent, according to the kind of the polymer. Among these solvents, a solvent containing at least an alcohol (particularly, methanol or the like) or water is preferred as the precipitation or reprecipitation solvent.

The amount of the precipitation or reprecipitation solvent used may be appropriately selected by taking into account the efficiency, yield and the like, but in general, the amount used is from 100 to 10,000 parts by mass, preferably from 200 to 2,000 parts by mass, more preferably from 300 to 1,000 parts by mass, per 100 parts by mass of the polymer solution.

The temperature at the precipitation or reprecipitation may be appropriately selected by taking into account the efficiency or operability but is usually on the order of 0 to 50° C., preferably in the vicinity of room temperature (for example, approximately from 20 to 35° C.). The precipitation or reprecipitation operation may be performed using a commonly employed mixing vessel such as stirring tank by a known method such as batch system and continuous system.

The precipitated or reprecipitated polymer is usually subjected to commonly employed solid-liquid separation such as filtration and centrifugation, then dried and used. The filtration is performed using a solvent-resistant filter element preferably under pressure. The drying is performed under atmospheric pressure or reduced pressure (preferably under reduced pressure) at a temperature of approximately from 30 to 100° C., preferably on the order of 30 to 50° C.

Incidentally, after the resin is once precipitated and separated, the resin may be again dissolved in a solvent and then put into contact with a solvent in which the resin is sparingly soluble or insoluble. That is, there may be used a method including, after the completion of radical polymerization reaction, bringing the polymer into contact with a solvent in which the polymer is sparingly soluble or insoluble, to precipitate a resin (step a), separating the resin from the solution (step b), anew dissolving the resin in a solvent to prepare a resin solution A (step c), bringing the resin solution A into contact with a solvent in which the resin is sparingly soluble or insoluble and which is in a volumetric amount of less than 10 times (preferably 5 times or less) the resin solution A, to precipitate a resin solid (step d), and separating the precipitated resin (step e).

Also, in order to keep the resin from aggregation or the like after preparation of the composition, as described, for example, in JP-A-2009-037108, a step of dissolving the synthesized resin in a solvent to make a solution and heating the solution at approximately from 30 to 90° C. for approximately from 30 minutes to 4 hours may be added.

The weight average molecular weight of the resin (A) for use in the present invention is, as described above, 7,000 or more, preferably from 7,000 to 200,000, more preferably from 7,000 to 50,000, still more preferably from 7,000 to 40,000, yet still more preferably from 7,000 to 30,000, in terms of polystyrene by the GPC method. If the weight average molecular weight is less than 7,000, the solubility for an organic developer becomes too high and a precise pattern may not be formed.

The polydispersity (molecular weight distribution) is usually from 1.0 to 3.0, preferably from 1.0 to 2.6, more preferably from 1.0 to 2.0, still more preferably from 1.4 to 2.0. As the molecular weight distribution is smaller, not only the resolution and resist profile are more excellent but also the side wall of the resist pattern is smoother and the roughness is more improved.

In the actinic ray-sensitive or radiation-sensitive resin composition of the present invention, the blending ratio of the resin (A) in the entire composition is preferably from 30 to 99 mass %, more preferably from 60 to 95 mass %, based on the total solid content.

Also, in the present invention, as for the resin (A), one kind may be used or a plurality of kinds may be used in combination. (In this specification, mass ratio is equal to weight ratio.)

### [2] Compounds (C1) and (C2)

The actinic ray-sensitive or radiation-sensitive resin composition for use in the present invention contains at least one compound of the compound (C1) and he compound (C2). The compound is not particularly limited as long as the actinic ray-sensitive or radiation-sensitive resin composition of the present invention contains at least one compound of the compound (C1) and the compound (C2), but preferably, the compound (C1) or the compound (C2) has an onium salt structure.

The compounds (C1) and (C2) are described in detail below.

### [2-1] Compound (C1)

The compound (C1) is, as described above, a compound containing a group capable of generating a first acidic functional group upon irradiation with an actinic ray or radiation (hereinafter, sometimes referred to as “group capable of generating a first acidic functional group”) and a group capable of generating a second acidic functional group different from the first acidic functional group upon irradiation with an actinic ray or radiation (hereinafter, sometimes referred to as “group capable of generating a second acidic functional group”).

The compound (C1) may be an ionic compound or a nonionic compound as long as it contains a group capable of generating a first acidic functional group upon irradiation with an actinic ray or radiation and a group capable of generating a second acidic functional group upon irradiation with an actinic ray or radiation, but the compound is preferably an ionic compound.

Also, as long as the compound (C1) contains a group capable of generating a first acidic functional group upon irradiation with an actinic ray or radiation and a group capable of generating a second acidic functional group upon irradiation with an actinic ray or radiation, the compound may further contain the same group as the group capable of generating a first acidic functional group or the group capable of generating a second acidic functional group and may further contain a group capable of generating a group different from the first acidic functional group and the second acidic functional group upon irradiation with an actinic ray or radiation.

In the case where the compound (C1) is an ionic compound, the compound (C1) preferably has, as the anion structure, an acid anion structure formed by desorbing a proton (removing a proton) from an acidic functional group generated by the compound (C1).

The compound (C1) is preferably a compound capable of generating, as the first acidic functional group and the second acidic functional group, groups different from each other selected from the group consisting of groups represented by the following formulae (Ca-1) to (Ca-19), upon irradiation with an actinic ray or radiation.

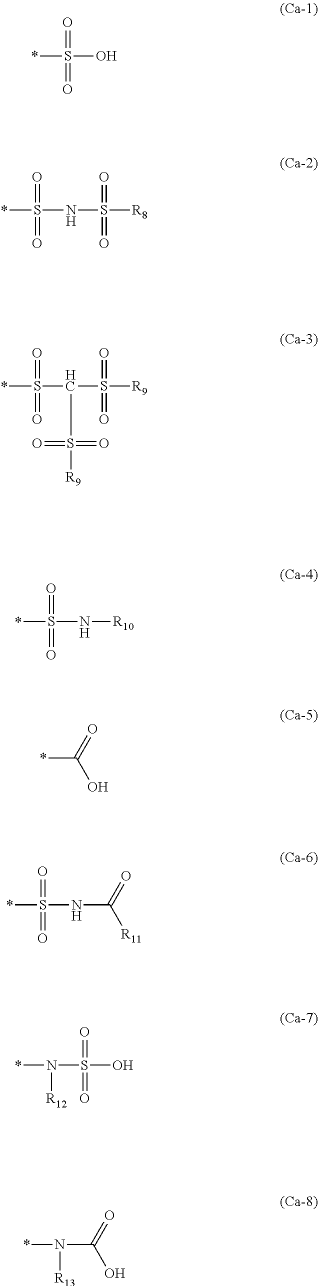

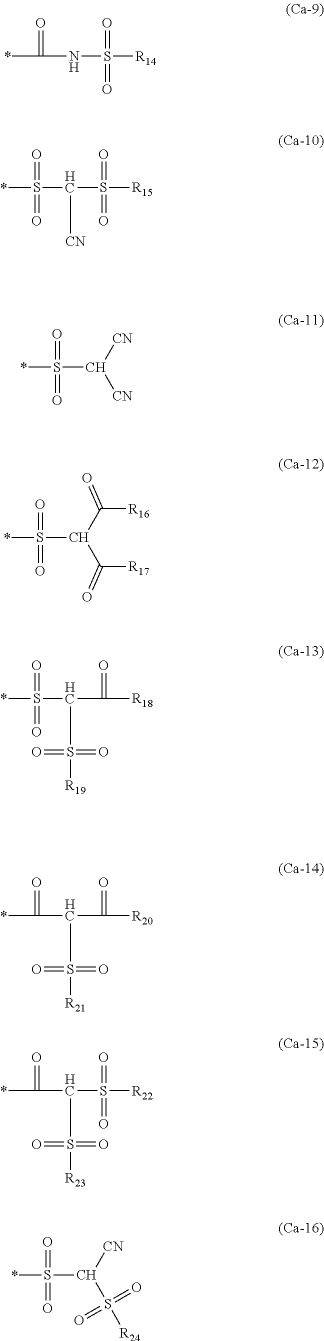

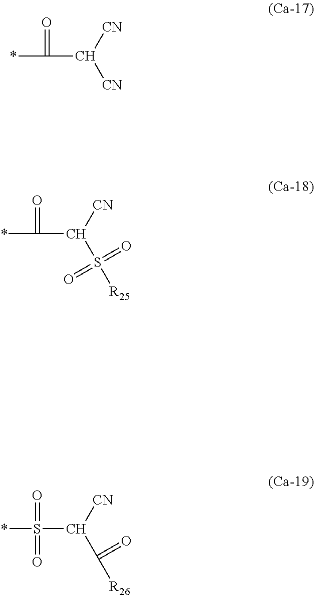

In formulae (Ca-2) to (Ca-4), (Ca-6) to (Ca-10), (Ca-12) to (Ca-16), (Ca-18) and (Ca-19), each of R₈, R₉, R₁₁ and R₁₄ to R₂₆ independently represents an alkyl group, a cycloalkyl group or an aryl group. R₁₀ represents a hydrogen atom, an alkyl group, a cycloalkyl group or an aryl group. Each of R₁₂ and R₁₃ independently represents a hydrogen atom, an alkyl group, an aryl group, or a single bond, alkylene group or arylene group capable of bonding to any one atom in the molecule to form a ring.

The alkyl group as R₈, R₉, R₁₁ and R₁₄ to R₂₆ is preferably a linear or branched alkyl group having a carbon number of 1 to 20 and may contain an oxygen atom, a sulfur atom or a nitrogen atom in the alkyl chain. The alkyl group specifically includes a linear alkyl group such as methyl group, ethyl group, n-propyl group, n-butyl group, n-pentyl group, n-hexyl group, n-octyl group, n-dodecyl group, n-tetradecyl group and n-octadecyl group, and a branched alkyl group such as isopropyl group, isobutyl group, tert-butyl group, neopentyl group and 2-ethylhexyl group. The alkyl group of R₈, R₉, R₁₁ and R₁₄ to R₂₆ may have a substituent, and examples of the alkyl group having a substituent include a cyanomethyl group, a 2,2,2-trifluoroethyl group, a methoxycarbonylmethyl group, and an ethoxycarbonylmethyl group.

The cycloalkyl group as R₈, R₉, R₁₁ and R₁₄ to R₂₆ is preferably a cycloalkyl group having a carbon number of 3 to 20, and the cycloalkyl group may contain an oxygen atom or a sulfur atom in the ring. Specific examples of the cycloalkyl group include a cyclopropyl group, a cyclopentyl group, a cyclohexyl group, a norbornyl group, and an adamantyl group.

The cycloalkyl group of R₈, R₉, R₁₁ and R₁₄ to R₂₆ may have a substituent, and examples of the substituent include an alkyl group and an alkoxy group.

The aryl group as R₈, R₉, R₁₁ and R₁₄ to R₂₆ is preferably an aryl group having a carbon number of 6 to 14, and examples thereof include a phenyl group, a naphthyl group, and a biphenyl group. The aryl group of R₈, R₉, R₁₁ and R₁₄ to R₂₆ may have a substituent, and preferred substituents include an alkyl group, a cycloalkyl group, an alkoxy group, a cycloalkoxy group, an aryloxy group, an alkylthio group, and an arylthio group.

Each of R₈, R₉, R₁₁ and R₁₄ to R₂₆ independently represents preferably a linear or branched alkyl group having a carbon number of 1 to 10, more preferably a linear or branched alkyl group having a carbon number of 1 to 8. The linear or branched alkyl group of R₈, R₉, R₁₁ and R₁₄ to R₂₆ preferably has a fluorine atom as a substituent.

Specific examples and preferred ranges of the alkyl group, cycloalkyl group and aryl group represented by R₁₀ are the same as those of the alkyl group, cycloalkyl group and aryl group of R₈, R₉, R₁₁ and R₁₄ to R₂₆.

R₁₀ preferably represents a linear or branched alkyl group having a carbon number of 1 to 10, a cyclohexyl group or an adamantyl group, more preferably a linear or branched alkyl group having a carbon number of 1 to 8.

Specific examples and preferred ranges of the alkyl group and aryl group represented by R₁₂ and R₁₃ are the same as those of the alkyl group and aryl group of R₈, R₉, R₁₁ and R₁₄ to R₂₆.

Specific examples and preferred ranges of the alkylene group and arylene group in the alkylene group and arylene group capable of bonding to any one atom in the molecule to form a ring, represented by R₁₂ and R₁₃, include those formed by removing one arbitrary hydrogen atom from the above-described alkyl group and aryl group as R₈, R₉, R₁₁ and R₁₄ to R₂₆.

Each of R₁₂ and R₁₃ independently represents preferably a linear or branched alkyl group having a carbon number of 1 to 10, more preferably a linear or branched alkyl group having a carbon number of 1 to 8.

The compound (C1) is more preferably a compound capable of generating a group selected from the group consisting of groups represented by the following formulae (Cb-1) to (Cb-4), upon irradiation with an actinic ray or radiation.

Also, the compound (C1) is still more preferably a compound containing a group capable of generating a group selected from the group consisting of groups represented by the following formulae (Cb-1) to (Cb-4) and a group capable of generating a group selected from the group consisting of groups represented by the following formulae (Cc-1) to (Cc-4), upon irradiation with an actinic ray or radiation.

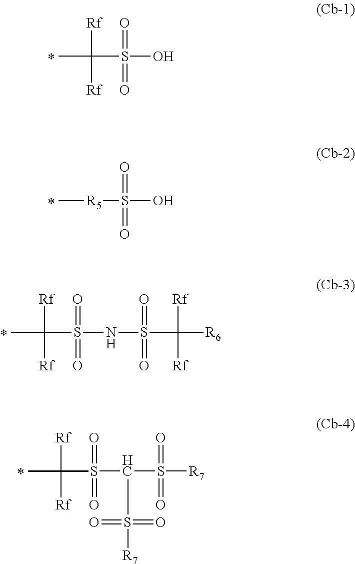

In formulae (Cb-1) to (Cb-4), each Rf independently represents a fluorine atom or an alkyl group substituted with at least one fluorine atom, R₅ represents an arylene group containing a fluorine atom or an alkyl group substituted with at least one fluorine atom, R₆ represents a hydrogen atom, a fluorine atom or an alkyl group, each R₇ independently represents an alkyl group, a cycloalkyl group or an aryl group, and * represents a bond.

Specific examples and preferred range of the alkyl group in the alkyl group substituted with at least one fluorine atom as Rf include those of the alkyl group as R₈, R₉, R₁₁ and R₁₄ to R₂₆.

Rf preferably represents a fluorine atom.

Specific examples and preferred range of the alkyl group in the arylene group containing an alkyl group substituted with at least one fluorine atom as R₅ include those of the alkyl group as R₈, R₉, R₁₁ and R₁₄ to R₂₆.

Specific examples and preferred range of the arylene group in the arylene group containing an alkyl group substituted with at least one fluorine atom as R₅ include those formed by removing one arbitrary hydrogen atom from the above-described aryl group as R₈, R₉, R₁₁ and R₁₄ to R₂₆.

R₅ is preferably a perfluorophenylene group.

Specific examples and preferred range of the alkyl group as R₆ include those of the alkyl group of R₈, R₉, R₁₁ and R₁₄ to R₂₆.

R₆ is preferably a fluorine atom or a perfluoroalkyl group.

Specific examples and preferred ranges of the alkyl group, cycloalkyl group and aryl group as R₇ are the same as those of the alkyl group, cycloalkyl group and aryl group of R₈, R₉, R₁₁ and R₁₄ to R₂₆.

R₇ is preferably a perfluoroalkyl group such as trifluoromethyl group, more preferably a trifluoromethyl group.

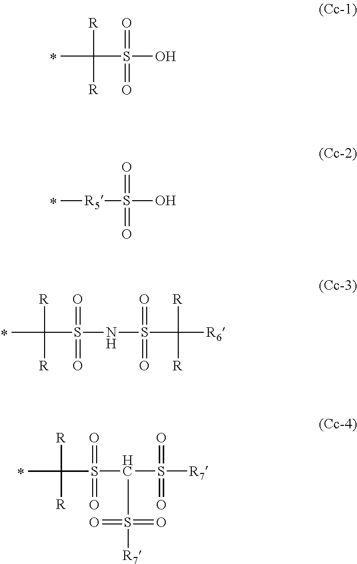

In formulae (Cc-1) to (Cc-4), each R independently represents an unsubstituted alkyl group, R₅′ represents an unsubstituted arylene group, R₆′ represents a hydrogen atom, a fluorine atom or an alkyl group, each R₇′ independently represents an alkyl group, a cycloalkyl group or an aryl group, and * represents a bond.

In formulae (Cc-1) to (Cc-4), the unsubstituted alkyl group as R is preferably a linear or branched alkyl group having a carbon number of 1 to 20 and may contain an oxygen atom, a sulfur atom or a nitrogen atom in the alkyl chain. The alkyl group specifically includes a linear alkyl group such as methyl group, ethyl group, n-propyl group, n-butyl group, n-pentyl group, n-hexyl group, n-octyl group, n-dodecyl group, n-tetradecyl group and n-octadecyl group, and a branched alkyl group such as isopropyl group, isobutyl group, tert-butyl group, neopentyl group and 2-ethylhexyl group.

In formulae (Cc-1) to (Cc-4), specific examples and preferred ranges of the arylene group, alkyl group, cycloalkyl group and aryl group of R₅′, R₆′ and R₇′ are the same as those recited respectively for the arylene group, alkyl group, cycloalkyl group and aryl group of R₅, R₆ and R₇ in formulae (Cb-1) to (Cb-4).

Specific examples of the anion structure in the group capable of generating a first acidic functional group and the group capable of generating a second acidic functional group when the compound (C1) is an ionic compound are illustrated below, but the scope of the present invention is not limited thereto.

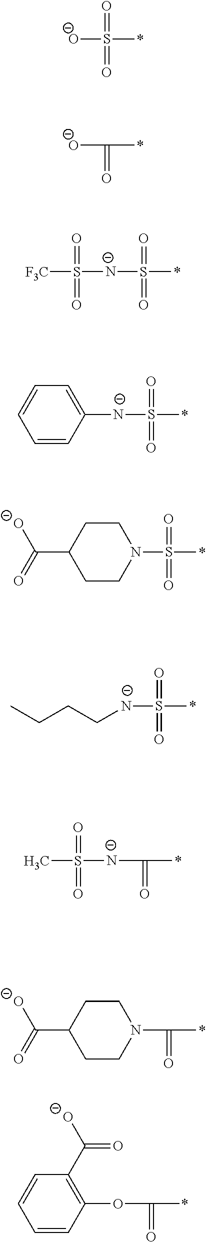

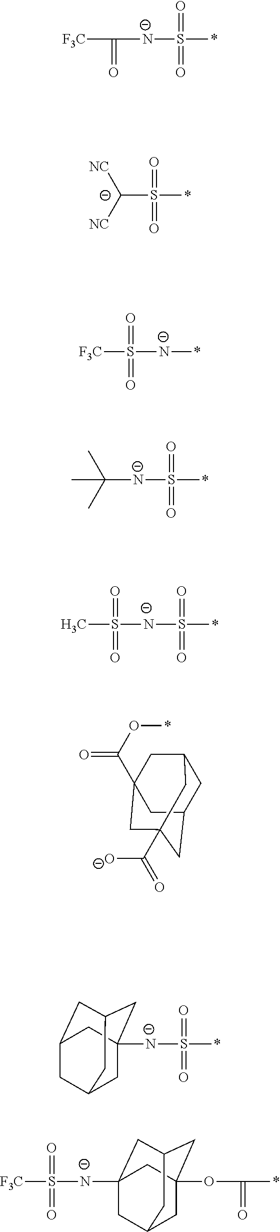

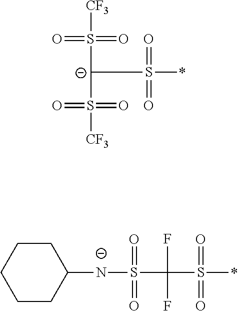 [SMILES: *S(=O)(=O)[C-](S(=O)(=O)C(F)(F)F)S(=O)(=O)C(F)(F)F ; *S(=O)(=O)C(F)(F)S(=O)(=O)[N-]C1CCCCC1]

In the compound (C1) for use in the present invention, the first acidic functional group and the second acidic functional group are different from each other, and the term “different from each other” encompasses a case where specific groups are different, for example, encompasses a case where both the first acidic functional group and the second acidic functional group in the compound (C1) have a structure represented by formula (Ca-2) but R₈ in formula (Ca-2) is different between those functional groups.

In the case where the compound (C1) is an ionic compound, the cation of the compound (C1) includes, for example, a cation represented by the following formula (ZI) or (ZII):

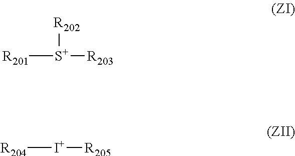

In formula (ZI), each of R₂₀₁, R₂₀₂ and R₂₀₃ independently represents an organic group.

The carbon number of the organic group as R₂₀₁, R₂₀₂ and R₂₀₃ is generally from 1 to 30, preferably from 1 to 20.

Two members out of R₂₀₁ to R₂₀₃ may combine to form a ring structure, and the ring may contain an oxygen atom, a sulfur atom, an ester bond, an amide bond or a carbonyl group. The group formed by combining two members out of R₂₀₁ to R₂₀₃ includes an alkylene group (such as butylene group and pentylene group).

The cation may be a cation having a plurality of structures represented by formula (ZI). For example, the cation may be a cation having a structure where at least one of R₂₀₁ to R₂₀₃ in a compound represented by formula (ZI) is bonded to at least one of R₂₀₁ to R₂₀₃ in another cation represented by formula (ZI) through a single bond or a linking group.

The organic group of R₂₀₁, R₂₀₂ and R₂₀₃ includes an aryl group (preferably having a carbon number of 6 to 15), a linear or branched alkyl group (preferably having a carbon number of 1 to 10), a cycloalkyl group (preferably having a carbon number of 3 to 15), and the like.

At least one of R₂₀₁, R₂₀₂ and R₂₀₃ is preferably an aryl group, and it is more preferred that those three members all are an aryl group. The aryl group may be a heteroaryl group such as indole residue and pyrrole residue, other than a phenyl group, a naphthyl group or the like.

The aryl group, alkyl group and cycloalkyl group as R₂₀₁, R₂₀₂ and R₂₀₃ may further have a substituent. Examples of the substituent include, but are not limited to, a nitro group, a halogen atom such as fluorine atom, a carboxyl group, a hydroxyl group, an amino group, a cyano group, an alkoxy group (preferably having a carbon number of 1 to 15), a cycloalkyl group (preferably having a carbon number of 3 to 15), an aryl group (preferably having a carbon number of 6 to 14), an alkoxycarbonyl group (preferably having a carbon number of 2 to 7), an acyl group (preferably having a carbon number of 2 to 12), and an alkoxycarbonyloxy group (preferably having a carbon number of 2 to 7).

Also, two members selected from R₂₀₁, R₂₀₂ and R₂₀₃ may combine through a single bond or a linking group. Examples of the linking group include, but are not limited to, an alkylene group (preferably having a carbon number of 1 to 3), —O—, —S—, —CO— and —SO₂—.

Preferred structures where at least one of R₂₀₂, R₂₀₂ and R₂₀₃ is not an aryl group include cation structures such as compounds illustrated in paragraphs 0046 and 0047 of JP-A-2004-233661 and paragraphs 0040 to 0046 of JP-A-2003-35948, compounds illustrated as formulae (I-1) to (I-70) in U.S. Patent Application Publication No. 2003/0224288A1, and compounds illustrated as formulae (IA-1) to (IA-54) and formulae (IB-1) to (IB-24) in U.S. Patent Application Publication No. 2003/0077540A1.

More preferred examples of the cation represented by formula (ZI) include a cation represented by formula (ZI-3) or (ZI-4) described below. First, the cation represented by formula (ZI-3) is described.

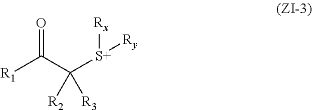

In formula (ZI-3),

R₁ represents an alkyl group, a cycloalkyl group, an alkoxy group, a cycloalkoxy group, an aryl group or an alkenyl group,

each of R₂ and R₃ independently represents a hydrogen atom, an alkyl group, a cycloalkyl group or an aryl group, and R₂ and R₃ may combine with each other to form a ring,

R₁ and R₂ may combine with each other to form a ring, and

each of R, and R, independently represents an alkyl group, a cycloalkyl group, an alkenyl group, an aryl group, a 2-oxoalkyl group, a 2-oxocycloalkyl group, an alkoxycarbonylalkyl group or an alkoxycarbonylcycloalkyl group, Rₓ and R_{y} may combine with each other to form a ring structure, and this ring structure may contain an oxygen atom, a nitrogen atom, a sulfur atom, a ketone group, an ether bond, an ester bond or an amide bond.

The alkyl group as R₁ is preferably a linear or branched alkyl group having a carbon number of 1 to 20 and may contain an oxygen atom, a sulfur atom or a nitrogen atom in the alkyl chain. The alkyl group specifically includes a linear alkyl group such as methyl group, ethyl group, n-propyl group, n-butyl group, n-pentyl group, n-hexyl group, n-octyl group, n-dodecyl group, n-tetradecyl group and n-octadecyl group, and a branched alkyl group such as isopropyl group, isobutyl group, tert-butyl group, neopentyl group and 2-ethylhexyl group. The alkyl group of R₁ may have a substituent, and examples of the alkyl group having a substituent include a cyanomethyl group, a 2,2,2-trifluoroethyl group, a methoxycarbonylmethyl group, and an ethoxycarbonylmethyl group.

The cycloalkyl group as R, is preferably a cycloalkyl group having a carbon number of 3 to 20 and may contain an oxygen atom or a sulfur atom in the ring. Specific examples of the cycloalkyl group include a cyclopropyl group, a cyclopentyl group, a cyclohexyl group, a norbornyl group, and an adamantyl group. The cycloalkyl group of R₁ may have a substituent, and examples of the substituent include an alkyl group and an alkoxy group.

The alkoxy group as R₁ is preferably an alkoxy group having a carbon number of 1 to 20 and specifically includes a methoxy group, an ethoxy group, an isopropyloxy group, a tert-butyloxy group, a tert-amyloxy group, and an n-butyloxy group. The alkoxy group of R₁ may have a substituent, and examples of the substituent include an alkyl group and a cycloalkyl group.

The cycloalkoxy group as R₁ is preferably a cycloalkoxy group having a carbon number of 3 to 20, and examples thereof include a cyclohexyloxy group, a norbomyloxy group, and an adamantyloxy group. The cycloalkoxy group of R₁ may have a substituent, and examples of the substituent include an alkyl group and a cycloalkyl group.

The aryl group as R₁ is preferably an aryl group having a carbon number of 6 to 14, and examples thereof include a phenyl group, a naphthyl group, and a biphenyl group. The aryl group of R₁ may have a substituent, and preferred substituents include an alkyl group, a cycloalkyl group, an alkoxy group, a cycloalkoxy group, an aryloxy group, an alkylthio group, and an arylthio group. In the case where the substituent is an alkyl group, a cycloalkyl group, an alkoxy group or a cycloalkoxy group, examples of these groups are the same as those of the alkyl group, cycloalkyl group, alkoxy group and cycloalkoxy group of R₁.

The alkenyl group as R₁ include a vinyl group and an allyl group.

Each of R₂ and R₃ represents a hydrogen atom, an alkyl group, a cycloalkyl group or an aryl group, and R₂ and R₃ may combine with each other to form a ring. However, at least one of R₂ and R₃ represents an alkyl group, a cycloalkyl group or an aryl group. Specific examples and preferred examples of the alkyl group, cycloalkyl group and aryl group of R₂ and R₃ are the same as specific examples and preferred examples described for R₁. In the case where R₂ and R₃ combine with each other to form a ring, the total number of carbon atoms contributing to the formation of a ring, contained in R₂ and R₃, is preferably from 4 to 7, more preferably 4 or 5.

R₁ and R₂ may combine with each other to form a ring. In the case where R₁ and R₂ combine with each other to form a ring, it is preferred that R₁ is an aryl group (preferably a phenyl or naphthyl group which may have a substituent) and R₂ is an alkylene group having a carbon number of 1 to 4 (preferably a methylene group or an ethylene group), and preferred substituents are the same as those of the substituent which may be substituted on an aryl group as R₁. In another preferred embodiment when R₁ and R₂ combine with each other to form a ring, R₁ is a vinyl group and R₂ is an alkylene group having a carbon number of 1 to 4.

The alkyl group represented by Rₓ and R_{y} is preferably an alkyl group having a carbon number of 1 to 15, and examples thereof include a methyl group, an ethyl group, a propyl group, an isopropyl group, an n-butyl group, an isobutyl group, a sec-butyl group, a pentyl group, a neopentyl group, a hexyl group, a heptyl group, an octyl group, a nonyl group, a decyl group, a undecyl group, a dodecyl group, a tridecyl group, a tetradecyl group, a pentadecyl group, a hexadecyl group, a heptadecyl group, an octadecyl group, a nonadecyl group, and an eicosyl group.

The cycloalkyl group represented by Rₓ and R_{y} is preferably a cycloalkyl group having a carbon number of 3 to 20, and examples thereof include a cyclopropyl group, a cyclopentyl group, a cyclohexyl group, a norbornyl group and an adamantyl group.

The alkenyl group represented by Rₓ and R_{y} is preferably an alkenyl group having a carbon number of 2 to 30, and examples thereof include a vinyl group, an allyl group and a styryl group.

The aryl group represented by Rₓ and R_{y} is preferably, for example, an aryl group having a carbon number of 6 to 20, and specific examples thereof include a phenyl group, a naphthyl group, an azulenyl group, an acenaphthylenyl group, a phenanthrenyl group, a penalenyl group, a phenanthracenyl group, a fluorenyl group, an anthracenyl group, a pyrenyl group, and a benzopyrenyl group. A phenyl group and a naphthyl group are preferred, and a phenyl group is more preferred.

The alkyl group moiety in the 2-oxoalkyl group and alkoxycarbonylalkyl group represented by Rₓ and R_{y} includes, for example, those enumerated above for Rₓ and R_{y}.

The cycloalkyl group moiety in the 2-oxocycloalkyl group and alkoxycarbonylcycloalkyl group represented by Rₓ and R_{y} includes, for example, those enumerated above for Rₓ and R_{y}.

The cation represented by formula (ZI-3) is preferably a cation represented by the following formula (ZI-3a) or (ZI-3b):

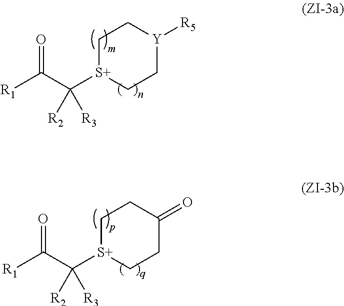

In formulae (ZI-3a) and (ZI-3b), R₁, R₂ and R₃ are as defined above in formula (ZI-3).

Y represents an oxygen atom, a sulfur atom or a nitrogen atom and is preferably an oxygen atom or a nitrogen atom. Each of m, n, p and q represents an integer and is preferably from 0 to 3, more preferably from 1 to 2, still more preferably 1. The alkylene group connecting S⁺ and Y may have a substituent, and preferred substituents include an alkyl group.

R₅ represents a monovalent organic group when Y is a nitrogen atom, and is not present when Y is an oxygen atom or a sulfur atom. R₅ is preferably a group containing an electron-withdrawing group, more preferably a group represented by the following formulae (ZI-3a-1) to (Z-3a-4):

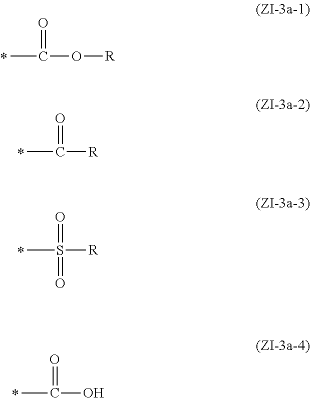

In formulae (ZI-3a-1) to (ZI-3a-3), R represents a hydrogen atom, an alkyl group, a cycloalkyl group or an aryl group and is preferably an alkyl group. Specific examples and preferred examples of the alkyl group, cycloalkyl group and aryl group of R are the same as specific examples and preferred examples described above for R₁ in formula (ZI-3).

In (ZI-3a-1) to (ZI-3a-4), * represents a bond connected to the nitrogen atom as Y in the compound represented by formula (ZI-3a).

when Y is a nitrogen atom, R₅ is preferably a group represented by —SO₂—R₄. R₄ represents an alkyl group, a cycloalkyl group or an aryl group and is preferably an alkyl group. Specific examples and preferred examples of the alkyl group, cycloalkyl group and aryl group of R₄ are the same as specific examples and preferred examples described above for R₁.

The compound represented by formula (ZI-3) is more preferably a compound represented by the following formula (ZI-3a′) or (ZI-3b′):

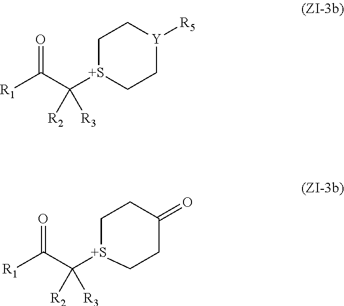

In formulae (ZI-3a′) and (ZI-3b′), R₁, R₂, R₃, Y and R₅ are as defined in formulae (ZI-3a) and (ZI-3b).

Specific examples of the cation represented by formula (ZI-3) are illustrated below.

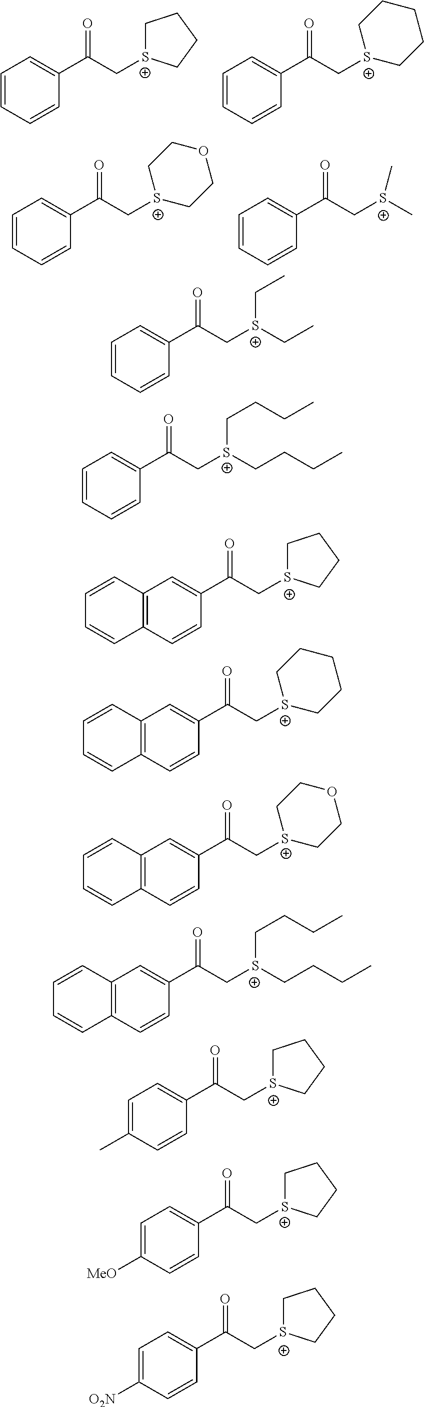

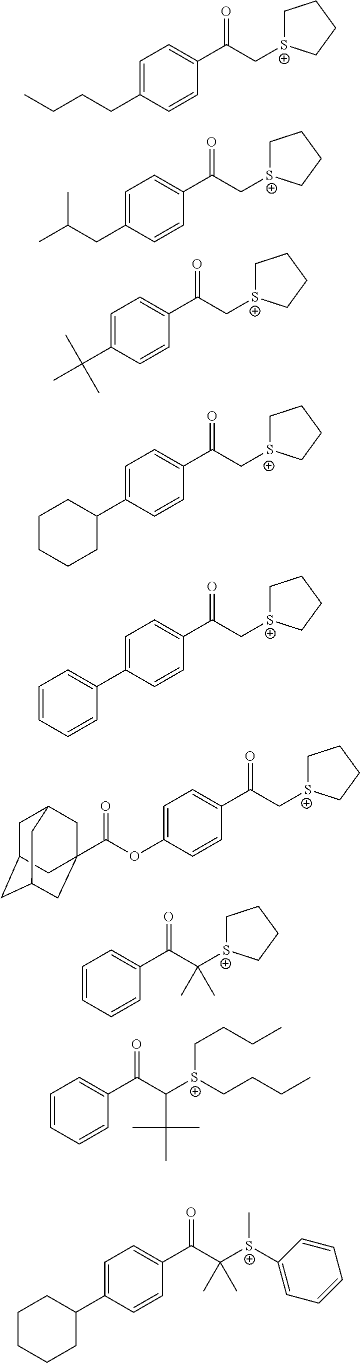

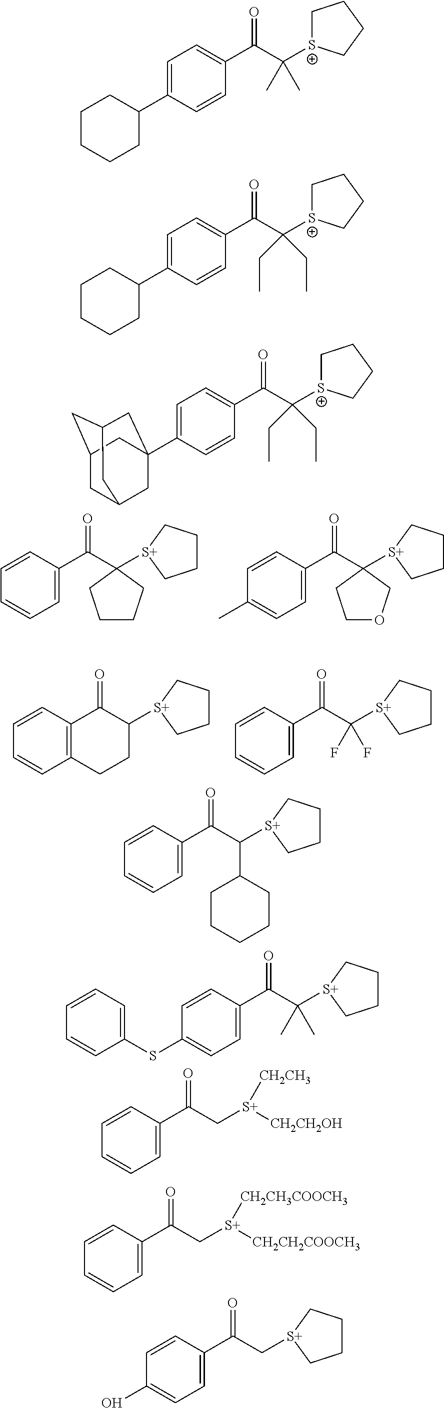

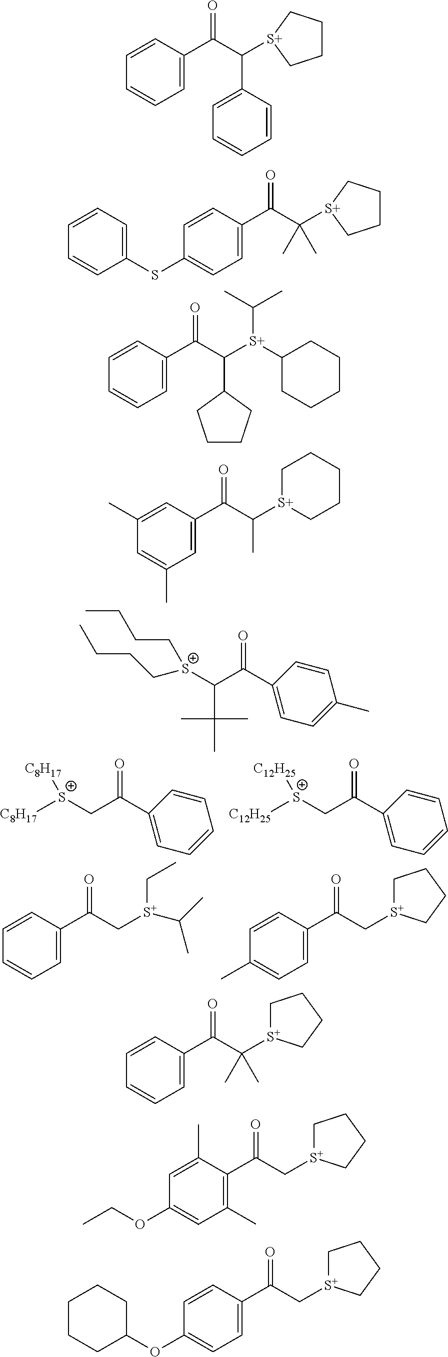

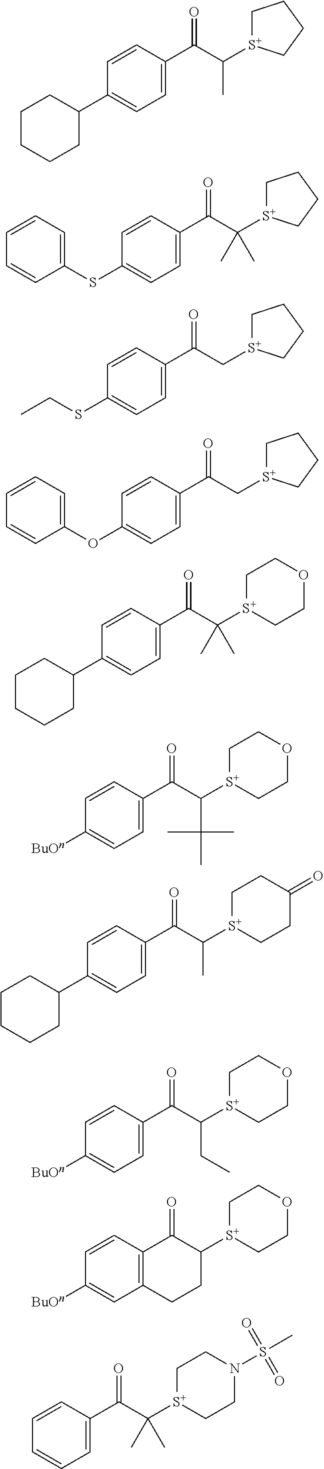

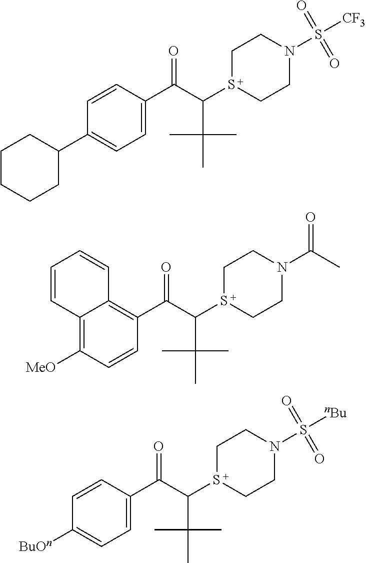

The cation represented by formula (ZI-4) is described below.

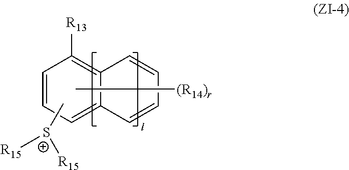

In formula (ZI-4), R₁₃ represents a hydrogen atom, a fluorine atom, a hydroxyl group, an alkyl group, a cycloalkyl group, an alkoxy group, an alkoxycarbonyl group or a group having a cycloalkyl group. These groups may have a substituent.

R₁₄ represents, when a plurality of R₁₄ are present, each independently represents, a hydroxyl group, an alkyl group, a cycloalkyl group, an alkoxy group, an alkoxycarbonyl group, an alkylcarbonyl group, an alkylsulfonyl group, a cycloalkylsulfonyl group or a group having a cycloalkyl group. These groups may have a substituent.

Each R₁₅ independently represents an alkyl group, a cycloalkyl group or a naphthyl group. Two R₁₅ may combine with each other to form a ring, and the ring may contain, as an atom constituting the ring, a heteroatom such as oxygen atom, sulfur atom and nitrogen atom. The groups above may have a substituent.

l represents an integer of 0 to 2.

r represents an integer of 0 to 8.

In formula (ZI-4), the alkyl group of R₁₃, R₁₄ and R₁₅ is linear or branched and is preferably an alkyl group having a carbon number of 1 to 10.

The cycloalkyl group of R₁₃, R₁₄ and R₁₅ includes a monocyclic or polycyclic cycloalkyl group.

The alkoxy group of R₁₃ and R₁₄ is linear or branched and is preferably an alkoxy group having a carbon number of 1 to 10.

The alkoxycarbonyl group of R₁₃ and R₁₄ is linear or branched and is preferably an alkoxycarbonyl group having a carbon number of 2 to 11.

The group having a cycloalkyl group of R₁₃ and R₁₄ includes a group having a monocyclic or polycyclic cycloalkyl group. These groups may further have a substituent.

Specific examples of the alkyl group in the alkylcarbonyl group of R₁₄ are the same as those described for the alkyl group of R₁₃ to R₁₅.

The alkylsulfonyl or cycloalkylsulfonyl group of R₁₄ is linear, branched or cyclic and is preferably an alkylsulfonyl or cycloalkylsulfonyl group having a carbon number of 1 to 10.

Examples of the substituent which may be substituted on each of the groups above include a halogen atom (e.g., fluorine atom), a hydroxyl group, a carboxyl group, a cyano group, a nitro group, an alkoxy group, an alkoxyalkyl group, an alkoxycarbonyl group, and an alkoxycarbonyloxy group.

The ring structure which may be formed by combining two R₁₅ with each other includes a 5- or 6-membered ring, preferably a 5-membered ring (that is, a tetrahydrothiophene ring or a 2,5-dihydrothiophene ring), formed by two R₁₅ together with the sulfur atom in formula (ZI-4) and may be fused with an aryl group or a cycloalkyl group. The divalent R₁₅ may have a substituent, and examples of the substituent include a hydroxyl group, a carboxyl group, a cyano group, a nitro group, an alkyl group, a cycloalkyl group, an alkoxy group, an alkoxyalkyl group, an alkoxycarbonyl group, and an alkoxycarbonyloxy group. As for the substituent on the ring structure, a plurality of substituents may be present, and these substituents may combine with each other to form a ring.

In formula (ZI-4), R₁₅ is preferably, for example, a methyl group, an ethyl group, a naphthyl group, or a divalent group capable of forming a tetrahydrothiophene ring structure together with the sulfur atom when two R₁₅ are combined with each other, more preferably a divalent group capable of forming a tetrahydrothiophene ring structure together with the sulfur atom when two R₁₅ are combined with each other.

The substituent which may be substituted on R₁₃ and R₁₄ is preferably a hydroxyl group, an alkoxy group, an alkoxycarbonyl group, or a halogen atom (particularly fluorine atom).

l is preferably 0 or 1, more preferably 1.

r is preferably from 0 to 2.

Specific examples of the cation structure in the cation represented by formula (ZI-3) or (ZI-4) include cation structures in the chemical structures illustrated in paragraphs 0046, 0047, 0072 to 0077 and 0107 to 0110 of JP-A-2011-53360 and cation structures in the chemical structures illustrated in paragraphs 0135 to 0137, 0151 and 0196 to 0199 of JP-A-2011-53430, in addition to the above-described cation structures such as compounds illustrated in JP-A-2004-233661, JP-A-2003-35948 and U.S. Patent Application Publication Nos. 2003/0224288A and 2003/0077540A1.

In formula (ZII), each of R₂₀₄ and R₂₀₅ independently represents an aryl group, an alkyl group or a cycloalkyl group.

Examples of the aryl group, alkyl group and cycloalkyl group of R₂₀₄ and R₂₀₅ are the same as those described above for the aryl group, alkyl group and cycloalkyl group of R₂₀₁ to R₂₀₃ in the compound (ZI).

The aryl group, alkyl group and cycloalkyl group of R₂₀₄ and R₂₀₅ may have a substituent. Examples of the substituent are also the same as those of the substituent which may be substituted on the aryl group, alkyl group and cycloalkyl group of R₂₀₁ to R₂₀₃ in the compound (ZI).

Other than the cation represented by formula (ZI-3) or (ZI-4), a cation represented by the following formula (I′) is also preferred as the acid generator. By virtue of using the cation represented by the following formula (I′), the transparency to exposure light is enhanced and LWR and DOF are improved.

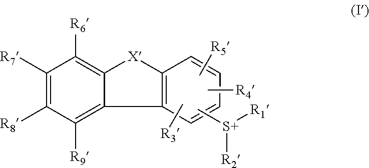

In formula (I′), X′ represents an oxygen atom, a sulfur atom or —N(Rx)-.

Each of R₁′ and R₂′ independently represents an alkyl group, a cycloalkyl group or an aryl group.

Each of R₃′ to R₉′ independently represents a hydrogen atom, an alkyl group, a cycloalkyl group, an alkoxy group, an alkoxycarbonyl group, an acyl group, an alkylcarbonyloxy group, an aryl group, an aryloxy group, an aryloxycarbonyl group or an arylcarbonyloxy group.

Rx represents a hydrogen atom, an alkyl group, a cycloalkyl group, an acyl group, an alkenyl group, an alkoxycarbonyl group, an aryl group, an arylcarbonyl group or an aryloxycarbonyl group.

R₁′ and R₂′ may combine with each other to form a ring. Also, any two or more members out of R₆′ to R₉′, a pair of R₃′ and R₉′, a pair of R₄′ and R₅′, a pair of R₅′ and Rₓ, or a pair of R₆′ and Rₓ may combine with each other to form a ring.

X′ is preferably a sulfur atom or —N(Rx)- from the standpoint of keeping the absorbancy (for example, absorbance at a wavelength of 193 nm) low.

The alkyl group as R₁′ to R₉′ and Rx may have a substituent and is preferably a linear or branched alkyl group having a carbon number of 1 to 20, and the alkyl group may contain an oxygen atom, a sulfur atom or a nitrogen atom in the alkyl chain. The alkyl group specifically includes a linear alkyl group such as methyl group, ethyl group, n-propyl group, n-butyl group, n-pentyl group, n-hexyl group, n-octyl group, n-dodecyl group, n-tetradecyl group and n-octadecyl group, and a branched alkyl group such as isopropyl group, isobutyl group, tert-butyl group, neopentyl group and 2-ethylhexyl group.

Examples of the alkyl group having a substituent for Rx include a cyanomethyl group, a 2,2,2-trifluoroethyl group, a methoxycarbonylmethyl group, and an ethoxycarbonylmethyl group.

Examples of the alkyl group having a substituent for R₁′ and R₂′ include a methoxyethyl group.

Other examples include a group formed by substituting a cycloalkyl group on a linear or branched alkyl group (for example, an adamantylmethyl group, an adamantylethyl group, a cyclohexylethyl group and a camphor residue).

The cycloalkyl group as R₁′ to R₉′ and Rx may have a substituent and is preferably a cycloalkyl group having a carbon number of 3 to 20, and the cycloalkyl group may contain an oxygen atom in the ring. Specific examples thereof include a cyclopropyl group, a cyclopentyl group, a cyclohexyl group, a norbornyl group and an adamantyl group.

The acyl group as R₃′ to R₉′ and Rx may have a substituent and is preferably an acyl group having a carbon number of 1 to 10. Specific examples thereof include an acetyl group, a propionyl group, and an isobutyryl group.

The alkenyl group as Rₓ is preferably an alkenyl group having a carbon number of 2 to 8, and examples thereof include a vinyl group, an allyl group, and a butenyl group.

The alkoxy group as R₃′ to R₉′ may have a substituent and is preferably an alkoxy group having a carbon number of 1 to 20. Specific examples thereof include a methoxy group, an ethoxy group, an isopropyloxy group, and a cyclohexyloxy group.

The alkoxycarbonyl group as R₃′ to R₉′ may have a substituent and is preferably an alkoxycarbonyl group having a carbon number of 2 to 20. Specific examples thereof include a methoxycarbonyl group, an ethoxycarbonyl group, an isopropyloxycarbonyl group, and a cyclohexyloxycarbonyl group.

The alkylcarbonyloxy group as R₃′ to R₉′ may have a substituent and is preferably an alkylcarbonyloxy group having a carbon number of 2 to 20. Specific examples thereof include a methylcarbonyloxy group, an ethylcarbonyloxy group, an isopropylcarbonyloxy group, and a cyclohexylcarbonyloxy group.

The aryl group as R₁′ to R₉′ and Rx may have a substituent and is preferably an aryl group having a carbon number of 6 to 14, and examples thereof include a phenyl group and a naphthyl group.

The aryloxy group as R₃′ to R₉′ may have a substituent and is preferably an aryloxy group having a carbon number of 6 to 14, and examples thereof include a phenyloxy group and a naphthyloxy group.

The aryloxycarbonyl group as R₃′ to R₉′ and Rx may have a substituent and is preferably an aryloxycarbonyl group having a carbon number of 7 to 15, and examples thereof include a phenyloxycarbonyl group and a naphthyloxycarbonyl group.

The arylcarbonyloxy group as R₃′ to R₉′ may have a substituent and is preferably an arylcarbonyloxy group having a carbon number of 7 to 15, and examples thereof include a phenylcarbonyloxy group and a naphthylcarbonyloxy group.

The arylcarbonyl group as Rx may have a substituent and is preferably an arylcarbonyl group having a carbon number of 7 to 15, and examples thereof include a phenylcarbonyl group and a naphthylcarbonyl group.

Examples of the substituent which each of the alkyl group as R₃′ to R₉′, the cycloalkyl group as R₁′ to R₉′ and Rₓ, the acyl group as R₃′ to R₉′ and Rₓ, the alkoxy group as R₃′ to R₉′, the alkoxycarbonyl group as R₃′ to R₉′, the alkylcarbonyloxy group as R₃′ to R₉′, the aryl group as R₁′ to R₉′ and Rₓ, the aryloxy group as R₃′ to R₉′, the aryloxycarbonyl group as R₃′ to R₉′ and Rₓ, the arylcarbonyloxy group as R₃′ to R₉′, and the arylcarbonyl group as Rx may further have include an alkyl group (may be linear, branched of cyclic, preferably having a carbon number of 1 to 12), an aryl group (preferably having a carbon number of 6 to 14), a nitro group, a halogen atom such as fluorine atom, a carboxyl group, a hydroxyl group, an amino group, a cyano group, an alkoxy group (preferably having a carbon number of 1 to 15), a cycloalkyl group (preferably having a carbon number of 3 to 15), and an acyl group (preferably having a carbon number of 2 to 12).

The ring structure which may be formed by combining R₁′ and R₂′ with each other includes a 5- or 6-membered ring, preferably a 5-membered ring (that is, a tetrahydrothiophene ring), formed by divalent R₁′ and R₂′ (for example, an ethylene group, a propylene group or a 1,2-cyclohexylene group) together with the sulfur atom in formula (I′). However, in view of decomposition efficiency for generation of an acid anion, R₁′ and R₂′ are preferably not combined with each other to form a ring.

The ring structure which may be formed by combining any two or more members out of R₆′ to R₉′, a pair of R₃′ and R₉′, a pair of R₄′ and R₅′, a pair of R₅′ and Rₓ, or a pair of R₆′ and Rₓ with each other is preferably a 5- or 6-membered ring, more preferably a 6-membered ring.

Each of R₁′ and R₂′ is preferably an alkyl group or an aryl group, among others.

Particularly preferred examples of R₃′ to R₉′ include an alkyl group which may have a substituent, and a hydrogen atom, but in the case of using the composition for an ArF resist, a hydrogen atom is more preferred in view of absorption intensity at 193 nm.

Rₓ is preferably an alkyl group or an acyl group, among others.

The compound (C1) is preferably a compound represented by the following formula (C-1):

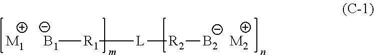

In formula (C-1),

each of M, and M₂ represents an organic counter cation structure,

B₁ represents the acid anion moiety of a first acidic functional group,

B₂ represents the acid anion moiety of a second acidic functional group different from the first acidic functional group,

each of R₁ and R₂ independently represents a single bond, an alkylene group, a cycloalkylene group or an arylene group,

L represents an (m+n)-valent linking group,

each of m and n represents an integer, and m≧n.

The first acidic functional group in the acid anion moiety of a first acidic functional group, as B₁ has the same meaning as the first acidic functional group in the compound (C1) above, and specific examples and preferred range are also the same.

The second acidic functional group in the acid anion moiety of a second acidic functional group different from the first acidic functional group, as B₂ has the same meaning as the second acidic functional group in the compound (C1) above.

Specific examples and preferred range of the organic counter cation structure as M₁ and M₂ are the same as those for the cation of the compound (C1) when the compound (C1) is an ionic compound.

Specific examples and preferred examples of the alkylene group, cycloalkylene group and arylene group as R₁ and R₂ include those formed by removing one arbitrary hydrogen atom from the above-described specific examples and preferred examples of the alkyl group, cycloalkyl group and aryl group as R₈, R₉, R₁₁ and R₁₄ to R₂₆. The alkylene group, cycloalkylene group and arylene group as R₁ and R₂ may have a substituent, and the substituent includes a halogen atom (preferably a fluorine atom).

m represents an integer and represents an integer of 1 to 4, preferably 1 or 2, more preferably 1.

n represents an integer and preferably represents an integer of 1 to 3, more preferably 1.

As for the (m+n)-valent linking groups represented by L, the divalent linking group when each of m and n is 1 includes, for example, a single bond, —COO—, —OCO—, —CO—, —O—, —S—, —SO—, —SO₂—, an alkylene group, a cycloalkylene group, an alkenylene group, and a group formed by combining two or more thereof.

The linking group when m+n is 3 or more includes the groups formed by removing (m+n-2) arbitrary hydrogen atoms from the above-described divalent linking groups except for single bond.

In formula (C-1), when m+n is 2, L is preferably a single bond or a divalent group represented by any one of the following formulae (L1) to (L6). Also, it is preferred that m+n is 3 and L represents a trivalent formula represented by any one of the following formulae (L7) to (L9).

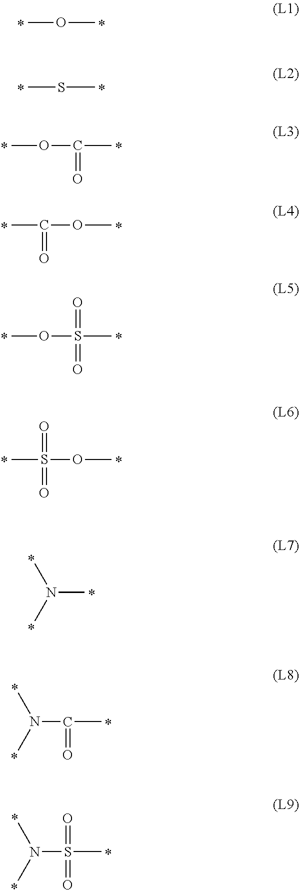

In the formulae above, * represents a bond.

m+n is preferably 2 or 3, m+n is more preferably 2, and it is still more preferred that m+n is 2 and L is a group represented by formula (L5) or (L9).

Specific examples of the compound represented by formula (C1) are illustrated below, but the present invention is not limited thereto.

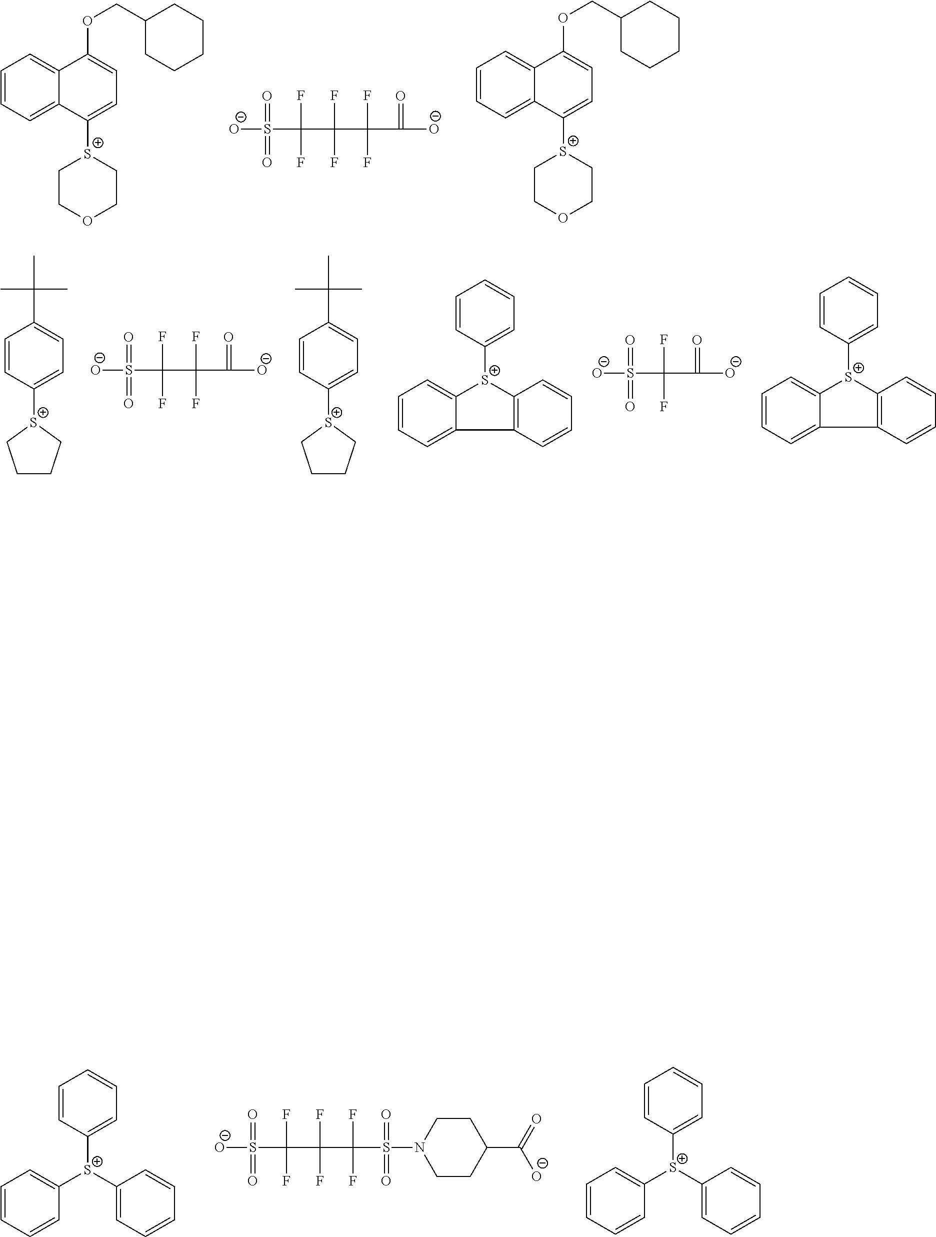

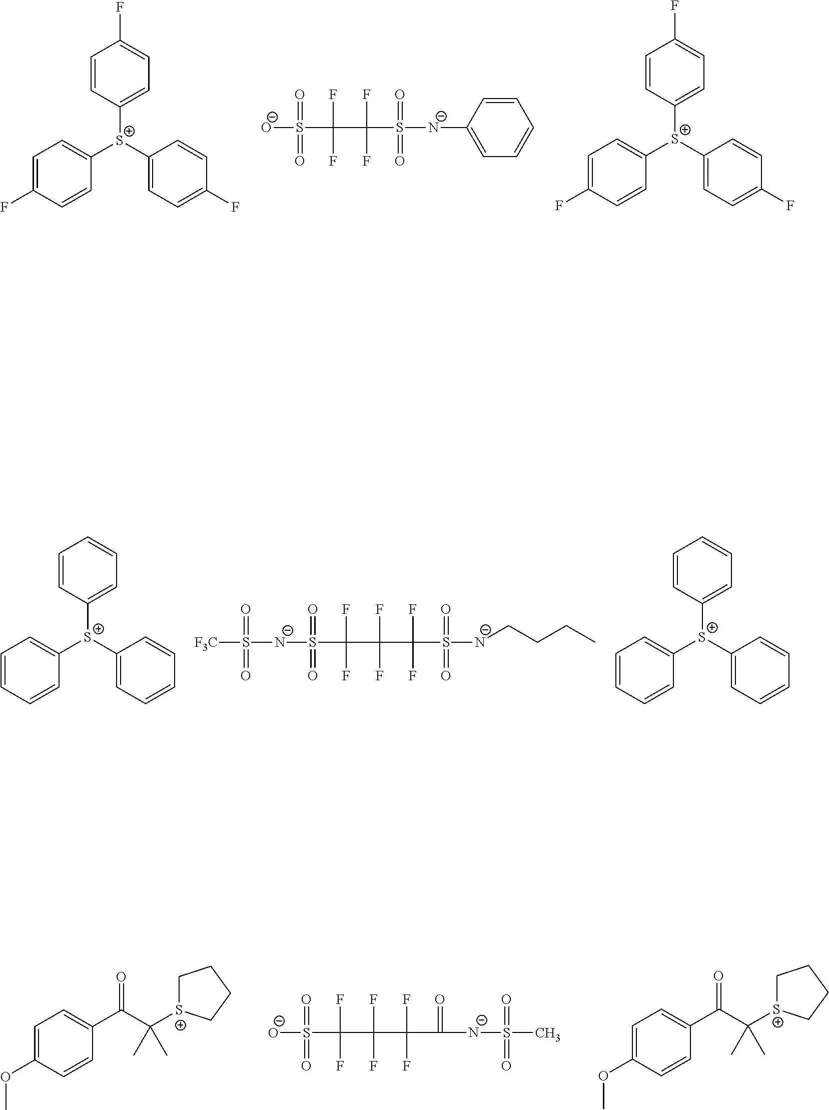

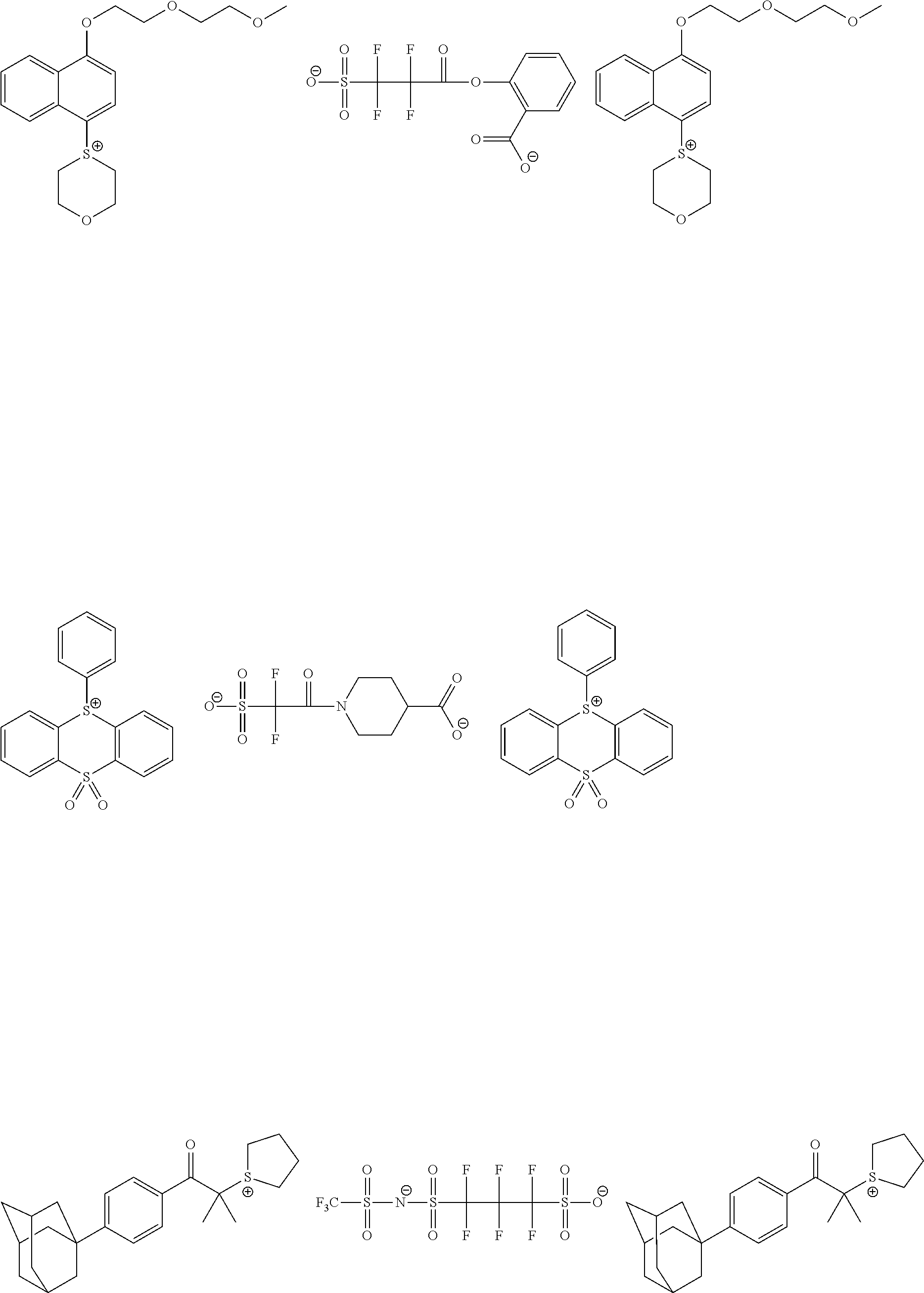

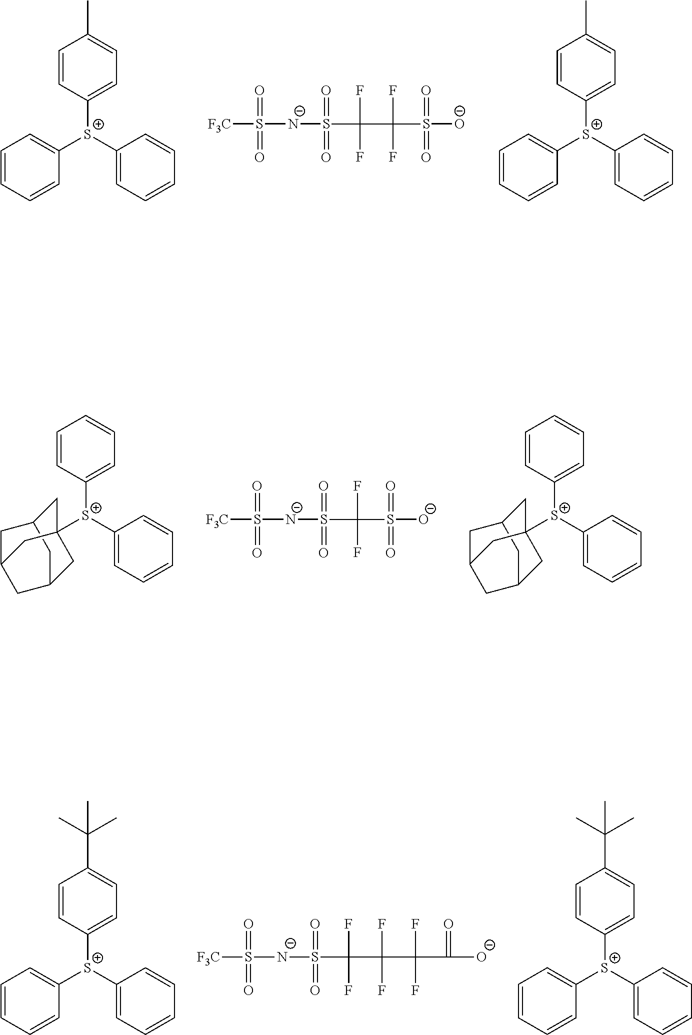

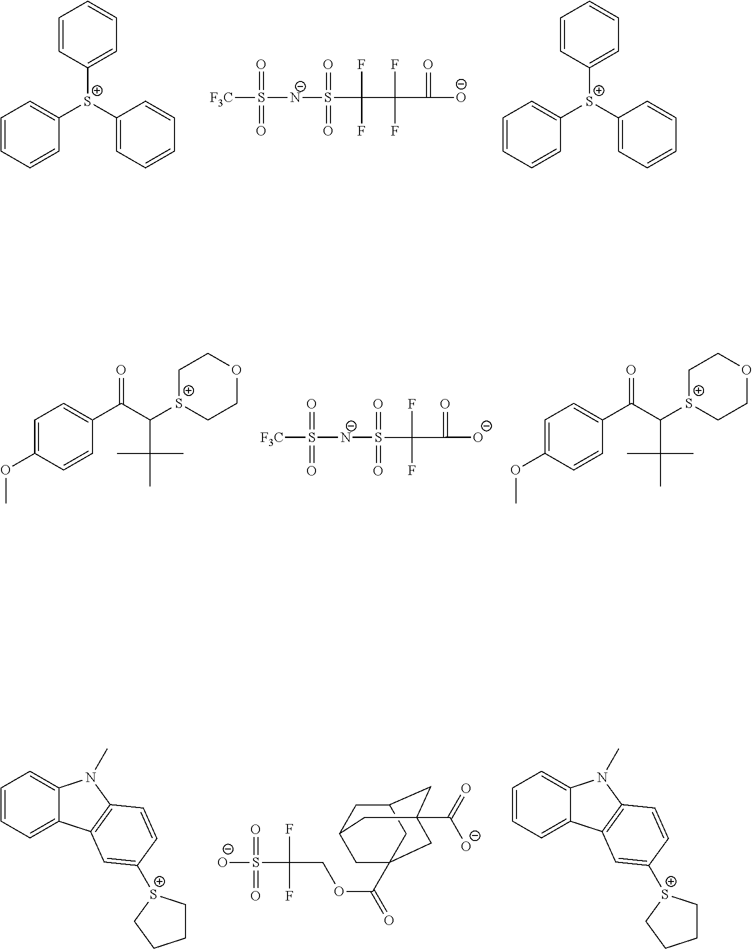

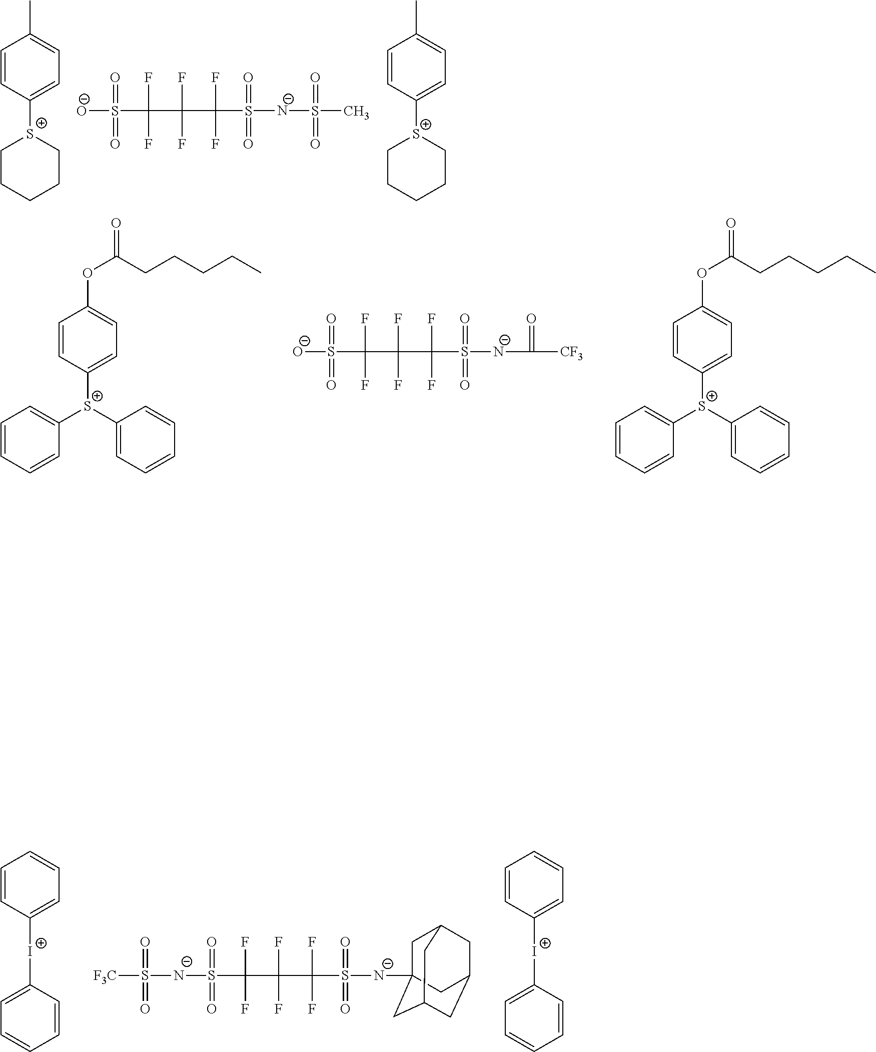

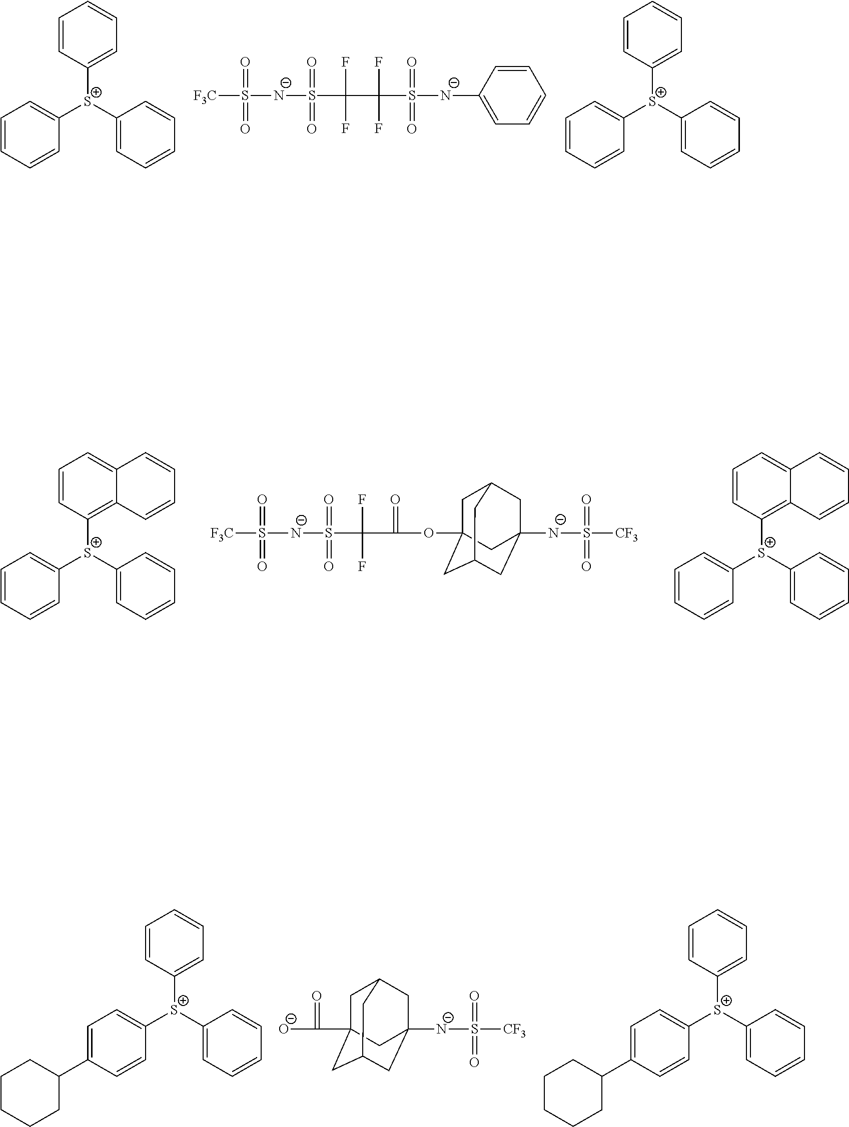

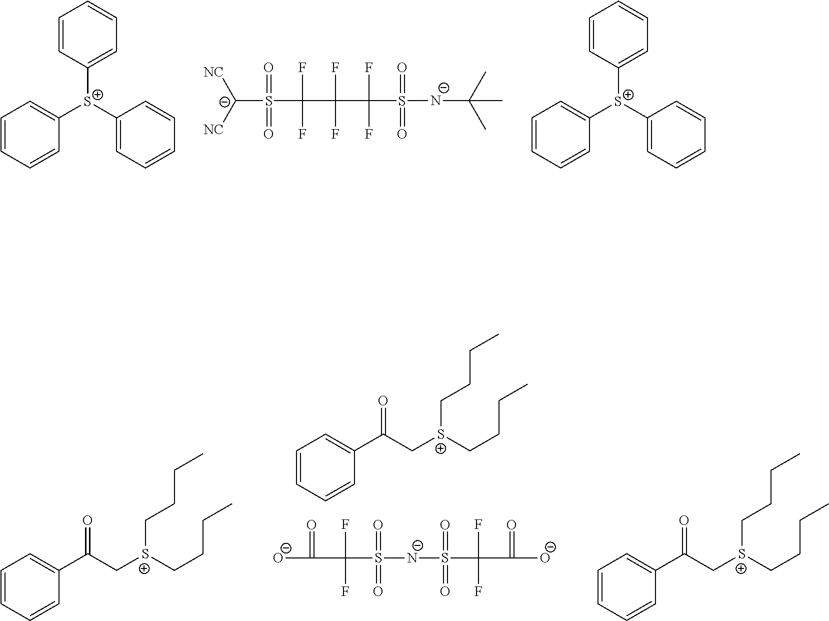

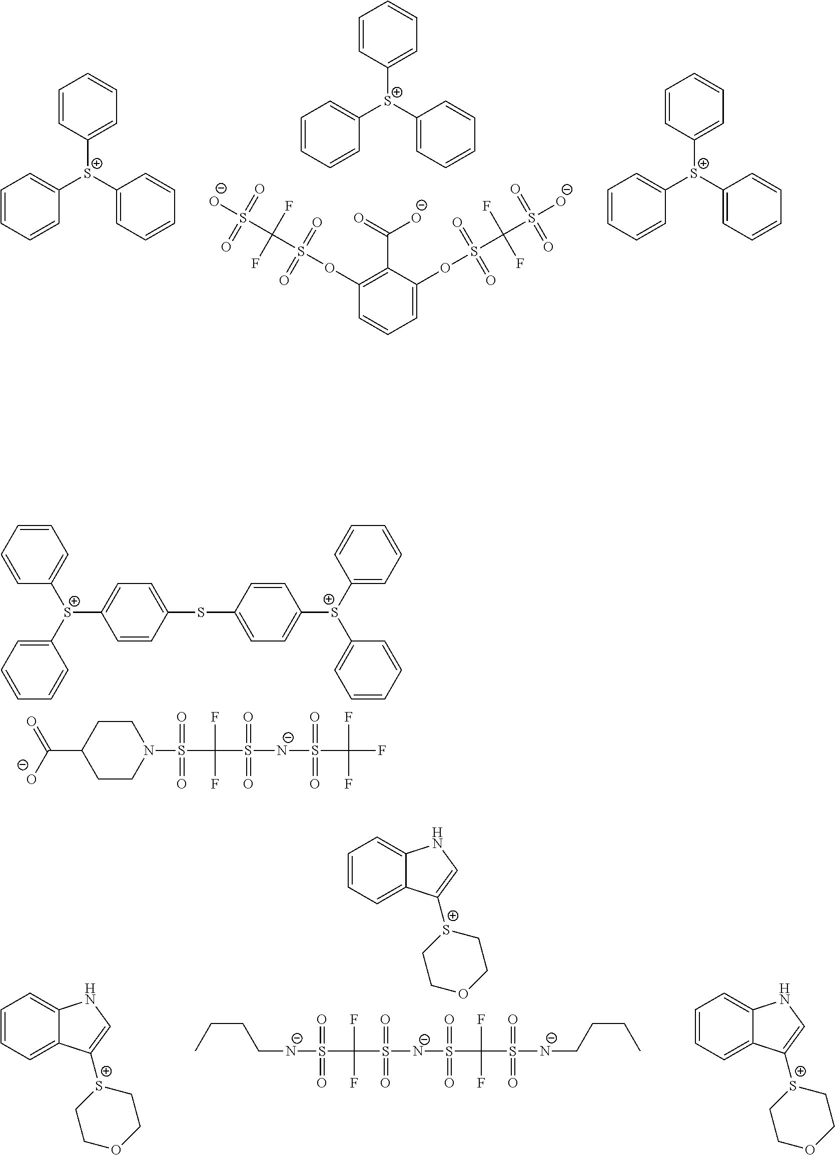

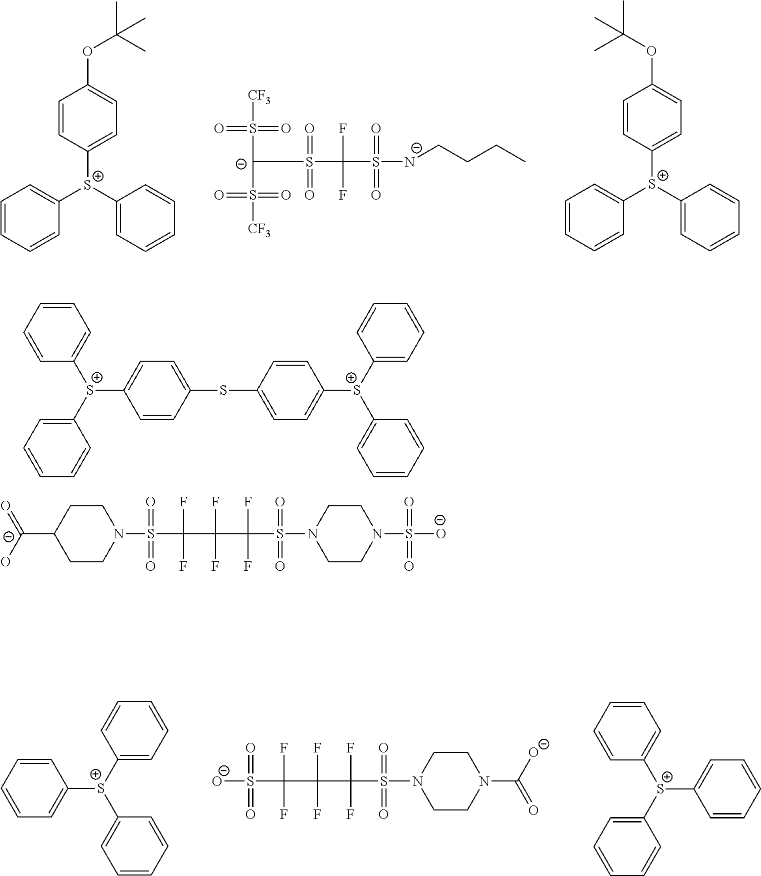

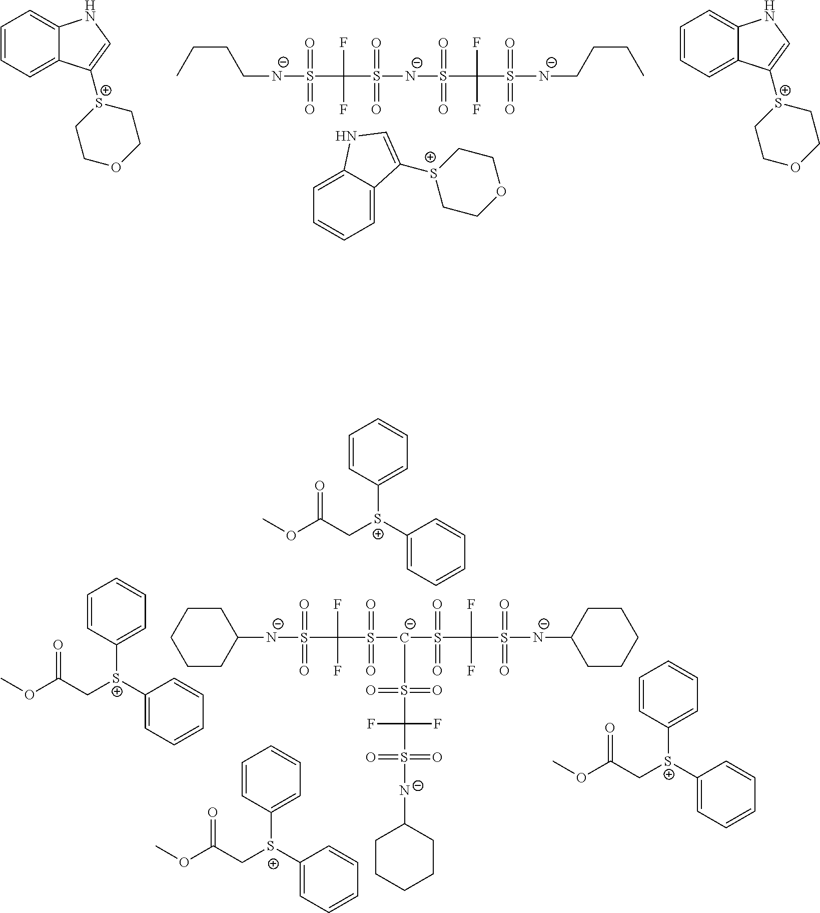

### [2-2] Compound (C2)

The compound (C2) is a compound containing two or more groups selected from the group consisting of a group capable of generating a structure represented by the following formula (a) upon irradiation with an actinic ray or radiation, a group capable of generating a structure represented by the following formula (b) upon irradiation with an actinic ray or radiation, a group capable of generating a structure represented by the following formula (c) upon irradiation with an actinic ray or radiation, and a group capable of generating a structure represented by the following formula (d) upon irradiation with an actinic ray or radiation:

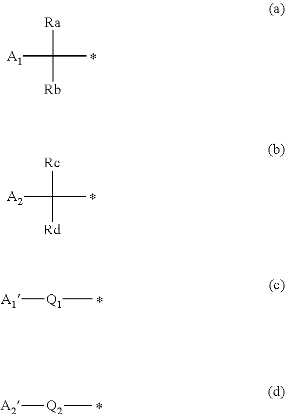

In formulae (a), (b), (c) and (d), A₁, A₂, A₁′ and A₂′ represent the same acidic functional group.

Each of Ra, Rb, Rc and Rd independently represents a hydrogen atom or a substituent.

Each of Q₁ and Q₂ represents a cyclic group.

However, the structure represented by formula (a) is different from the structure represented by formula (b) and the structure represented by formula (c) is different from the structure represented by formula (d). In other words, the linking group represented by —C(Ra)(Rb)- in the structure represented by formula (a) is different from the linking group represented by —C(Rc)(Rd)- in the structure represented by formula (b), and the linking group represented by Q₁ in the structure represented by formula (c) is different from the linking group represented by Q₂ in the structure represented by formula (d).

* represents a bond.

Here, the expression “the structure represented by formula (a) is different from the structure represented by formula (b)” is a proviso when the compound (C2) is a compound containing a group capable of generating a structure represented by formula (a) and a group capable of generating a structure represented by formula (b).

Similarly, the expression “the structure represented by formula (c) is different from the structure represented by formula (d)” is a proviso when the compound (C2) is a compound containing a group capable of generating a structure represented by formula (c) and a group capable of generating a structure represented by formula (d).

The compound (C2) may be an ionic compound or a nonionic compound as long as it contains two or more kinds of groups selected from the group consisting of a group capable of generating a structure represented by formula (a) upon irradiation with an actinic ray or radiation (hereinafter, sometimes referred to as “group capable of generating a structure represented by formula (a)”), a group capable of generating a structure represented by formula (b) upon irradiation with an actinic ray or radiation (hereinafter, sometimes referred to as “group capable of generating a structure represented by formula (b)”), a group capable of generating a structure represented by formula (c) upon irradiation with an actinic ray or radiation (hereinafter, sometimes referred to as “group capable of generating a structure represented by formula (c)”), and a group capable of generating a structure represented by formula (d) upon irradiation with an actinic ray or radiation (hereinafter, sometimes referred to as “group capable of generating a structure represented by formula (d)”), but the compound is preferably an ionic compound.

Also, as long as the compound (C2) contains two or more groups selected from the group consisting of a group capable of generating a structure represented by formula (a), a group capable of generating a structure represented by formula (b), a group capable of generating a structure represented by formula (c), and a group capable of generating a structure represented by formula (d), the compound may further contain the same group as any one of those two or more kinds of group and may contain a group capable of generating a structure different from the structure represented by formula (a), (b). (c) or (d) upon irradiation with an actinic ray or radiation.

In the case where the compound (C2) is an ionic compound, the compound (C2) preferably has, as the anion structure, two or more kinds of acid anion structures selected from the group consisting of a structure formed by removing a proton from the acidic functional group of the structure represented by formula (a), a structure formed by removing a proton from the acidic functional group of the structure represented by formula (b), a structure formed by removing a proton from the acidic functional group of the structure represented by formula (c), and a structure formed by removing a proton from the acidic functional group of the structure represented by formula (d).

Specific examples of the substituents represented by Ra, Rb, Rc and Rd include a nitro group, a halogen atom such as fluorine atom, a carboxyl group, a hydroxyl group, an amino group, a cyano group, an alkyl group (may be linear or branched, preferably having a carbon number of 1 to 20), an alkoxy group (preferably having a carbon number of 1 to 15), a cycloalkyl group (preferably having a carbon number of 3 to 15), an aryl group (preferably having a carbon number of 6 to 14), an alkoxycarbonyl group (preferably having a carbon number of 2 to 7), an acyl group (preferably having a carbon number of 2 to 12), an alkoxycarbonyloxy group (preferably having a carbon number of 2 to 7), an alkylthio group (preferably having a carbon number of 1 to 15), an alkylsulfonyl group (preferably having a carbon number of 1 to 15), an alkyliminosulfonyl group (preferably having a carbon number of 2 to 15), an aryloxysulfonyl group (preferably having a carbon number of 6 to 20, an alkylaryloxysulfonyl group (preferably having a carbon number of 7 to 20), a cycloalkylaryloxysulfonyl group (preferably having a carbon number of 10 to 20), an alkyloxyalkyloxy group (preferably having a carbon number of 5 to 20, and a cycloalkylalkyloxyalkyloxy group (preferably having a carbon number of 8 to 20). The aryl group or the ring structure in each group may further have an alkyl group (preferably having a carbon number of 1 to 15) as a substituent.

Specific examples and preferred range of the alkyl group in the alkyl fluoride as at least one member of Ra and Rb include those described above for the alkyl group as R₈, R₉, R₁₁ and R₁₄ to R₂₆.

Specific examples and preferred range of the alkyl group in the unsubstituted alkyl group as Ra and Rb include those described above for the alkyl group as R₈, R₉, R₁₁ and R₁₄ to R₂₆.

The cyclic group represented by Q₁ and Q₂ includes an alicyclic group, an arylene group, and a heterocyclic group (encompassing not only a heterocyclic group having aromaticity but also a heterocyclic group not having aromaticity).

The alicyclic group is preferably an alicyclic group having a carbon number of 3 to 20, and the alicyclic group may be monocyclic or polycyclic. A monocyclic cycloalkyl group such as cyclopentyl group, cyclohexyl group and cyclooctyl group, and a polycyclic cycloalkyl group such as norbornyl group, tricyclodecanyl group, tetracyclodecanyl group, tetracyclododecanyl group and adamantyl group, are preferred.

The arylene group includes an arylene group having a carbon number of 6 to 30 and is a group derived from a benzene ring, a naphthalene ring, a phenanthrene ring or an anthracene ring, particularly a group derived from a benzene ring.

The heterocyclic group includes a heterocyclic group having a carbon number of 6 to 30 and includes heterocyclic groups derived from a furan ring, a thiophene ring, a benzofuran ring, a benzothiophene ring, a dibenzofuran ring, a dibenzothiophene ring and a pyridine ring. Among these, heterocyclic groups derived from a furan ring, a thiophene ring and a pyridine ring are preferred.

The above-described substituents may further have a substituent, and examples of the substituent include an alkyl group (may be linear, branched or cyclic, preferably having a carbon number of 1 to 12), an aryl group (preferably having a carbon number of 6 to 14), a nitro group, a halogen atom such as fluorine atom, a carboxyl group, a hydroxyl group, an amino group, a cyano group, an alkoxy group (preferably having a carbon number of 1 to 15), a cycloalkyl group (preferably having a carbon number of 3 to 15), and an acyl group (preferably having a carbon number of 2 to 12).

The compound (C2) is more preferably a compound capable of generating, as A in formula (a), A₂ in formula (b), A₁′ in formula (c) and A₂′ in formula (d), the same groups as each other selected from the group consisting of groups represented by the following formulae (Ca-1) to (Ca-19), upon irradiation with an actinic ray or radiation:

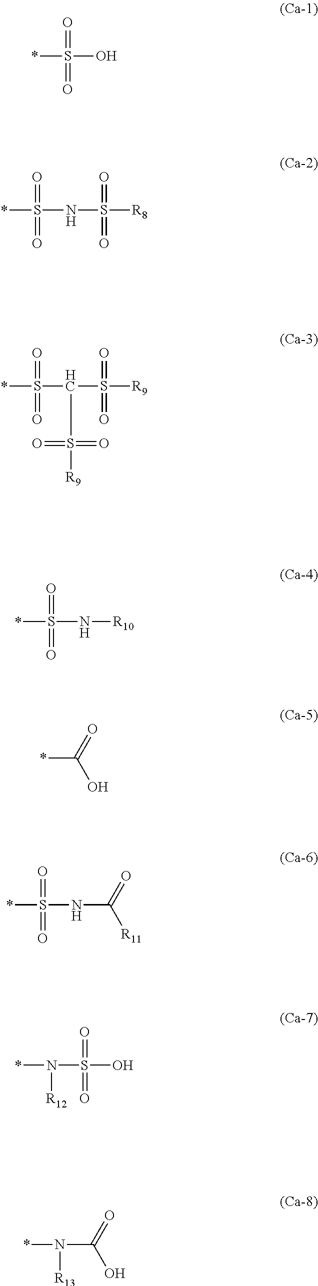

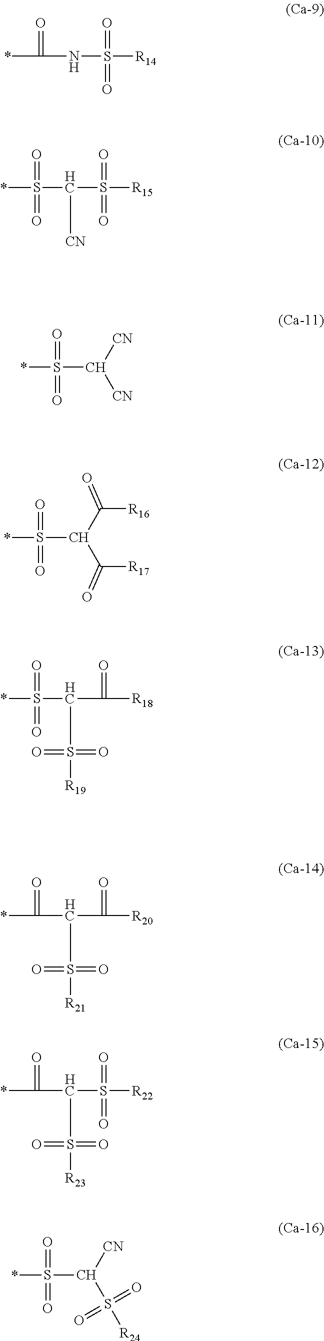

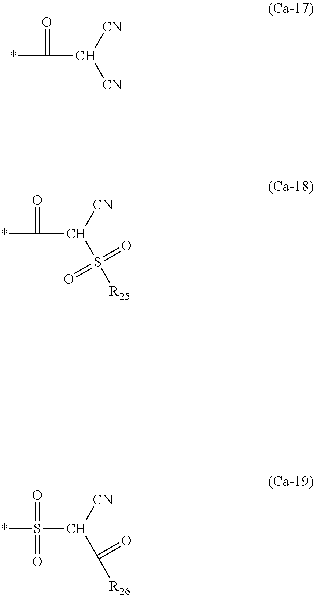

In formulae (Ca-2) to (Ca-4), (Ca-6) to (Ca-10), (Ca-12) to (Ca-16), (Ca-18) and (Ca-19), definitions, specific examples and preferred ranges of R₈ to R₂₆ are the same as those in the groups represented by formulae (Ca-2) to (Ca-4), (Ca-6) to (Ca-10), (Ca-12) to (Ca-16), (Ca-18) and (Ca-19) which may be contained in the compound (C1).

The compound (C2) is more preferably a compound capable of generating a group selected from the group consisting of groups represented by the following formulae (Cb-1) to (Cb-4), upon irradiation with an actinic ray or radiation, still more preferably a compound containing a group capable of generating a group selected from the group consisting of groups represented by the following formulae (Cb-1) to (Cb-4) and a group capable of generating a group selected from the group consisting of groups represented by the following formulae (Cc-1) to (Cc-4):

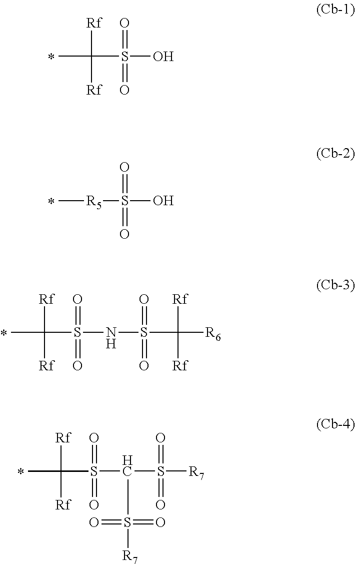

In formulae (Cb-1) to (Cb-4), each Rf independently represents a fluorine atom or an alkyl group substituted with at least one fluorine atom, R₅ represents a fluorine atom or an arylene group containing an alkyl group substituted with at least one fluorine atom, R₆ represents a hydrogen atom, a fluorine atom or an alkyl group, each R₇ independently represents an alkyl group, a cycloalkyl group or an aryl group, and * represents a bond.

In formulae (Cb-1) to (Cb-4), Rf, R₅, R₆ and R₇ are the same as Rf, R₅, R₆ and R₇ which may be contained in the compound (C1) above.

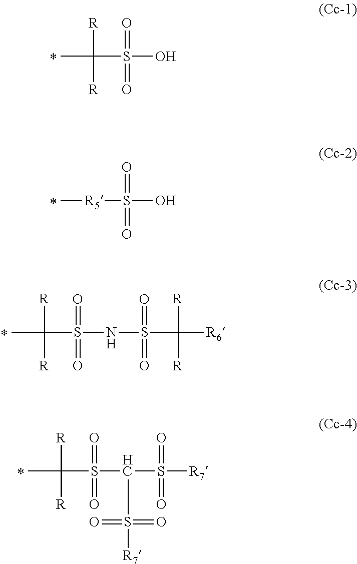

In formulae (Cc-1) to (Cc-4), each R independently represents an unsubstituted alkyl group, R₅′ represents an unsubstituted arylene group, R₆′ represents a hydrogen atom, a fluorine atom or an alkyl group, each R₇′ independently represents an alkyl group, a cycloalkyl group or an aryl group, and * represents a bond.

In formulae (Cc-1) to (Cc-4), specific examples and preferred ranges of R, R₅′, R₆′ and R₇′ are the same as those in the groups represented by formulae (Cc-1) to (Cc-4) which may be contained in the compound (C1).

The compound (C2) is preferably a compound containing a group capable of generating a structure represented by formula (a) and a group capable of generating a structure represented by formula (b). In this case, it is preferred that at least either one of Ra and Rb in formula (a) represents a fluorine atom or an alkyl fluoride group and each of Rc and Rd in formula (b) independently represents a hydrogen atom or an alkyl group not substituted with a fluorine atom (more preferably an unsubstituted alkyl group).

The compound (C2) is also preferably a compound containing a group capable of generating a structure represented by formula (c) and a group capable of generating a structure represented by formula (d). In this case, it is preferred that Qt is a cyclic group substituted with at least one fluorine atom and Q₂ is an unsubstituted cyclic group, and it is more preferred that Q₁ is an arylene group substituted with at least one fluorine atom or perfluoroalkyl group and Q₂ is an unsubstituted arylene group.

Specific examples of, when the compound (C2) is an ionic compound, the structure formed by removing a proton from the acidic functional group of the structure represented by formula (a), the structure formed by removing a proton from the acidic functional group of the structure represented by formula (b), the structure formed by removing a proton from the acidic functional group of the structure represented by formula (c), and the structure formed by removing a proton from the acidic functional group of the structure represented by formula (d), are illustrated below, but the present invention is not limited thereto.

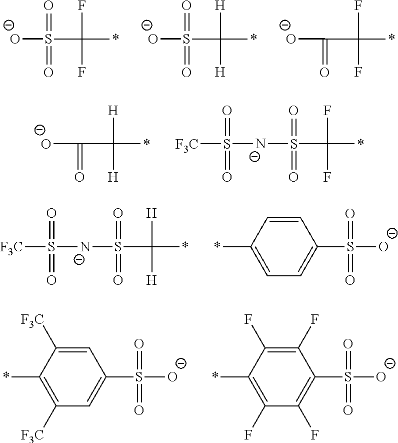

A₁, A₂, A₁′ and A₂′ represent the same acidic functional group, and the term “the same” indicates that specific groups are the same, and indicates, for example, that both A₁ and A₂ in the compound (C2) have a structure represented by formula (Ca-2) and R₈ in formula (Ca-2) is the same therebetween.

In the case where the compound (C2) is an ionic compound, examples of the cation in the compound (C2) are the same as those of the cation in the compound (C1) above.

The compound (C2) is preferably a compound represented by the following formula (C-2):

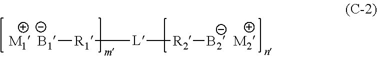

In formula (C-2), M₁′, M₂′, R₁′, R₂′, L′, m′ and n′ have the same meanings as M₁, M₂, R₁, R₂, L, m and n in formula (C-1), and m′≧n′.

B₁′ and B₂′ have the same meanings as different kinds of acid anion structures selected from the group consisting of an acid anion structure of a structure represented by formula (a) in the compound (C2), an acid anion structure of a structure represented by formula (b) in the compound (C2), an acid anion structure of a structure represented by formula (c) in the compound (C2), and an acid anion structure of a structure represented by formula (d) in the compound (C2). Here, the term “different kinds of” indicates that B₁′ and B₂′ represent acid anion structures represented by different formulae from each other, and indicates, for example, that B₁′ represents an acid anion structure of a structure represented by formula (a) and B₂′ represents an acid anion structure of a structure represented by formula (b).

Specific examples and preferred ranges of M₁′, M₂′, R₁′, R₂, L′, m′ and n′ are the same as those of M₁, M₂, R₁, R₂, L, m and n in formula (C-1).

Specific examples of the compound (C2) are illustrated below, but the present invention is not limited thereto.

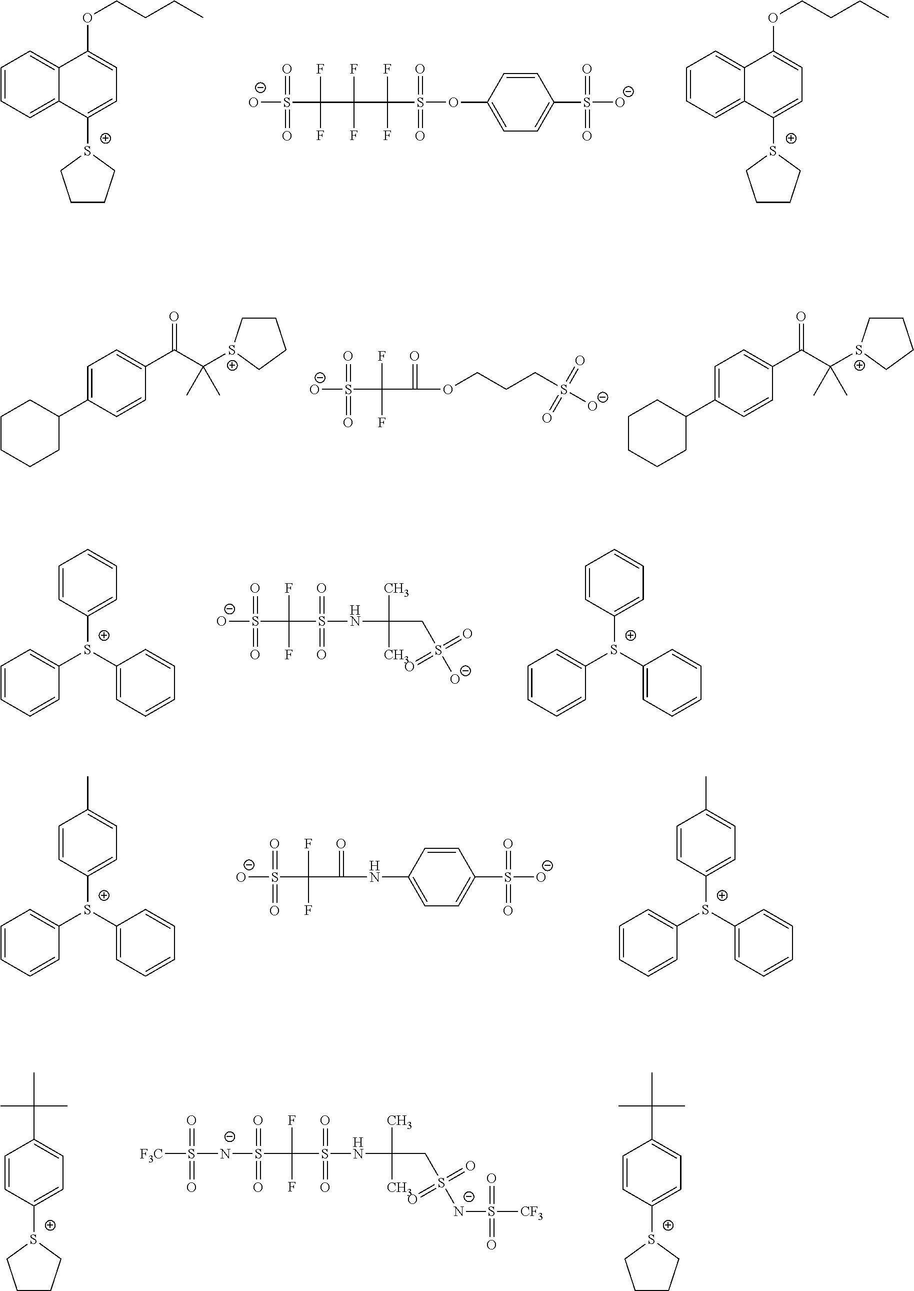

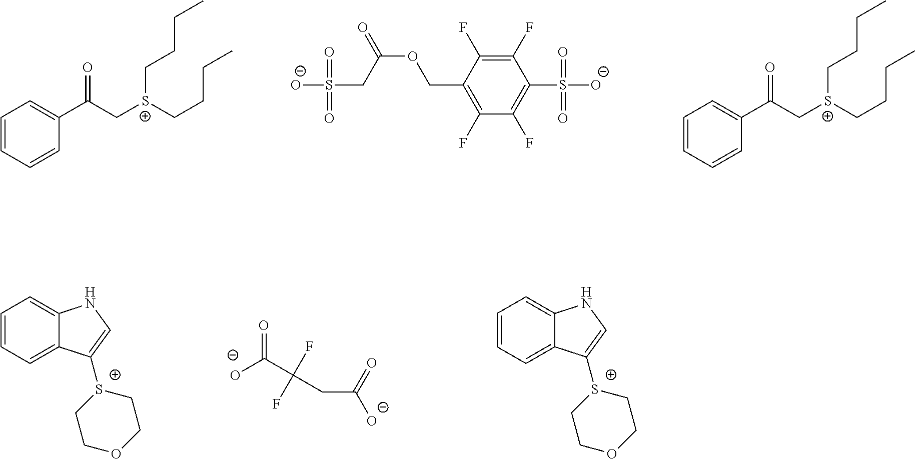 [SMILES: CCCC[S+](CCCC)CC(=O)c1ccccc1 ; O=C(CS(=O)(=O)[O-])OCc1c(F)c(F)c(S(=O)(=O)[O-])c(F)c1F ; CCCC[S+](CCCC)CC(=O)c1ccccc1 ; c1ccc2c([S+]3CCOCC3)c[nH]c2c1 ; O=C([O-])CC(F)(F)C(=O)[O-] ; c1ccc2c([S+]3CCOCC3)c[nH]c2c1]

The content of at least one of the compounds (C1) and (C2) (in the case of a plurality of kinds of compounds are present, the total thereof) is preferably from 0.001 to 30 mass %, more preferably from 0.01 to 20 mass %, based on the solid content of the actinic ray-sensitive or radiation-sensitive resin composition.

Both the compounds (C1) and (C2) are typically a low molecular compound.

The “low molecular compound” as used herein means to be different from a polymer compound having a repeating unit formed by polymerizing a monomer.

That is, the non-polymeric compound for use in the present invention is not a so-called polymer or oligomer obtained from a compound (monomer) having an unsaturated bond by cleaving the unsaturated bond with use of an initiator and growing the bond through a chain reaction, but is a compound having a fixed molecular weight (a compound having substantially no molecular weight distribution).

The molecular weight of the compound (A) is not particularly limited but is preferably from 500 to 5,000, more preferably from 600 to 4,000, still more preferably from 700 to 3,000.

The synthesis method for the compounds represented by formulae (C-1) and (C-2) includes a method of neutralizing a corresponding acid or performing a salt exchange reaction from a salt of a corresponding acid. Specifically, for example, the method described in JP-A-2011-37825 can be used.

[3](B) Compound Capable of Generating an Acid Upon Irradiation with an Actinic Ray or Radiation

The composition of the present invention may contain a compound capable of generating an acid upon irradiation with an actinic ray or radiation (hereinafter, sometimes referred to as “acid generator” or “compound (B)”), which is different from the compounds (C1) and (C2).

The acid generator is not limited as long as it is a known acid generator, and the acid generator may be an ionic compound composed of an anion and a cation, or a nonionic compound. In the case where the acid generator is an ionic compound, preferred range and specific examples of the cation contained in the acid generator are the same as those of the cation when the compound (C1) or (C2) is an ionic compound.

In the case where the acid generator is an ionic compound, the anion contained in the acid generator is preferably a non-nucleophilic anion (an anion having an extremely low ability of causing a nucleophilic reaction).

The anion contained in the acid generator includes, for example, a sulfonate anion (such as aliphatic sulfonate anion, aromatic sulfonate anion and a camphorsulfonate anion), a carboxylate anion (such as aliphatic carboxylate anion, aromatic carboxylate anion and aralkylcarboxylate anion), a sulfonylimide anion, a bis(alkylsulfonyl)imide anion, and tris(alkylsulfonyl)methide anion.

The aliphatic moiety in the aliphatic sulfonate anion and aliphatic carboxylate anion may be an alkyl group or a cycloalkyl group and is preferably a linear or branched alkyl group having a carbon number of 1 to 30 or a cycloalkyl group having a carbon number of 3 to 30.

The aromatic group in the aromatic sulfonate anion and aromatic carboxylate anion is preferably an aryl group having a carbon number of 6 to 14, and examples thereof include a phenyl group, a tolyl group and a naphthyl group.

The above-described alkyl group, cycloalkyl group and aryl group may have a substituent. Specific examples of the substituent include a nitro group, a halogen atom such as fluorine atom, a carboxyl group, a hydroxyl group, an amino group, a cyano group, an alkoxy group (preferably having a carbon number of 1 to 15), a cycloalkyl group (preferably having a carbon number of 3 to 15), an aryl group (preferably having a carbon number of 6 to 14), an alkoxycarbonyl group (preferably having a carbon number of 2 to 7), an acyl group (preferably having a carbon number of 2 to 12), an alkoxycarbonyloxy group (preferably having a carbon number of 2 to 7), an alkylthio group (preferably having a carbon number of 1 to 15), an alkylsulfonyl group (preferably having a carbon number of 1 to 15), an alkyliminosulfonyl group (preferably having a carbon number of 2 to 15), an aryloxysulfonyl group (preferably having a carbon number of 6 to 20), an alkylaryloxysulfonyl group (preferably having a carbon number of 7 to 20), a cycloalkylaryloxysulfonyl group (preferably having a carbon number of 10 to 20), an alkyloxyalkyloxy group (preferably having a carbon number of 5 to 20), and a cycloalkylalkyloxyalkyloxy group (preferably having a carbon number of 8 to 20). The aryl group or ring structure in each group may further have, as a substituent, an alkyl group (preferably having a carbon number of 1 to 15).

The aralkyl group in the aralkylcarboxylate anion is preferably an aralkyl group having a carbon number of 7 to 12, and examples thereof include a benzyl group, a phenethyl group, a naphthylmethyl group, a naphthylethyl group, and a naphthylbutyl group.

The sulfonylimide anion includes, for example, saccharin anion.

The alkyl group in the bis(alkylsulfonyl)imide anion and tris(alkylsulfonyl)methide anion is preferably an alkyl group having a carbon number of 1 to 5, and examples of the substituent on this alky group includes a halogen atom, a halogen atom-substituted alkyl group, an alkoxy group, an alkylthio group, an alkyloxysulfonyl group, an aryloxysulfonyl group, and a cycloalkylaryloxysulfonyl group, with a fluorine atom and a fluorine atom-substituted alkyl group being preferred.

Other anions contained in the acid generator include, for example, fluorinated phosphorus (e.g., PF₆⁻), fluorinated boron (e.g., BF₄⁻), and fluorinated antimony (e.g., SbF₆⁻).

The anion contained in the acid generator is preferably an aliphatic sulfonate anion substituted with a fluorine atom at least on the α-position of sulfonic acid, an aromatic sulfonate anion substituted with a fluorine atom or a fluorine atom-containing group, a bis(alkylsulfonyl)imide anion in which the alkyl group is substituted with a fluorine atom, or a tris(alkylsulfonyl)methide anion in which the alkyl group is substituted with a fluorine atom. The non-nucleophilic anion is more preferably a perfluoroaliphatic sulfonate anion (preferably having a carbon number of 4 to 8) or a benzenesulfonate anion having a fluorine atom, still more preferably nonafluorobutanesulfonate anion, perfluorooctanesulfonate anion, pentafluorobenzenesulfonate anion or 3,5-bis(trifluoromethyl)benzenesulfonate anion.

In terms of acid intensity, pKa of the acid generated is preferably −1 or less for enhancing the sensitivity.

The non-nucleophilic acid can be an anion capable of producing an organic acid represented by the following formula (V) or (VI). The compound (B) is preferably a compound capable of generating an organic acid represented by the following formula (V) or (VI) upon irradiation with an actinic ray or radiation:

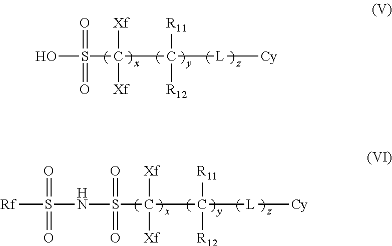

In formulae (V) and (VI),

each Xf independently represents a fluorine atom or an alkyl group substituted with at least one fluorine atom,

each L independently represents a divalent linking group,

each of R₁₁ and R₁₂ independently represents a hydrogen atom, a fluorine atom or an alkyl group,

Cy represents a cyclic organic group,

Rf represents a fluorine atom-containing group,

x represents an integer of 1 to 20,

y represents an integer of 0 to 10, and

z represents an integer of 0 to 10.

Xf represents a fluorine atom or an alkyl group substituted with at least one fluorine atom. The carbon number of the alkyl group is preferably from 1 to 10, more preferably from 1 to 4. Also, the alkyl group substituted with at least one fluorine atom is preferably a perfluoroalkyl group.

Xf is preferably a fluorine atom or a perfluoroalkyl group having a carbon number of 1 to 4. More specifically, Xf is preferably a fluorine atom, CF₃, C₂F₅, C₃F₇, C₄F₉, C₅F₁₁, C₆F₁₃, C₇F₁₅, C₈F₁₇, CH₂CF₃, CH₂CH₂CF₃, CH₂C₂F₅, CH₂CH₂C₂F₅, CH₂C₃F₇, CH₂CH₂C₃F₇, CH₂C₄F₉ or CH₂CH₂C₄F₉, more preferably a fluorine atom or CF₃, and it is still more preferred that both Xf are a fluorine atom.

Each of R₁₁ and R₁₂ independently represents a hydrogen atom, a fluorine atom or an alkyl group. The alkyl group may have a substituent (preferably fluorine atom) and is preferably an alkyl group having a carbon number of 1 to 4, more preferably a perfluoroalkyl group having a carbon number of 1 to 4. Specific examples of the alkyl group having a substituent of R₁₁ and R₁₂ include CF₃, C₂F₅, C₃F₇, C₄F₉, C₅F₁₁, C₆F₁₃, C₇F₁₅, C₈F₁₇, CH₂CF₃, CH₂CH₂CF₃, CH₂C₂F₅, CH₂CH₂C₂F₅, CH₂C₃F₇, CH₂CH₂C₃F₇, CH₂C₄F₉ and CH₂CH₂C₄F₉, with CF₃ being preferred.

L represents a divalent linking group. This divalent linking group includes, for example, —COO—, —OCO—, —CONH—, —NHCO—, —CO—, —O—, —S—, —SO—, —SO₂—, an alkylene group (preferably having a carbon number of 1 to 6), a cycloalkylene group (preferably having a carbon number of 3 to 10), an alkenylene group (preferably having a carbon number of 2 to 6), and a divalent linking group formed by combining a plurality of these members. Among these, —COO—, —OCO—, —CONH—, —NHCO—, —CO—, —O—, —SO₂—, —COO-alkylene group-, —OCO— alkylene group-, —CONH-alkylene group- and —NHCO-alkylene group- are preferred, and —COO—, —OCO—, —CONH—, —SO₂—, —COO-alkylene group- and —OCO-alkylene group- are more preferred.

Cy represents a cyclic organic group. The cyclic organic group includes, for example, an alicyclic group, an aryl group, and a heterocyclic group

The alicyclic group may be monocyclic or polycyclic. The monocyclic alicyclic group includes, for example, a monocyclic cycloalkyl group such as cyclopentyl group, cylohexyl group and cyclooctyl group. The polycyclic alicyclic group includes, for example, a polycyclic cycloalkyl group such as norbornyl group, tricyclodecanyl group, tetracyclodecanyl group, tetracyclododecanyl group, and adamantyl group. Above all, an alicyclic group having a bulky structure with a carbon number of 7 or more, such as norbornyl group, tricyclodccanyl group, tetracyclodecanyl group, tetracyclododecanyl group and adamantyl group, is preferred from the standpoint of suppressing in-film diffusion in the PEB (post-exposure baking) step and improving MEEF (Mask Error Enhancement Factor).

The aryl group may be monocyclic or polycyclic. This aryl group includes, for example, a phenyl group, a naphthyl group, a phenanthryl group, and an anthryl group. Among these, a naphthyl group is preferred because of its relatively low absorbance at 193 nm.

The heterocyclic group may be monocyclic or polycyclic, but a polycyclic heterocyclic group can more suppress diffusion of an acid. Also, the heterocyclic group may have aromaticity or may not have aromaticity. The heterocyclic ring having aromaticity includes, for example, a furan ring, a thiophene ring, a benzofuran ring, a benzothiophene ring, a dibenzofuran ring, a dibenzothiophene ring, and a pyridine ring. The heterocyclic ring not having aromaticity includes, for example, a tetrahydropyran ring, a lactone ring, and a decahydroisoquinoline ring. The heterocyclic ring in the heterocyclic group is preferably a furan ring, a thiophene ring, a pyridine ring or a decahydroisoquinoline ring. Examples of the lactone ring include lactone structures exemplified in the resin (A) above.

The above-described cyclic organic group may have a substituent, and the substituent includes, for example, an alkyl group (may be linear or branched, preferably having a carbon number of 1 to 12), a cycloalkyl group (may be monocyclic, polycyclic or spirocyclic, preferably having a carbon number of 3 to 20), an aryl group (preferably having a carbon number of 6 to 14), a hydroxyl group, an alkoxy group, an ester group, an amido group, a urethane group, a ureido group, a thioether group, a sulfonamido group, and a sulfonic acid ester group. Incidentally, the carbon constituting the cyclic organic group (the carbon contributing to ring formation) may be carbonyl carbon.

x is preferably from 1 to 8, more preferably from 1 to 4, still more preferably 1. y is preferably from 0 to 4, more preferably 0. z is preferably from 0 to 8, more preferably from 0 to 4.

The fluorine atom-containing group represented by Rf includes, for example, an alkyl group having at least one fluorine atom, a cycloalkyl group having at least one fluorine atom, and an aryl group having at least one fluorine atom.

These alkyl group, cycloalkyl group and aryl group may be substituted with a fluorine atom or may be substituted with another substituent containing a fluorine atom. In the case where Rf is a cycloalkyl group having at least one fluorine atom or an aryl group having at least one fluorine atom, the another substituent containing a fluorine atom includes, for example, an alkyl group substituted with at least one fluorine atom.

Also, these alkyl group, cycloalkyl group and aryl group may be further substituted with a substituent not containing a fluorine atom. This substituent includes, for example, those not containing a fluorine atom out of the groups described above for Cy.

Examples of the alkyl group having at least one fluorine atom represented by Rf are the same as those described above as the alkyl group substituted with at least one fluorine atom represented by Xf. The cycloalkyl group having at least one fluorine atom represented by Rf includes, for example, a perfluorocyclopentyl group and a perfluorocyclohexyl group. The aryl group having at least one fluorine atom represented by Rf includes, for example, a perfluorophenyl group.

As the anion contained in the acid generator, a sulfonate anion represented by the following formula (B-1) is also preferred:

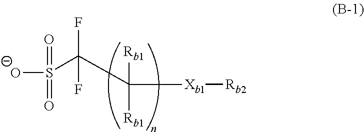

In formula (B-1), each R_{b1} independently represents a hydrogen atom, a fluorine atom or a trifluoromethyl group (CF₃).

n represents an integer of 0 to 4.

n is preferably an integer of 0 to 3, more preferably 0 or 1.

X_{b1} represents a single bond, an alkylene group, an ether bond, an ester bond (—OCO— or —COO—), a sulfonic acid ester bond (—OSO₂— or —SO₃—), or a combination thereof.

X_{b1} is preferably an ester bond (—OCO— or —COO—) or a sulfonic acid ester bond (—OSO₂— or —SO₃—), more preferably an ester bond (—OCO— or —COO—).

R_{b2} represents an organic group having a carbon number of 6 or more.

The organic group having a carbon number of 6 or more of R_{b2} is preferably a bulky group, and examples thereof include an alkyl group, an alicyclic group, an aryl group, and a heterocyclic group, each having a carbon number of 6 or more.

The alkyl group having a carbon number of 6 or more of R_{b2} may be linear or branched and is preferably a linear or branched alkyl group having a carbon number of 6 to 20, and examples thereof include a linear or branched hexyl group, a linear or branched heptyl group, and a linear or branched octyl group. In view of bulkiness, a branched alkyl group is preferred.

The alicyclic group having a carbon number of 6 or more of R_{b2} may be monocyclic or polycyclic, and the monocyclic alicyclic group includes, for example, a monocyclic cycloalkyl group such as cyclohexyl group and cyclooctyl group. The polycyclic alicyclic group includes, for example, a polycyclic cycloalkyl group such as norbornyl group, tricyclodecanyl group, tetracyclodecanyl group, tetracyclododecanyl group and adamantyl group. Above all, an alicyclic group having a bulky structure with a carbon number of 7 or more, such as norbornyl group, tricyclodecanyl group, tetracyclodecanyl group, tetracyclododecanyl group and adamantyl group, is preferred from the standpoint of suppressing in-film diffusion in the PEB (post-exposure baking) step and improving MEEF (Mask Error Enhancement Factor).

The aryl group having a carbon number of 6 or more of R_{b2} may be monocyclic or polycyclic. This aryl group includes, for example, a phenyl group, a naphthyl group, a phenanthryl group, and an anthryl group. Among these, a naphthyl group is preferred because of its relatively low absorbance at 193 nm.

The heterocyclic group having a carbon number of 6 or more of R_{b2} may be monocyclic or polycyclic, but a polycyclic heterocyclic group can more suppress diffusion of an acid. Also, the heterocyclic group may have aromaticity or may not have aromaticity. The heterocyclic ring having aromaticity includes, for example, a benzofuran ring, a benzothiophene ring, a dibenzofuran ring, and a dibenzothiophene ring. The heterocyclic ring not having aromaticity includes, for example, a tetrahydropyran ring, a lactone ring, a sultone ring, and a decahydroisoquinoline ring.

The substituent having a carbon number of 6 or more of R₂ may further have a substituent. This further substituent includes, for example, an alkyl group (may be linear or branched, preferably having a carbon number of 1 to 12), a cycloalkyl group (may be monocyclic, polycyclic or spirocyclic, preferably having a carbon number of 3 to 20), an aryl group (preferably having a carbon number of 6 to 14), a hydroxy group, an alkoxy group, an ester group, an amido group, a urethane group, a ureido group, a thioether group, a sulfonamido group, and a sulfonic acid ester group. Incidentally, the carbon constituting the above-described alicyclic group, aryl group or heterocyclic group (the carbon contributing to ring formation) may be carbonyl carbon.

Specific examples of the sulfonate anion structure represented by formula (B-1) are illustrated below, but the present invention is not limited thereto.

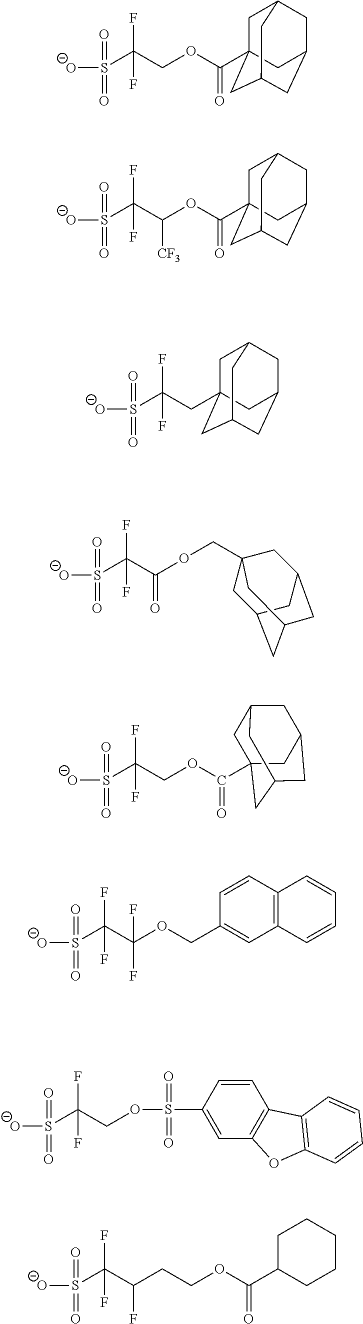

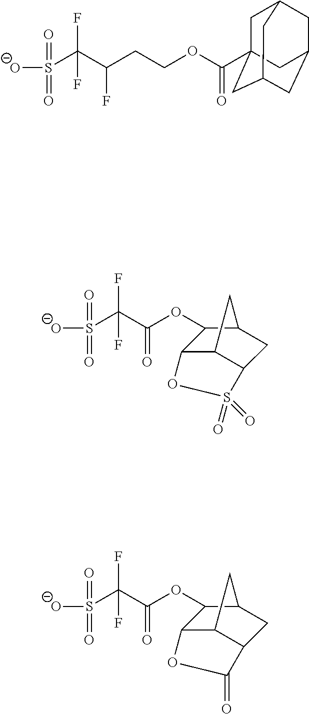 [SMILES: O=C(OCCC(F)C(F)(F)S(=O)(=O)[O-])C12CC3CC(CC(C3)C1)C2 ; O=C(OC1C2CC3C1OS(=O)(=O)C3C2)C(F)(F)S(=O)(=O)[O-] ; O=C1OC2C3CC(CC13)C2OC(=O)C(F)(F)S(=O)(=O)[O-]]

As the anion contained in the acid generator, a sulfonate anion represented by the following formula (A-I) is also preferred:

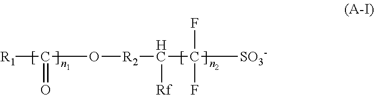

In formula (A-I),

R₁ is an alkyl group, a monovalent alicyclic hydrocarbon group, an aryl group or a heteroaryl group,

R₂ is a divalent linking group,

Rf is a fluorine atom or an alkyl group substituted with at least one fluorine atom, and

each of n₁ and n₂ is independently 0 or 1.

The alkyl group represented by R₁ is preferably an alkyl group having a carbon number of 1 to 20, more preferably an alkyl group having a carbon number of 1 to 10, still more preferably an alkyl group having a carbon number of 1 to 5, yet still more preferably an alkyl group having a carbon number of 1 to 4.

The alkyl group above may have a substituent (preferably fluorine atom), and the alkyl group having a substituent is preferably an alkyl group having a carbon number of 1 to 5 and substituted with at least one fluorine atom, more preferably a perfluoroalkyl group having a carbon number of 1 to 5.

The alkyl group represented by R₁ is preferably a methyl group, an ethyl group or a trifluoromethyl group, more preferably a methyl group or an ethyl group.

The monovalent alicyclic hydrocarbon group represented by R₁ preferably has a carbon number of 5 or more. Also, the carbon number of the monovalent alicyclic hydrocarbon group is preferably 20 or less, more preferably 15 or less. The monovalent alicyclic hydrocarbon group may be a monocyclic alicyclic hydrocarbon group or a polycyclic alicyclic hydrocarbon group. A part of —CH₂— of the alicyclic hydrocarbon group may be substituted for by —O— or —C(═O)—.

The monocyclic alicyclic hydrocarbon group is preferably an alicyclic hydrocarbon group having a carbon number of 5 to 12, and examples thereof include a cyclopentyl group, a cyclohexyl group, a cycloheptyl group, a cyclooctyl group, a cyclododecanyl group, a cyclopentenyl group, a cyclohexenyl group, a cyclooctadienyl group, and a piperidine ring group, with a cyclopentyl group, a cyclohexyl group and a cyclooctyl group being preferred.

The polycyclic alicyclic hydrocarbon group is preferably an alicyclic hydrocarbon group having a carbon number of 10 to 20.

The aryl group represented by R₁ preferably has a carbon number of 6 or more. Also, the carbon number of the aryl group is preferably 20 or less, more preferably 15 or less.

The heteroaryl group represented by R₁ preferably has a carbon number of 2 or more. Also, the carbon number of the heteroaryl group is preferably 20 or less, more preferably 15 or less.

These aryl group and heteroaryl group may be a monocyclic aryl group and a monocyclic heteroaryl group, or a polycyclic aryl group and a polycyclic heteroaryl group.

Examples of the monocyclic aryl group include a phenyl group.

Examples of the polycyclic aryl group include a naphthyl group and an anthracenyl group.

Examples of the monocyclic heteroaryl group include a pyridyl group, a thienyl group and a furanyl group.

Examples of the polycyclic heteroaryl group include a quinolyl group and an isoquinolyl group.

The monovalent alicyclic hydrocarbon group, aryl group and heteroaryl group as R₁ may further have a substituent, and this further substituent include a hydroxyl group, a halogen atom (such as fluorine atom, chlorine atom, bromine atom and iodine atom), a nitro group, a cyano group, an amido group, a sulfonamido group, an alkyl group, an alkoxy group, an alkoxycarbonyl group, an acyl group such as formyl group, acetyl group and benzoyl group, an acyloxy group such as acetoxy group and butyryloxy group, and a carboxy group.

Among others, R₁ is preferably a cyclohexyl group or an adamantyl group.

The divalent linking group represented by R₂ is not particularly limited but includes —COO—, —OCO—, —CO—, —O—, —S—, —SO—, —SO₂—, an alkylene group (preferably an alkylene group having a carbon number of 1 to 30), a cycloalkylene group (preferably a cycloalkylene group having a carbon number of 3 to 30), an alkenylene group (preferably an alkenylene group having a carbon number of 2 to 30), an arylene group (preferably an arylene group having a carbon number of 6 to 30), a heteroarylene group (preferably a heteroarylene group having a carbon number of 2 to 30), and a group formed by combining two or more thereof. These alkylene group, cycloakylene group, alkenylene group, arylene group and heteroarylene group may further have a substituent, and specific examples of the substituent are the same as those described above for the substituent which may be further substituted on the monovalent alicyclic hydrocarbon group, aryl group and heteroaryl group of R₁.

The divalent linking group represented by R₂ is preferably an alkylene group, a cycloalkylene group, an alkenylene group, an arylene group or a heteroarylene group, more preferably an alkylene group, still more preferably an alkylene group having a carbon number of 1 to 10, yet still more preferably an alkylene group having a carbon number of 1 to 5.

Rf is a fluorine atom or an alkyl group substituted with at least one fluorine atom. The carbon number of this alkyl group is preferably from 1 to 30, more preferably from 1 to 10, still more preferably from 1 to 4. Also, the alkyl group substituted with at least one fluorine atom is preferably a perfluoroalkyl group.

Rf is preferably a fluorine atom or a perfluoroalkyl group having a carbon number of 1 to 4. More specifically, Rf is preferably a fluorine atom or CF₃.

n₁ is preferably 1.

n₂ is preferably 1.

Specific preferred examples of the sulfonate anion represented by formula (A-I) are illustrated below, but the present invention is not limited thereto.

### [Sulfonate Anion Represented by Formula (A-I)]

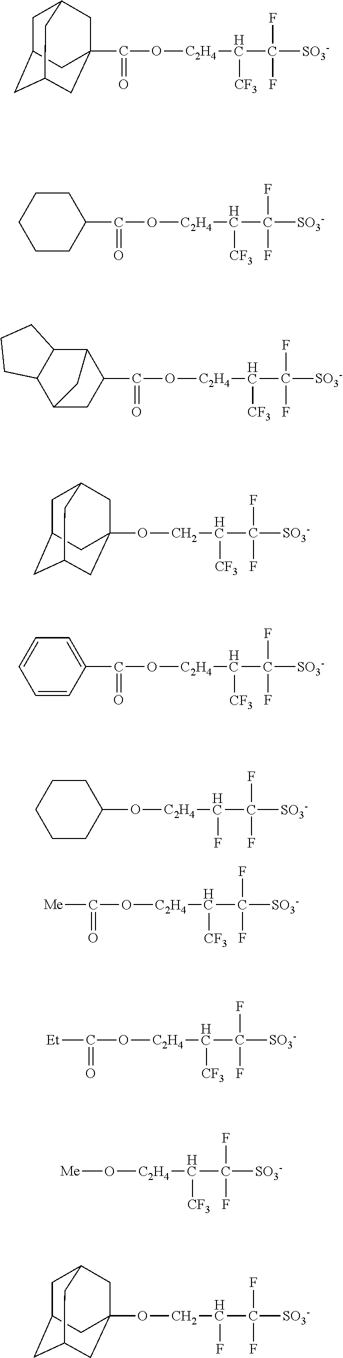

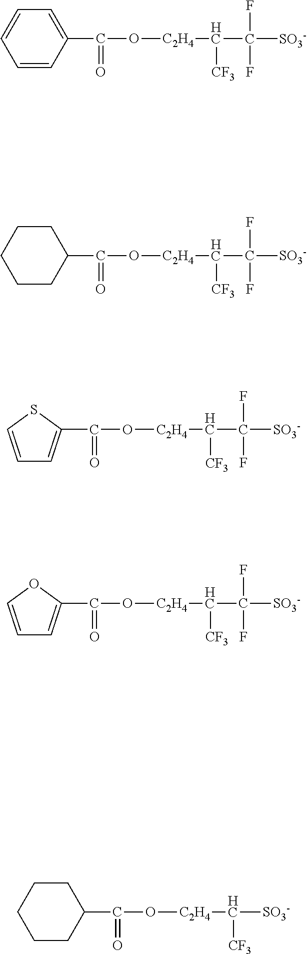

A preferred embodiment of the acid generator includes a compound represented by the following formula (ZIII):

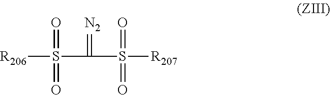

Each of R₂₀₆ and R₂₀₇ independently represents an aryl group, an alkyl group or a cycloalkyl group.

Examples of the aryl group, alkyl group and cycloalkyl group of R₂₀₆ and R₂₀₇ are the same as those described above for the aryl group, alkyl group and cycloalkyl group of R₂₀₁ to R₂₀₃ in the compound (ZI).

The aryl group, alkyl group and cycloalkyl group of R₂₀₆ and R₂₀₇ may have a substituent. Examples of the substituent are also the same as those of the substituent which may be substituted on the aryl group, alkyl group and cycloalkyl group of R₂₀₁ to R₂₀₃ in the compound (ZI).

The acid generator further includes a compound represented by the following formula (ZV):

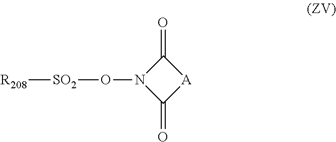

In formula (ZV), R₂₀₈ represents an alkyl group, a cycloalkyl group or an aryl group.

A represents an alkylene group, an alkenylene group or an arylene group.

Specific examples of the aryl group of R₂₀₈ are the same as specific examples of the aryl group of R₂₀₁ to R₂₀₃ in formula (ZI).

Specific examples of the alkyl group and cycloalkyl group of R₂₀₈ are the same as specific examples of the alkyl group and cycloalkyl group of R₂₀₁ to R₂₀₃ in formula (ZI).

The alkylene group of A includes an alkylene group having a carbon number of 1 to 12 (for example, a methylene group, an ethylene group, a propylene group, an isopropylene group, a butylene group and an isobutylene group); the alkenylene group of A includes an alkenylene group having a carbon number of 2 to 12 (for example, a vinylene group, a propenylene group and a butenylene group); and the arylene group of A includes an arylene group having a carbon number of 6 to 10 (for example, a phenylene group, a tolylene group and a naphthylene group).

As for the compound (B), the fluorine content ratio represented by (total mass of all fluorine atoms contained in the compound)/(total mass of all atoms contained in the compound) is preferably 0.30 or less, more preferably 0.25 or less, still more preferably 0.20 or less, yet still more preferably 0.15 or less, and most preferably 0.10 or less.

Among acid generators, particularly preferred examples are illustrated below.

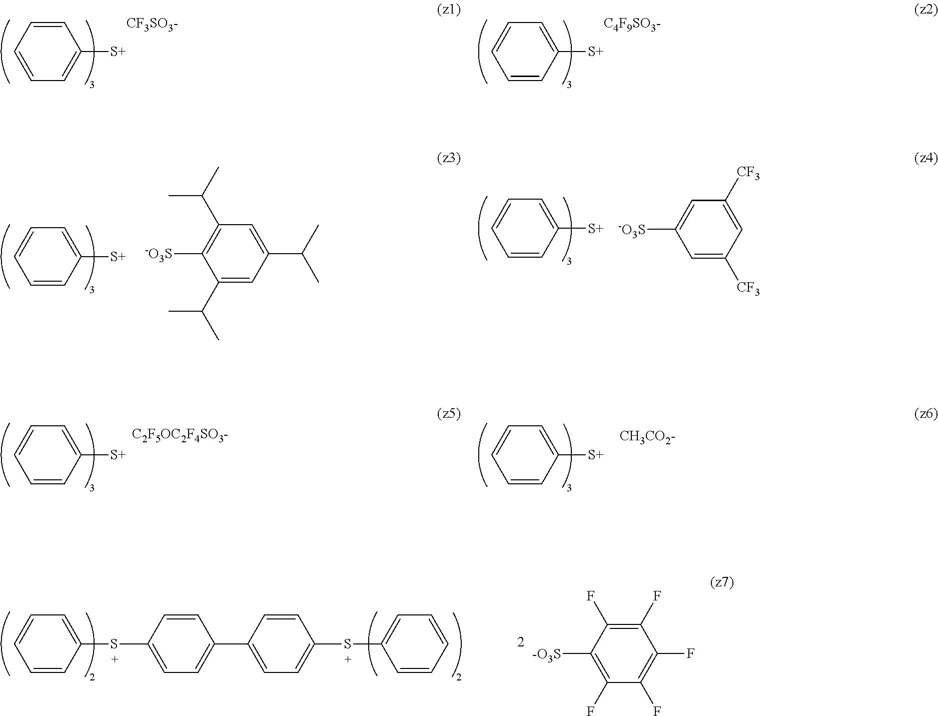

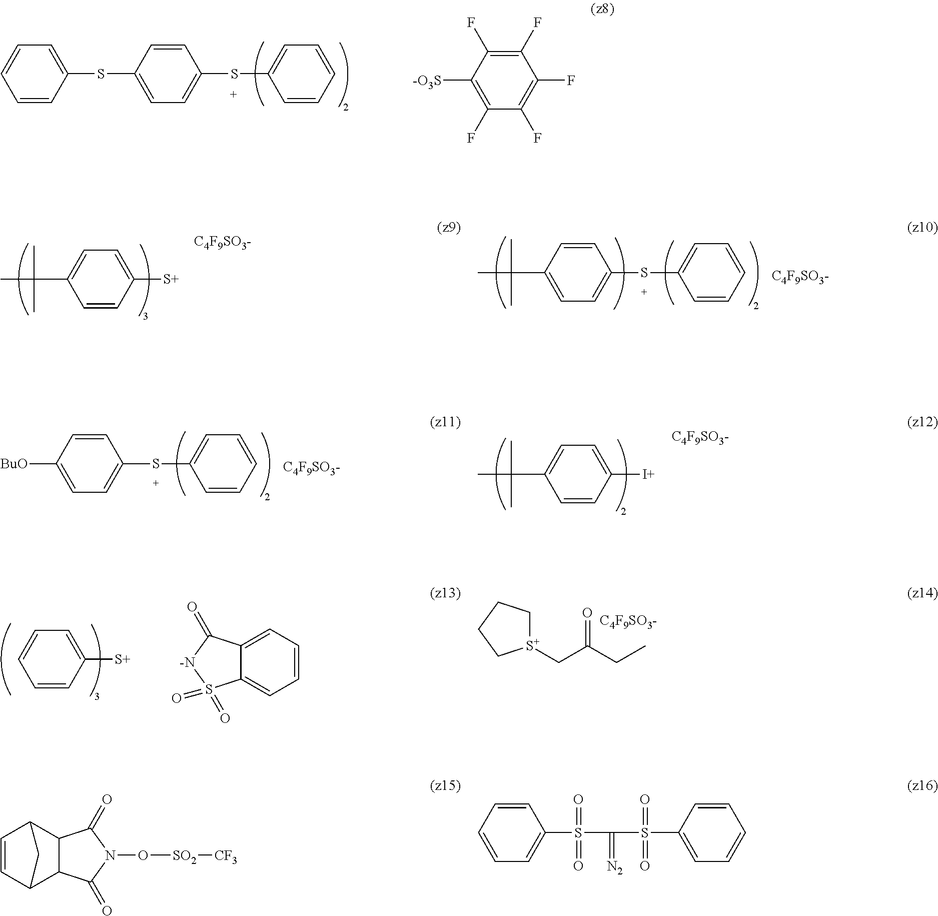

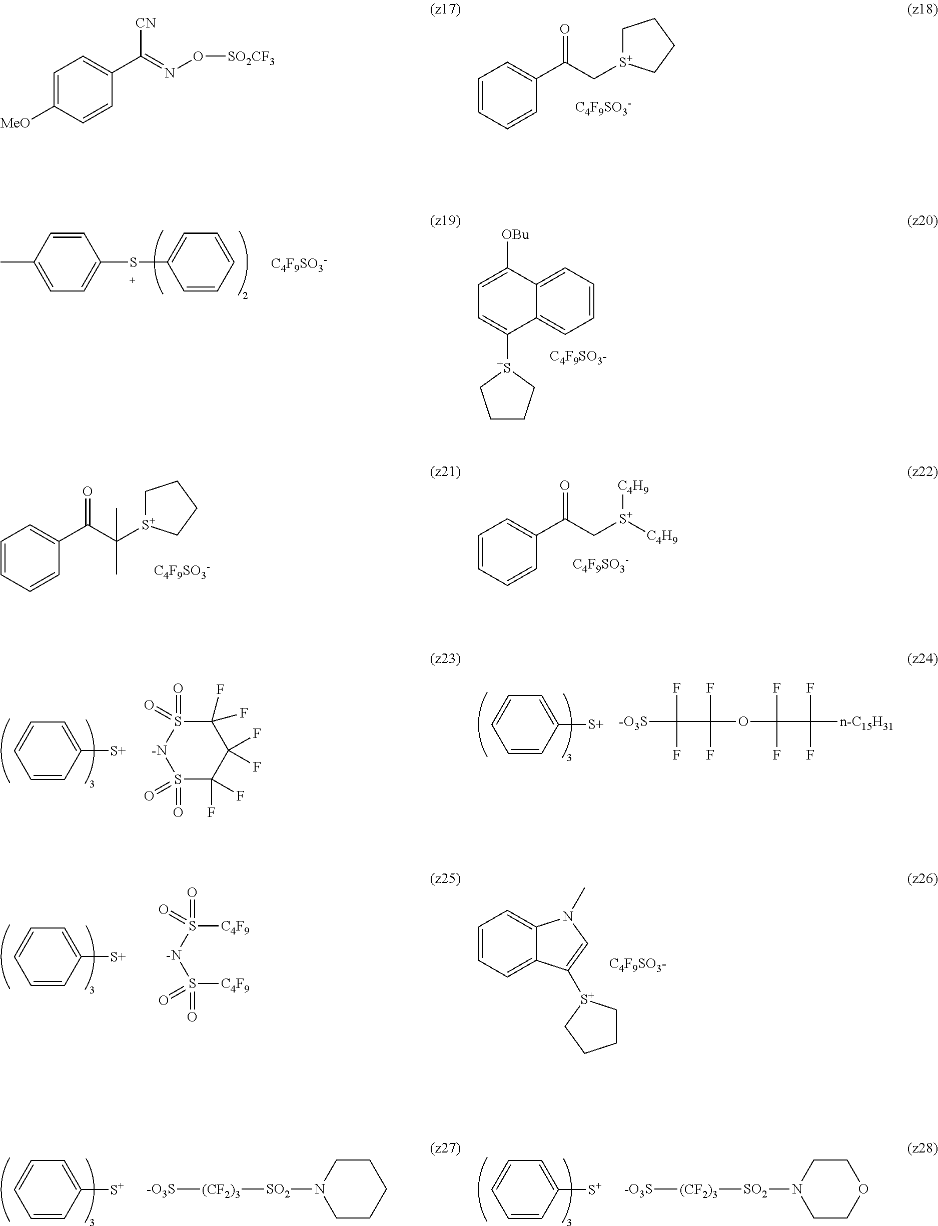

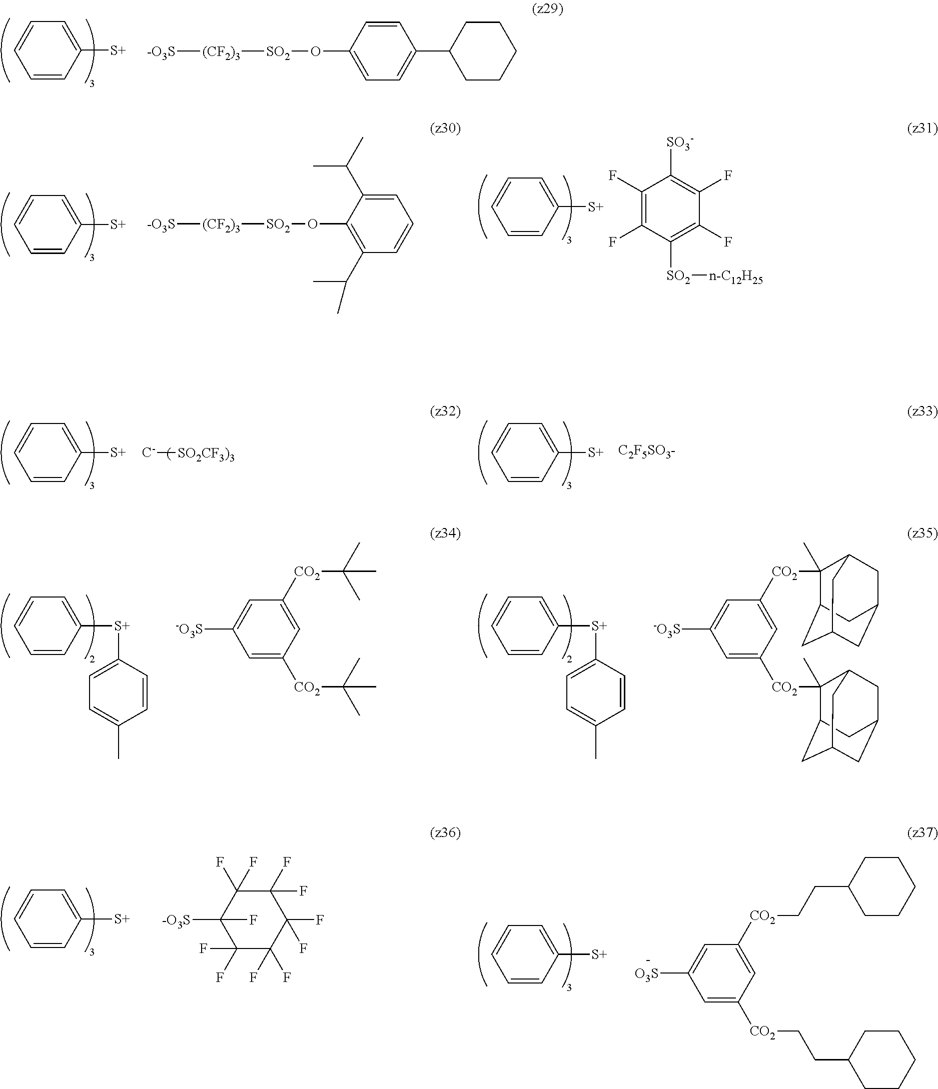

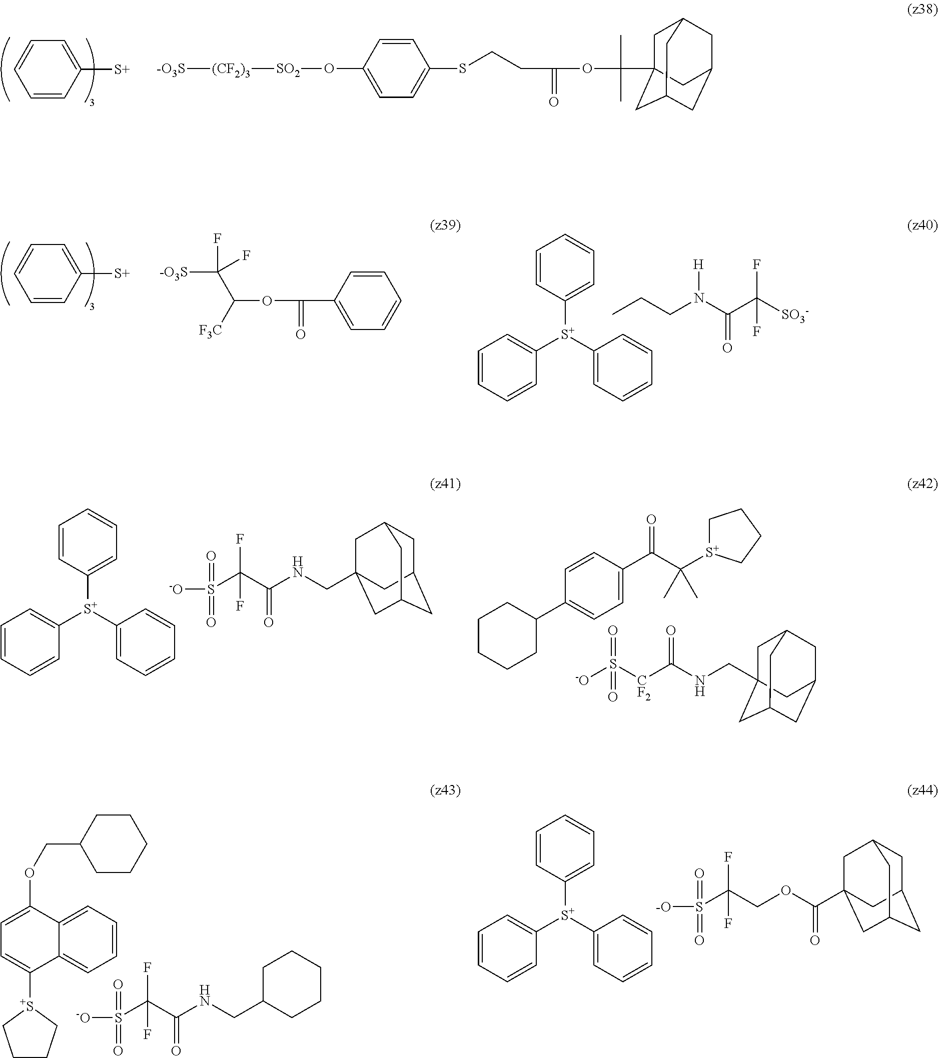

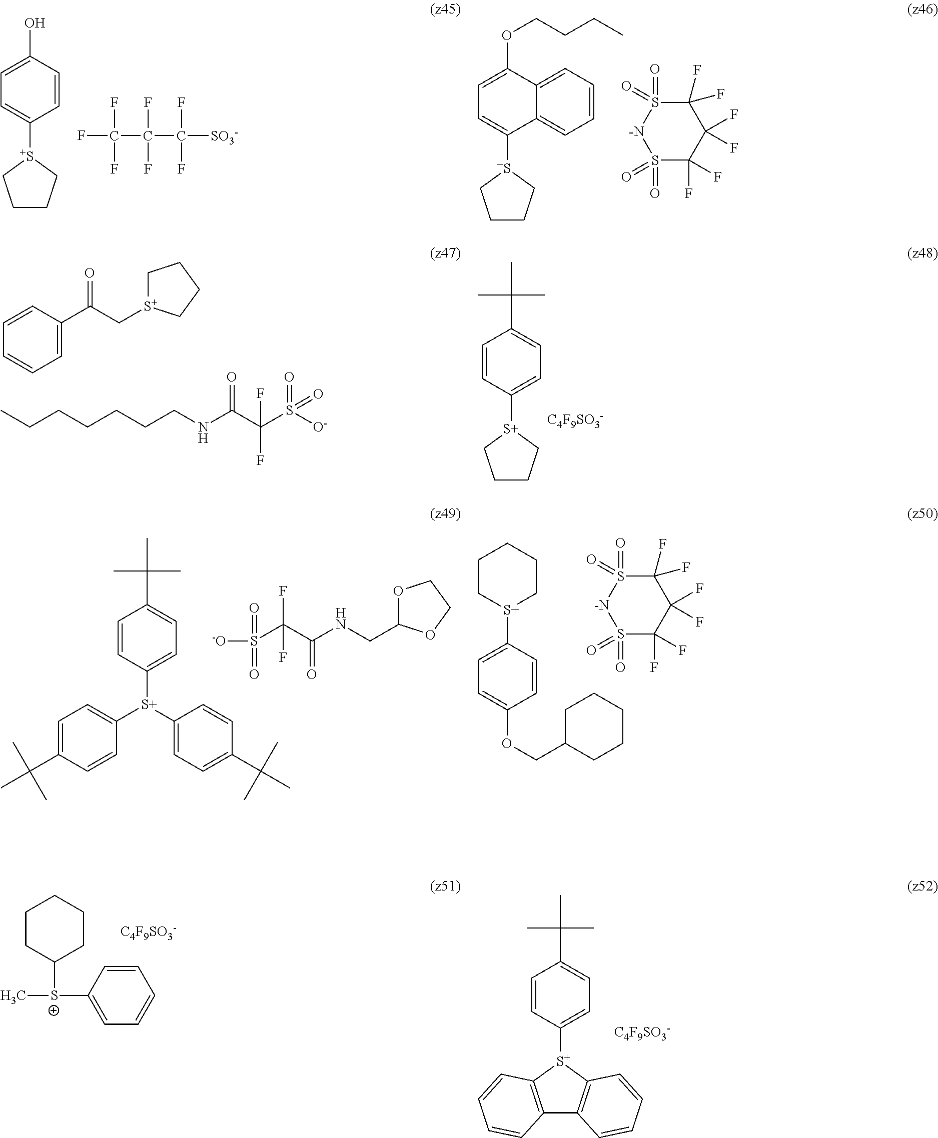

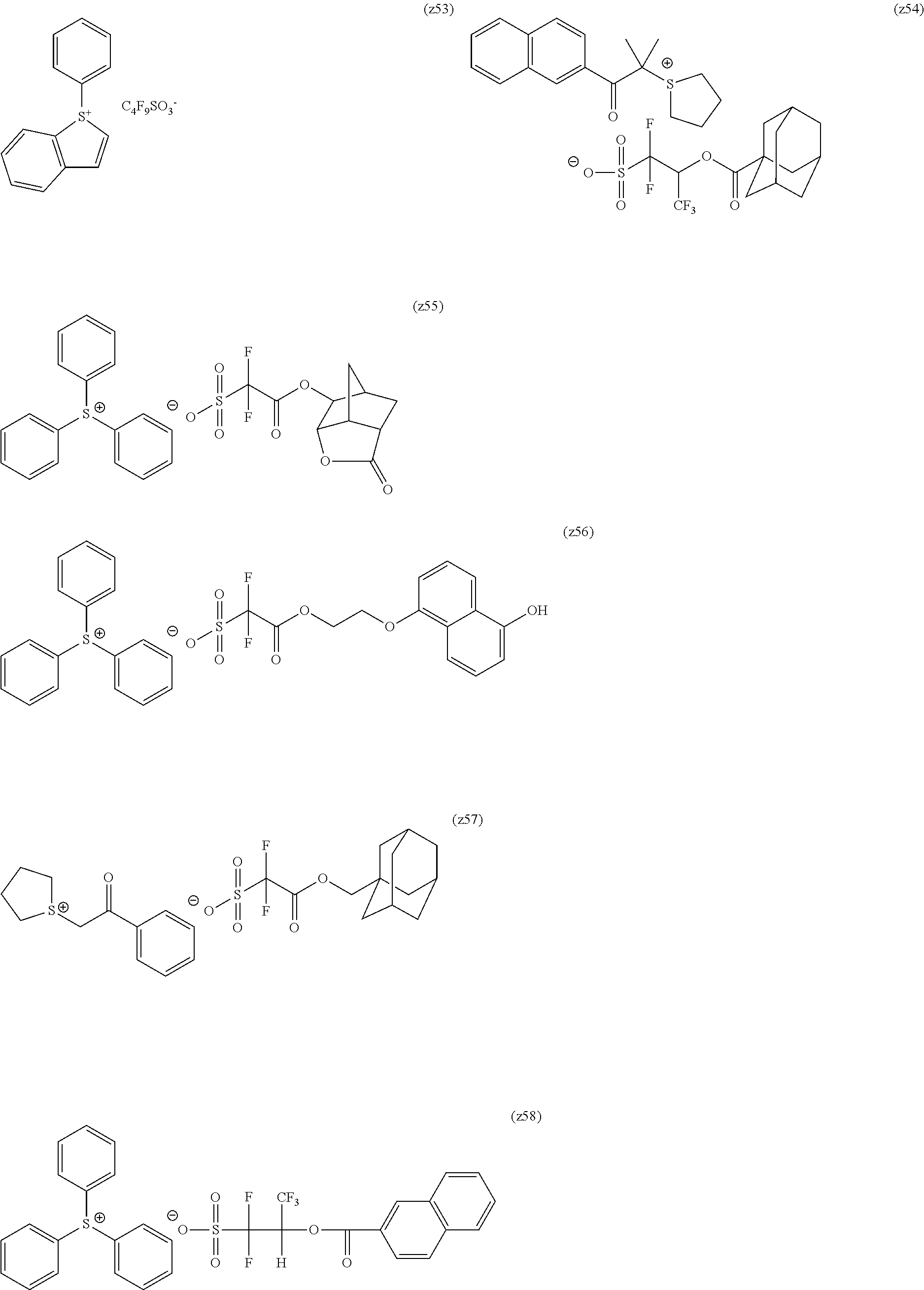

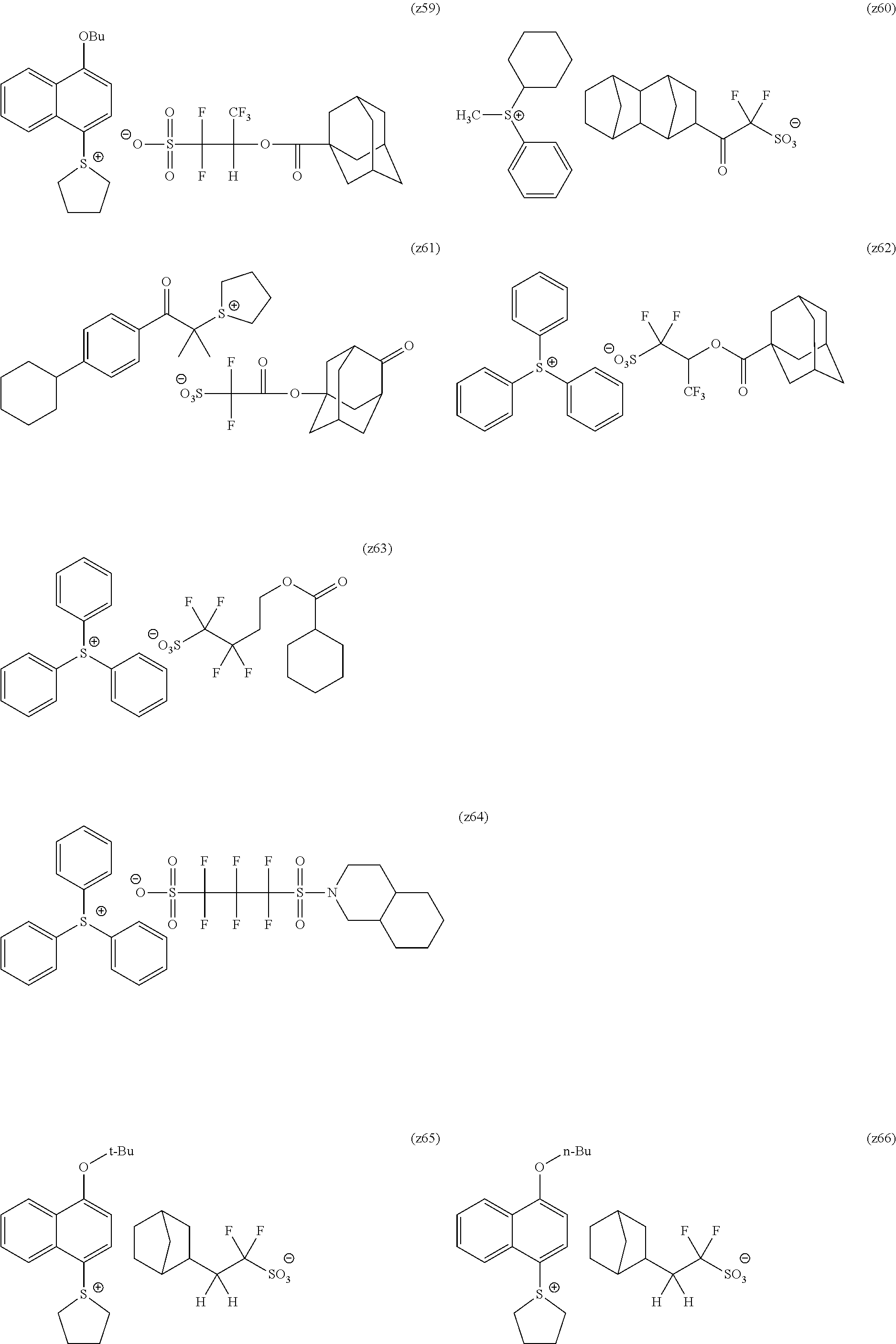

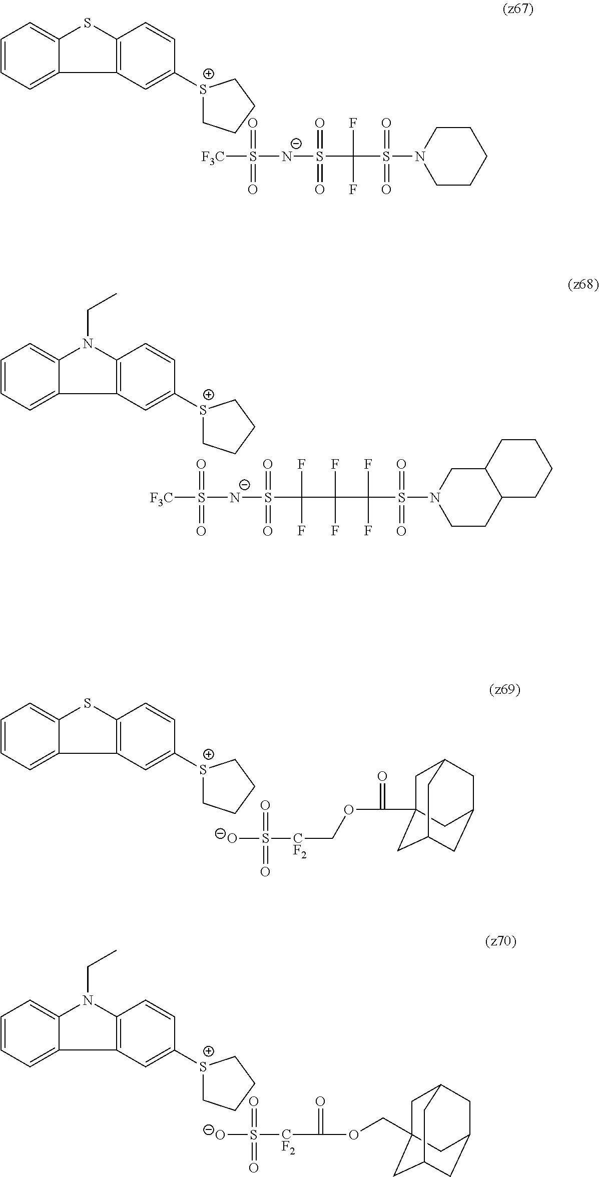

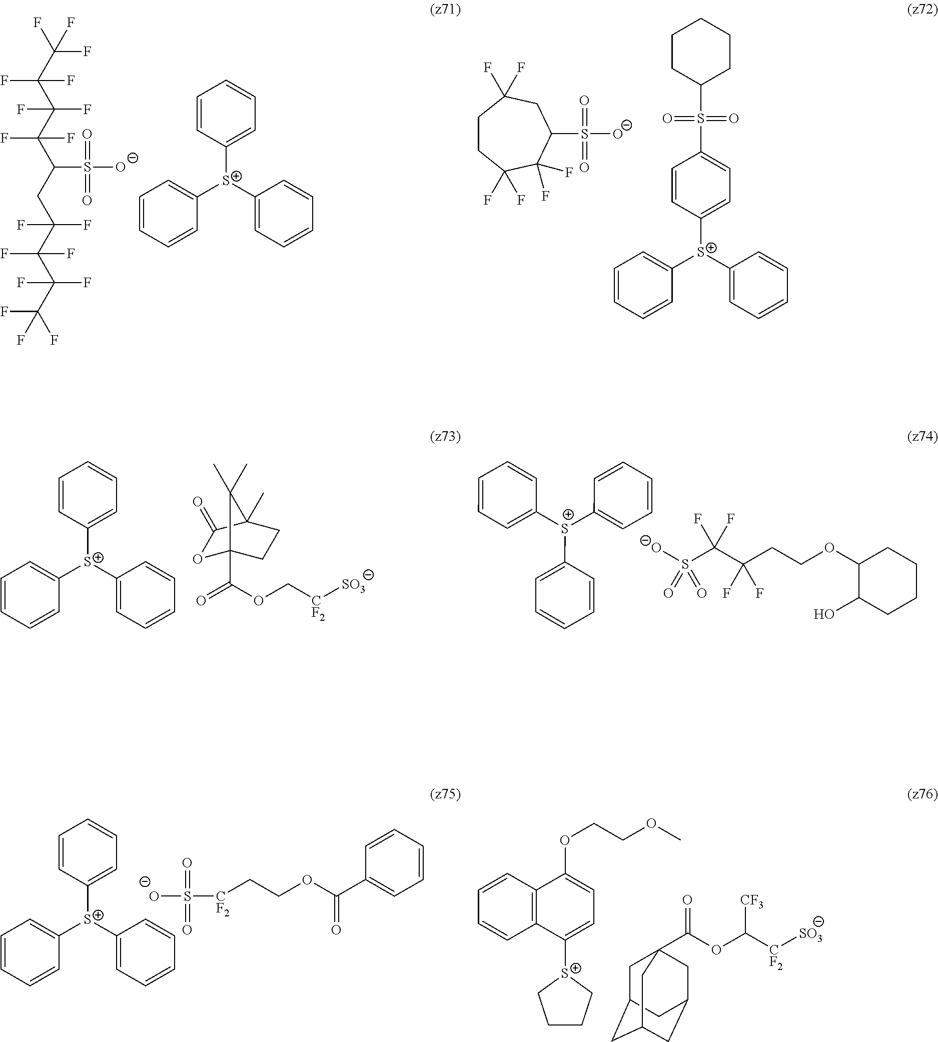

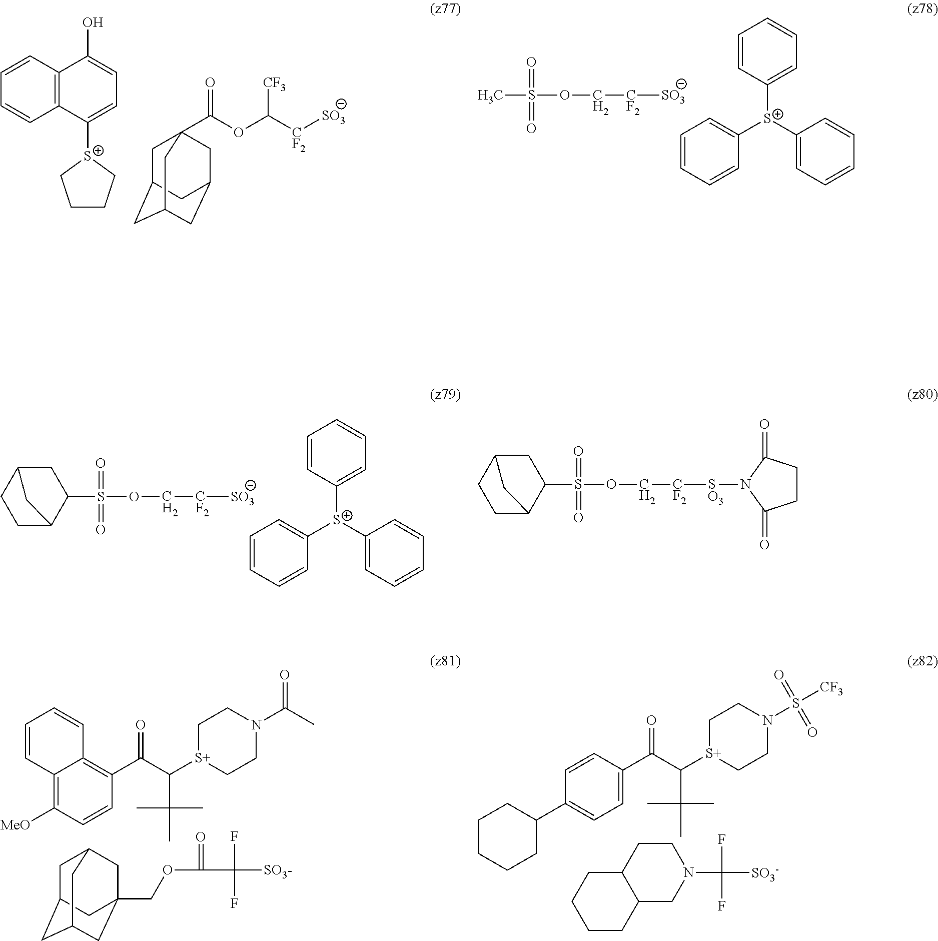

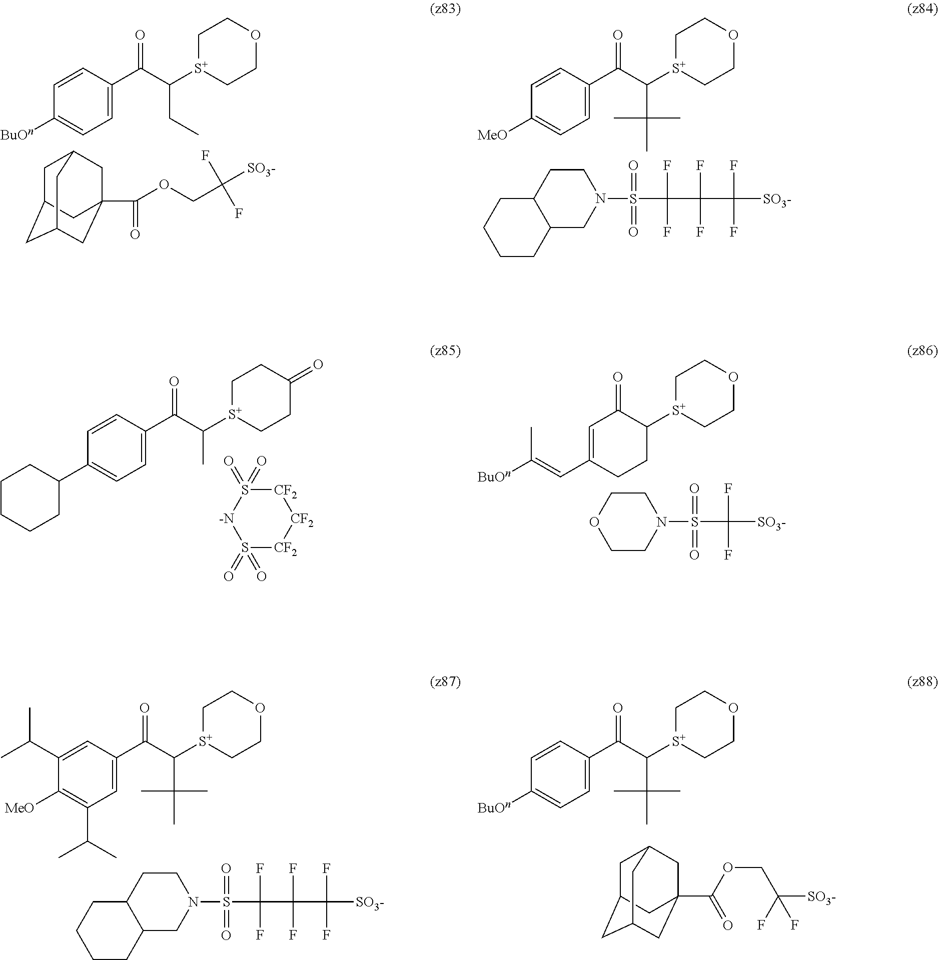

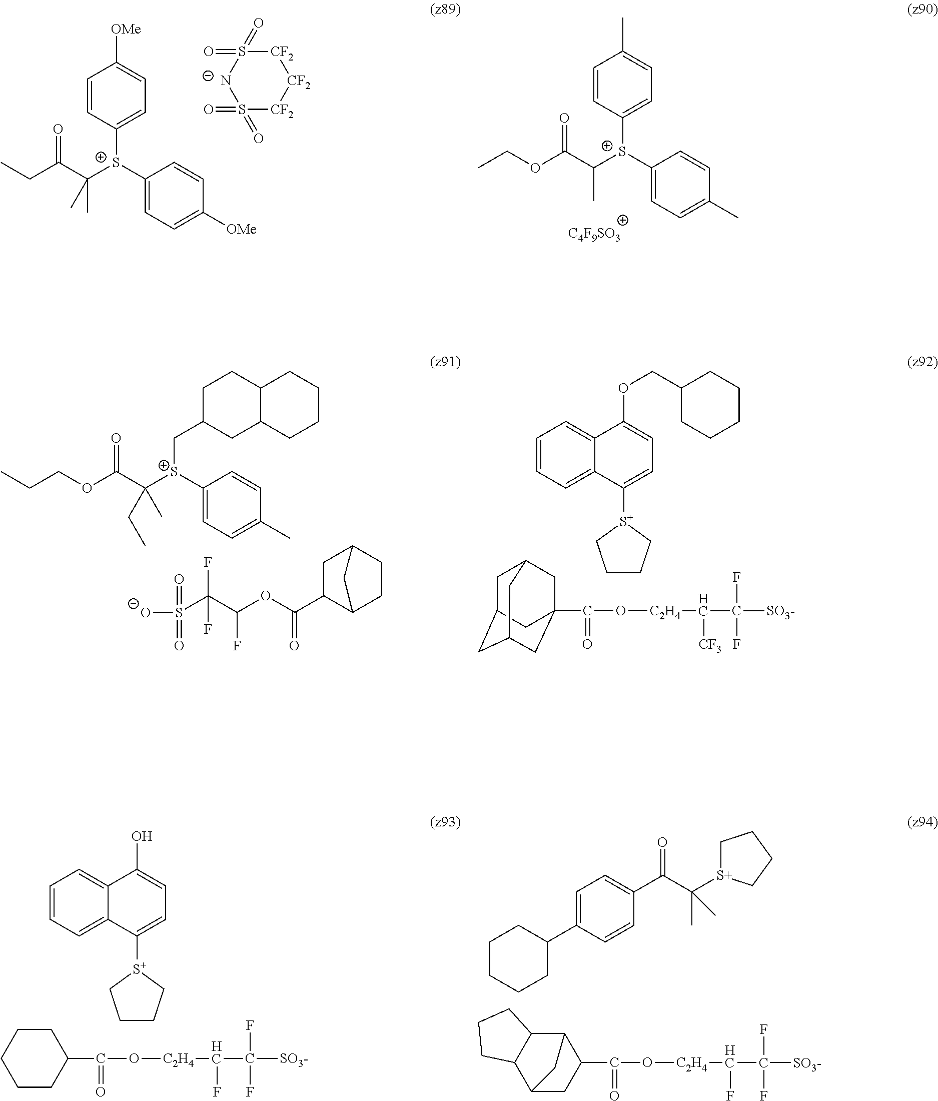

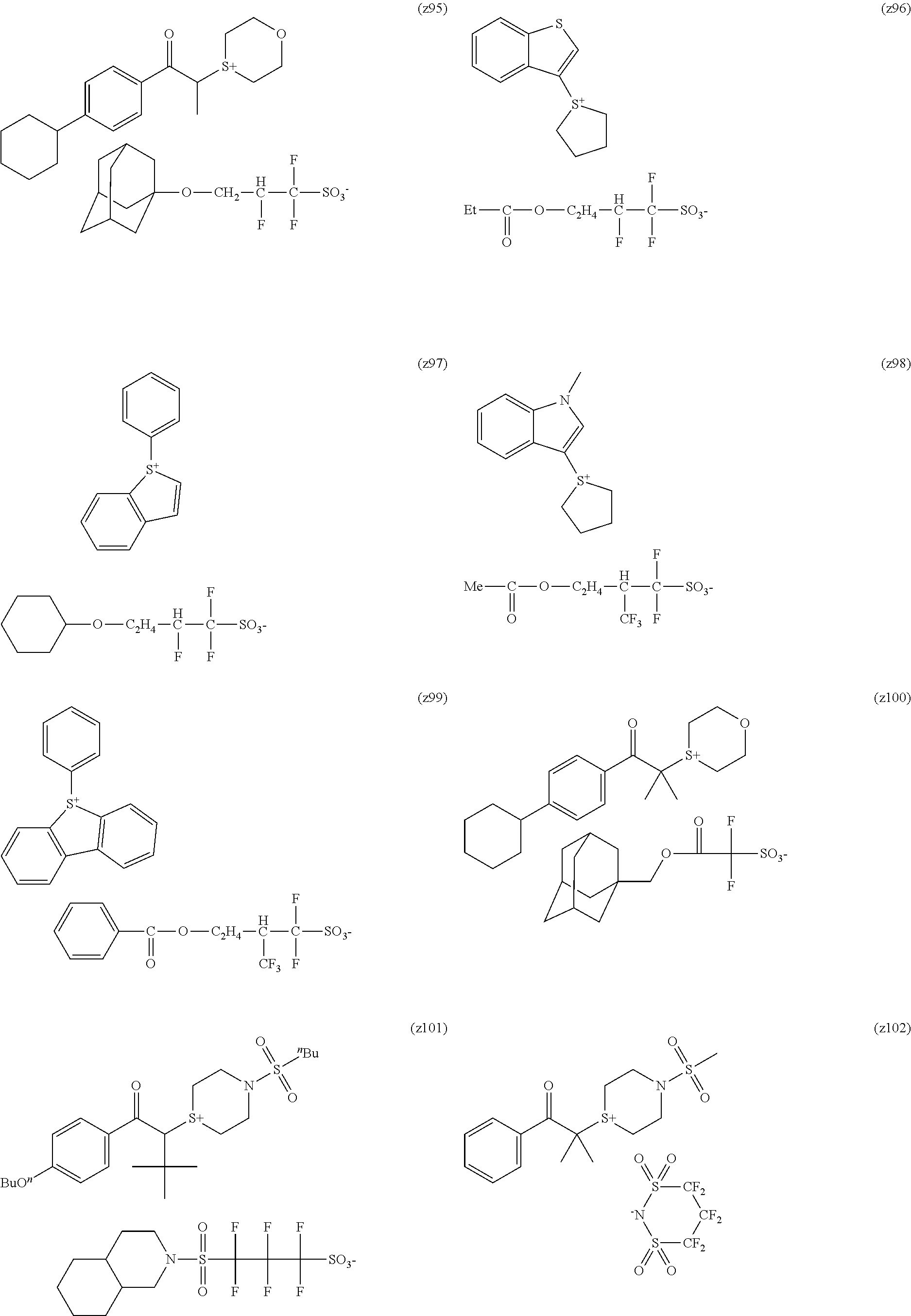

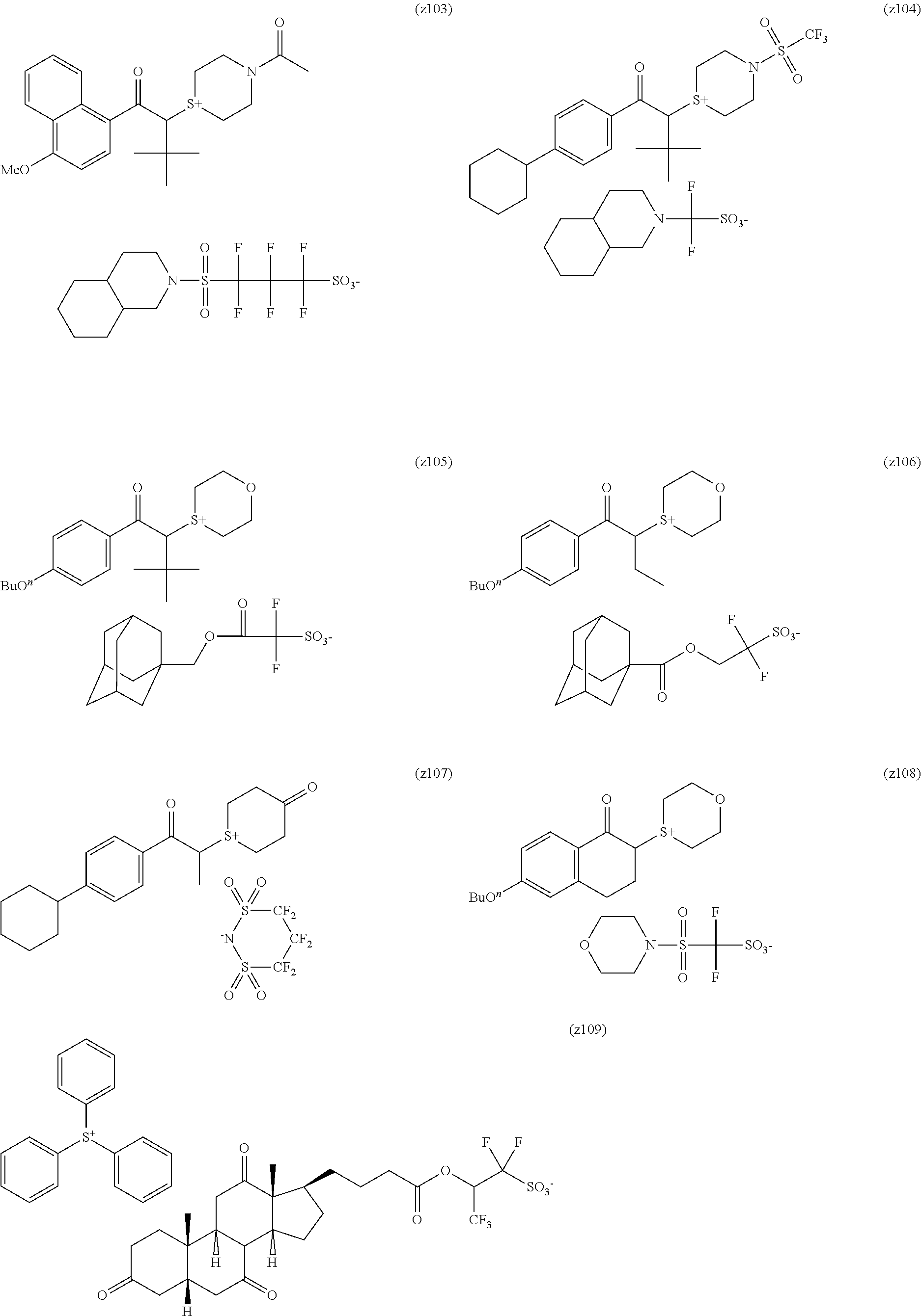

As for the acid generator, one kind may be used alone, or two or more kinds may be used in combination.

The content of the acid generator in the composition is preferably from 0 to 35 mass %, more preferably from 3 to 30 mass %, still more preferably from 5 to 25 mass %, based on the total solid content of the composition.

### [4](D) Hydrophobic Resin

The actinic ray-sensitive or radiation-sensitive resin composition of the present invention may contain a hydrophobic resin (hereinafter, sometimes referred to as “hydrophobic resin (D)” or simply as “resin (D)”) particularly when the composition is applied to immersion exposure. Incidentally, the hydrophobic resin (D) is preferably different from the resin (A).

The hydrophobic resin (D) is unevenly distributed to the film surface layer and when the immersion medium is water, the static/dynamic contact angle on the resist film surface for water as well as the followability of immersion liquid can be enhanced.

The hydrophobic resin (D) is preferably designed, as described above, to be unevenly distributed to the interface but unlike a surfactant, need not have necessarily a hydrophilic group in the molecule and may not contribute to uniform mixing of polar/nonpolar substances.

In view of uneven distribution to the film surface layer, the hydrophobic resin (D) preferably contains any one or more of “a fluorine atom”, “a silicon atom” and “a CH₃ partial structure contained in the side chain moiety of the resin”, more preferably two or more thereof.

In the case where the hydrophobic resin (D) contains a fluorine atom and/or a silicon atom, the fluorine atom and/or silicon atom in the hydrophobic resin (D) may be contained in the main chain of the resin or may be contained in the side chain.

In the case where the hydrophobic resin (D) contains a fluorine atom, the resin is preferably a resin containing a fluorine atom-containing alkyl group, a fluorine atom-containing cycloalkyl group or a fluorine atom-containing aryl group, as a fluorine atom-containing partial structure.

The fluorine atom-containing alkyl group (preferably having a carbon number of 1 to 10, more preferably a carbon number of 1 to 4) is a linear or branched alkyl group with at least one hydrogen atom being substituted for by a fluorine atom and may further have a substituent other than fluorine atom.

The fluorine atom-containing cycloalkyl group is a monocyclic or polycyclic cycloalkyl group with at least one hydrogen atom being substituted for by a fluorine atom and may further have a substituent other than fluorine atom.

The fluorine atom-containing aryl group is an aryl group such as phenyl group or naphthyl group with at least one hydrogen atom being substituted for by a fluorine atom and may further have a substituent other than fluorine atom.

As the fluorine atom-containing alkyl group, fluorine atom-containing cycloalkyl group and fluorine atom-containing aryl group, the groups represented by the following formulae (F2) to (F4) are preferred, but the present invention is not limited thereto.

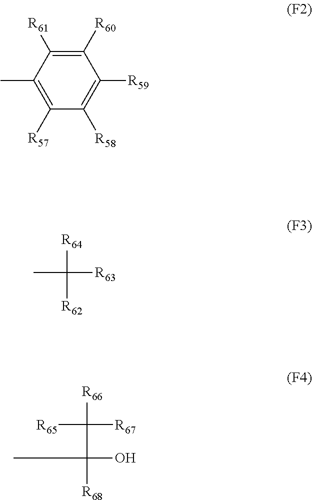

In formulae (F2) to (F4), each of R₅₇ to R₆₈ independently represents a hydrogen atom, a fluorine atom or an alkyl group (linear or branched), provided that at least one of R₅₇ to R₆₁, at least one of R₆₂, to R₆₄, and at least one of R₆₃ to R₆₈ each independently represents a fluorine atom or an alkyl group (preferably having a carbon number of 1 to 4) with at least one hydrogen atom being substituted for by a fluorine atom.

It is preferred that all of R₅₇ to R₆₁ and R₆₅ to R₆₇ are a fluorine atom. Each of R₆₂, R₆₃ and R₆₈ is preferably an alkyl group (preferably having a carbon number of 1 to 4) with at least one hydrogen atom being substituted for by a fluorine atom, more preferably a perfluoroalkyl group having a carbon number of 1 to 4. R₆₂ and R₆₃ may combine with each other to form a ring.

Specific examples of the group represented by formula (F2) include a p-fluorophenyl group, a pentafluorophenyl group, and a 3,5-di(trifluoromethyl)phenyl group.

Specific examples of the group represented by formula (F3) include a trifluoromethyl group, a pentafluoropropyl group, a pentafluoroethyl group, a heptafluorobutyl group, a hexafluoroisopropyl group, a heptafluoroisopropyl group, a hexafluoro(2-methyl)isopropyl group, a nonafluorobutyl group, an octafluoroisobutyl group, a nonafluorohexyl group, a nonafluoro-tert-butyl group, a perfluoroisopentyl group, a perfluorooctyl group, a perfluoro(trimethyl)hexyl group, a 2,2,33-tetrafluorocyclobutyl group, and a perfluorocyclohexyl group. Among these, a hexafluoroisopropyl group, a heptafluoroisopropyl group, a hexafluoro(2-methyl)isopropyl group, an octafluoroisobutyl group, a nonafluoro-tert-butyl group and a perfluoroisopentyl group are preferred, and a hexafluoroisopropyl group and a heptafluoroisopropyl group are more preferred.

Specific examples of the group represented by formula (F4) include —C(CF₃)₂OH, —C(C₂F₅)₂OH, —C(CF₃)(CH₃)OH and —CH(CF₃)OH, with —C(CF₃)₂OH being preferred.

The fluorine atom-containing partial structure may be bonded directly to the main chain or may be bonded to the main chain through a group selected from the group consisting of an alkylene group, a phenylene group, an ether bond, a thioether bond, a carbonyl group, an ester bond, an amide bond, a urethane bond and a ureylene bond, or a group formed by combining two or more of these members.

Specific examples of the repeating unit having a fluorine atom are illustrated below, but the present invention is not limited thereto.

In specific examples, X₁ represents a hydrogen atom, —CH₃, —F or —CF₃, X₂ represents —F or —CF₃.

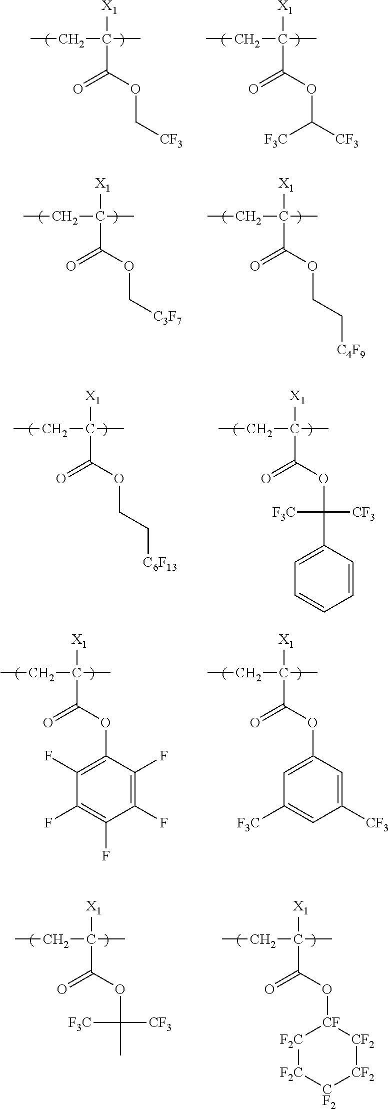

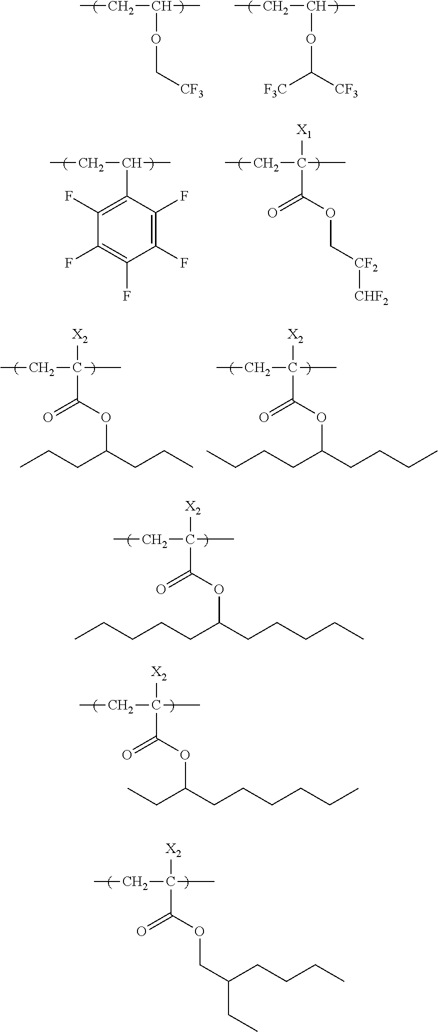

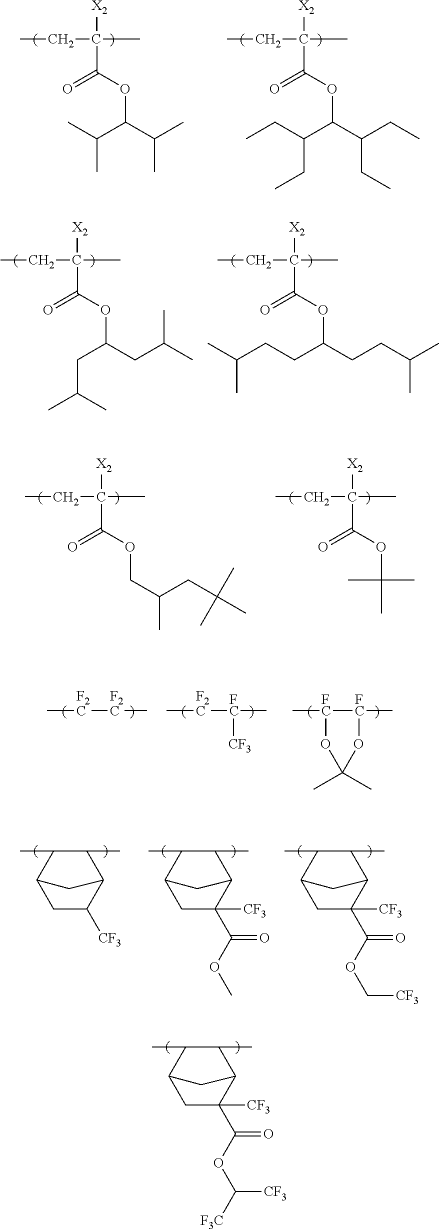

The hydrophobic resin (D) may contain a silicon atom. The resin is preferably a resin having an alkylsilyl structure (preferably a trialkylsilyl group) or a cyclic siloxane structure, as a silicon atom-containing partial structure.

Specific examples of the alkylsilyl structure and cyclic siloxane structure include the groups represented by the following formulae (CS-1) to (CS-3):

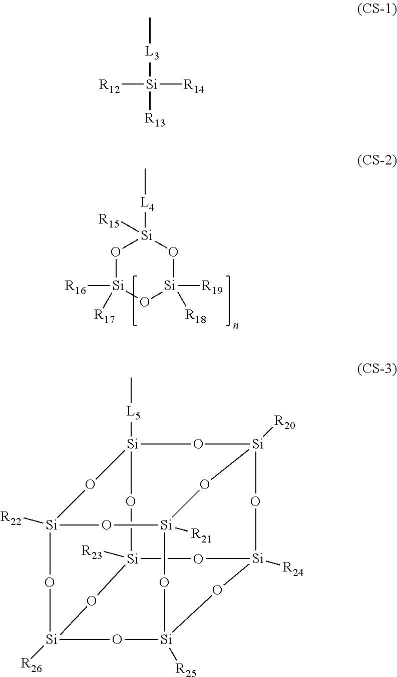

In formulae (CS-1) to (CS-3), each of R₁₂ to R₂₆ independently represents a linear or branched alkyl group (preferably having a carbon number of 1 to 20) or a cycloalkyl group (preferably having a carbon number of 3 to 20).

Each of L₃ to L₅ represents a single bond or a divalent linking group. The divalent linking group includes a single member or a combination of two or more members (preferably having a total carbon number of 12 or less), selected from the group consisting of an alkylene group, a phenylene group, an ether bond, a thioether bond, a carbonyl group, an ester bond, an amide bond, a urethane bond and a urea bond.

n represents an integer of 1 to 5. n is preferably an integer of 2 to 4.

Specific examples of the repeating unit having a group represented by formulae (CS-1) to (CS-3) are illustrated below, but the present invention is not limited thereto. In specific examples, X₁ represents a hydrogen atom, —CH₃, —F or —CF₃.

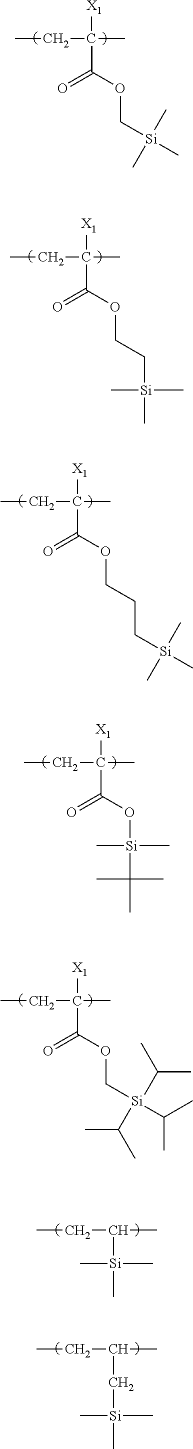

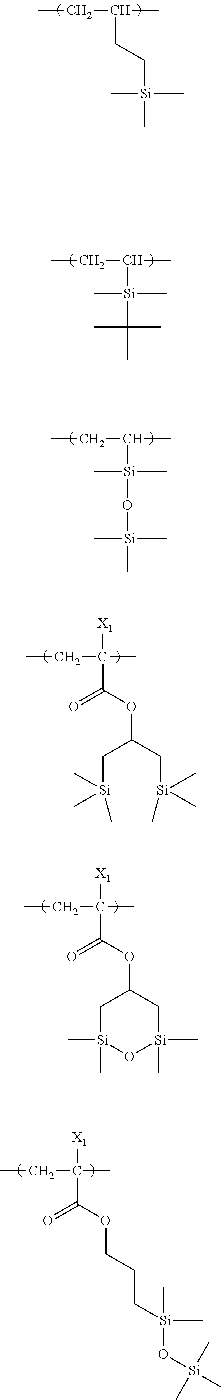

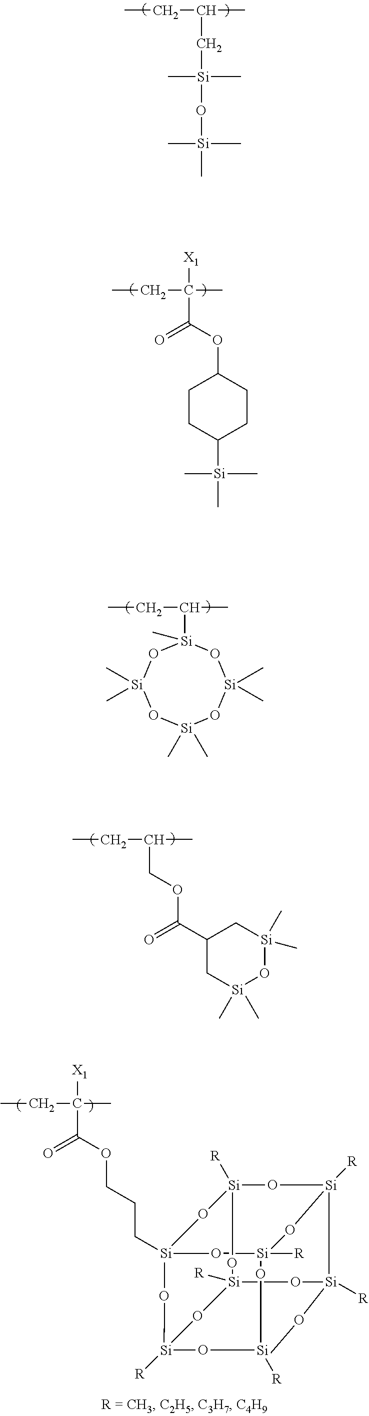

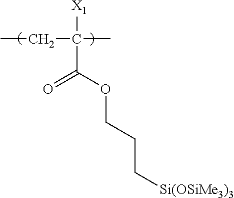

In addition, it is also preferred that, as described above, the hydrophobic resin (D) contains a CH₃ partial structure in the side chain moiety.

Here, the CH₃ partial structure contained in the side chain moiety of the resin (D) (hereinafter, sometimes simply referred to as “side chain CH₃ partial structure”) encompasses a CH₃ partial structure contained in an ethyl group, a propyl group and the like.

On the other hand, a methyl group bonded directly to the main chain of the resin (D) (for example, an α-methyl group of a repeating unit having a methacrylic acid structure) little contributes to surface localization of the resin (D) due to the effect of the main chain and therefore, is not encompassed by the CH₃ partial structure of the present invention.

More specifically, in the case where the resin (D) contains, for example, a repeating unit derived from a monomer containing a polymerizable moiety having a carbon-carbon double bond, such as repeating unit represented by the following formula (M), and where R₁₁ to R₁₄ are CH₃ “itself”, this CH₃ is not encompassed by the CH₃ partial structure contained in the side chain moiety of the present invention.

On the other hand, a CH₃ partial structure connected to the C—C main chain through some atom comes under the CH₃ partial structure of the present invention. For example, when R₁₁ is an ethyl group (CH₂CH₃), this is counted as having “one” CH₃ partial structure of the present invention.

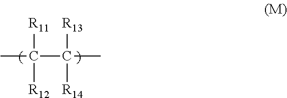

In formula (M), each of R₁₁ to R₁₄ independently represents a side chain moiety.

Examples of the side chain moiety of R₁₁ to R₁₄ include a hydrogen atom and a monovalent organic group.

Examples of the monovalent organic group of R₁₁ to R₁₄ include an alkyl group, a cycloalkyl group, an aryl group, an alkyloxycarbonyl group, a cycloalkyloxycarbonyl group, an aryloxycarbonyl group, an alkylaminocarbonyl group, a cycloalkylaminocarbonyl group, and an arylaminocarbonyl group, and these groups may further have a substituent.

The hydrophobic resin (D) is preferably a resin containing a repeating unit having a CH₃ partial structure in the side chain moiety, and it is more preferred to contain, as such a repeating unit, (x) at least one repeating unit out of a repeating unit represented by the following formula (II) and a repeating unit represented by the following formula (III).

The repeating unit represented by formula (II) is described in detail below.

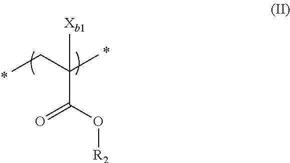

In formula (II), X_{b1} represents a hydrogen atom, an alkyl group, a cyano group or a halogen atom, and R₂ represents an organic group having one or more CH₃ partial structures and being stable to acid. Here, the organic group stable to acid is, more specifically, preferably an organic group not containing the “group capable of decomposing by the action of an acid to produce a polar group” described in the resin (A) above.

The alkyl group of X_{b1} is preferably an alkyl group having a carbon number of 1 to 4, and examples thereof include a methyl group, an ethyl group, a propyl group, a hydroxymethyl group, and a trifluoromethyl group, with a methyl group being preferred.

X_{b1} is preferably a hydrogen atom or a methyl group.

R₂ includes an alkyl group, a cycloalkyl group, an alkenyl group, a cycloalkenyl group, an aryl group, and an aralkyl group, each having one or more CH₃ partial structures. These cycloalkyl group, alkenyl group, cycloalkenyl group, aryl group and aralkyl group may further have an alkyl group as a substituent.

R₂ is preferably an alkyl group or an alkyl-substituted cycloalkyl group, each having one or more CHI partial structures.

The organic group having one or more CH₃ partial structures and being stable to acid of R₂ preferably contains from two to ten, more preferably from two to eight, CH₃ partial structures.

The alkyl group having one or more CH₃ partial structures of R₂ is preferably a branched alkyl group having a carbon number of 3 to 20. Specific preferred examples of the alkyl group include an isopropyl group, an isobutyl group, a 3-pentyl group, a 2-methyl-3-butyl group, a 3-hexyl group, a 2-methyl-3-pentyl group, a 3-methyl-4-hexyl group, a 3,5-dimethyl-4-pentyl group, an isooctyl group, a 2,4,4-trimethylpentyl group, a 2-ethylhexyl group, a 2,6-dimethylheptyl group, a 1,5-dimethyl-3-heptyl group, and a 2,3,5,7-tetramethyl-4-heptyl group. Among these, an isobutyl group, a tert-butyl group, a 2-methyl-3-butyl group, a 2-methyl-3-pentyl group, a 3-methyl-4-hexyl group, a 3,5-dimethyl-4-pentyl group, a 2,4,4-trimethylpentyl group, a 2-ethylhexyl group, a 2,6-dimethyheptyl group, a 1,5-dimethyl-3-heptyl group, and a 2,3,5,7-tetramethyl-4-heptyl group are more preferred.

The cycloalkyl group having one or more CH₃ partial structures of R₂ may be monocyclic or polycyclic and specifically includes a group having a carbon number of 5 or more and containing a monocyclo, bicyclo, tricyclo or tetracyclo structure or the like. The carbon number thereof is preferably from 6 to 30, more preferably from 7 to 25. The cycloalkyl group is preferably an adamantyl group, a noradamantyl group, a decalin residue, a tricyclodecanyl group, a tetracyclododecanyl group, a norbornyl group, a cedrol group, a cyclopentyl group, a cyclohexyl group, a cycloheptyl group, a cyclooctyl group, a cyclodecanyl group or a cyclododecanyl group, more preferably an adamantyl group, a norbornyl group, a cyclohexyl group, a cyclopentyl group, a tetracyclododecanyl group or a tricyclodecanyl group, still more preferably a norbornyl group, a cyclopentyl group or a cyclohexyl group.

The alkenyl group having one or more CH₃ partial structures of R₂ is preferably a linear or branched alkenyl group having a carbon number of 1 to 20, more preferably a branched alkenyl group.

The aryl group having one or more CH₃ partial structures of R₂ is preferably an aryl group having a carbon number of 6 to 20, and examples thereof include a phenyl group and a naphthyl group, with a phenyl group being preferred.

The aralkyl group having one or more CH₃ partial structures of R₂ is preferably an aralkyl group having a carbon number of 7 to 12, and examples thereof include a benzyl group, a phenethyl group and a naphthylmethyl group.

Specific examples of the hydrocarbon group having two or more CH₃ partial structures of R₂ include an isopropyl group, an isobutyl group, a tert-butyl group, a 3-pentyl group, a 2-methyl-3-butyl group, a 3-hexyl group, a 2,3-dimethyl-2-butyl group, a 2-methyl-3-pentyl group, a 3-methyl-4-hexyl group, a 3,5-dimethyl-4-pentyl group, an isooctyl group, a 2,4,4-trimethylpentyl group, a 2-ethylhexyl group, a 2,6-dimethylheptyl group, a 1,5-dimethyl-3-heptyl group, a 2,3,5,7-tetramethyl-4-heptyl group, a 3,5-dimethylcyclohexyl group, a 4-isopropylcyclohexyl group, a 4-tert-butylcyclohexyl group, and an isobornyl group. Among these, an isobutyl group, a tert-butyl group, a 2-methyl-3-butyl group, a 2,3-dimethyl-2-butyl group, a 2-methyl-3-pentyl group, a 3-methyl-4-hexyl group, a 3,5-dimethyl-4-pentyl group, a 2,4,4-trimethylpentyl group, a 2-ethylhexyl group, a 2,6-dimethylheptyl group, a 1,5-dimethyl-3-heptyl group, a 2,3,5,7-tetramethyl-4-heptyl group, a 3,5-dimethylcyclohexyl group, a 3,5-di-tert-butylcyclohexyl group, a 4-isopropylcyclohexyl group, a 4-tert-butylcyclohexyl group and an isobornyl group are preferred.

Specific preferred examples of the repeating unit represented by formula (U) are illustrated below, but the present invention is not limited thereto.

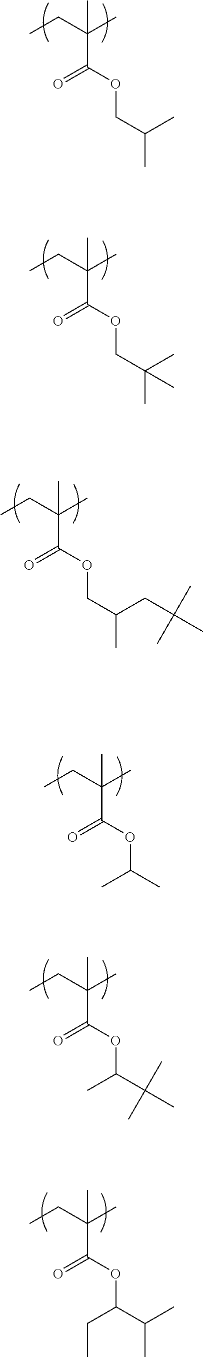

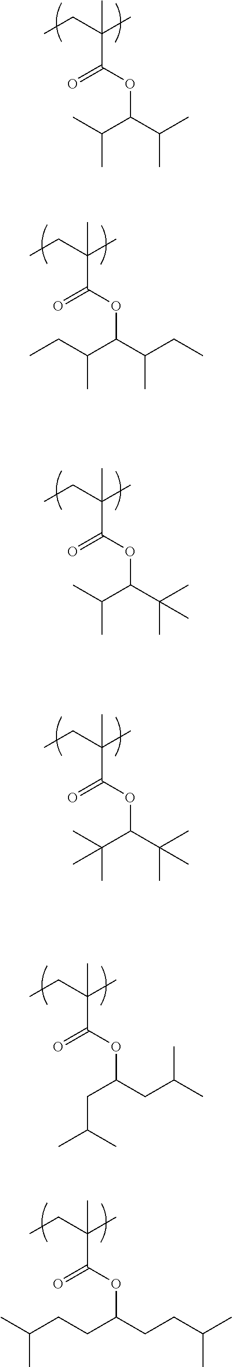

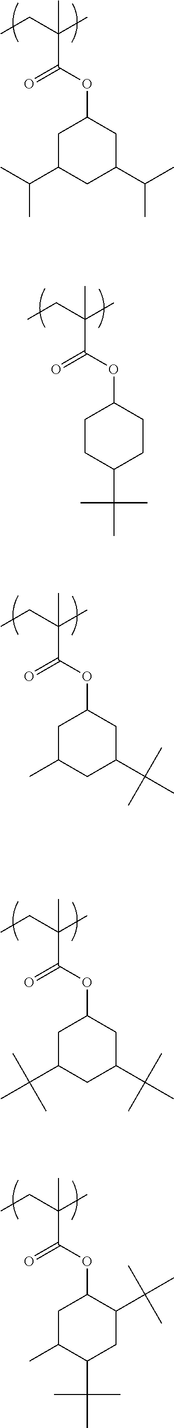

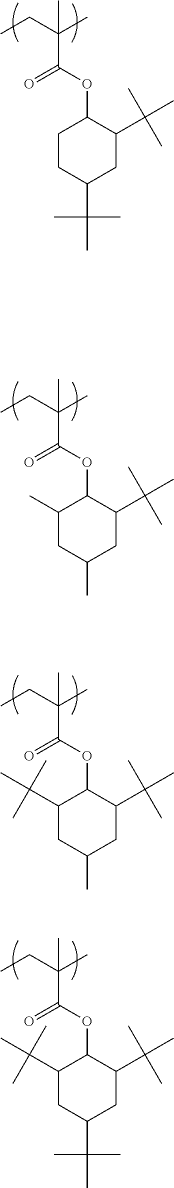

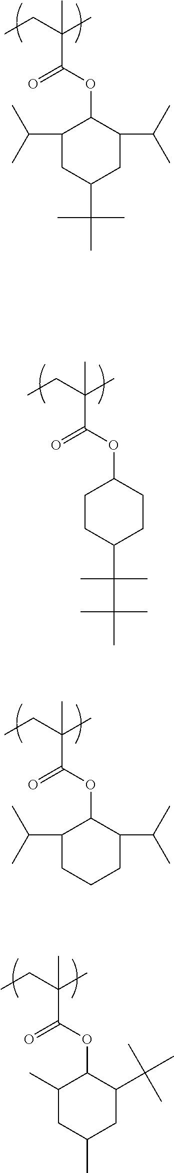

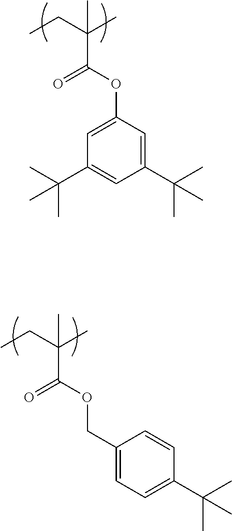

The repeating unit represented by formula (II) is preferably a repeating unit stable to acid (non-acid-decomposable) and specifically, is preferably a repeating unit not having a group capable of decomposing by the action of an acid to produce a polar group.

The repeating unit represented by formula (III) is described in detail below.

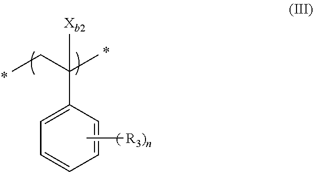

In formula (III), X_{b2} represents a hydrogen atom, an alkyl group, a cyano group or a halogen atom, R₃ represents an organic group having one or more CH₃ partial structures and being stable to acid, and n represents an integer of 1 to 5.

The alkyl group of X_{b2} is preferably an alkyl group having a carbon number of 1 to 4, and examples thereof include a methyl group, an ethyl group, a propyl group, a hydroxymethyl group, and a trifluoromethyl group. A hydrogen atom is preferred.

X_{b2} is preferably a hydrogen atom.

R₃ is an organic group stable to acid and therefore, more specifically, is preferably an organic group not containing the “group capable of decomposing by the action of an acid to produce a polar group” described in the resin (A).

R₃ includes an alkyl group having one or more CH₃ partial structures.

The organic group having one or more CH₃ partial structures and being stable to acid of R₃ preferably contains from one to ten, more preferably from one to eight, still more preferably from one to four, CH₃ partial structures.

The alkyl group having one or more CH₃ partial structures of R₃ is preferably a branched alkyl group having a carbon number of 3 to 20. Specific preferred examples of the alkyl group include an isopropyl group, an isobutyl group, a 3-pentyl group, a 2-methyl-3-butyl group, a 3-hexyl group, a 2-methyl-3-pentyl group, a 3-methyl-4-hexyl group, a 3,5-dimethyl-4-pentyl group, an isooctyl group, a 2,4,4-trimethylpentyl group, a 2-ethylhexyl group, a 2,6-dimethylheptyl group, a 1,5-dimethyl-3-heptyl group, and a 2,3,5,7-tetramethyl-4-heptyl group. Among these, an isobutyl group, a tert-butyl group, a 2-methyl-3-butyl group, a 2-methyl-3-pentyl group, a 3-methyl-4-hexyl group, a 3,5-dimethyl-4-pentyl group, a 2,4,4-trimethylpentyl group, a 2-ethylhexyl group, a 2,6-dimethylheptyl group, a 1,5-dimethyl-3-heptyl group and a 2,3,5,7-tetramethyl-4-heptyl group are more preferred.

Specific examples of the alkyl group having two or more CH₃ partial structures of R₃ include an isopropyl group, an isobutyl group, a tert-butyl group, a 3-pentyl group, a 2.3-dimethylbutyl group, a 2-methyl-3-butyl group, a 3-hexyl group, a 2-methyl-3-pentyl group, a 3-methyl-4-hexyl group, a 3,5-dimethyl-4-pentyl group, an isooctyl group, a 2,4,4-trimethylpentyl group, a 2-ethylhexyl group, a 2,6-dimethylheptyl group, a 1,5-dimethyl-3-heptyl group, and a 2,3,5,7-tetramethyl-4-heptyl group. Among these, those having a carbon number of 5 to 20, that is, an isobutyl group, a tert-butyl group, a 2-methyl-3-butyl group, a 2-methyl-3-pentyl group, a 3-methyl-4-hexyl group, a 3,5-dimethyl-4-pentyl group, a 2,4,4-trimethylpentyl group, a 2-ethylhexyl group, a 2,6-dimethylheptyl group, a 1,5-dimethyl-3-heptyl group, a 2,3,5,7-tetramethyl-4-heptyl group and a 2,6-dimethylheptyl group, are preferred. n represents an integer of 1 to 5, preferably an integer of 1 to 3, more preferably 1 or 2.

Specific preferred examples of the repeating unit represented by formula (III) are illustrated below, but the present invention is not limited thereto.

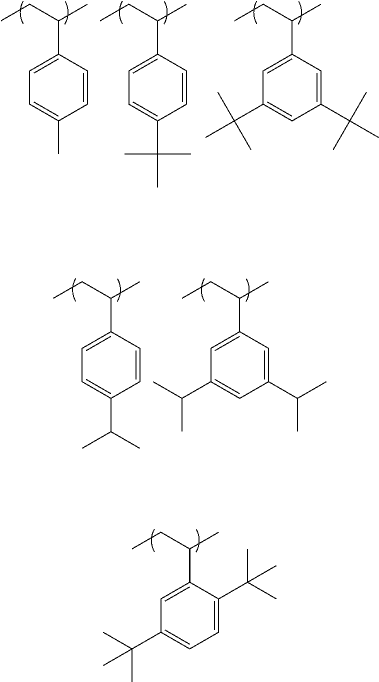

The repeating unit represented by formula (III) is preferably a repeating unit stable to acid (non-acid-decomposable) and specifically, is preferably a repeating unit not having a group capable of decomposing by the action of an acid to produce a polar group.

In the case where the resin (D) contains a CH₃ partial structure in the side chain moiety and furthermore, does not have a fluorine atom and a silicon atom, the content of the (x) at least one repeating unit out of a repeating unit represented by formula (II) and a repeating unit represented by formula (III) is preferably 90 mol % or more, more preferably 95 mol % or more, based on all repeating units in the resin (C). The content is usually 100 mol % or less based on all repeating units in the resin (C).

When the resin (D) contains the (x) at least one repeating unit out of a repeating unit represented by formula (II) and a repeating unit represented by formula (III) in a ratio of 90 mol % or more based on all repeating units in the resin (D), the surface free energy of the resin (C) is increased and in turn, the resin (D) is less likely to be unevenly distributed to the surface of the resist film, as a result, the static/dynamic contact angle of the resist film for water can be unfailingly raised and the followability of immersion liquid can be enhanced.

Furthermore, in both of (i) a case of containing a fluorine atom and/or a silicon atom and (ii) a case of containing a CH₃ partial structure in the side chain moiety, the hydrophobic resin (D) may contain at least one group selected from the group consisting of the following (x) to (z). Such a group is suitably used particularly when the composition of the present invention is used for an alkali developing process.

(x) An acid group
(y) A lactone structure-containing group, an acid anhydride group, or an acid imide group
(z) A group capable of decomposing by the action of an acid

Examples of the acid group (x) include a phenolic hydroxyl group, a carboxylic acid group, a fluorinated alcohol group, a sulfonic acid group, a sulfonamide group, a sulfonylimide group, an (alkylsulfonyl)(alkylcarbonyl)methylene group, an (alkylsulfonyl)(alkylcarbonyl)imide group, a bis(alkylcarbonyl)methylene group, a bis(alkylcarbonyl)imide group, a bis(alkylsulfonyl)methylene group, a bis(alkylsulfonyl)imide group, a tris(alkylcarbonyl)methylene group, and a tris(alkylsulfonyl)methylene group.

Preferred acid groups include a fluorinated alcohol group (preferably hexafluoroisopropanol), a sulfonimide group, and a bis(alkylcarbonyl)methylene group.

Examples of the (x) repeating unit having an acid group include a repeating unit where the acid group is directly bonded to the main chain of the resin, such as repeating unit by an acrylic acid or a methacrylic acid, and a repeating unit where the acid group is bonded to the main chain of the resin through a linking group, and the acid group may also be introduced into the polymer chain terminal by using an acid group-containing polymerization initiator or chain transfer agent at the polymerization. All of these cases are preferred. The (x) repeating unit having an acid group may have at least either a fluorine atom or a silicon atom.

The content of the (x) repeating unit having an acid group is preferably from 1 to 50 mol %, more preferably from 3 to 35 mol %, still more preferably from 5 to 20 mol %, based on all repeating units in the hydrophobic resin (D).

Specific examples of the (x) repeating unit having an acid group are illustrated below, but the present invention is not limited thereto. In the formulae, Rx represents a hydrogen atom, CH₃, CF₃ or CH₂OH.

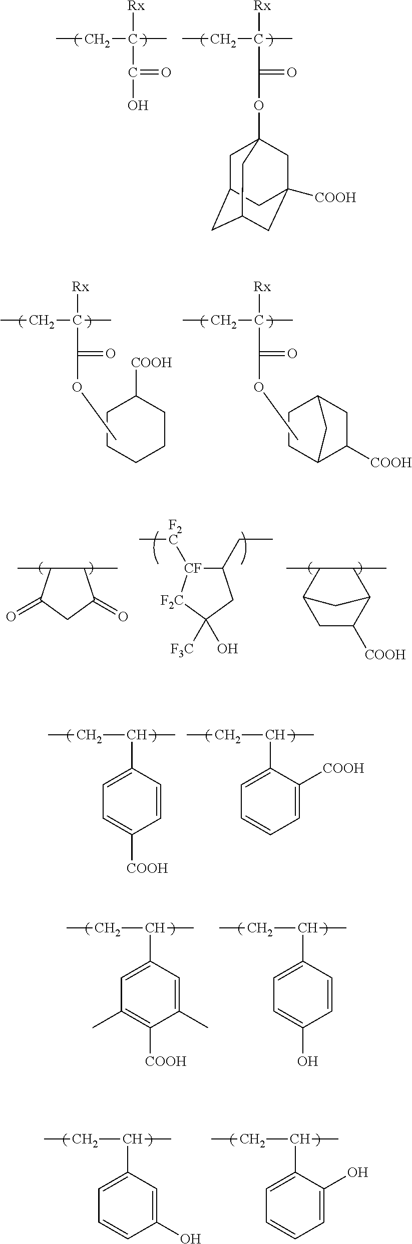

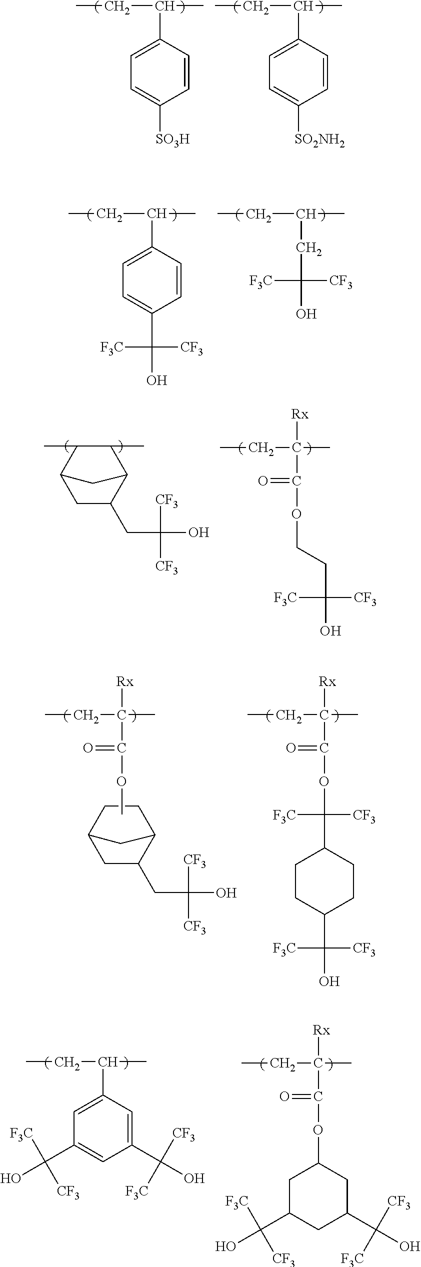

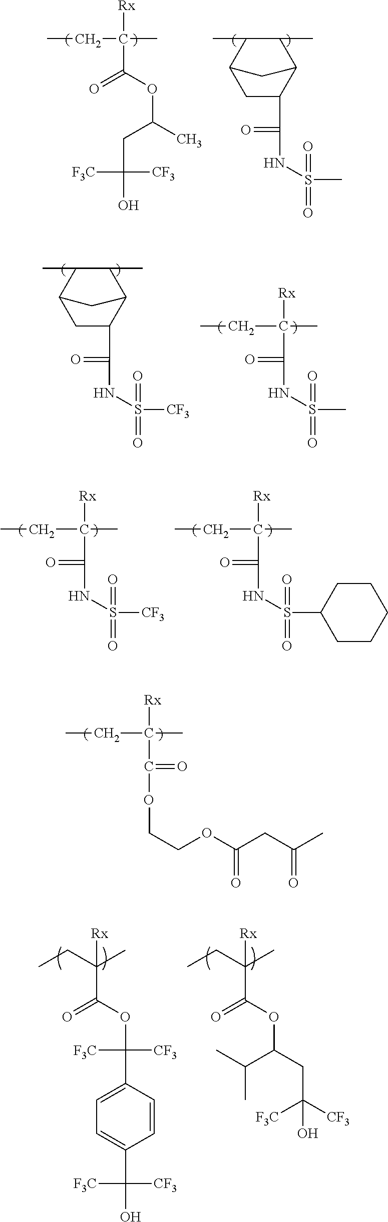

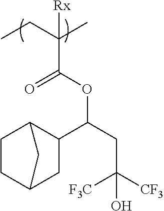

The (y) lactone structure-containing group, acid anhydride group or acid imide group is preferably a lactone structure-containing group.

The repeating unit containing such a group is, for example, a repeating unit where the group is directly bonded to the main chain of the resin, such as repeating unit by an acrylic acid ester or a methacrylic acid ester. This repeating unit may be a repeating unit where the group is bonded to the main chain of the resin through a linking group. Alternatively, in this repeating unit, the group may be introduced into the terminal of the resin by using a polymerization initiator or chain transfer agent containing the group at the polymerization.

Examples of the repeating unit having a lactone structure-containing group are the same as those of the repeating unit having a lactone structure described above in the paragraph of the acid-decomposable resin (A). Also, repeating units disclosed in paragraph [0725] of U.S. Patent Application Publication No. 2012/0135348A1 may also be suitably used. Preferred examples of the repeating unit having a lactone structure-containing group include the repeating units contained in HR-66 to HR-80 illustrated later.

The content of the repeating unit having a lactone structure-containing group, an acid anhydride group or an acid imide group is preferably from 1 to 100 mol %, more preferably from 3 to 98 mol %, still more preferably from 5 to 95 mol %, based on all repeating units in the hydrophobic resin (D).

Examples of the repeating unit having (z) a group capable of decomposing by the action of an acid, contained in the hydrophobic resin (D), are the same as those of the repeating unit having an acid-decomposable group described in the resin (A). The repeating unit having (z) a group capable of decomposing by the action of an acid may contain at least either a fluorine atom or a silicon atom. In the hydrophobic resin (D), the content of the repeating unit having (z) a group capable of decomposing by the action of an acid is preferably from 1 to 80 mol %, more preferably from 10 to 80 mol %, still more preferably from 20 to 60 mol %, based on all repeating units in the resin (D).

The hydrophobic resin (D) may further contain a repeating unit represented by the following formula (III):

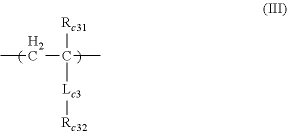

In formula (III), R_{c31} represents a hydrogen atom, an alkyl group (which may be substituted with a fluorine atom or the like), a cyano group or a —CH₂—O—R_{ac2} group, wherein R_{ac2} represents a hydrogen atom, an alkyl group or an acyl group. R_{c31} is preferably a hydrogen atom, a methyl group, a hydroxymethyl group or a trifluoromethyl group, more preferably a hydrogen atom or a methyl group.

R_{c32} represents a group having an alkyl group, a cycloalkyl group, an alkenyl group, a cycloalkenyl group or an aryl group. These groups may be substituted with a fluorine atom or a silicon atom-containing group.

L_{c3} represents a single bond or a divalent linking group.

In formula (III), the alkyl group of R_{c32} is preferably a linear or branched alkyl group having a carbon number of 3 to 20.

The cycloalkyl group is preferably a cycloalkyl group having a carbon number of 3 to 20.

The alkenyl group is preferably an alkenyl group having a carbon number of 3 to 20.

The cycloalkenyl group is preferably a cycloalkenyl group having a carbon number of 3 to 20.

The aryl group is preferably an aryl group having a carbon number of 6 to 20, more preferably a phenyl group or a naphthyl group, and these groups may have a substituent.

R_{c32} is preferably an unsubstituted alkyl group or an alkyl group substituted with a fluorine atom.

The divalent linking group of L_{c3} is preferably an alkylene group (preferably having a carbon number of 1 to 5), an ether bond, a phenylene group or an ester bond (a group represented by —COO—).

The content of the repeating unit represented by formula (III) is preferably from 1 to 100 mol %, more preferably from 10 to 90 mol %, still more preferably from 30 to 70 mol %, based on all repeating units in the hydrophobic resin.

It is also preferred that the hydrophobic resin (D) further contains a repeating unit represented by the following formula (CII-AB):

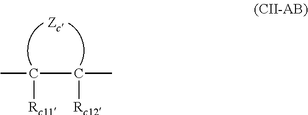

In formula (CII-AB),

each of R_{c11}′ and R_{c12}′ independently represents a hydrogen atom, a cyano group, a halogen atom or an alkyl group, and

Z_{c}′ represents an atomic group for forming an alicyclic structure containing two carbon atoms (C—C) to which Z_{c}′ is bonded.

The content of the repeating unit represented by formula (CII-AB) is preferably from 1 to 100 mol %, more preferably from 10 to 90 mol %, still more preferably from 30 to 70 mol %, based on all repeating units in the hydrophobic resin.

Specific examples of the repeating units represented by formulae (III) and (CII-AB) are illustrated below, but the present invention is not limited thereto. In the formulae, Ra represents H, CH₃, CH₂OH, CF₃ or CN.

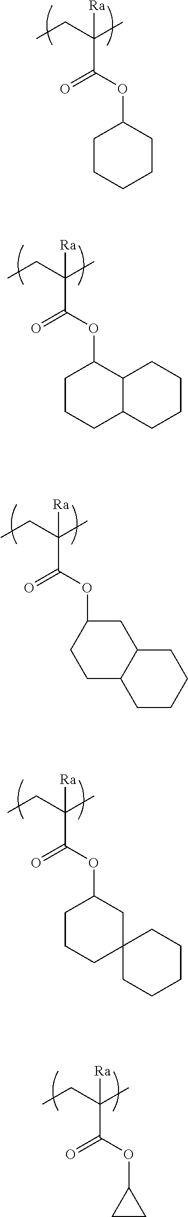

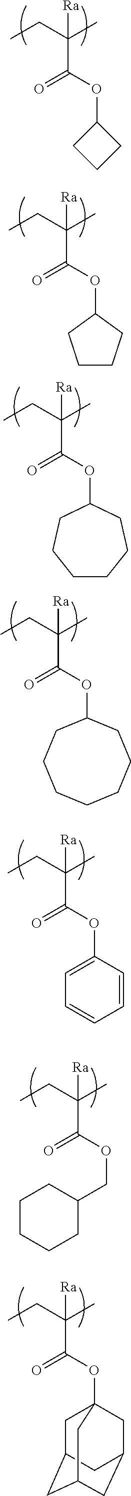

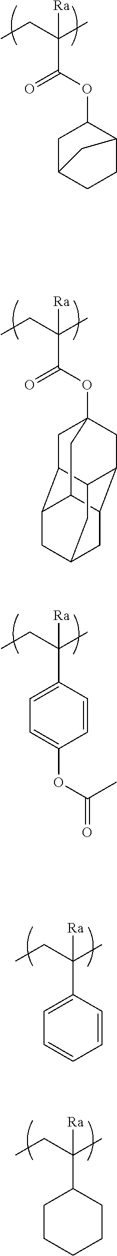

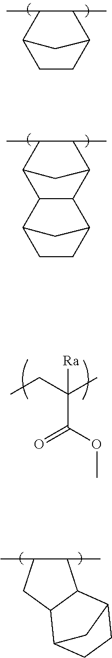

In the case where the hydrophobic resin (D) contains a fluorine atom, the fluorine atom content is preferably from 5 to 80 mass %, more preferably from 10 to 80 mass %, based on the weight average molecular weight of the hydrophobic resin (D). Also, the fluorine atom-containing repeating unit preferably accounts for 10 to 100 mol %, more preferably from 30 to 100 mol %, based on all repeating units contained in the hydrophobic resin (D).

In the case where the hydrophobic resin (D) contains a silicon atom, the silicon atom content is preferably from 2 to 50 mass %, more preferably from 2 to 30 mass %, based on the weight average molecular weight of the hydrophobic resin (D). Also, the silicon atom-containing repeating unit preferably accounts for 10 to 100 mol %, more preferably from 20 to 100 mol %, based on all repeating units contained in the hydrophobic resin (D).

On the other hand, particularly when the resin (D) contains a CH₃ partial structure in the side chain moiety, an embodiment where the resin (D) contains substantially no fluorine atom and no silicon atom is also preferred, and in this case, specifically, the content of the repeating unit having a fluorine atom or a silicon atom is, based on all repeating units in the resin (D), preferably 5 mol % or less, more preferably 3 mol % or less, still more preferably 1 mol % or less, and ideally 0 mol %, that is, not containing a fluorine atom and a silicon atom. Also, the resin (D) preferably consists of substantially only a repeating unit composed of only an atom selected from a carbon atom, an oxygen atom, a hydrogen atom, a nitrogen atom and a sulfur atom. More specifically, the repeating unit composed of only an atom selected from a carbon atom, an oxygen atom, a hydrogen atom, a nitrogen atom and a sulfur atom preferably accounts for 95 mol % or more, more preferably 97 mol % or more, still more preferably 99 mol % or more, and ideally 100 mol %, based on all repeating units in the resin (D).

The weight average molecular weight of the hydrophobic resin (D) is, in terms of standard polystyrene, preferably from 1,000 to 100,000, more preferably from 1,000 to 50,000, still more preferably from 2,000 to 15,000.

As for the hydrophobic resin (D), one resin may be used, or a plurality of resins may be used in combination.

The content of the hydrophobic resin (D) in the composition is preferably from 0.01 to 10 mass %, more preferably from 0.05 to 8 mass %, still more preferably from 0.1 to 7 mass %, based on the total solid content of the composition of the present invention.

In the hydrophobic resin (D), similarly to the resin (A), it is of course preferred that the content of impurities such as metal is small, but the content of residual monomers or oligomer components is also preferably from 0.01 to 5 mass %, more preferably from 0.01 to 3 mass %, still more preferably from 0.05 to 1 mass %. Within this range, an actinic ray-sensitive or radiation-sensitive resin composition free from in-liquid extraneous substances and changes with aging of sensitivity or the like can be obtained. Furthermore, in view of resolution, resist profile, side wall of resist pattern, roughness and the like, the molecular weight distribution (Mw/Mn, sometimes referred to as “polydispersity”) is preferably from 1 to 5, more preferably from 1 to 3, still more preferably from 1 to 2.

As the hydrophobic resin (D), various commercially products may be used, or the resin may be synthesized by a conventional method (for example, radical polymerization). Examples of the general synthesis method include a batch polymerization method of dissolving monomer species and an initiator in a solvent and heating the solution, thereby effecting the polymerization, and a dropping polymerization method of adding dropwise a solution containing monomer species and an initiator to a heated solvent over 1 to 10 hours. A dropping polymerization method is preferred.

The reaction solvent, the polymerization initiator, the reaction conditions (such as temperature and concentration) and the method for purification after reaction are the same as those described for the resin (A), but in the synthesis of the hydrophobic resin (D), the concentration at the reaction is preferably from 30 to 50 mass %.

Specific examples of the hydrophobic resin (D) are illustrated below. Also, the molar ratio of repeating units (corresponding to respective repeating units starting from the left), weight average molecular weight and polydispersity of each resin are shown in the Tables later.

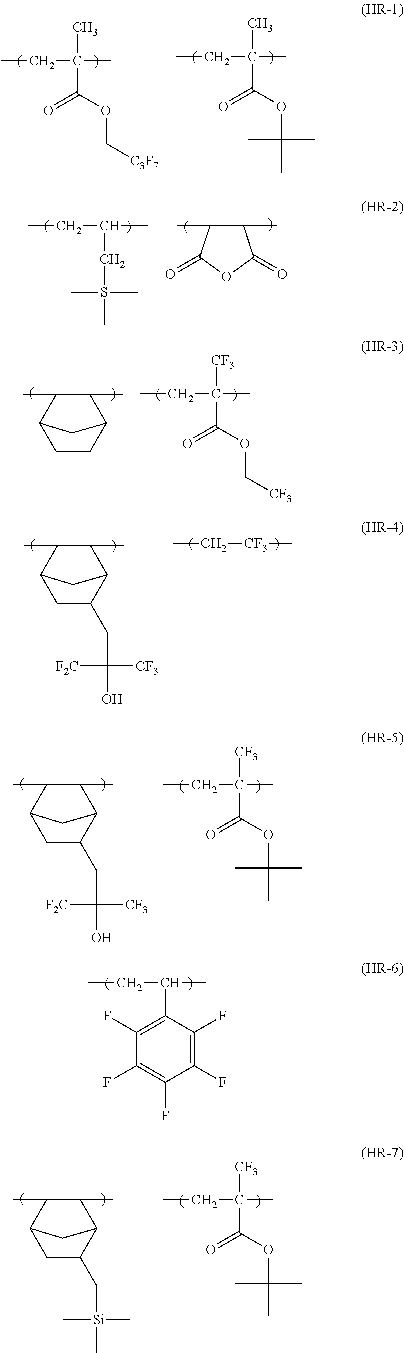

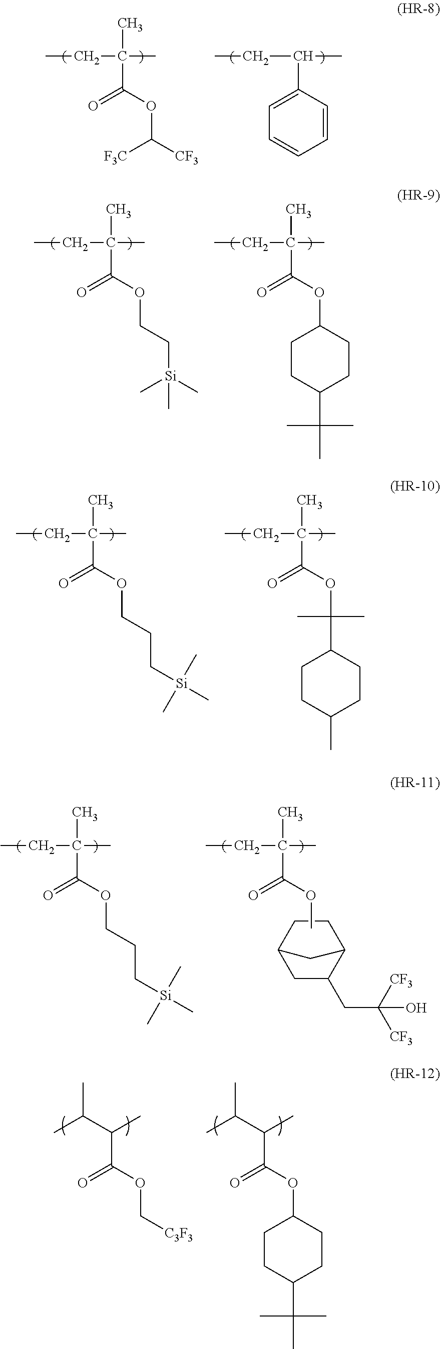

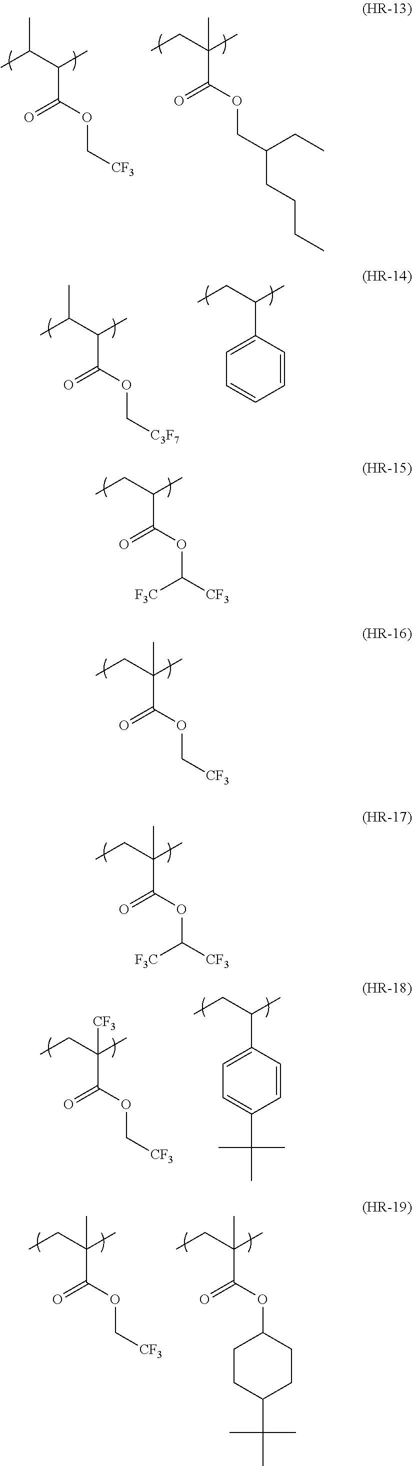

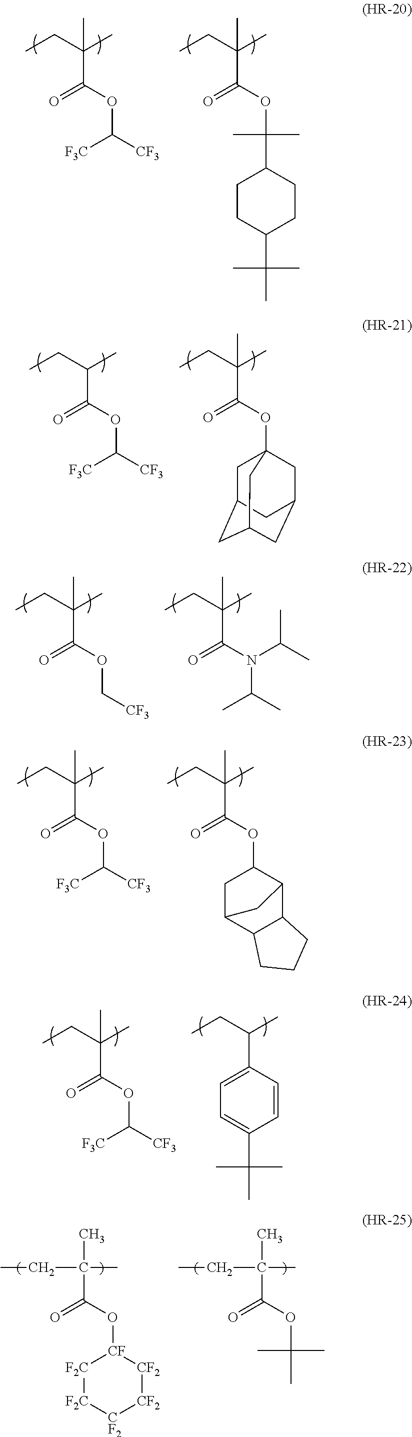

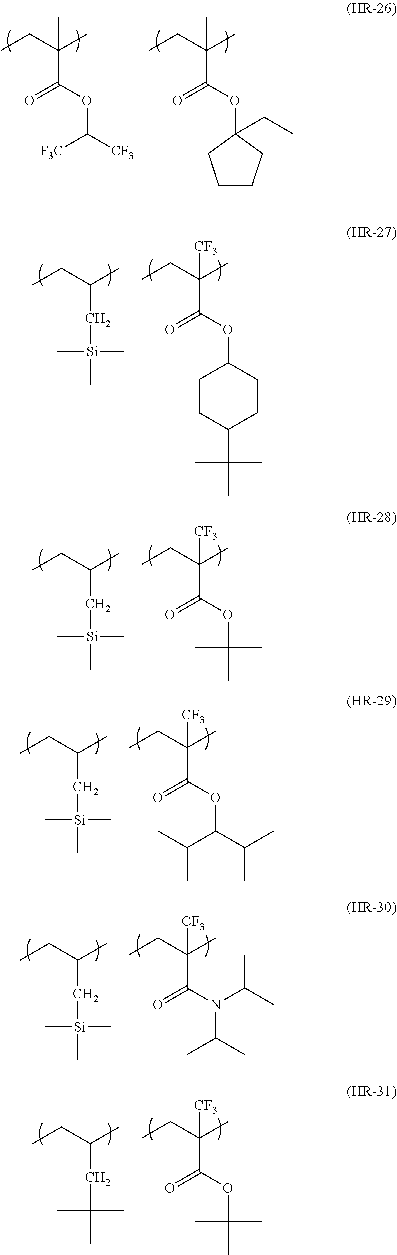

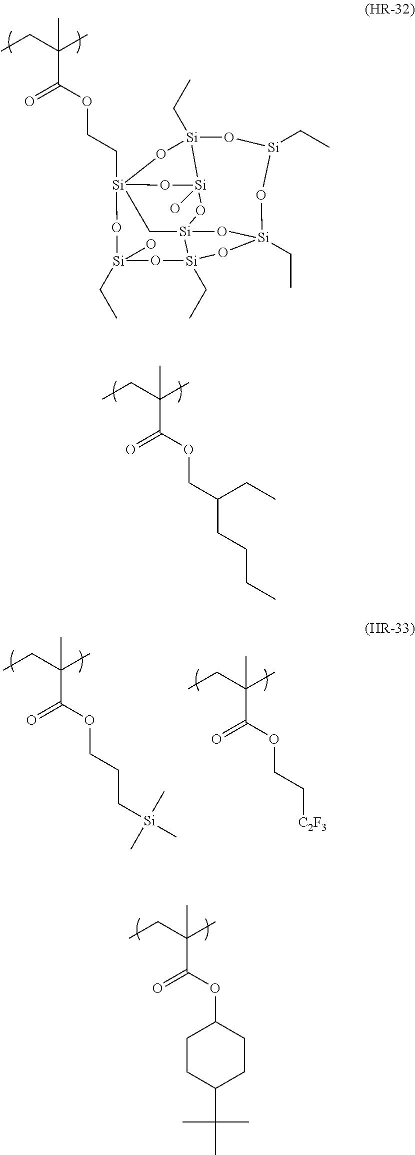

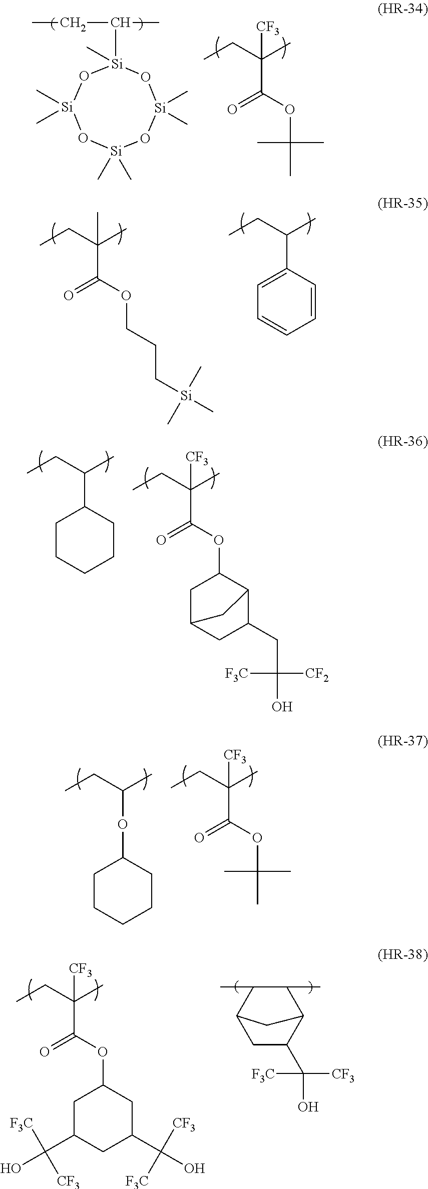

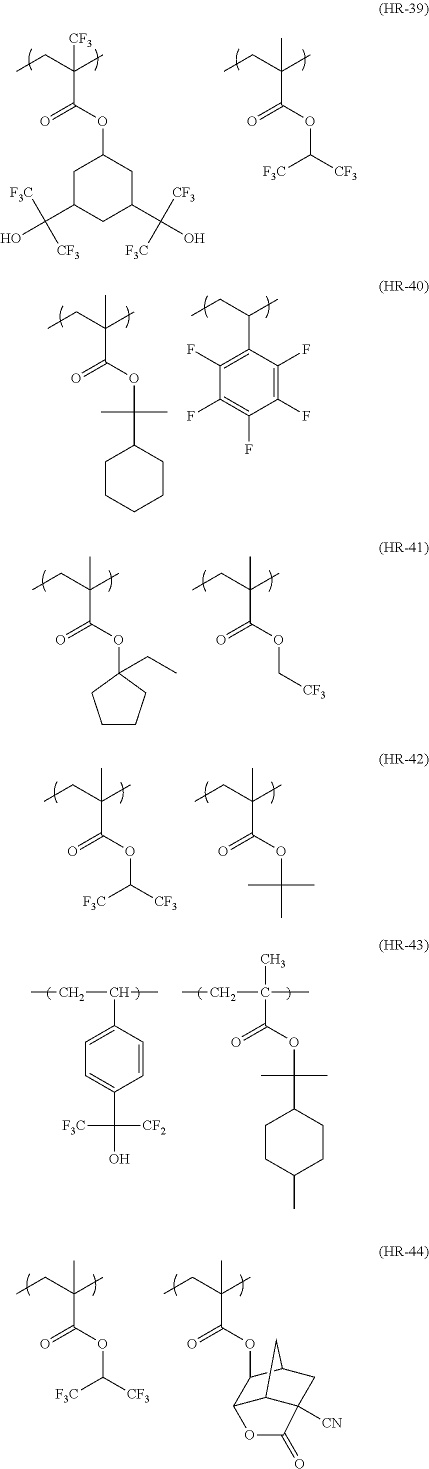

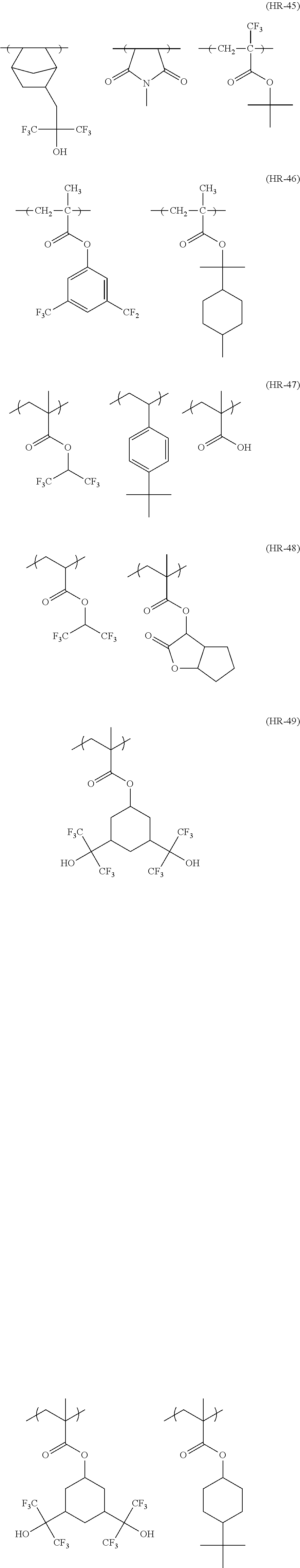

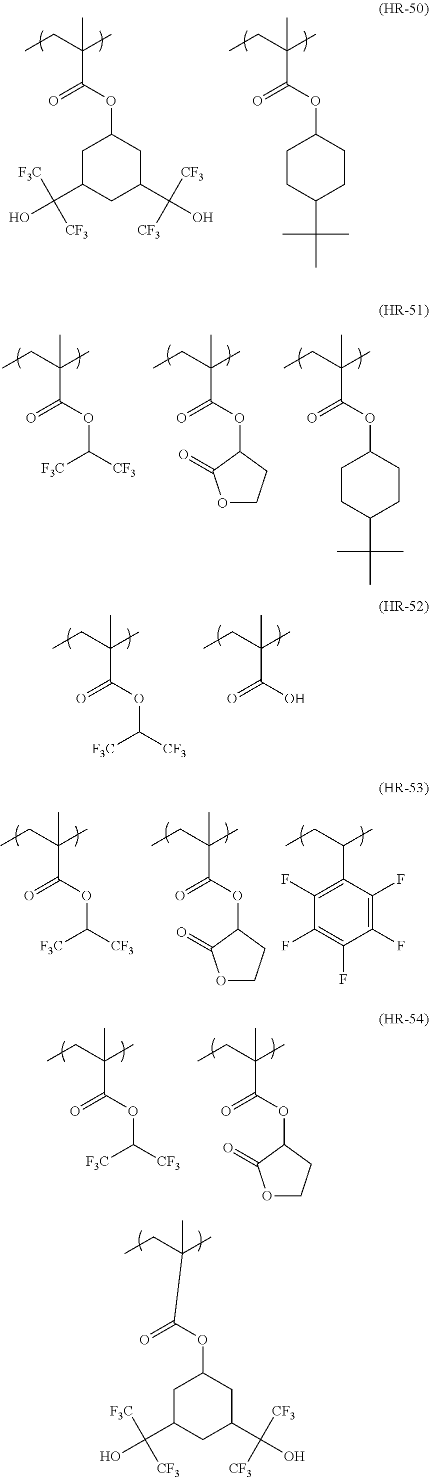

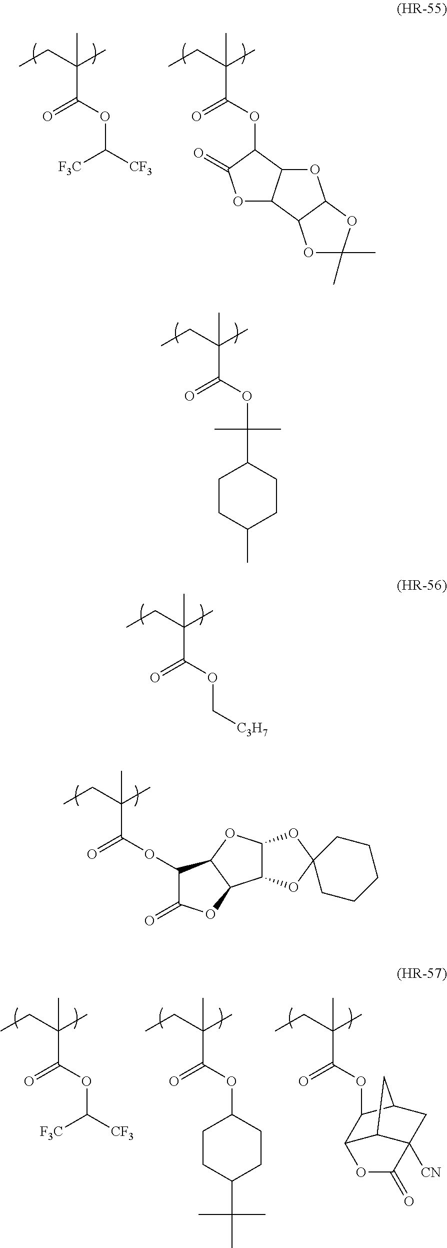

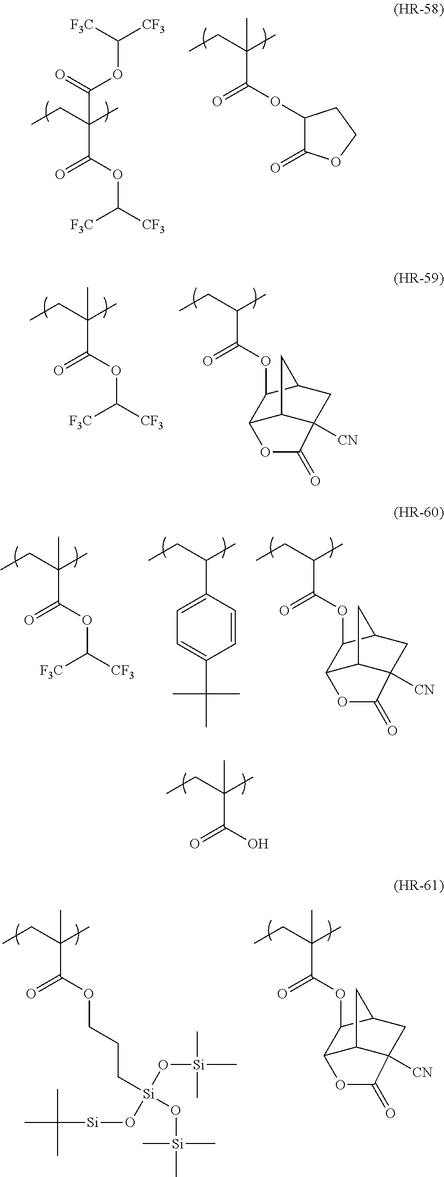

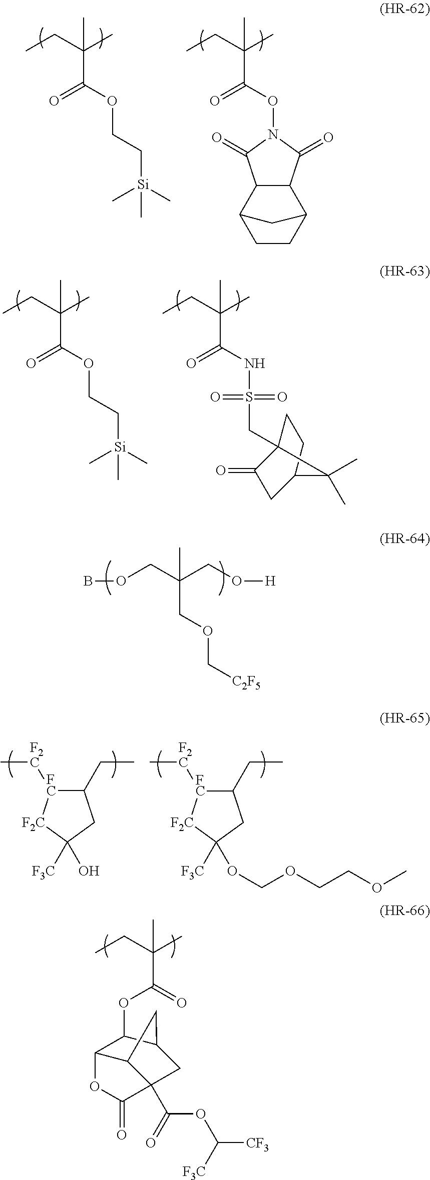

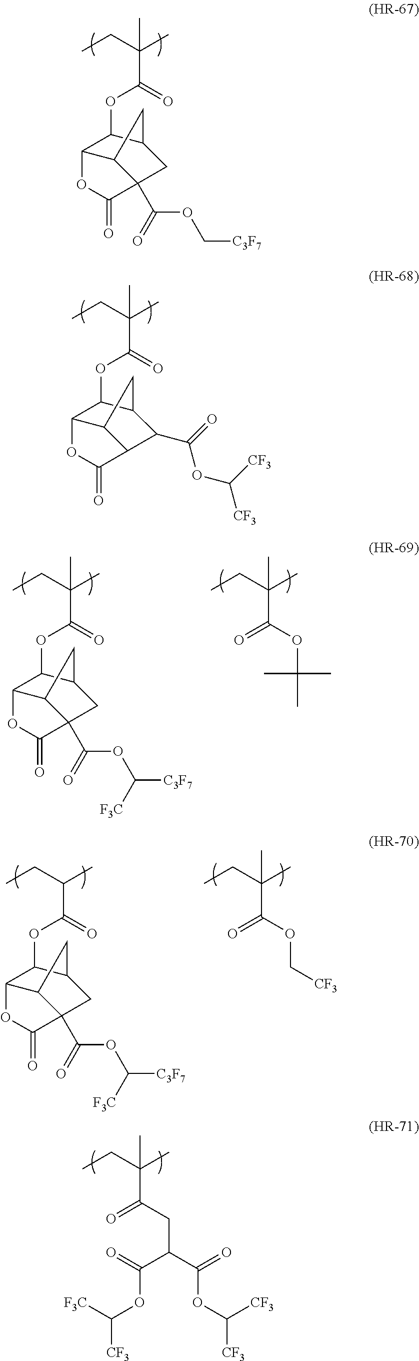

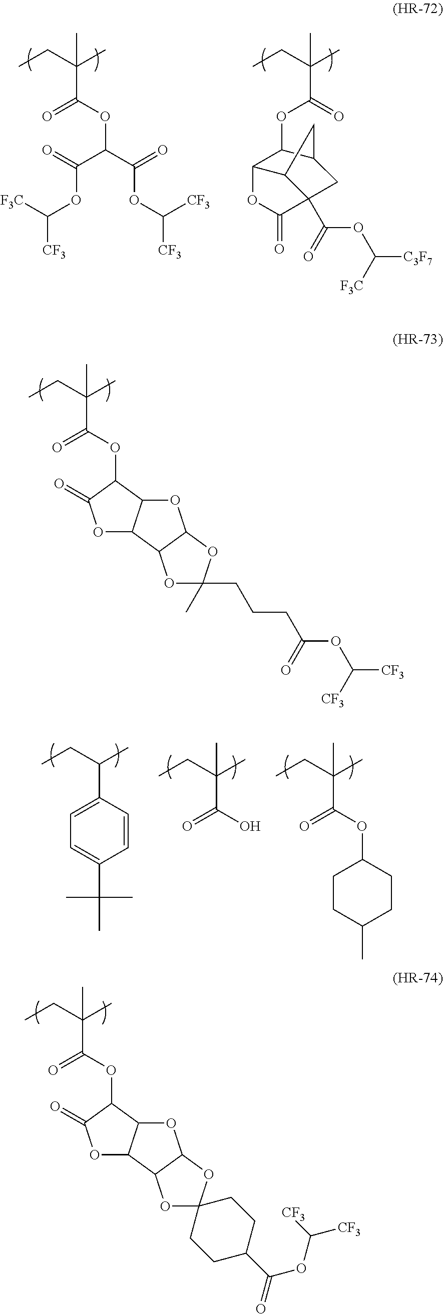

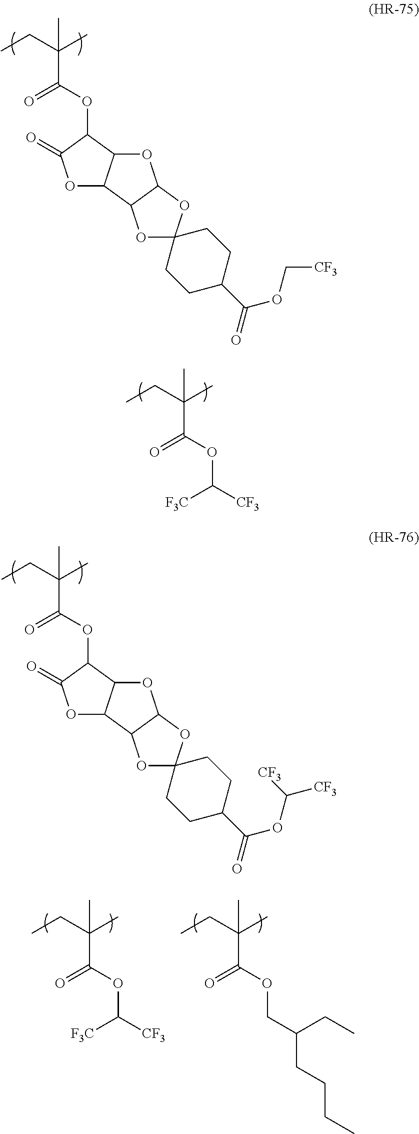

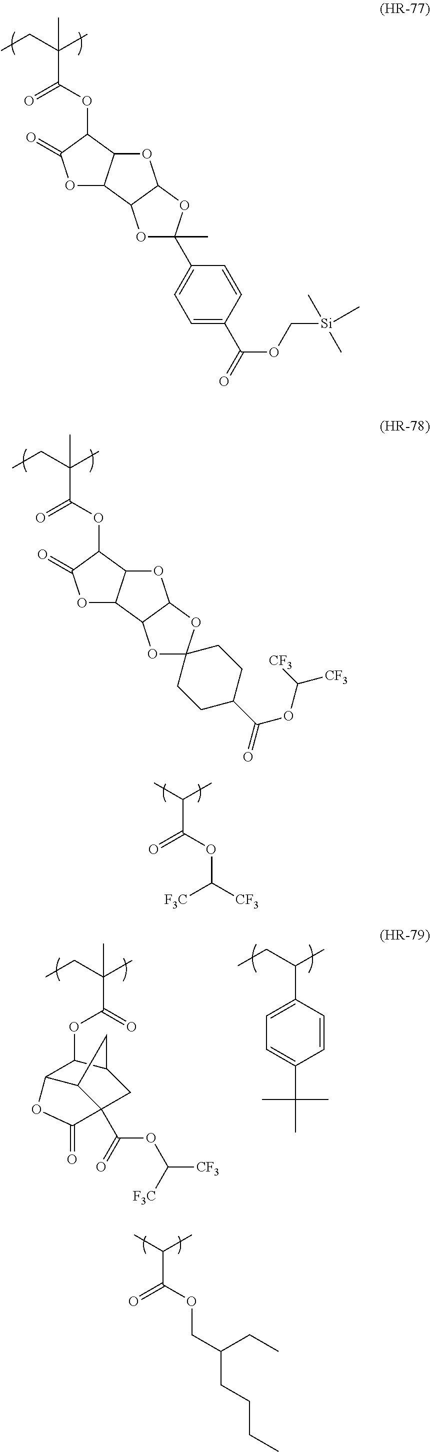

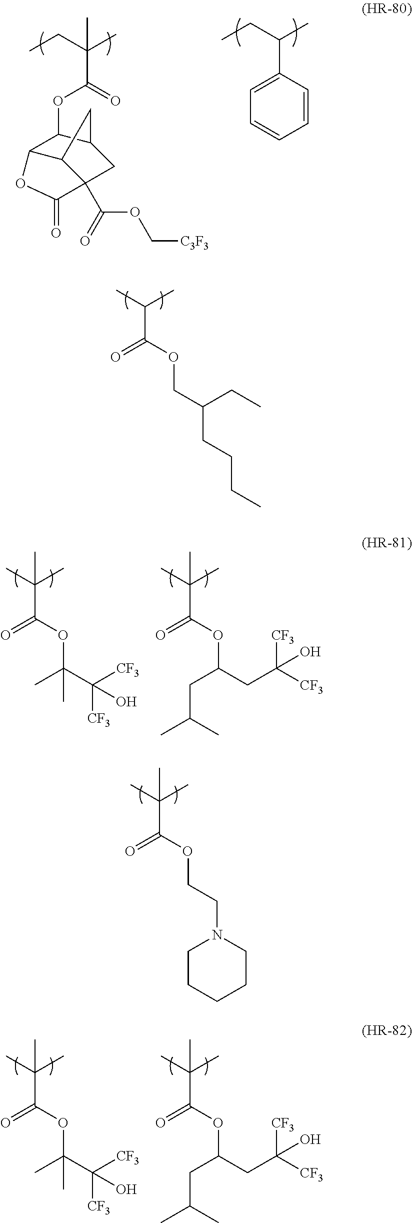

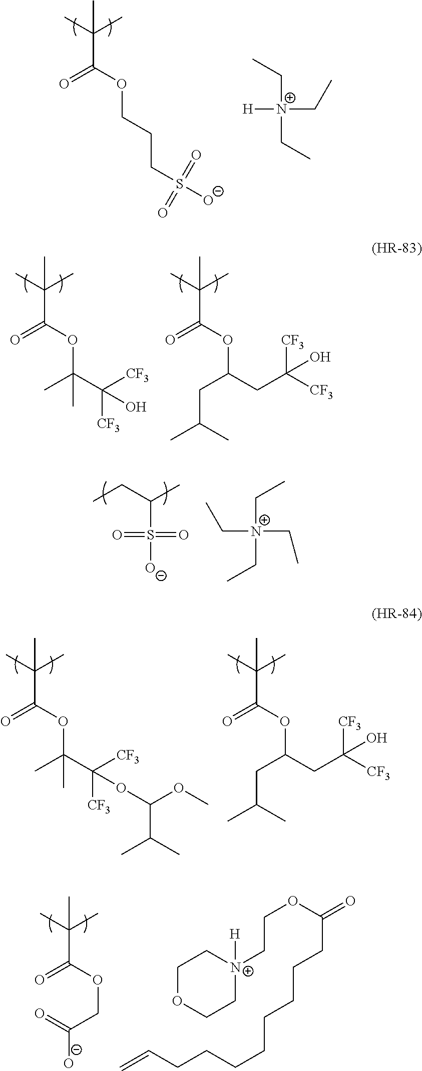

| Resin | Composition | Mw | Mw/Mn |
|---|---|---|---|
| HR-1 | 50/50 | 4900 | 1.4 |
| HR-2 | 50/50 | 5100 | 1.6 |
| HR-3 | 50/50 | 4800 | 1.5 |
| HR-4 | 50/50 | 5300 | 1.6 |
| HR-5 | 50/50 | 4500 | 1.4 |
| HR-6 | 100 | 5500 | 1.6 |
| HR-7 | 50/50 | 5800 | 1.9 |
| HR-8 | 50/50 | 4200 | 1.3 |
| HR-9 | 50/50 | 5500 | 1.8 |
| HR-10 | 40/60 | 7500 | 1.6 |
| HR-11 | 70/30 | 6600 | 1.8 |
| HR-12 | 40/60 | 3900 | 1.3 |
| HR-13 | 50/50 | 9500 | 1.8 |
| HR-14 | 50/50 | 5300 | 1.6 |
| HR-15 | 100 | 6200 | 1.2 |
| HR-16 | 100 | 5600 | 1.6 |
| HR-17 | 100 | 4400 | 1.3 |
| HR-18 | 50/50 | 4300 | 1.3 |
| HR-19 | 50/50 | 6500 | 1.6 |
| HR-20 | 30/70 | 6500 | 1.5 |
| HR-21 | 50/50 | 6000 | 1.6 |
| HR-22 | 50/50 | 3000 | 1.2 |
| HR-23 | 54/50 | 5000 | 1.5 |
| HR-24 | 50/50 | 4500 | 1.4 |
| HR-25 | 30/70 | 5000 | 1.4 |
| HR-26 | 50/50 | 5500 | 1.6 |
| HR-27 | 54/50 | 3500 | 1.3 |
| HR-28 | 50/50 | 6200 | 1.4 |
| HR-29 | 50/50 | 6500 | 1.6 |
| HR-30 | 50/50 | 6500 | 1.6 |
| HR-31 | 50/50 | 4500 | 1.4 |
| HR-32 | 30/70 | 5000 | 1.6 |
| HR-33 | 30/30/40 | 6500 | 1.8 |
| HR-34 | 50/50 | 4000 | 1.3 |
| HR-35 | 50/50 | 6500 | 1.7 |
| HR-36 | 50/50 | 6000 | 1.5 |
| HR-37 | 50/50 | 5000 | 1.6 |
| HR-38 | 50/50 | 4000 | 1.4 |
| HR-39 | 20/80 | 6000 | 1.4 |
| HR-40 | 50/50 | 7000 | 1.4 |
| HR-41 | 50/50 | 6500 | 1.6 |
| HR-42 | 50/50 | 5200 | 1.6 |
| HR-43 | 50/50 | 6000 | 1.4 |
| HR-44 | 70/30 | 5500 | 1.6 |
| HR-45 | 50/20/30 | 4200 | 1.4 |
| HR-46 | 30/70 | 7500 | 1.6 |
| HR-47 | 40/58/2 | 4300 | 1.4 |
| HR-48 | 50/50 | 6800 | 1.6 |
| HR-49 | 100 | 6500 | 1.5 |
| HR-50 | 50/50 | 6600 | 1.6 |
| HR-51 | 30/20/50 | 6800 | 1.7 |
| HR-52 | 95/5 | 5900 | 1.6 |
| HR-53 | 40/30/30 | 4500 | 1.3 |
| HR-54 | 50/30/20 | 6500 | 1.8 |
| HR-55 | 30/40/30 | 7000 | 1.5 |
| HR-56 | 60/40 | 5500 | 1.7 |
| HR-57 | 40/40/20 | 4000 | 1.3 |
| HR-58 | 60/40 | 3800 | 1.4 |
| HR-59 | 80/20 | 7400 | 1.6 |
| HR-60 | 40/44/15/5 | 4800 | 1.5 |
| HR-61 | 60/40 | 5600 | 1.5 |
| HR-62 | 50/50 | 5900 | 2.1 |
| HR-63 | 80/20 | 7000 | 1.7 |
| HR-64 | 100 | 5500 | 1.8 |
| HR-65 | 50/50 | 9500 | 1.9 |
| HR-66 | 100 | 6000 | 1.5 |
| HR-67 | 100 | 6000 | 1.4 |
| HR-68 | 100 | 9000 | 1.5 |
| HR-69 | 60/40 | 8000 | 1.3 |
| HR-70 | 80/20 | 5000 | 1.4 |
| HR-71 | 100 | 9500 | 1.5 |
| HR-72 | 40/60 | 8000 | 1.4 |
| HR-73 | 55/30/5/10 | 8000 | 1.3 |
| HR-74 | 100 | 13000 | 1.4 |
| HR-75 | 70/30 | 8000 | 1.3 |
| HR-76 | 50/40/10 | 500 | 1.5 |
| HR-77 | 100 | 9000 | 1.6 |
| HR-78 | 80/20 | 3500 | 1.4 |
| HR-79 | 90/8/2 | 13000 | 1.5 |
| HR-80 | 85/10/5 | 5000 | 1.5 |
| HR-81 | 35/60/5 | 8600 | 1.99 |
| HR-82 | 35/60/5 | 8700 | 1.71 |
| HR-83 | 35/60/5 | 8100 | 1.81 |
| HR-84 | 35/60/5 | 8900 | 1.89 |

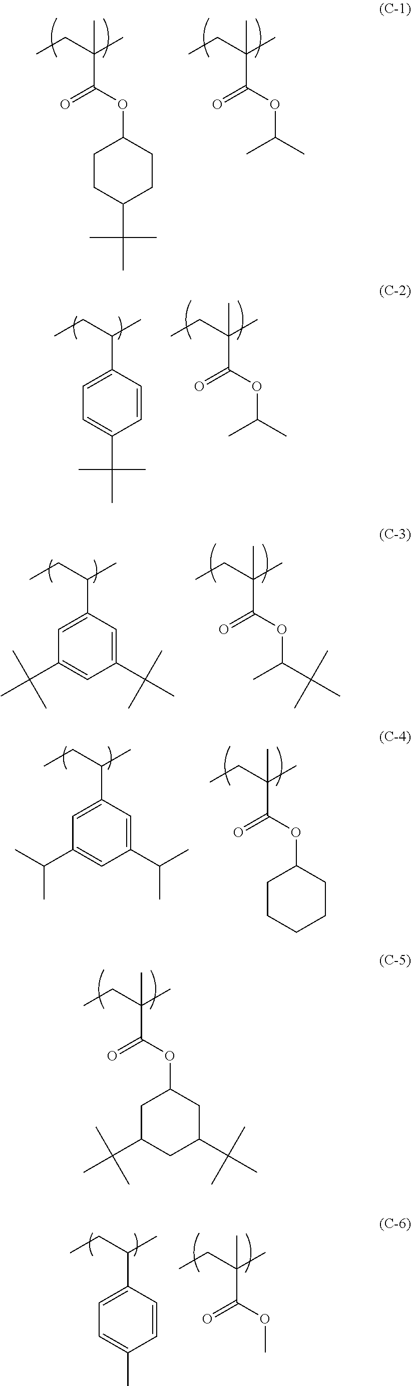

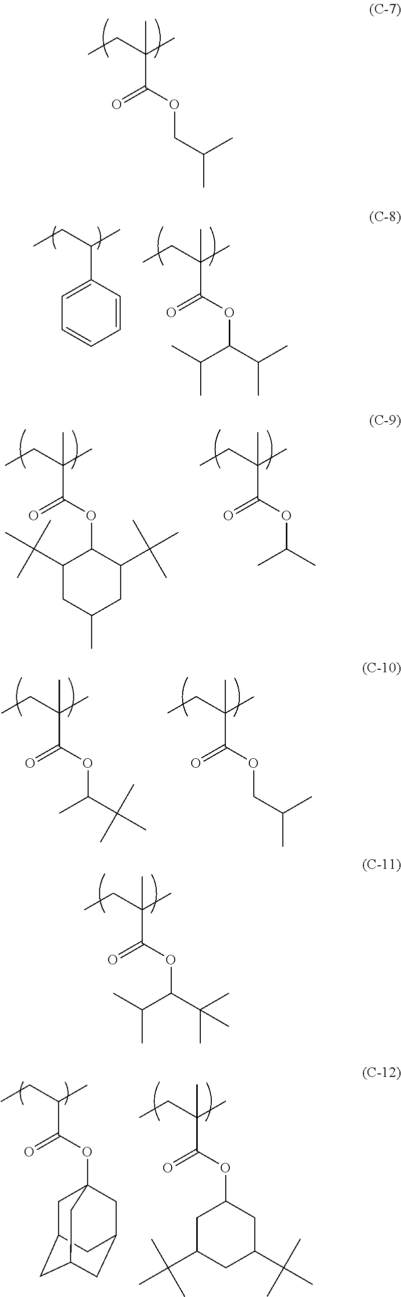

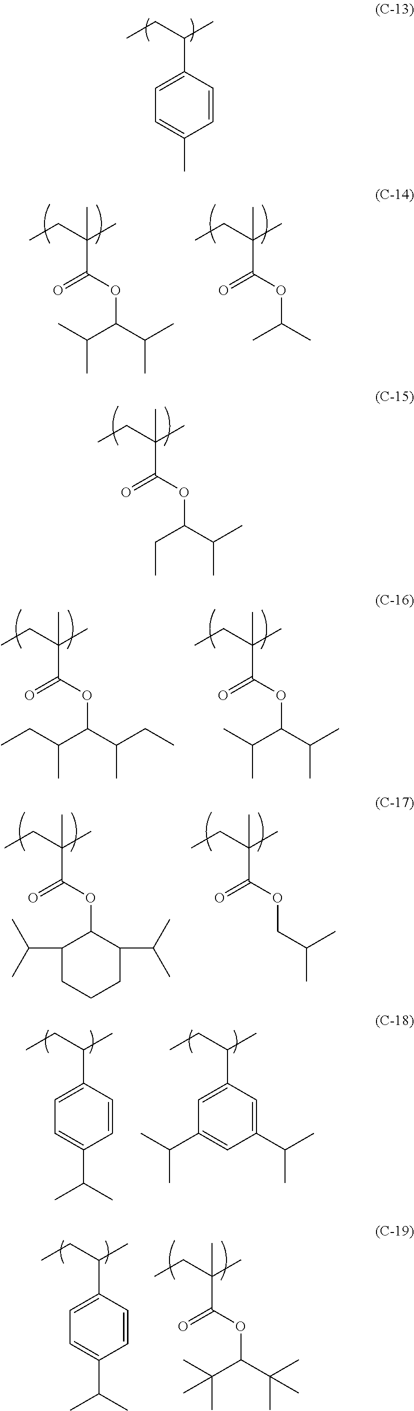

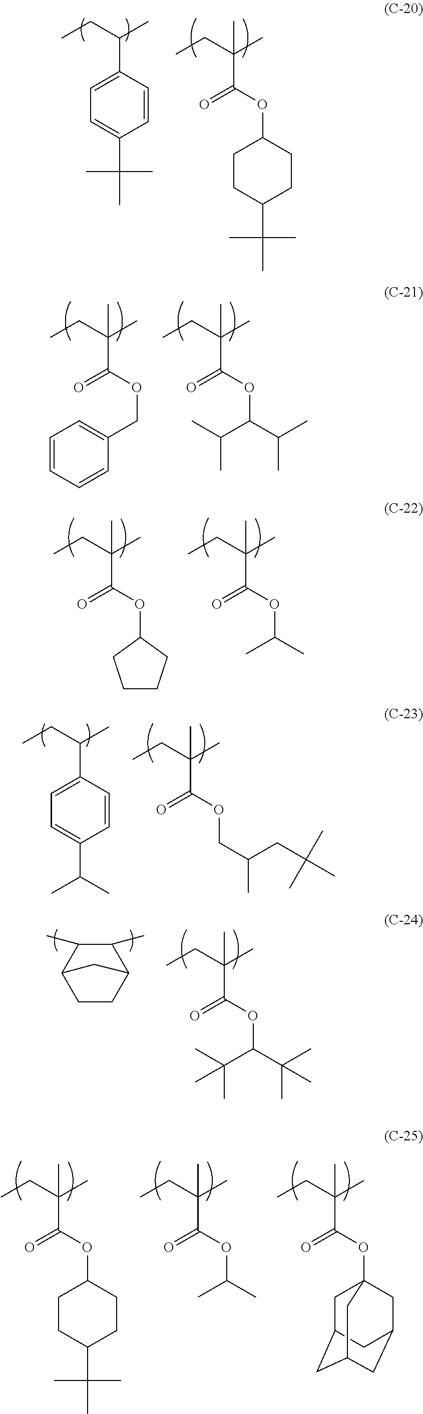

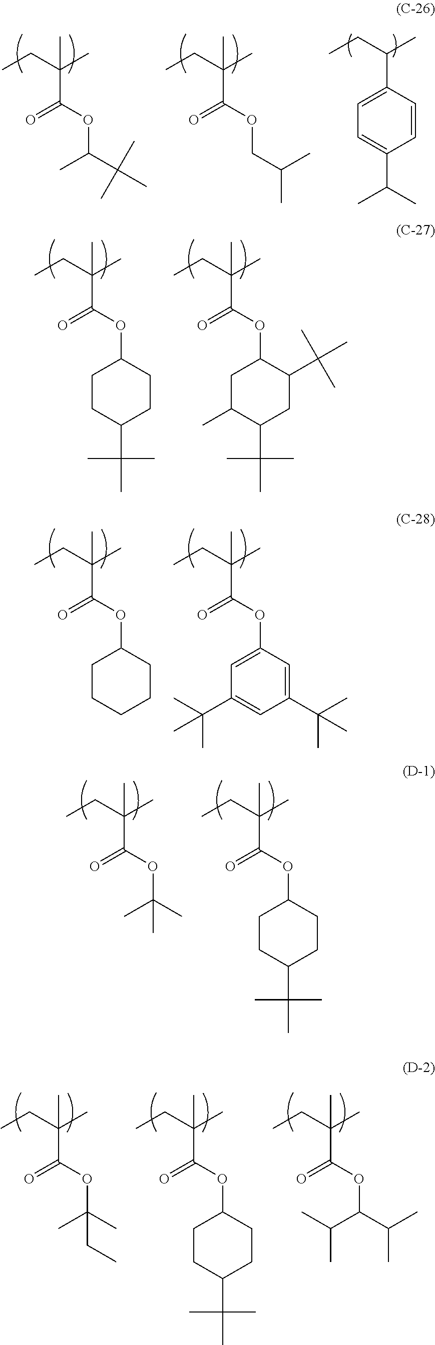

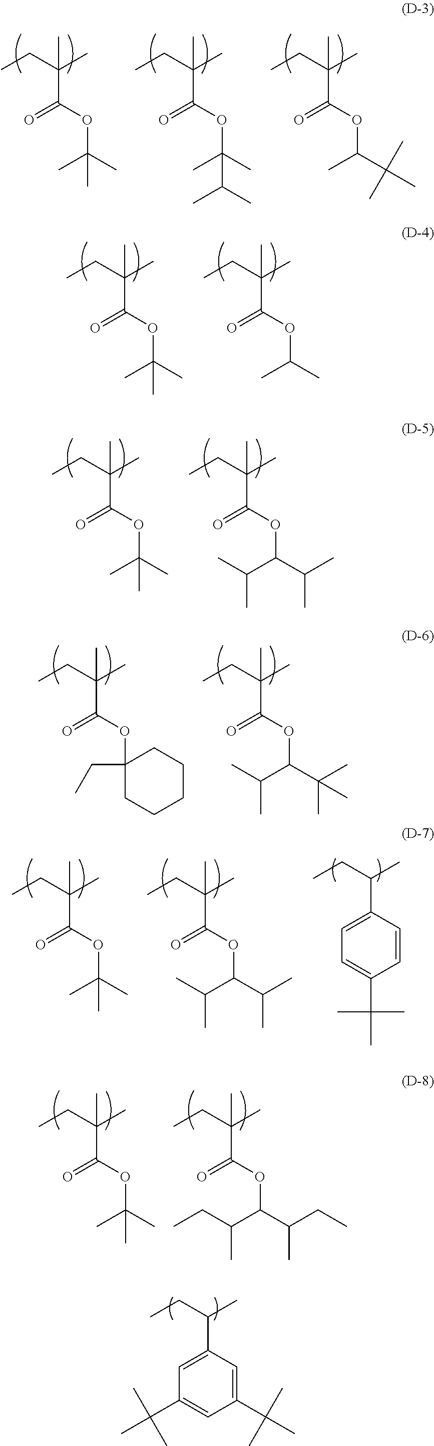

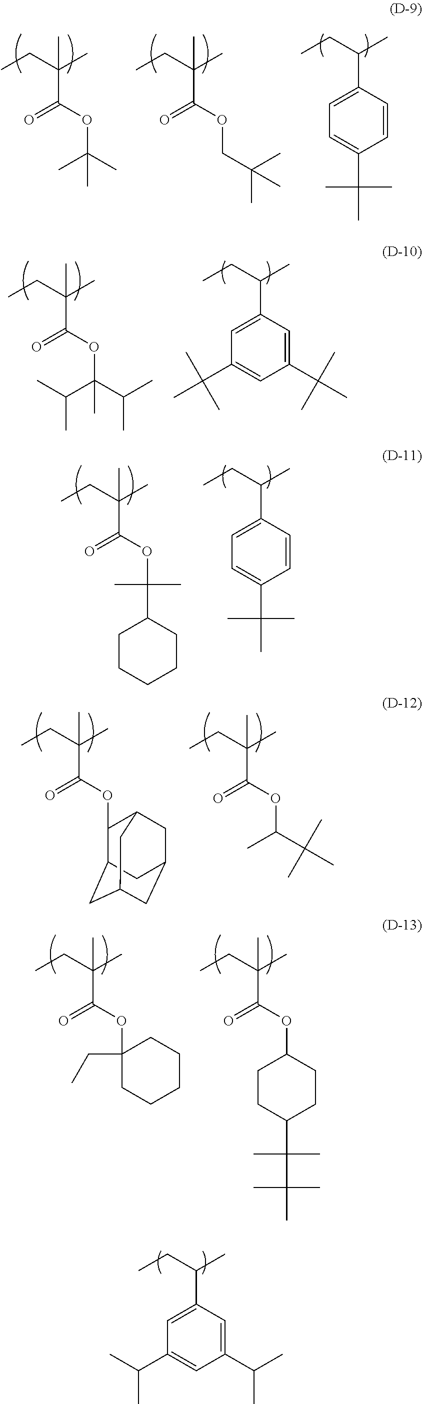

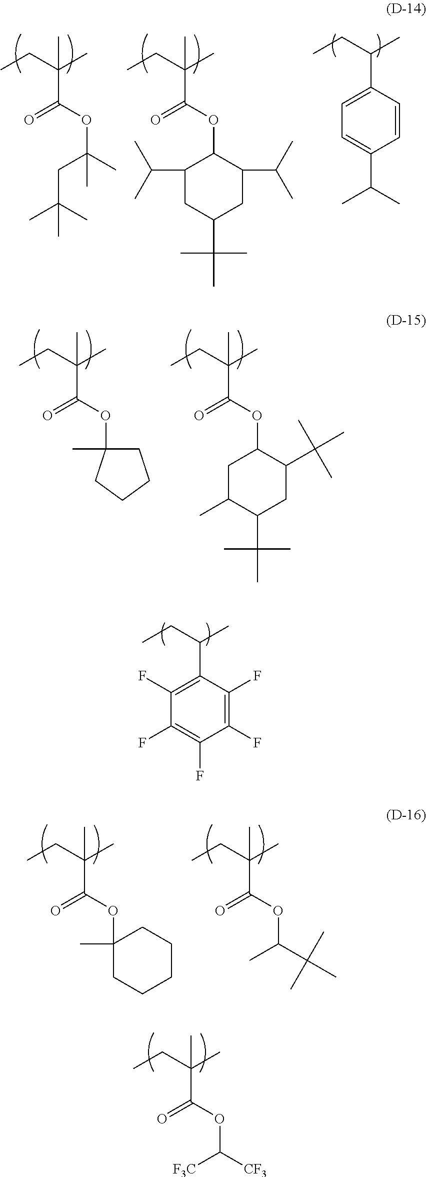

| Resin | Composition | Mw | Mw/Mn |
|---|---|---|---|
| C-1 | 50/50 | 9600 | 1.74 |
| C-2 | 60/40 | 34500 | 1.43 |
| C-3 | 30/70 | 19300 | 1.69 |
| C-4 | 90/10 | 26400 | 1.41 |
| C-5 | 100 | 27600 | 1.87 |
| C-6 | 80/20 | 4400 | 1.96 |
| C-7 | 100 | 16300 | 1.83 |
| C-8 | 5/95 | 24500 | 1.79 |
| C-9 | 20/80 | 15400 | 1.68 |
| C-10 | 50/50 | 23800 | 1.46 |
| C-11 | 100 | 22400 | 1.57 |
| C-12 | 10/90 | 21600 | 1.52 |
| C-13 | 100 | 28400 | 1.58 |
| C-14 | 50/50 | 16700 | 1.82 |
| C-15 | 100 | 23400 | 1.73 |
| C-16 | 60/40 | 18600 | 1.44 |
| C-17 | 80/20 | 12300 | 1.78 |
| C-18 | 40/60 | 18400 | 1.58 |
| C-19 | 70/30 | 12400 | 1.49 |
| C-20 | 50/50 | 23500 | 1.94 |
| C-21 | 10/90 | 7600 | 1.75 |
| C-22 | 5/95 | 14100 | 1.39 |
| C-23 | 50/50 | 17900 | 1.61 |
| C-24 | 10/90 | 24600 | 1.72 |
| C-25 | 50/40/10 | 23500 | 1.65 |
| C-26 | 60/30/10 | 13100 | 1.51 |
| C-27 | 50/50 | 21200 | 1.84 |
| C-28 | 10/90 | 19500 | 1.66 |
| D-1 | 50/50 | 16500 | 1.72 |
| D-2 | 10/50/40 | 18000 | 1.77 |
| D-3 | 5/50/45 | 27100 | 1.69 |
| D-4 | 20/80 | 26500 | 1.79 |
| D-5 | 10/90 | 24700 | 1.83 |
| D-6 | 10/90 | 15700 | 1.99 |
| D-7 | 5/90/5 | 21500 | 1.92 |
| D-8 | 5/60/35 | 17700 | 2.10 |
| D-9 | 35/35/30 | 25100 | 2.02 |
| D-10 | 70/30 | 19700 | 1.85 |
| D-11 | 75/25 | 23700 | 1.80 |
| D-12 | 10/90 | 20100 | 2.02 |
| D-13 | 5/35/60 | 30100 | 2.17 |
| D-14 | 5/45/50 | 22900 | 2.02 |
| D-15 | 15/75/10 | 28600 | 1.81 |
| D-16 | 25/55/20 | 27400 | 1.87 |

### [5-1](N) Basic Compound

The actinic ray-sensitive or radiation-sensitive resin composition of the present invention preferably contains a basic compound so as to reduce the change in performance with aging from exposure to heating.

Preferred basic compounds include compounds having a structure represented by the following formulae (A) to (E):

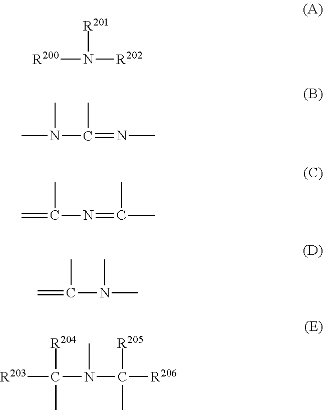

In formulae (A) and (E), each of R²⁰⁰, R²⁰¹ and R²⁰², which may be the same as or different from each other, represents a hydrogen atom, an alkyl group (preferably having a carbon number of 1 to 20), a cycloalkyl group (preferably having a carbon number of 3 to 20) or an aryl group (having a carbon number of 6 to 20), and R²⁰¹ and R²⁰² may combine with each other to form a ring.

Each of R²⁰³, R²⁰⁴, R²⁰⁵ and R²⁰⁶, which may be the same as or different from each other, represents an alkyl group having a carbon number of 1 to 20.

As for the alkyl group, the alkyl group having a substituent is preferably an aminoalkyl group having a carbon number of 1 to 20, a hydroxyalkyl group having a carbon number of 1 to 20, or a cyanoalkyl group having a carbon number of 1 to 20.

The alkyl group in formulae (A) and (E) is more preferably unsubstituted.

Preferred examples of the compound include guanidine, aminopyrrolidine, pyrazole, pyrazoline, piperazine, aminomorpholine, aminoalkylmorpholine, and piperidine. More preferred examples of the compound include a compound having an imidazole structure, a diazabicyclo structure, an onium hydroxide structure, an onium carboxylate structure, a trialkylamine structure, an aniline structure or a pyridine structure; an alkylamine derivative having a hydroxyl group and/or an ether bond; and an aniline derivative having a hydroxyl group and/or an ether bond.

Examples of the compound having an imidazole structure include imidazole, 2,4,5-triphenylimidazole, benzimidazole, and 2-phenylbenzimidazole. Examples of the compound having a diazabicyclo structure include 1,4-diazabicyclo[2,2,2]octane, 1,5-diazabicyclo[4,3,0]non-5-ene, and 1,8-diazabicyclo[5,4,0]undec-7-ene. Examples of the compound having an onium hydroxide structure include a tetrabutylanunonium hydroxide, a triarylsulfonium hydroxide, a phenacylsulfonium hydroxide, and a sulfonium hydroxide having a 2-oxoalkyl group, specifically, triphenylsulfonium hydroxide, tris(tert-butylphenyl)sulfonium hydroxide, bis(tert-butylphenyl)iodonium hydroxide, phenacylthiophenium hydroxide and 2-oxopropylthiophenium hydroxide. The compound having an onium carboxylate structure is a compound where the anion moiety of the compound having an onium hydroxide structure is changed to a carboxylate, and examples thereof include acetate, adamantane-1-carboxylate, and perfluoroalkyl carboxylate. Examples of the compound having a trialkylamine structure include tri(n-butyl)amine and tri(n-octyl)amine. Examples of the aniline compound include 2,6-diisopropylaniline, N,N-dimethylaniline, N,N-dibutylaniline, and N,N-dihexylaniline. Examples of the alkylamine derivative having a hydroxyl group and/or an ether bond include ethanolamine, diethanolamine, triethanolamine, N-phenyldiethanolamine, and tris(methoxyethoxyethyl)amine. Examples of the aniline derivative having a hydroxyl group and/or an ether bond include N,N-bis(hydroxyethyl)aniline.

Other preferred basic compounds include a phenoxy group-containing amine compound, a phenoxy group-containing ammonium salt compound, a sulfonic acid ester group-containing amine compound, and a sulfonic acid ester group-containing ammonium salt compound. Examples of these compounds include Compounds (C1-1) to (C3-3) illustrated in paragraph [0066] of U.S. Patent Application Publication No. 2007/0224539A1.

The following compounds are also preferred as the basic compound.

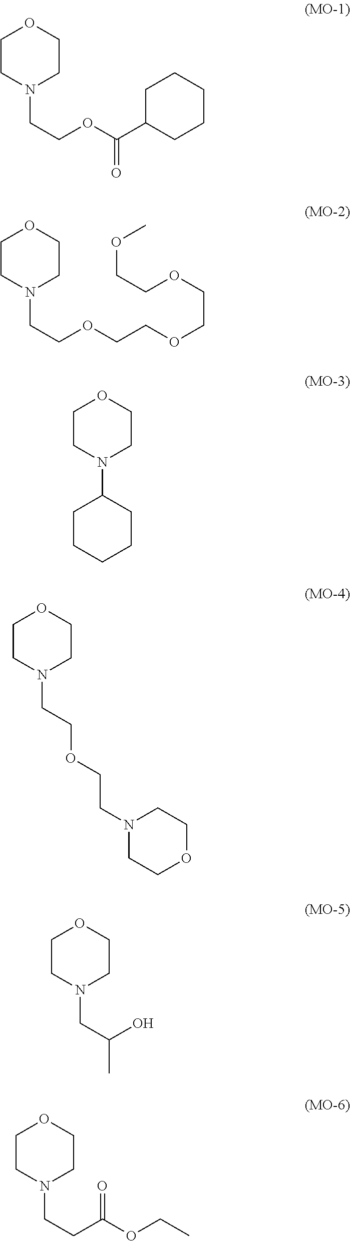

In addition to the compounds described above, for example, compounds described in [0180] to [0225] of JP-A-2011-22560. [0218] to [0219] of JP-A-2012-137735, and [0416] to [0438] of International Publication WO2011/158687A1, pamphlet, may also be used as the basic compound. The basic compound may also be a basic compound or an ammonium salt compound, whose basicity decreases upon irradiation with an actinic ray or radiation.

As for these basic compounds, one kind may be used alone, or two or more kinds may be used in combination.

The composition of the present invention may or may not contain a basic compound, but in the case of containing a basic compound, the content percentage thereof is usually from 0.001 to 10 mass %, preferably from 0.01 to 5 mass %, based on the solid content of the actinic ray-sensitive or radiation-sensitive resin composition.

The ratio between the acid generator (including the acid generator (A′)) and the basic compound used in the composition is preferably acid generator/basic compound (molar ratio)=from 2.5 to 300. That is, the molar ratio is preferably 2.5 or more in view of sensitivity and resolution and is preferably 300 or less from the standpoint of suppressing the reduction in resolution due to thickening of the resist pattern with aging after exposure until heat treatment. The acid generator/basic compound (molar ratio) is more preferably from 5.0 to 200, still more preferably from 7.0 to 150.

The basic resin is preferably used, in terms of the molar ratio to the low molecular compound (D) described in item [4] later, in a ratio of low molecular compound (D)/basic compound=from 100/0 to 10/90, more preferably from 100/0 to 30/70, still more preferably from 100/0 to 50/50.

Incidentally, the basic compound as used herein excludes (C) a low molecular compound containing a nitrogen atom and having a group capable of leaving by the action of an acid, which is described later.

[5-2](N′) Compound Having a Basic Functional Group or an Ammonium Group and a Group Capable of Generating an Acidic Functional Group Upon Irradiation with an Actinic Ray or Radiation

The actinic ray-sensitive or radiation-sensitive resin composition in the present invention is typically, preferably (N′−1) a compound containing a nitrogen atom-containing basic functional group or an ammonium group and having a group capable of generating an acidic functional group upon irradiation with an actinic ray or radiation, described in JP-A-2006-330098 and JP-A-2011-100105. That is, the compound (N′) is a basic compound having a basic functional group and a group capable of generating an acidic functional group upon irradiation with an actinic ray or radiation, or an ammonium salt compound having an ammonium group and a group capable of generating an acidic functional group upon irradiation with an actinic ray or radiation.

Specific examples of the compound (N′) are illustrated below, but the present invention is not limited thereto.

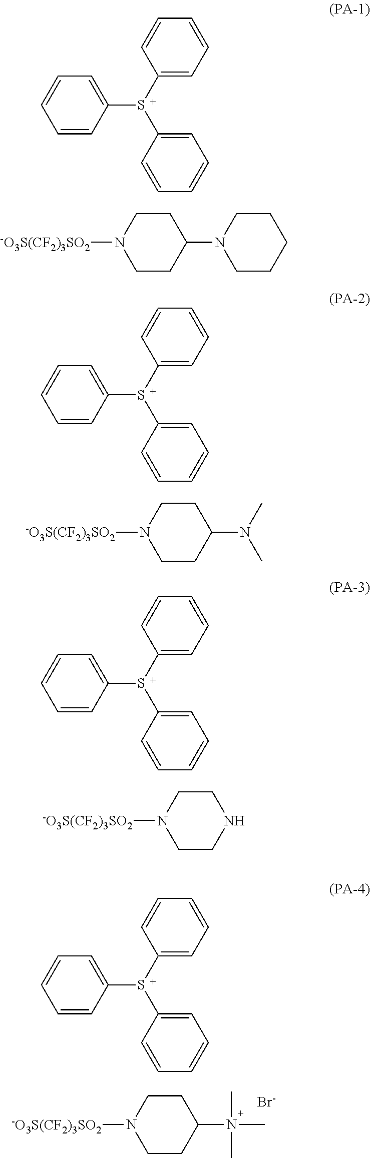

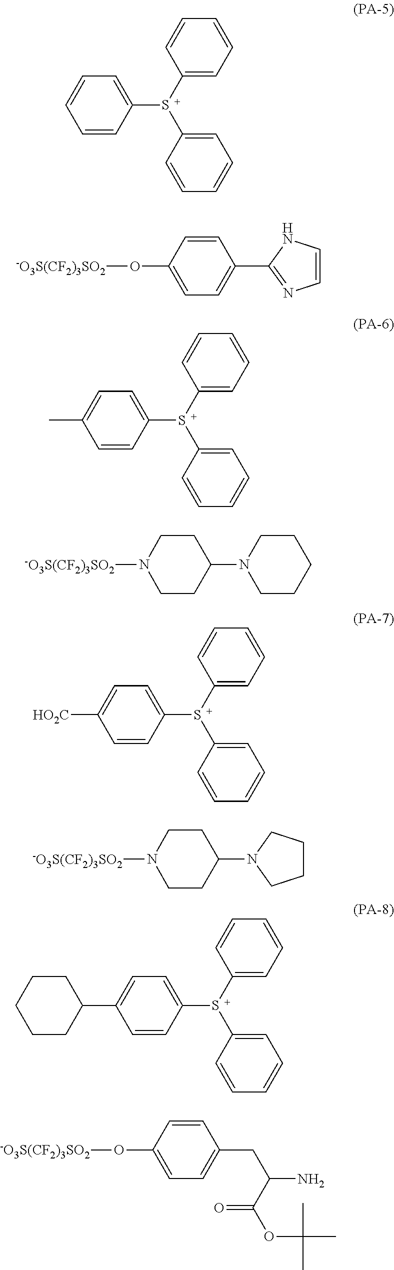

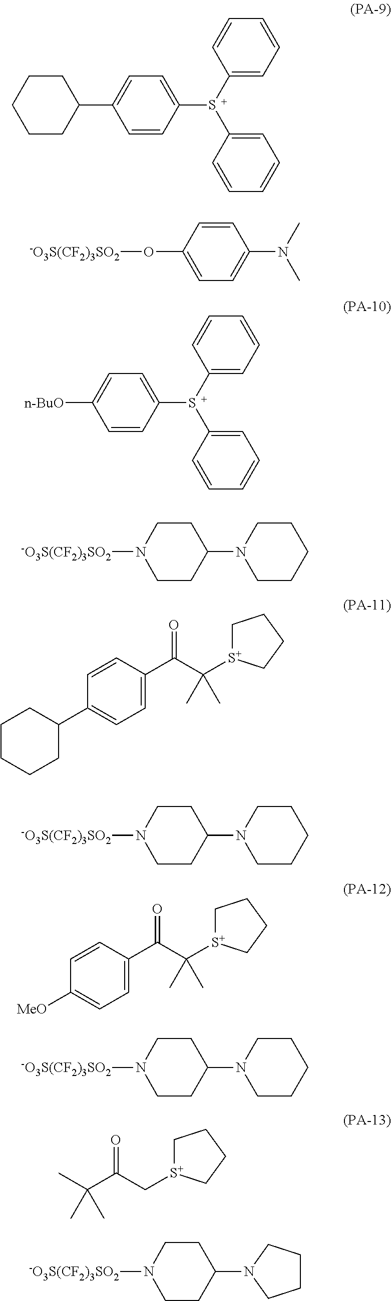

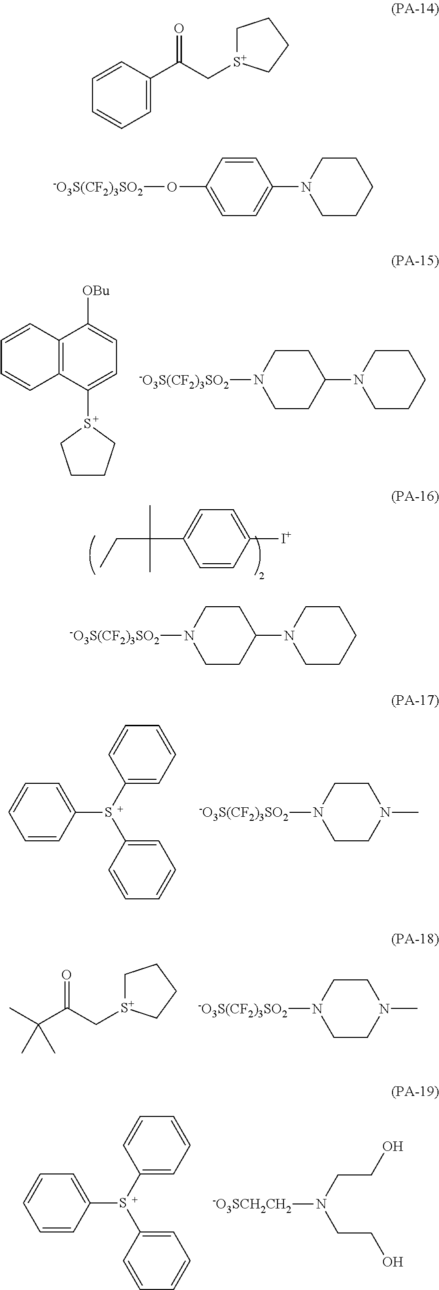

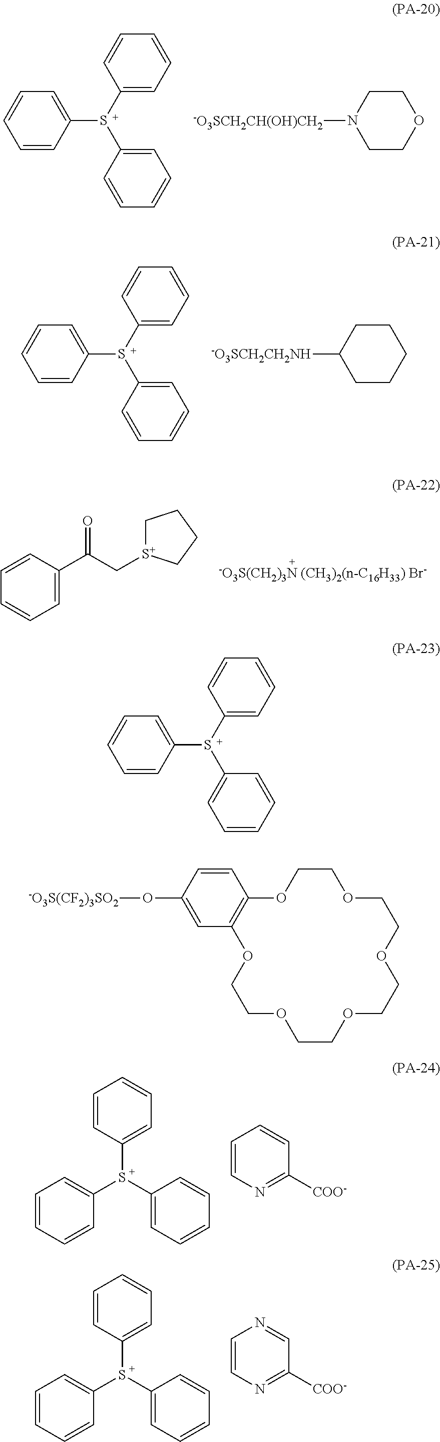

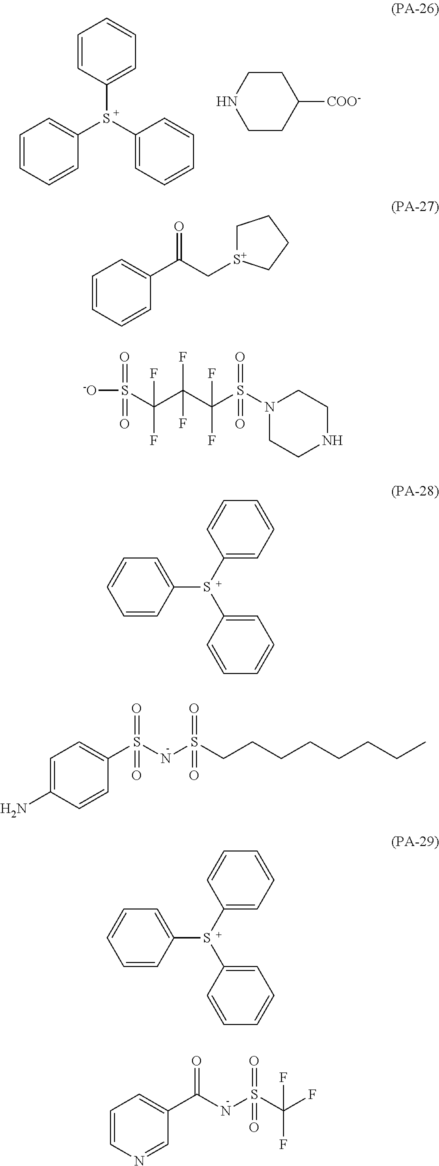

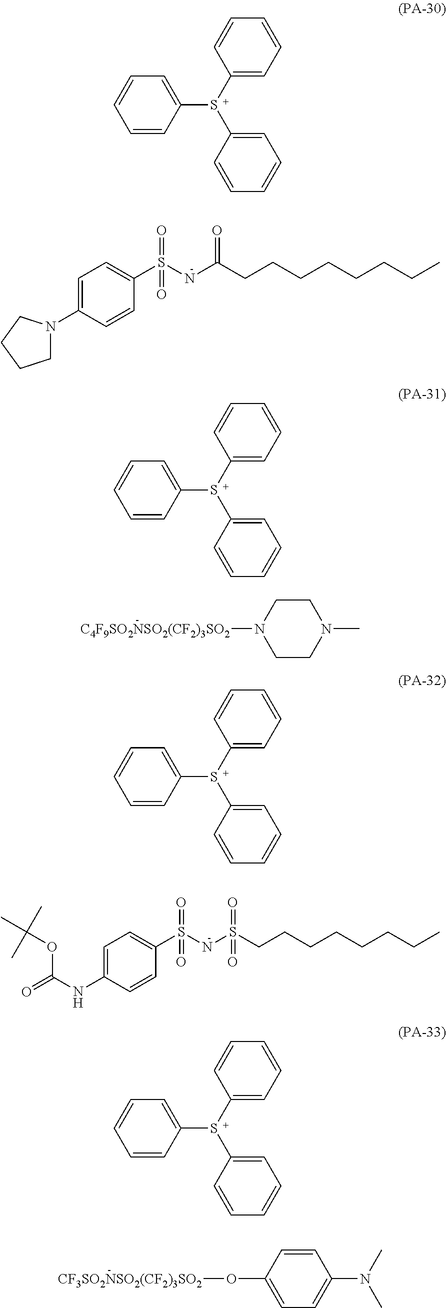

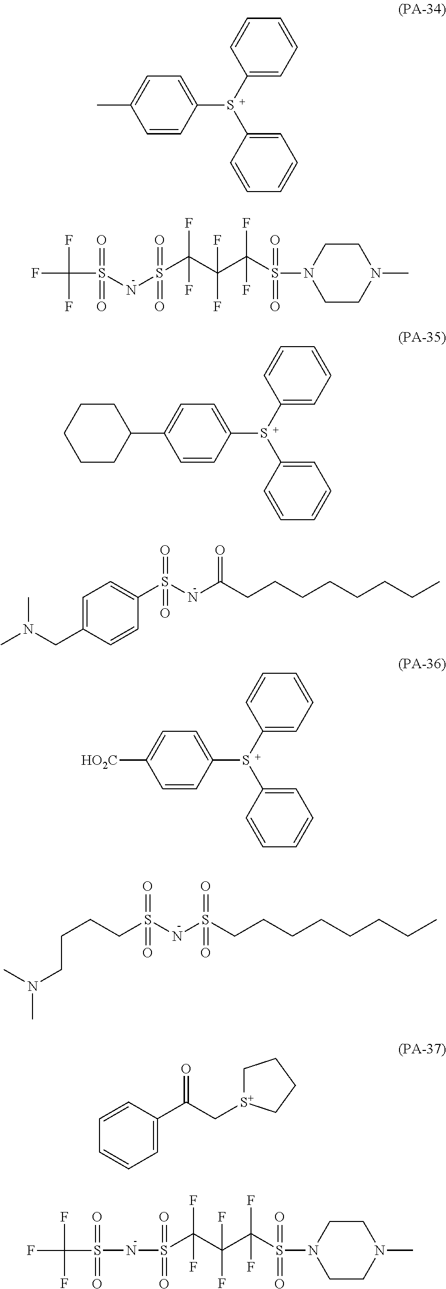

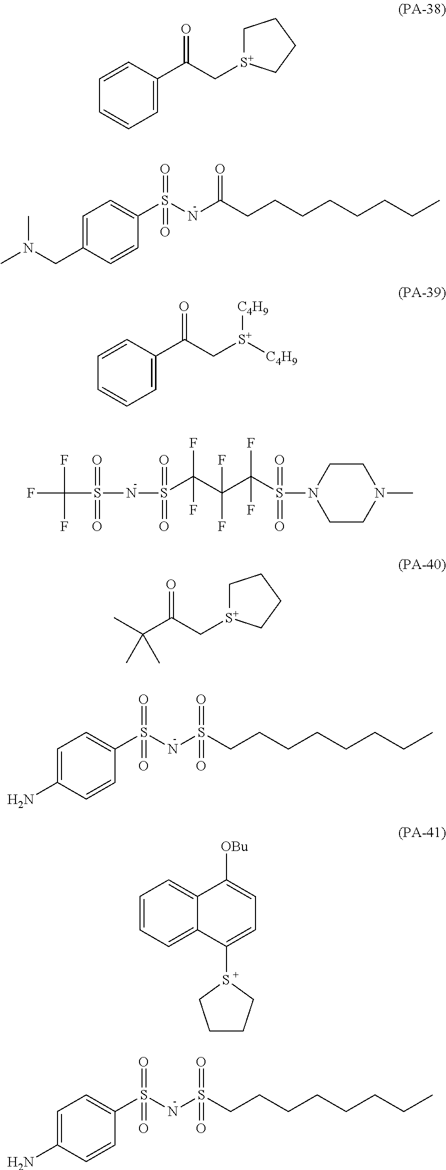

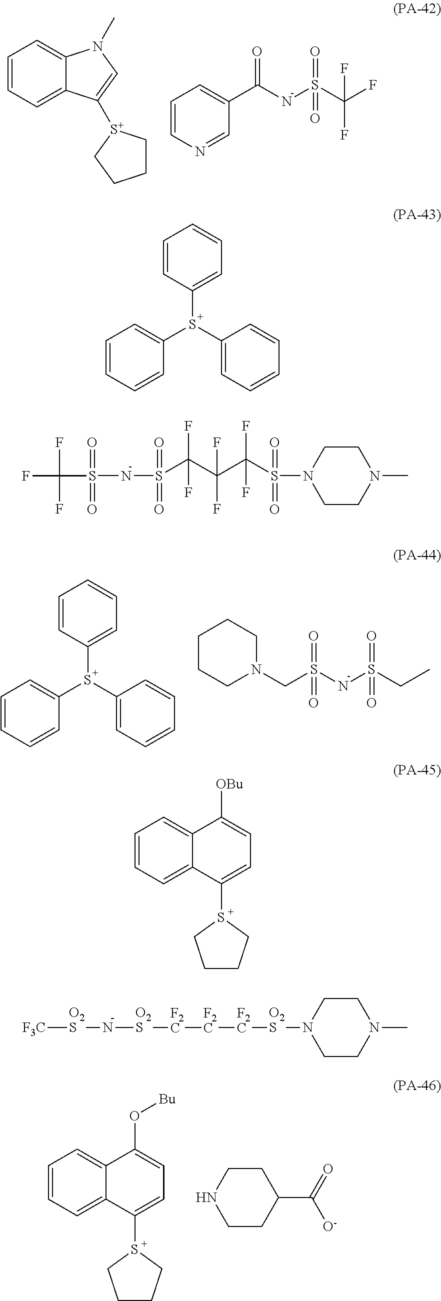

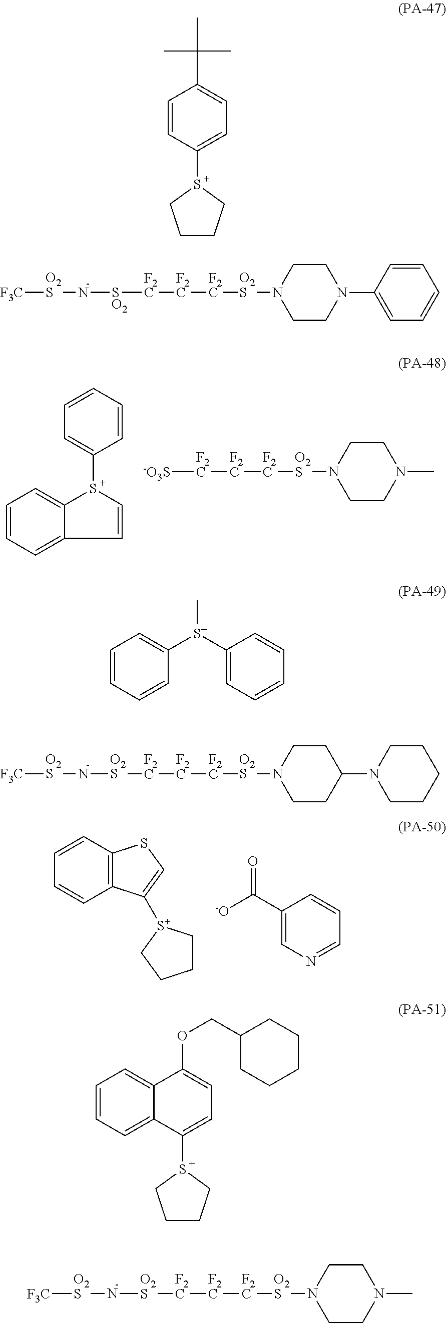

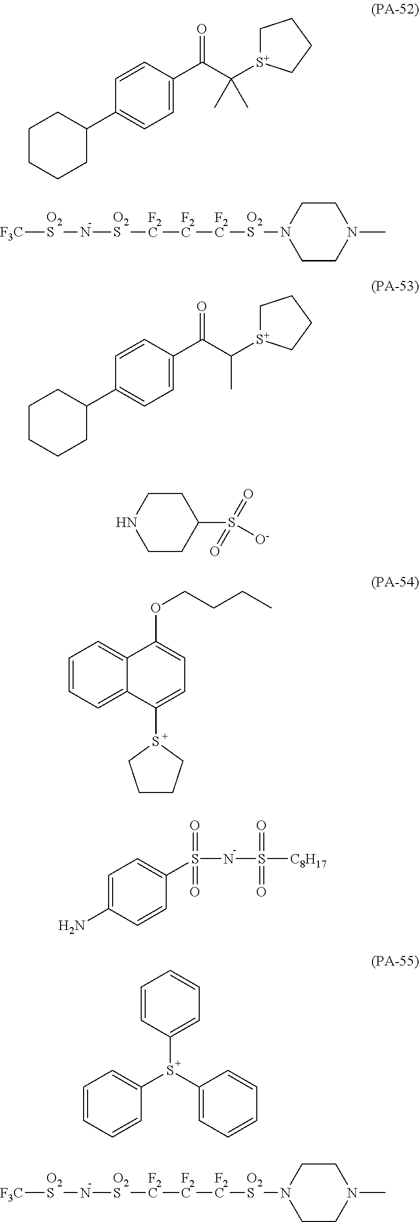

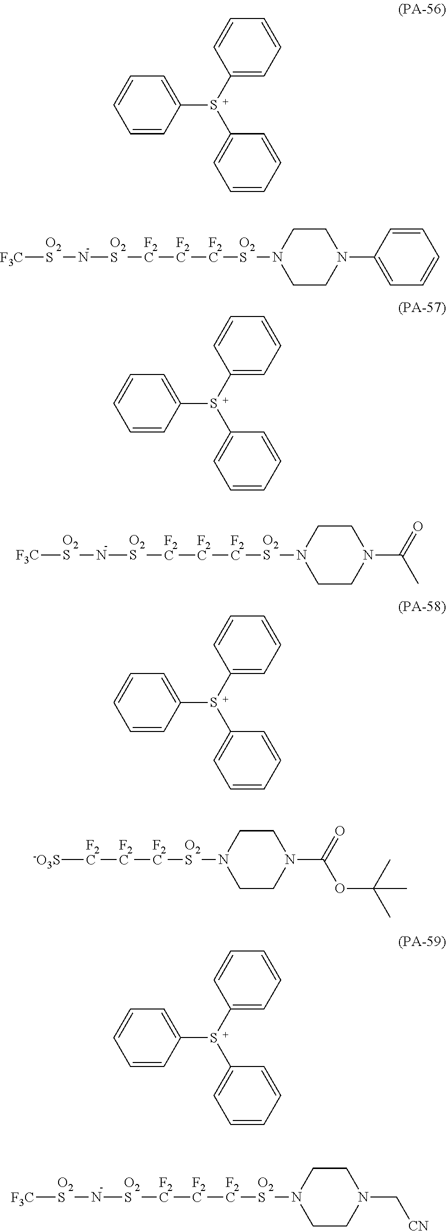

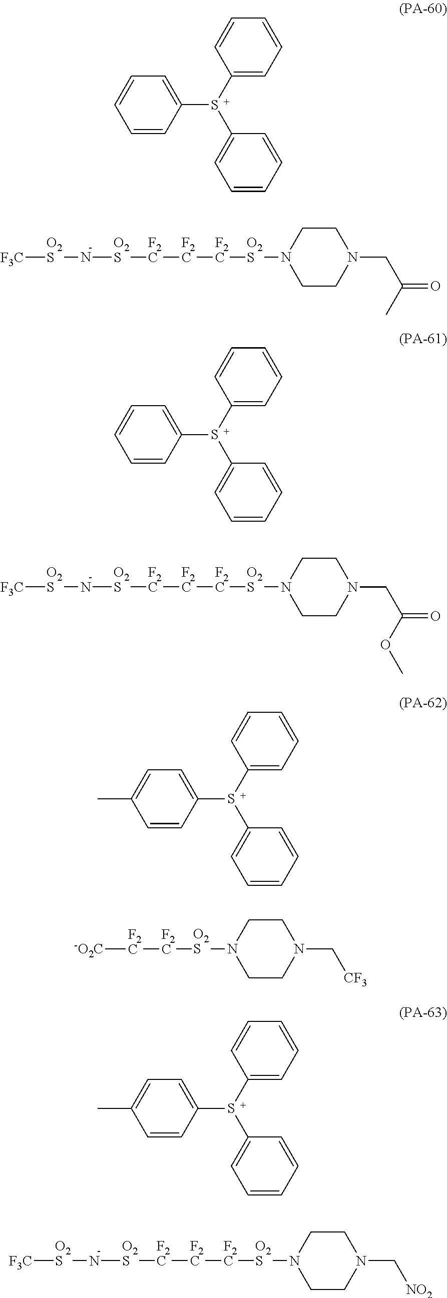

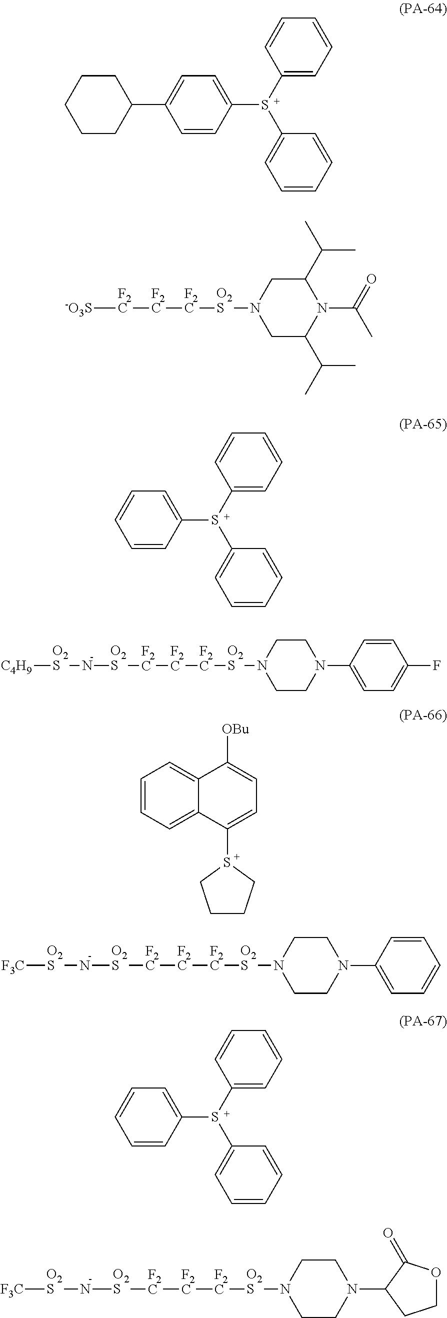

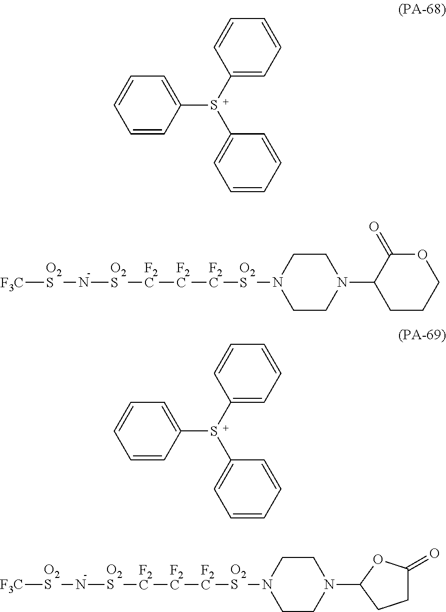

In particular, the synthesis of these compounds may be performed in accordance with synthesis examples and the like in JP-A-2006-330098 and JP-A-2011-100105.

The actinic ray-sensitive or radiation-sensitive resin composition of the present invention may or may not contain the compound (E), but in the case of containing the compound (E), the content thereof is preferably from 0.1 to 20 mass %, more preferably from 0.1 to 10 mass %, based on the solid content of the actinic ray-sensitive or radiation-sensitive resin composition.

### [5-3] Low Molecular Compound Containing a Nitrogen Atom and Having a Group Capable of Leaving by the Action of an Acid

The composition of the present invention may contain a compound containing a nitrogen atom and having a group capable of leaving by the action of an acid (hereinafter, sometimes referred to as ‘compound (N″)’).

The group capable of leaving by the action of an acid is not particularly limited but is preferably an acetal group, a carbonate group, a carbamate group, a tertiary ester group, a tertiary hydroxyl group or a hemiaminal ether group, more preferably a carbamate group or a hemiaminal ether group.

The molecular weight of the (N″) compound having a group capable of leaving by the action of an acid is preferably from 100 to 1,000, more preferably from 100 to 700, still more preferably from 100 to 500.

The compound (N″) is preferably an amine derivative having on the nitrogen atom a group capable of leaving by the action of an acid.

The compound (N″) may have a protective group-containing carbamate group on the nitrogen atom. The protective group constituting the carbamate group can be represented by the following formula (d-1):

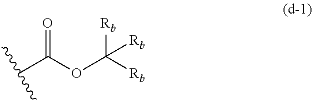

In formula (d-1), each Rb independently represents a hydrogen atom, an alkyl group (preferably having a carbon number of 1 to 10), a cycloalkyl group (preferably having a carbon number of 3 to 30), an aryl group (preferably having a carbon number of 3 to 30), an aralkyl group (preferably having a carbon number of 1 to 10) or an alkoxyalkyl group (preferably having a carbon number of 1 to 10). Respective Rb may combine with each other to form a ring.

Each of the alkyl group, cycloalkyl group, aryl group and aralkyl group represented by Rb may be substituted with a functional group such as hydroxyl group, cyano group, amino group, pyrrolidino group, piperidino group, morpholino group and oxo group, an alkoxy group or a halogen atom. The same applies to the alkoxyalkyl group represented by Rb.

Rb is preferably a linear or branched alkyl group, a cycloalkyl group or an aryl group, more preferably a linear or branched alkyl group or a cycloalkyl group.

Examples of the ring formed by combining two Rb with each other include an alicyclic hydrocarbon group, an aromatic hydrocarbon group, a heterocyclic hydrocarbon group, and derivatives thereof.

Specific structures of the group represented by formula (d-1) include, but are not limited to, structures disclosed in paragraph [0466] of U.S. Patent Application Publication No. 2012/0135348A1.

Among others, the compound (N″) is preferably a compound having a structure represented by the following formula (6):

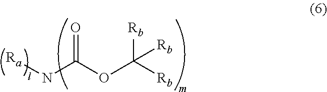

In formula (6), Ra represents a hydrogen atom, an alkyl group, a cycloalkyl group, an aryl group or an aralkyl group. Also, when l is 2, two Ra may be the same as or different from one another, and two Ra may combine with each other to form a heterocyclic ring together with the nitrogen atom in the formula. The heterocyclic ring may contain a heteroatom other than the nitrogen atom in the formula.

Rb has the same meaning as Rb in formula (d-1), and preferred examples are also the same.

l represents an integer of 0 to 2, m represents an integer of 1 to 3, and these satisfy l+m=3.

In formula (6), the alkyl group, cycloalkyl group, aryl group and aralkyl group of Ra may be substituted with the same group as the group described above as a group which may be substituted on the alkyl group, cycloalkyl group, aryl group and aralkyl group of Rb.

Preferred examples of the alkyl group, cycloalkyl group, aryl group and aralkyl group of Ra (these alkyl group, cycloalkyl group, aryl group and aralkyl group may be substituted with the above-described group) are the same as preferred examples of the groups described above for Rb.

The heterocyclic ring formed by combining Ra with each other preferably has a carbon number of 20 or less, and examples thereof include a group derived from a heterocyclic compound such as pyrrolidine, piperidine, morpholine, 1,4,5,6-tetrahydropyrimidine, 1,2,3,4-tetrahydroquinoline, 1,2,3,6-tetrahydropyridine, homopiperazine, 4-azabenzimidazole, benzotriazole, 5-azabenzotriazole, 1H-1,2,3-triazole, 1,4,7-triazacyclononane, tetrazole, 7-azaindole, indazole, benzimidazole, imidazo[1,2-a]pyridine, (1S,4S)-(+)-2,5-diazabicyclo[2.2.1]heptane, 1,5,7-triazabicyclo[4.4.0]dec-5-ene, indole, indoline, 1,2,3,4-tetrahydroquinoxaline, perhydroquinoline and 1,5,9-triazacyclododecane, and a group where the group derived from a heterocyclic compound is substituted with one or more kinds of or one or more groups of linear or branched alkane-derived groups, cycloalkane-derived groups, aromatic compound-derived groups, heterocyclic compound-derived groups, and functional groups such as hydroxyl group, cyano group, amino group, pyrrolidino group, piperidino group, morpholino group and oxo group.

Specific examples of the compound (N″) particularly preferred in the present invention include, but are not limited to, compounds disclosed in paragraph [0475] of U.S. Patent Application Publication No. 2012/0135348A1.

The compound represented by formula (6) can be synthesized by referring to, for example, JP-A-2007-298569 and JP-A-2009-199021.

In the present invention, as for the (C) low molecular weight compound having on the nitrogen atom a group capable of leaving by the action of an acid, one compound may be used alone, or two or more compounds may be mixed and used.

The content of the compound (C) in the actinic ray-sensitive or radiation-sensitive resin composition of the present invention is preferably from 0.001 to 20 mass %, more preferably from 0.001 to 10 mass %, still more preferably from 0.01 to 5 mass %, based on the total solid content of the composition.

### [6](E) Solvent

The solvent which can be used at the preparation of the actinic ray-sensitive or radiation-sensitive resin composition of the present invention includes, for example, an organic solvent such as alkylene glycol monoalkyl ether carboxylate, alkylene glycol monoalkyl ether, alkyl lactate, alkyl alkoxypropionate, cyclic lactone (preferably having a carbon number of 4 to 10), monoketone compound (preferably having a carbon number of 4 to 10) which may contain a ring, alkylene carbonate, alkyl alkoxyacetate and alkyl pyruvate.

Specific examples of these solvents include those described in paragraphs [0441] to [0455] of U.S. Patent Application Publication No. 2008/0187860.

In the present invention, a mixed solvent prepared by mixing a solvent containing a hydroxyl group in the structure and a solvent not containing a hydroxyl group may be used as the organic solvent.

The solvent containing a hydroxyl group and the solvent not containing a hydroxyl group may be appropriately selected from the compounds exemplified above, but the solvent containing a hydroxyl group is preferably an alkylene glycol monoalkyl ether, an alkyl lactate or the like, more preferably propylene glycol monomethyl ether (PGME, another name: 1-methoxy-2-propanol) or ethyl lactate. The solvent not containing a hydroxyl group is preferably an alkylene glycol monoalkyl ether acetate, an alkyl alkoxypropionate, a monoketone compound which may contain a ring, a cyclic lactone, an alkyl acetate or the like and among these, more preferably propylene glycol monomethyl ether acetate (PGMEA, another name: 1-methoxy-2-acetoxypropane), ethyl ethoxypropionate, 2-heptanone, γ-butyrolactone, cyclohexanone or butyl acetate, most preferably propylene glycol monomethyl ether acetate, ethyl ethoxypropionate or 2-heptanone.

The mixing ratio (by mass) of the solvent containing a hydroxyl group to the solvent not containing a hydroxyl group is from 1/99 to 99/1, preferably from 10/90 to 90/10, more preferably from 20/80 to 60/40. A mixed solvent in which the solvent not containing a hydroxyl group accounts for 50 mass % or more is particularly preferred in view of coating uniformity.

The solvent preferably contains propylene glycol monomethyl ether acetate and is preferably a solvent containing propylene glycol monomethyl ether acetate (PGMEA) alone or a mixed solvent of two or more kinds of solvents containing propylene glycol monomethyl ether acetate (PGMEA). Specific preferred examples of the mixed solvent include, but are not limited to, a mixed solvent containing PGMEA and a ketone-based solvent (such as cyclohexanone and 2-heptanone), a mixed solvent containing PGMEA and a lactone-based solvent (such as γ-butyrolactone), a mixed solvent containing PGMEA and PGME, a mixed solvent containing three kinds of solvents of PGMEA, a ketone-based solvent and a lactone-based solvent, a mixed solvent containing three kinds of solvents of PGMEA, PGME and a lactone-based solvent, and a mixed solvent containing three kinds of solvents of PGMEA, PGME and a ketone-based solvent.

### [7](F) Surfactant

The actinic ray-sensitive or radiation-sensitive resin composition of the present invention may or may not further contain a surfactant, but in the case of containing a surfactant, it is preferred to contain any one of fluorine-containing and/or silicon-containing surfactants (a fluorine-containing surfactant, a silicon-containing surfactant and a surfactant containing both a fluorine atom and a silicon atom), or two or more thereof.

By containing the surfactant, the actinic ray-sensitive or radiation-sensitive resin composition of the present invention can give a resist pattern improved in the sensitivity, resolution and adherence and reduced in the development defect when using an exposure light source of 250 nm or less, particularly 220 nm or less.

The fluorine-containing and/or silicon-containing surfactants include surfactants described in paragraph [0276] of U.S. Patent Application Publication No. 2008/0248425, for example, EFtop EF301 and EF303 (produced by Shin-Akita Kasei K.K.); Florad FC430, 431 and 4430 (produced by Sumitomo 3M Inc.); Megaface F171, F173, F176, F189, F113, F110, F177, F120 and R08 (produced by DIC Corporation); Surflon S-382, SC101, 102, 103, 104, 105 and 106, and KH-20 (produced by Asahi Glass Co., Ltd.); Troysol S-366 (produced by Troy Chemical); GF-300 and GF-150 (produced by Toagosei Chemical Industry Co., Ltd.); Surflon S-393 (produced by Seimi Chemical Co., Ltd.); EFtop EF121, EF122A, EF122B, RF122C, EF125M, EF135M, EF351, EF352, EF801, EF802 and EF601 (produced by JEMCO Inc.); PF636, PF656, PF6320 and PF6520 (produced by OMNOVA); and FTX-204G, 208G, 218G, 230G, 204D, 208D, 212D, 218D and 222D (produced by NEOS Co., Ltd.). In addition, Polysiloxane Polymer KP-341 (produced by Shin-Etsu Chemical Co., Ltd.) may also be used as the silicon-containing surfactant.

Other than those known surfactants, a surfactant using a polymer having a fluoro-aliphatic group derived from a fluoro-aliphatic compound which is produced by a telomerization process (also called a telomer process) or an oligomerization process (also called an oligomer process), may be used. The fluoro-aliphatic compound can be synthesized by the method described in JP-A-2002-90991.

Examples of the surfactant coming under the surfactant above include Megaface F178, F-470, F-473, F-475, F-476 and F-472 (produced by DIC Corporation); a copolymer of a C₆F₁₃ group-containing acrylate (or methacrylate) with a (poly(oxyalkylene)) acrylate (or methacrylate); and a copolymer of a C₃F₇ group-containing acrylate (or methacrylate) with a (poly(oxyethylene)) acrylate (or methacrylate) and a (poly(oxypropylene)) acrylate (or methacrylate).

In the present invention, surfactants other than the fluorine-containing and/or silicon-containing surfactant, described in paragraph [0280] of U.S. Patent Application Publication No. 2008/0248425, may also be used.

One of these surfactants may be used alone, or some of them may be used in combination.

In the case where the actinic ray-sensitive or radiation-sensitive resin composition contains a surfactant, the amount of the surfactant used is preferably from 0.0001 to 2 mass %, more preferably from 0.0005 to 1 mass %, based on the total amount of the actinic ray-sensitive or radiation-sensitive resin composition (excluding the solvent).

On the other hand, when the amount of the surfactant added is set to be 10 ppm or less based on the total amount of the actinic ray-sensitive or radiation-sensitive resin composition (excluding the solvent), the hydrophobic resin is more unevenly distributed to the surface, whereby the resist film surface can be made more hydrophobic and the followability of water at the immersion exposure can be enhanced.

### [8] Pattern Forming Method

The pattern forming method according to the present invention is described below.

The pattern forming method (preferably a negative pattern forming method) of the present invention comprises at least:

(i) a step of forming a film (resist film) by using the actinic ray-sensitive or radiation-sensitive resin composition of the present invention,
(ii) a step of irradiating (exposing) the film with an actinic ray or radiation, and
(iii) a step of developing the film irradiated with an actinic ray or radiation by using a developer (preferably an organic solvent-containing developer).

The exposure in the step (ii) may be immersion exposure.

The pattern forming method of the present invention preferably includes (iv) a heating step after the exposure step (ii).

The pattern forming method of the present invention may further include (v) a step of performing development by using an alkali developer.

In the pattern forming method of the present invention, the exposure step (ii) may be performed a plurality of times.

In the pattern forming method of the present invention, the heating step (iv) may be performed a plurality of times.

The resist film of the present invention is formed from the above-described actinic ray-sensitive or radiation-sensitive resin composition of the present invention and, more specifically, is preferably a film formed by coating the actinic ray-sensitive or radiation-sensitive resin composition on a substrate. In the pattern forming method of the present invention, the step of forming a film on a substrate by using the actinic ray-sensitive or radiation-sensitive resin composition, the step of exposing the film, and the development step can be performed by generally known methods.

It is also preferred to include, after film formation, a preheating step (PB; Prebake) before entering the exposure step.

Furthermore, it is also preferred to include a post-exposure heating step (PEB; Post Exposure Bake) after the exposure step but before the development step.

As for the heating temperature, both PB and PEB are preferably performed at 70 to 130° C., more preferably at 80 to 120° C.

The heating time is preferably from 30 to 300 seconds, more preferably from 30 to 180 seconds, still more preferably from 30 to 90 seconds.

The heating can be performed using a device attached to an ordinary exposure/developing machine or may be performed using a hot plate or the like.

Thanks to baking, the reaction in the exposed area is accelerated, and the sensitivity and pattern profile are improved.

The light source wavelength used for the exposure apparatus in the present invention is not limited but includes, for example, near infrared light, visible light, ultraviolet light, far ultraviolet light, extreme-ultraviolet light, X-ray and electron beam and is preferably far ultraviolet light at a wavelength of 250 nm or less, more preferably 220 nm or less, still more preferably from 1 to 200 nm. Specific examples thereof include KrF excimer laser (248 nm), ArF excimer laser (193 nm), F₂ excimer laser (157 nm), X-ray, EUV (13 nm) and electron beam. Among these, KrF excimer laser, ArF excimer laser, EUV and electron beam are preferred, and ArF excimer laser is more preferred.

In the step of performing exposure of the present invention, an immersion exposure method can be applied. The immersion exposure method can be combined with a super-resolution technology such as phase-shift method and modified illumination method.

In the case of performing immersion exposure, a step of washing the film surface with an aqueous chemical solution may be performed (1) after forming a film on a substrate but before the step of exposing the film and/or (2) after the step of exposing the film through an immersion liquid but before the step of heating the film.

The immersion liquid is preferably a liquid being transparent to light at the exposure wavelength and having as small a temperature coefficient of refractive index as possible in order to minimize the distortion of an optical image projected on the film. Particularly, when the exposure light source is ArF excimer laser (wavelength: 193 nm), water is preferably used in view of availability and ease of handling, in addition to the above-described aspects.

In the case of using water, an additive (liquid) capable of decreasing the surface tension of water and increasing the interface activity may be added in a small ratio. This additive is preferably an additive that does not dissolve the resist layer on the wafer and at the same time, gives only a negligible effect on the optical coat at the undersurface of the lens element.

Such an additive is preferably, for example, an aliphatic alcohol having a refractive index substantially equal to that of water, and specific examples thereof include methyl alcohol, ethyl alcohol and isopropyl alcohol. By virtue of adding an alcohol having a refractive index substantially equal to that of water, even when the alcohol component in water is evaporated and its content concentration is changed, the change in the refractive index of the liquid as a whole can be advantageously made very small.

On the other hand, if a substance opaque to light at 193 nm or an impurity greatly differing in the refractive index from water is mingled, this incurs distortion of the optical image projected on the resist. Therefore, the water used is preferably distilled water.

Furthermore, pure water after filtration through an ion exchange filter or the like may also be used.

The electrical resistance of water used as the immersion liquid is preferably 18.3 MΩcm or more, and TOC (total organic carbon) is preferably 20 ppb or less. The water is preferably subjected to a deaeration treatment.

Also, the lithography performance can be enhanced by raising the refractive index of the immersion liquid. From such a standpoint, an additive for raising the refractive index may be added to water, or heavy water (D₂O) may be used in place of water.

The receding contact angle of the resist film formed using the actinic ray-sensitive or radiation-sensitive resin composition of the present invention is 70° or more at a temperature of 23±3° C. and a humidity of 45±5%, and when exposing the film through an immersion medium, the receding contact angle is preferably 75° C. or more, more preferably from 75 to 85°.

If the receding contact angle is too small, the composition cannot be suitably used when exposing the film through an immersion medium and at the same time, the effect of reducing the watermark defect cannot be sufficiently brought out. In order to realize the preferred receding contact angle, it is preferred to incorporate the above-described hydrophobic resin (HR) into the actinic ray-sensitive or radiation-sensitive composition. Alternatively, the receding contact angle may be increased by forming a coating layer (so-called “topcoat”) from a hydrophobic resin composition on the resist film.

In the immersion exposure step, the immersion liquid must move on a wafer by conforming to the movement of an exposure head that is scanning the wafer at a high speed to form an exposure pattern. Therefore, the contact angle of the immersion liquid for the resist film in a dynamic state is important, and the resist is required to have a performance allowing the immersion liquid to follow the high-speed scanning of an exposure head with no remaining of a liquid droplet.

In the present invention, the substrate on which the film is formed is not particularly limited, and an inorganic substrate such as silicon, SiN, SiO₂ and SiN, a coating-type inorganic substrate such as SOG, or a substrate generally used in the process of producing a semiconductor such as IC or producing a liquid crystal device or a circuit board such as thermal head or in the lithography of other photo-fabrication processes, can be used. If desired, an organic antireflection film may be formed between the resist film and the substrate. As the antireflection film, a known organic or inorganic antireflection film can be appropriately used.

In the case where the pattern forming method of the present invention includes a step of performing development by using an alkali developer, the alkali developer which can be used is not particularly limited, but in general, an aqueous 2.38 mass % tetramethylammonium hydroxide solution is preferred. Also, the alkaline aqueous solution may be used after adding thereto alcohols and a surfactant each in an appropriate amount.

The alkali concentration of the alkali developer is usually from 0.1 to 20 mass %.

The pH of the alkali developer is usually from 10.0 to 15.0.

As for the rinsing solution in the rinsing treatment performed after the alkali development, pure water is used and may be used after adding thereto an appropriate amount of a surfactant.

Incidentally, by combining development using an organic solvent-containing developer and development using an alkali developer, a pattern half the optical image of a mask pattern described, for example, in U.S. Pat. No. 8,227,183 can also be obtained.

After the development or rinsing treatment, a treatment of removing the developer or rinsing solution adhering on the pattern by a supercritical fluid may be performed.

As for the developer usable in the step of performing development by using an organic solvent-containing developer (hereinafter, sometimes referred to as an “organic developer”), which is included in the pattern forming method of the present invention, a polar solvent such as ketone-based solvent, ester-based solvent, alcohol-based solvent, amide-based solvent and ether-based solvent, or a hydrocarbon-based solvent can be used.

The ketone-based solvent includes, for example, 1-octanone, 2-octanone, 1-nonanone, 2-nonanone, acetone, 2-heptanone (methyl amyl ketone), 4-heptanone, 1-hexanone, 2-hexanone, diisobutyl ketone, cyclohexanone, methylcyclohexanone, phenylacetone, methyl ethyl ketone, methyl isobutyl ketone, acetyl acetone, acetonyl acetone, ionone, diacetonyl alcohol, acetyl carbinol, acetophenone, methyl naphthyl ketone, isophorone, and propylene carbonate.

The ester-based solvent includes, for example, methyl acetate, butyl acetate, ethyl acetate, isopropyl acetate, pentyl acetate, isopentyl acetate, amyl acetate, propylene glycol monomethyl ether acetate, ethylene glycol monoethyl ether acetate, diethylene glycol monobutyl ether acetate, diethylene glycol monoethyl ether acetate, ethyl-3-ethoxypropionate, 3-methoxybutyl acetate, 3-methyl-3-methoxybutyl acetate, methyl formate, ethyl formate, butyl formate, propyl formate, ethyl lactate, butyl lactate, and propyl lactate.

The alcohol-based solvent includes, for example, an alcohol such as methyl alcohol, ethyl alcohol, n-propyl alcohol, isopropyl alcohol, n-butyl alcohol, sec-butyl alcohol, tert-butyl alcohol, isobutyl alcohol, n-hexyl alcohol, n-heptyl alcohol, n-octyl alcohol and n-decanol; a glycol-based solvent such as ethylene glycol, diethylene glycol and triethylene glycol; and a glycol ether-based solvent such as ethylene glycol monomethyl ether, propylene glycol monomethyl ether, ethylene glycol monoethyl ether, propylene glycol monoethyl ether, diethylene glycol monomethyl ether, triethylene glycol monoethyl ether and methoxymethyl butanol.

The ether-based solvent includes, for example, dioxane and tetrahydrofuran, in addition to the glycol ether-based solvents above.

The amide-based solvent which can be used includes, for example, N-methyl-2-pyrrolidone, N,N-dimethylacetamide, N,N-dimethylformamide, hexamethylphosphoric triamide, and 1,3-dimethyl-2-imidazolidinone.

The hydrocarbon-based solvent includes, for example, an aromatic hydrocarbon-based solvent such as toluene and xylene, and an aliphatic hydrocarbon-based solvent such as pentane, hexane, octane and decane.

A plurality of these solvents may be mixed, or the solvent may be used by mixing it with a solvent other than those described above or with water. However, in order to sufficiently bring out the effects of the present invention, the water content percentage in the entire developer is preferably less than 10 mass %, and it is more preferred to contain substantially no water.

That is, the amount of the organic solvent used in the organic developer is preferably from 90 to 100 mass %, more preferably from 95 to 100 mass %, based on the total amount of the developer.

In particular, the organic developer is preferably a developer containing at least one kind of an organic solvent selected from the group consisting of a ketone-based solvent, an ester-based solvent, an alcohol-based solvent, an amide-based solvent and an ether-based solvent.

The vapor pressure at 20° C. of the organic developer is preferably 5 kPa or less, more preferably 3 kPa or less, still more preferably 2 kPa or less. By setting the vapor pressure of the organic developer to 5 kPa or less, evaporation of the developer on a substrate or in a development cup is suppressed and the temperature uniformity in the wafer plane is enhanced, as a result, the dimensional uniformity in the wafer plane is improved.

In the organic developer, an appropriate amount of a surfactant can be added, if desired.

The surfactant is not particularly limited but, for example, ionic or nonionic fluorine-containing and/or silicon-containing surfactants can be used. These fluorine-containing and/or silicon-containing surfactants include, for example, surfactants described in JP-A-62-36663, JP-A-61-226746, JP-A-61-226745, JP-A-62-170950, JP-A-63-34540, JP-A-7-230165, JP-A-8-62834, JP-A-9-54432, JP-A-9-5988 and U.S. Pat. Nos. 5,405,720, 5,360.692, 5,529,881, 5,296,330, 5,436,098, 5,576,143, 5,294,511 and 5,824,451. A nonionic surfactant is preferred. The nonionic surfactant is not particularly limited, but use of a fluorine-containing surfactant or a silicon-containing surfactant is more preferred.

The amount of the surfactant used is usually from 0.001 to 5 mass %, preferably from 0.005 to 2 mass %, more preferably from 0.01 to 0.5 mass %, based on the total amount of the developer.

As regards the developing method, for example, a method of dipping the substrate in a bath filled with the developer for a fixed time (dipping method), a method of raising the developer on the substrate surface by the effect of a surface tension and keeping it still for a fixed time, thereby performing the development (puddling method), a method of spraying the developer on the substrate surface (spraying method), and a method of continuously ejecting the developer on the substrate spinning at a constant speed while scanning with a developer ejecting nozzle at a constant rate (dynamic dispense method) may be applied.

Also, the organic developer may contain a basic compound, if desired. The basic compound includes, for example, a nitrogen-containing basic compound, and examples thereof include nitrogen-containing compounds recited particularly in paragraphs [0021] to [0063] of JP-A-2013-11833. By containing a basic compound in the organic developer, for example, a rise in the contrast and suppression of film loss can be expected at the development.

In the case where the above-described various developing methods include a step of ejecting the developer toward the resist film from a development nozzle of a developing apparatus, the ejection pressure of the developer ejected (the flow velocity per unit area of the developer ejected) is preferably 2 mL/sec/mm² or less, more preferably 1.5 mL/sec/mm² or less, still more preferably 1 mL/sec/mm² or less. The lower limit of the flow velocity is not particularly limited but in view of throughput, is preferably 0.2 mL/sec/mm² or more.

By setting the ejection pressure of the ejected developer to the range above, pattern defects attributable to the resist scum after development can be greatly reduced.

Details of this mechanism are not clearly known, but it is considered that thanks to the ejection pressure in the above-described range, the pressure imposed on the resist film by the developer becomes small and the resist film or resist pattern is kept from inadvertent chipping or collapse.

Here, the ejection pressure (mL/sec/mm²) of the developer is a value at the outlet of a development nozzle in a developing apparatus.

The method for adjusting the ejection pressure of the developer includes, for example, a method of adjusting the ejection pressure by a pump or the like, and a method of adjusting the pressure by the supply from a pressurized tank.

After the step of performing development by using an organic solvent-containing developer, a step of stopping the development while replacing the solvent with another solvent may be practiced.

The pattern forming method preferably includes a step of rinsing the film by using a rinsing solution after the step of performing development by using an organic solvent-containing developer.

The rinsing solution used in the rinsing step after the step of performing development by using an organic solvent-containing developer is not particularly limited as long as it does not dissolve the resist pattern, and a solution containing a general organic solvent may be used. As the rinsing solution, it is preferred to use a rinsing solution containing at least one kind of an organic solvent selected from the group consisting of a hydrocarbon-based solvent, a ketone-based solvent, an ester-based solvent, an alcohol-based solvent, an amide-based solvent and an ether-based solvent.

Specific examples of the hydrocarbon-based solvent, ketone-based solvent, ester-based solvent, alcohol-based solvent, amide-based solvent and ether-based solvent are the same as those described above for the organic solvent-containing developer.

After the step of performing development by using an organic solvent-containing developer, more preferably, a step of rinsing the film by using a rinsing solution containing at least one kind of an organic solvent selected from the group consisting of a ketone-based solvent, an ester-based solvent, an alcohol-based solvent and an amide-based solvent is preformed; still more preferably, a step of rinsing the film by using a rinsing solution containing an alcohol-based solvent or an ester-based solvent is performed; yet still more preferably, a step of rinsing the film by using a rinsing solution containing a monohydric alcohol is performed; and most preferably, a step of rinsing the film by using a rinsing solution containing a monohydric alcohol having a carbon number of 5 or more is performed.

The monohydric alcohol used in the rinsing step includes a linear, branched or cyclic monohydric alcohol, and specifically, 1-butanol, 2-butanol, 3-methyl-1-butanol, tert-butyl alcohol, 1-pentanol, 2-pentanol, 1-hexanol, 4-methyl-2-pentanol, 1-heptanol, 1-octanol, 2-hexanol, cyclopentanol, 2-heptanol, 2-octanol, 3-hexanol, 3-heptanol, 3-octanol, 4-octanol and the like can be used. As the particularly preferred monohydric alcohol having a carbon number of 5 or more, 1-hexanol, 2-hexanol, 4-methyl-2-pentanol, 1-pentanol, 3-methyl-1-butanol and the like can be used.

A plurality of these components may be mixed, or the solvent may be used by mixing it with an organic solvent other than those described above.

The water content percentage in the rinsing solution is preferably 10 mass % or less, more preferably 5 mass % or less, still more preferably 3 mass % or less. By setting the water content percentage to 10 mass % or less, good development characteristics can be obtained.

The vapor pressure at 20° C. of the rinsing solution used after the step of performing development by using an organic solvent-containing developer is preferably from 0.05 to 5 kPa, more preferably from 0.1 to 5 kPa, and most preferably from 0.12 to 3 kPa. By setting the vapor pressure of the rinsing solution to be from 0.05 to 5 kPa, the temperature uniformity in the wafer plane is enhanced and moreover, swelling due to permeation of the rinsing solution is suppressed, as a result, the dimensional uniformity in the wafer plane is improved.

The rinsing solution may also be used after adding thereto an appropriate amount of a surfactant.

In the rinsing step, the wafer subjected to development using an organic solvent-containing developer is rinsed by using a rinsing solution containing the above-described organic solvent. The method for rinsing treatment is not particularly limited but, for example, a method of continuously ejecting the rinsing solution on the substrate spinning at a constant speed (spin coating method), a method of dipping the substrate in a bath filled with the rinsing solution for a fixed time (dipping method), and a method of spraying the rinsing solution on the substrate surface (spraying method) can be applied. Above all, it is preferred to perform the rinsing treatment by the spin coating method and after the rinsing, remove the rinsing solution from the substrate surface by spinning the substrate at a rotational speed of 2,000 to 4,000 rpm. It is also preferred to include a heating step (Post Bake) after the rinsing step. The developer and rinsing solution remaining between patterns and in the inside of the pattern are removed by the baking. The heating step after the rinsing step is performed at usually from 40 to 160° C., preferably from 70 to 95° C., for usually from 10 seconds to 3 minutes, preferably from 30 to 90 seconds.

As the alkali developer in the step of performing development by using an alkali developer, for example, an alkaline aqueous solution of inorganic alkalis such as sodium hydroxide, potassium hydroxide, sodium carbonate, sodium silicate, sodium metasilicate and aqueous ammonia, primary amines such as ethylamine and n-propylamine, secondary amines such as diethylamine and di-n-butylamine, tertiary amines such as triethylamine and methyldiethylamine, alcohol amines such as dimethylethanolamine and triethanolamine, quaternary ammonium salts such as tetramethylammonium hydroxide and tetraethylammonium hydroxide, or cyclic amines such as pyrrole and piperidine, can be used.

Furthermore, the alkaline aqueous solution above may also be used after adding thereto alcohols and a surfactant each in an appropriate amount.

The alkali concentration of the alkali developer is usually from 0.1 to 20 mass %.

The pH of the alkali developer is usually from 10.0 to 15.0.

In particular, an aqueous 2.38 mass % tetramethylammonium hydroxide solution is preferred.

As the rinsing solution in the rinsing treatment performed after the alkali development, pure water is used and may be used after adding thereto an appropriate amount of a surfactant.

Also, after the development or rinsing treatment, a treatment of removing the developer or rinsing solution adhering on the pattern by a supercritical fluid may be performed.

The pattern obtained by the pattern forming method of the present invention is in general suitably used as an etching mask or the like of a semiconductor device but is used for other uses. Other uses include, for example, a formation of a guide pattern (see, for example, ACS Nano, Vol. 4, No. 8, pp. 4815-4823) in DSA (Directed Self-Assembly), so-called use as a core for a spacer process (see, for example, JP-A-3-270227 and JP-A-2013-164509).

The present invention also relates to a method for manufacturing an electronic device, comprising the pattern forming method of the present invention, and an electronic device manufactured by this manufacturing method.

The electronic device of the present invention is suitably mounted on electric electronic equipment (such as home electronics, OA•media equipment, optics and communication equipment).

### EXAMPLES

### Synthesis of Resin (P−1)

In a nitrogen stream, a three-neck flask was charged with 66.9 g of cyclohexanone and heated at 80° C. Subsequently, a solution obtained by dissolving the below-shown Monomer 1 (14.8 g) and Monomer 2 (18.9 g) in cyclohexanone (124.4 g) to prepare a monomer solution and furthermore, adding and dissolving 0.55 g (2.0 mol % based on the total amount of monomers) of polymerization initiator V-601 (produced by Wako Pure Chemical Industries, Ltd.) in the monomer solution was added dropwise to the flask over 6 hours. After the completion ofdropwise addition, the reaction was further allowed to proceed at 80° C. for 2 hours. The reaction solution was left to cool and then added dropwise to a mixed solvent of 1,418 g of heptane/157.6 g of ethyl acetate, and the precipitated powder was collected by filtration and dried to obtain 26.9 g of Resin (P-1). The weight average molecular weight of Resin (P−1) as determined from GPC (carrier: tetrahydrofuran (THF)) was 21,500, the polydispersity (Mw/Mn) was 1.68, and the compositional ratio (molar ratio) measured by ¹³C-NMR was 40/60.

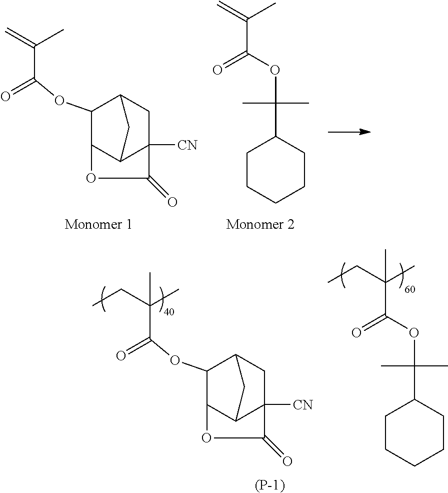

Resins (P-2) to (P-17) were synthesized in the same manner as Resin (P−1).

The structure, compositional ratio (molar ratio) of repeating units, weight average molecular weight and polydispersity of each of the resins synthesized are shown below.

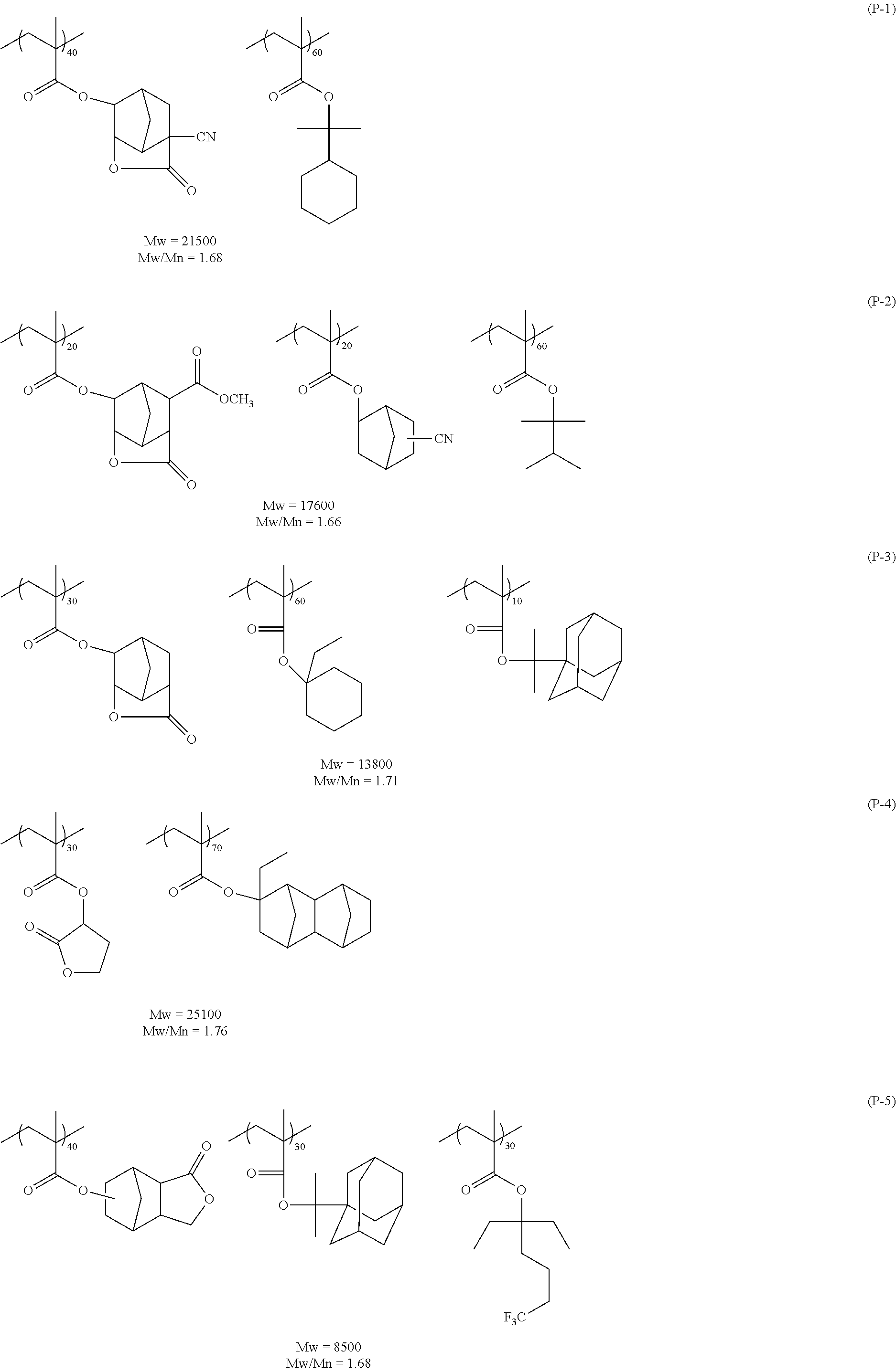

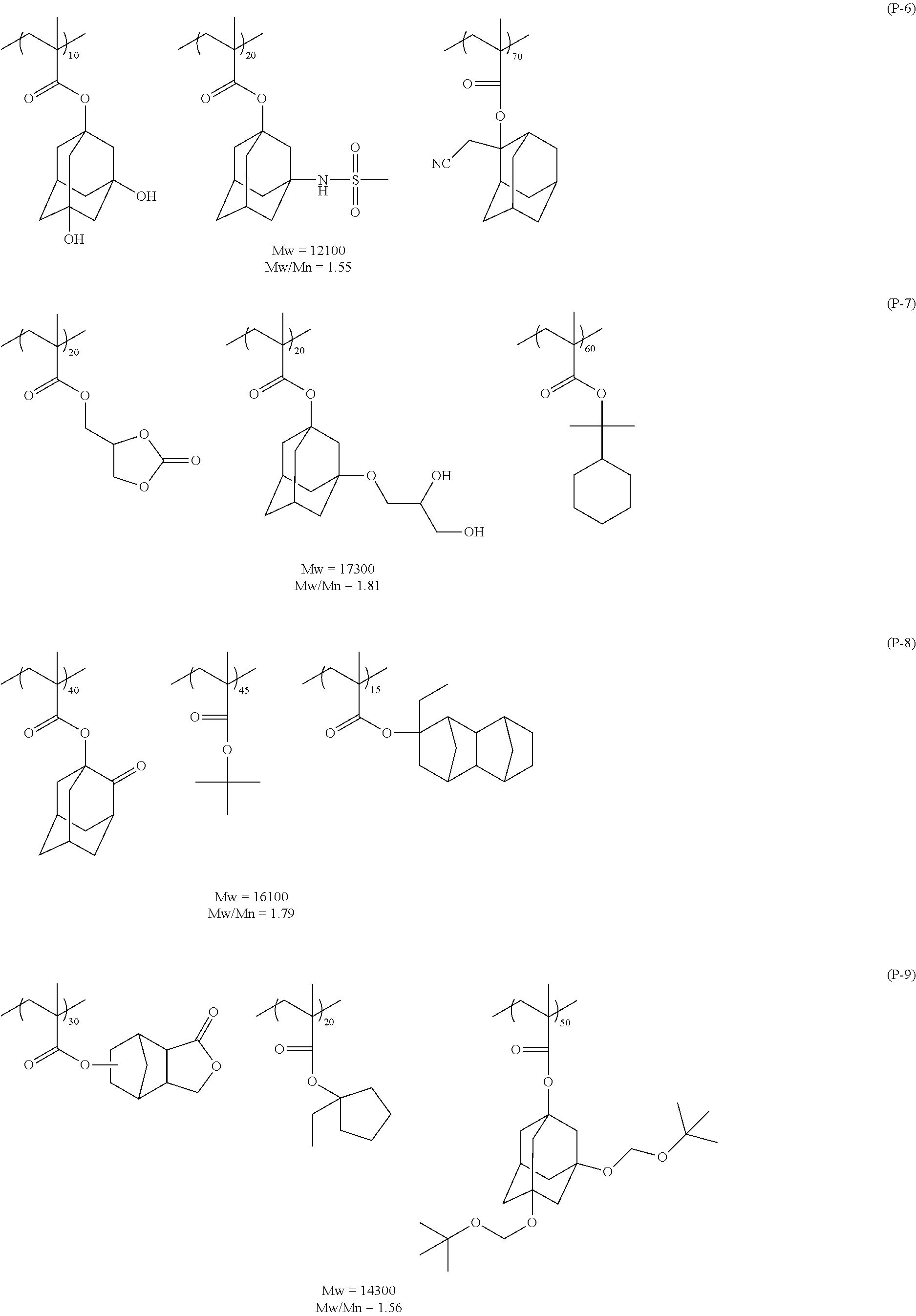

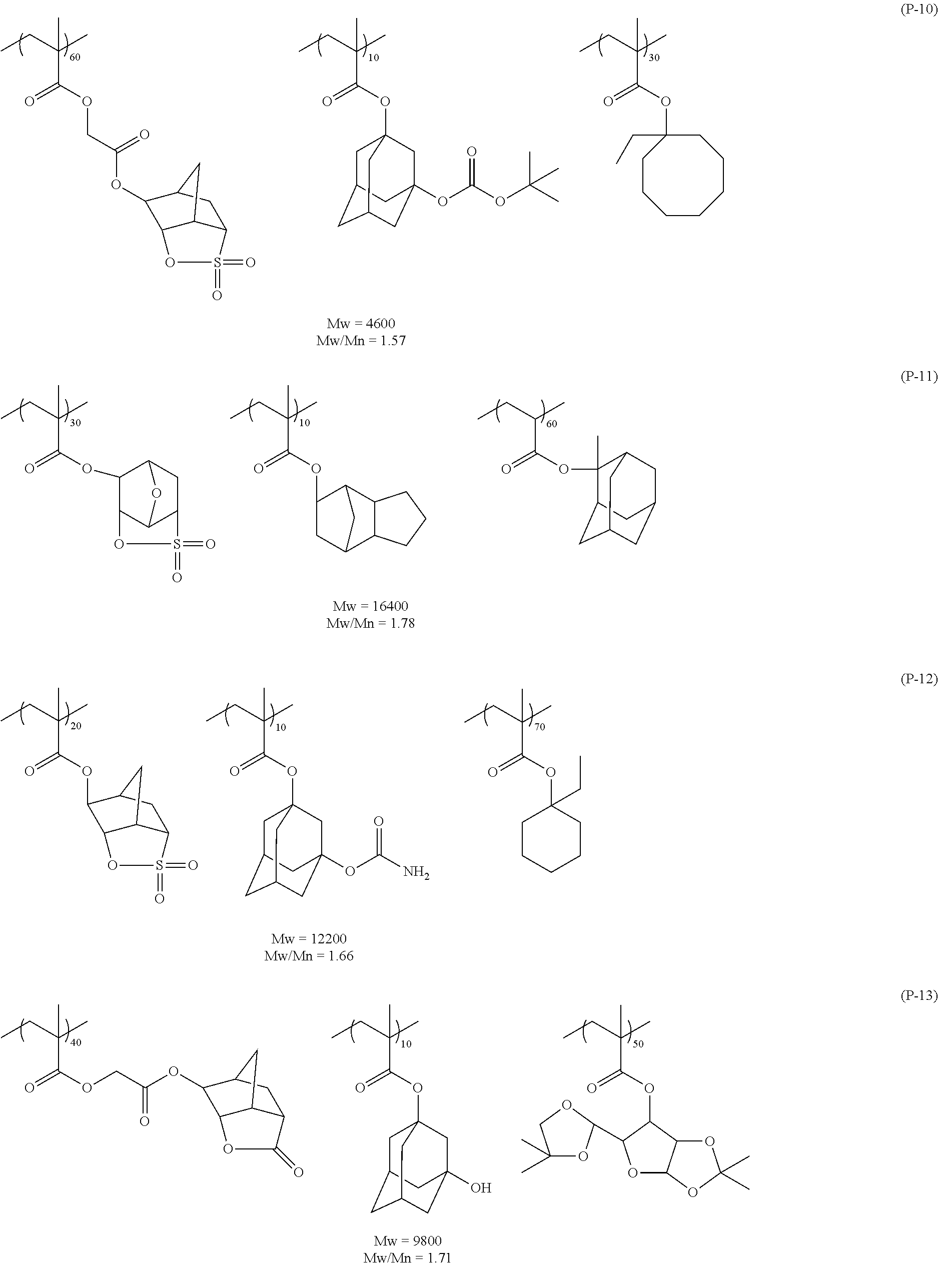

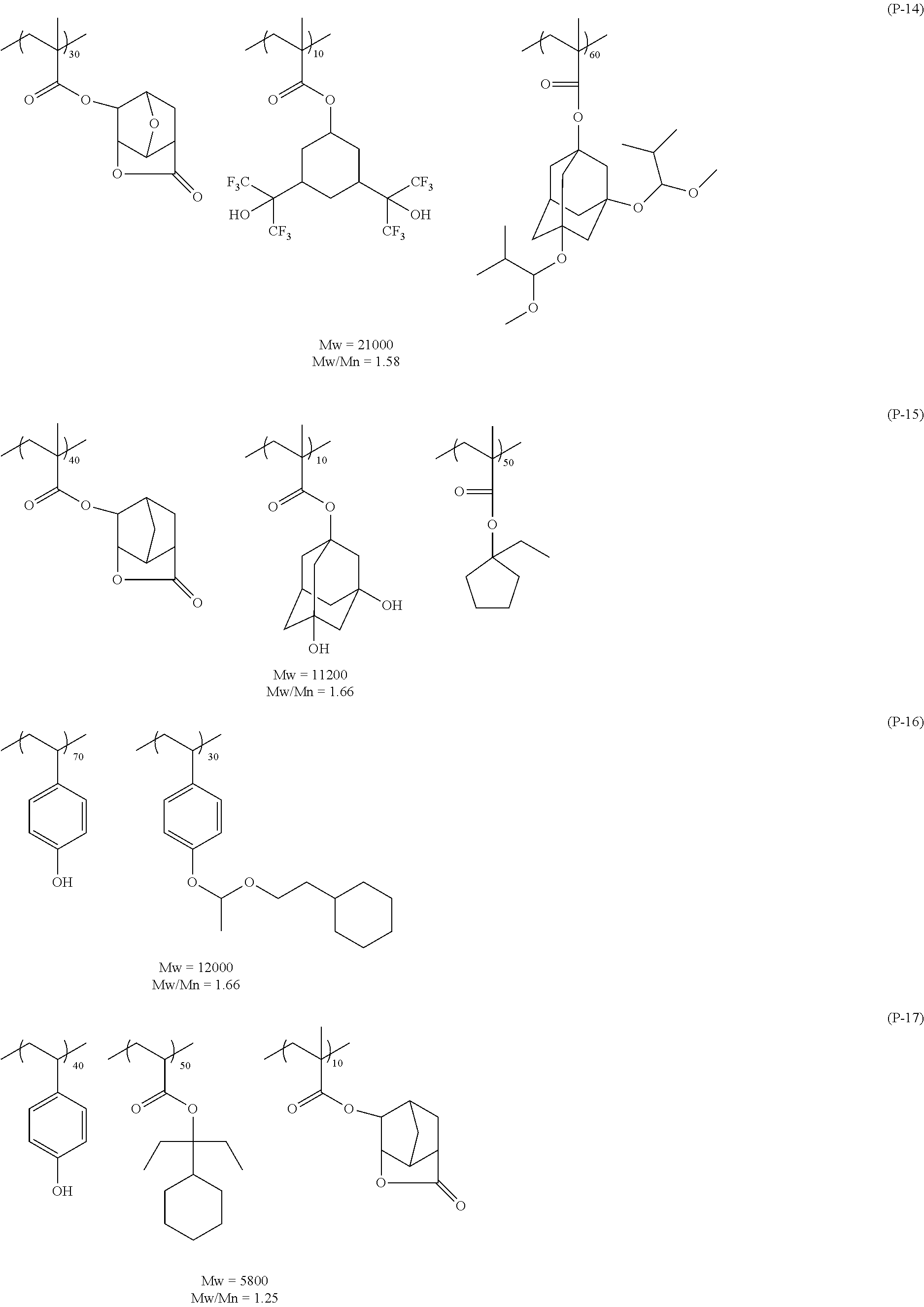

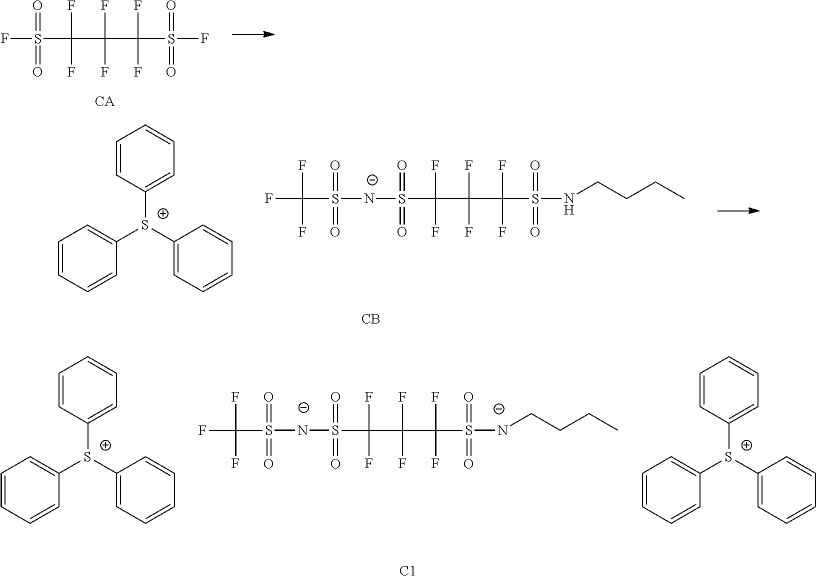

### Synthesis of Compound CB

In a 500-ml three-neck flask, 20.0 g (198 mmol) of triethylamine and 4.7 g (32 mmol) of trifluoromethanesulfonamide were mixed with 10 g of THF and after cooling the mixture to 0° C., 10.0 g (32 mmol) of Compound CA was added dropwise. The resulting solution was stirred at room temperature for 5 hours and 4.7 g (64 mmol) of butylamine was added, followed by stirring at room temperature for 70 hours. To this reaction solution, 200 g of 1 N hydrochloric acid, 10.9 g (32 mmol) of triphenylsulfonium bromide and 200 g of chloroform were added, and the organic layer was separated. Furthermore, the organic layer was washed with 200 g of deionized water five times (200 g×5 times), and the organic layer was then concentrated to obtain 12.4 g (yield: 51.6%) of Compound CB.

### Synthesis of Compound (C−1)

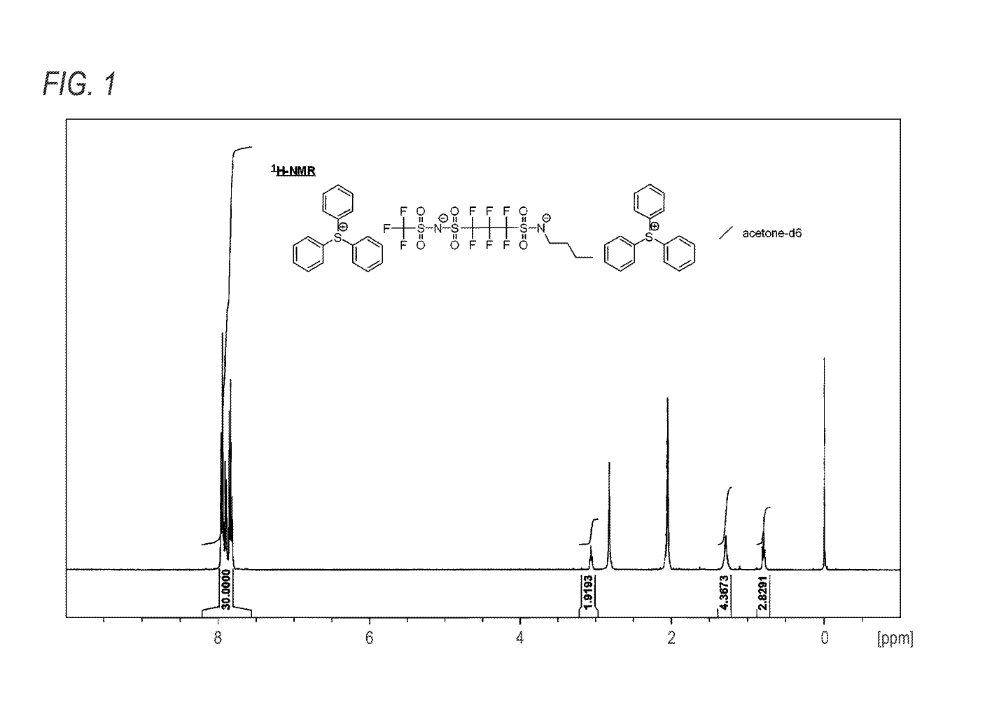

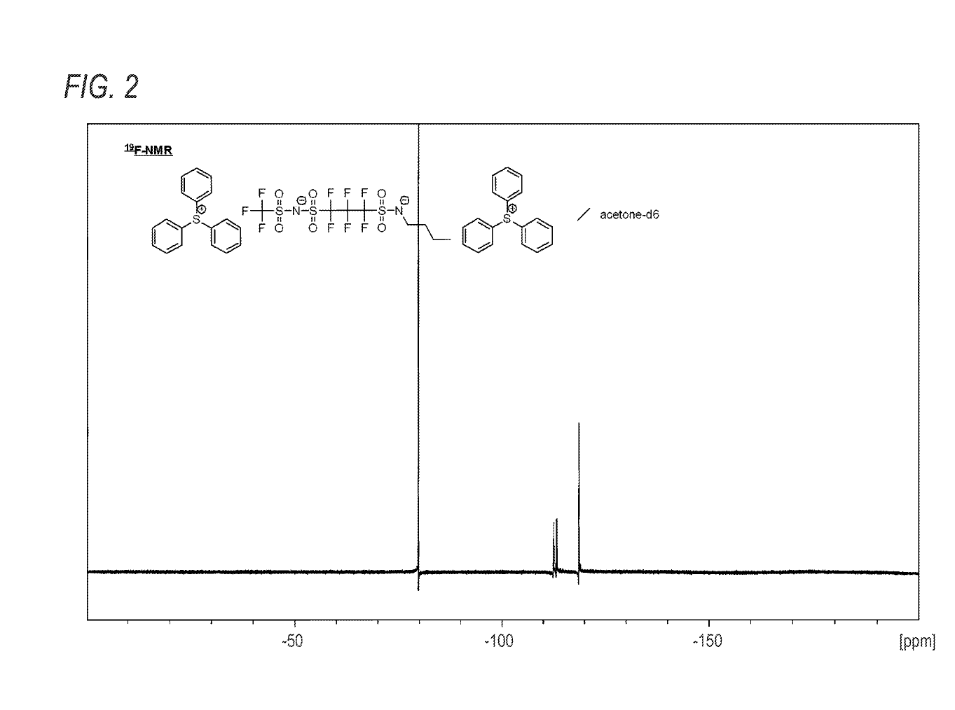

In a 500-ml eggplant flask, 10 g (13 mmol) of Compound CB was dissolved in 100 g of methylene chloride, and 100 g of an aqueous 1 N sodium hydroxide solution and 11.3 g (13 mmol) of triphenylsulfonium bromide were added thereto, followed by stirring at room temperature for 1 hour. Subsequently, the organic layer was separated and furthermore, the organic layer was washed twice by using 100 g of deionized water and then concentrated to obtain 4.6 g (34.2%) of Compound (C-1). The ¹H-NMR chart and the ¹⁹F-NMR chart of Compound (C-1) are shown in FIGS. 1 and 2, respectively.

Compounds (C-2) to (C-7) were synthesized in the same manner as Compound (C-1).

The structures of Compounds (C-1) to (C-7) are shown below.

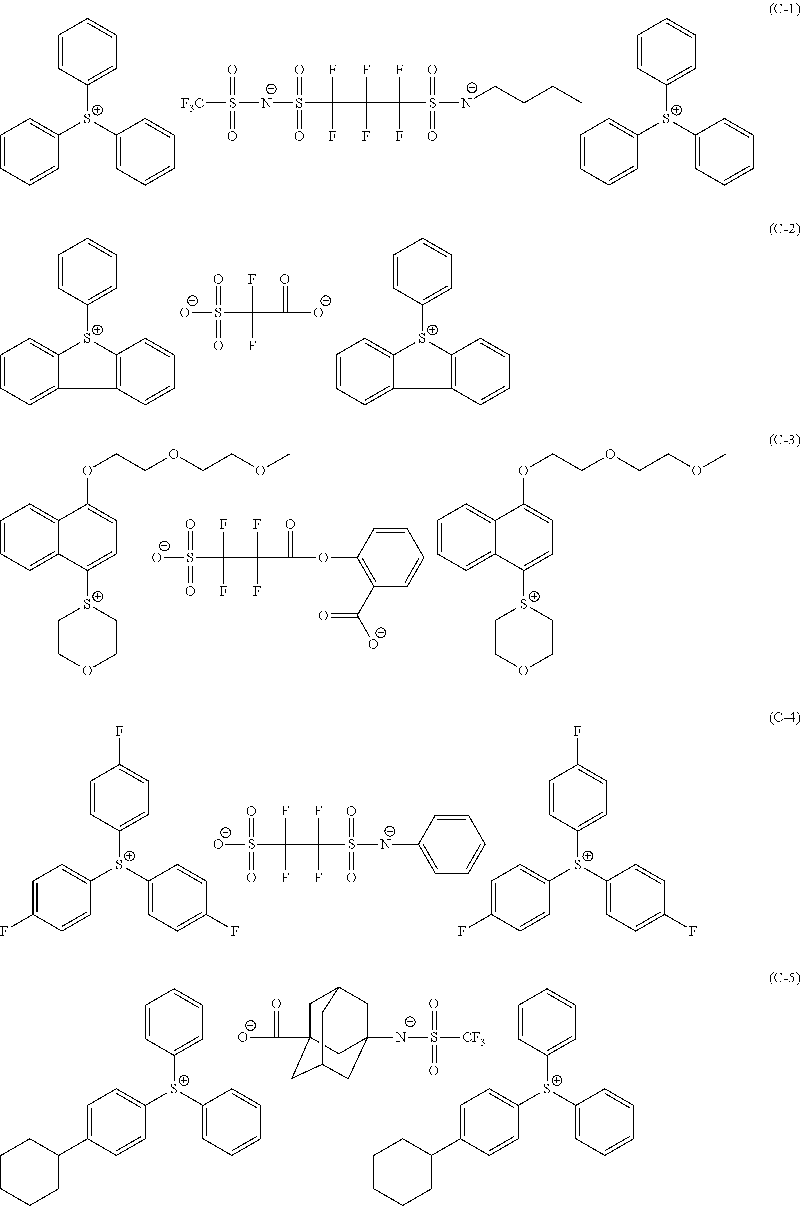

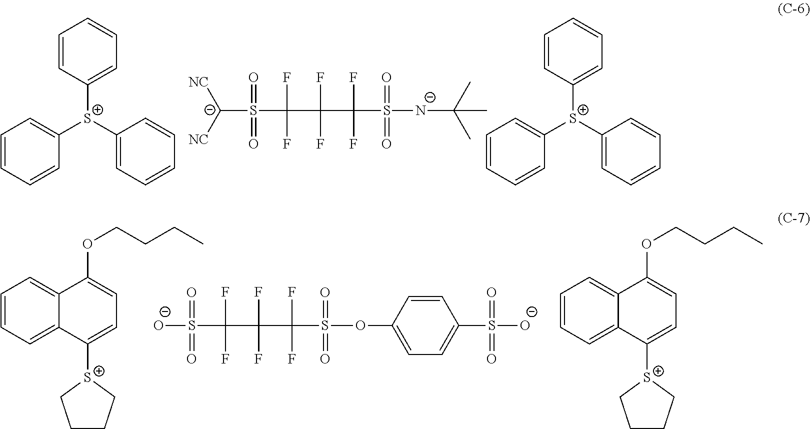 [SMILES: c1ccc([S+](c2ccccc2)c2ccccc2)cc1 ; CC(C)(C)[N-]S(=O)(=O)C(F)(F)C(F)(F)C(F)(F)S(=O)(=O)[C-](C#N)C#N ; c1ccc([S+](c2ccccc2)c2ccccc2)cc1 ; CCCCOc1ccc([S+]2CCCC2)c2ccccc12 ; O=S(=O)([O-])c1ccc(OS(=O)(=O)C(F)(F)C(F)(F)C(F)(F)S(=O)(=O)[O-])cc1 ; CCCCOc1ccc([S+]2CCCC2)c2ccccc12]

The compounds (B) used in Examples are shown below.

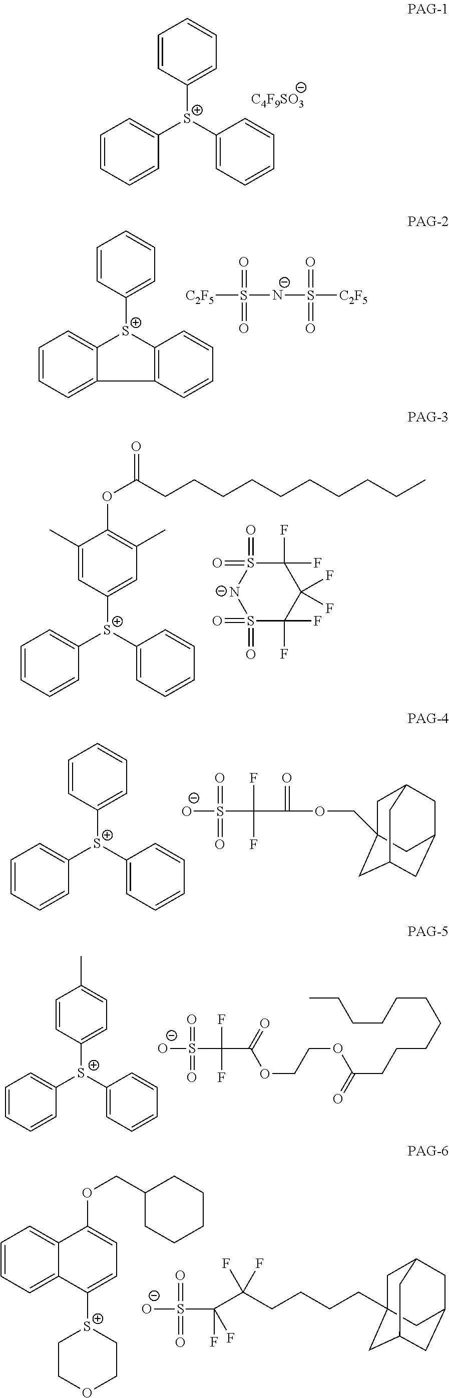

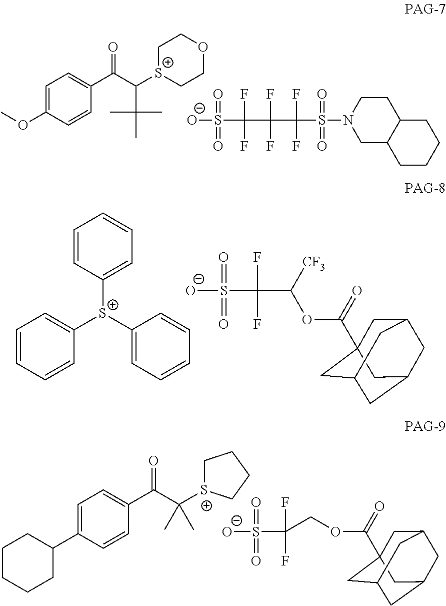 [SMILES: COc1ccc(C(=O)C([S+]2CCOCC2)C(C)(C)C)cc1 ; O=S(=O)([O-])C(F)(F)C(F)(F)C(F)(F)S(=O)(=O)N1CCC2CCCCC2C1 ; c1ccc([S+](c2ccccc2)c2ccccc2)cc1 ; O=C(OC(C(F)(F)F)C(F)(F)S(=O)(=O)[O-])C12CC3CC(CC(C3)C1)C2 ; CC(C)(C(=O)c1ccc(C2CCCCC2)cc1)[S+]1CCCC1 ; O=C(OCC(F)(F)S(=O)(=O)[O-])C12CC3CC(CC(C3)C1)C2]

The basic compounds (N) used in Examples are shown below.

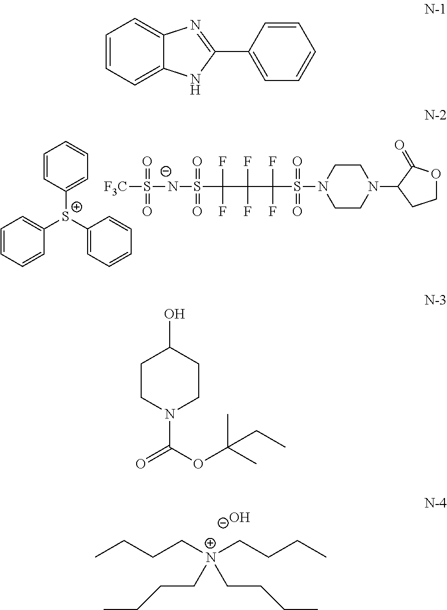

### <ArF Immersion Exposure, Organic Solvent Development>

### (Preparation of Resist)

The components shown in Table 6 below were dissolved in the solvent shown in the same Table to give a concentration of 3.8 mass % as the solid content, and each of the obtained solutions was filtered through a polyethylene filter having a pore size of 0.03 μm to prepare an actinic ray-sensitive or radiation-sensitive resin composition (resist composition). An organic antireflection film, ARC29SR (produced by Nissan Chemical Industries, Ltd.), was coated on a silicon wafer and baked at 205° C. for 60 seconds to form an antireflection film having a thickness of 95 nm, and the actinic ray-sensitive or radiation-sensitive resin composition was coated thereon and baked (PB: Prebake) at 100° C. over 60 seconds to form a resist film having a thickness of 100 nm.

The obtained wafer was patternwise exposed through a square-array halftone mask having a hole size of 45 nm and a hole-to-hole pitch of 90 nm by using an ArF excimer laser immersion scanner (XT1700i, manufactured by ASML, NA: 1.20, C-Quad, outer sigma: 0.900, inner sigma: 0.812, XY deflection). As the immersion liquid, ultrapure water was used. Thereafter, the resist film was heated at 105° C. for 60 seconds (PEB: Post Exposure Bake), developed by puddling the organic developer shown in Table 6 for 30 seconds, and then rinsed by puddling the rising solution shown in Table 6 for 30 seconds. Subsequently, the wafer was spun at a rotational speed of 4,000 rpm for 30 seconds, whereby a contact hole pattern of 45 nm was obtained.

### [Exposure Latitude (EL, %)]

The hole size was observed by a critical dimension scanning electron microscope (SEM, S-9380II, manufactured by Hitachi, Ltd.), and the optimum exposure dose when resolving a contact hole pattern having a hole size of 45 nm was taken as the sensitivity (Eₒₚₜ) (mJ/cm²). Based on the determined optimum exposure dose (Eₒₚₜ), the exposure dose when giving a target hole size value 45 nm±10% (that is, 40.5 nm and 49.5 nm) was determined. Thereafter, the exposure latitude (EL, %) defined by the following formula was calculated. As the value of EL is larger, the performance change due to change in the exposure dose is smaller and this is better.

[EL (%)]=[(exposure dose when the hole size becomes 40.5 nm)−(exposure dose when the hole size becomes 49.5 nm)]/Eₒₚₜ

[Local Pattern Dimension Uniformity (Local CDU, nm)]

Within one shot exposed at the optimum exposure dose determined in the evaluation of exposure latitude, arbitrary 25 holes in each of 20 regions spaced apart by a gap of 1 μm, that is, 500 holes in total, were measured for the hole size. The standard deviation thereof was determined, and 3σ was computed therefrom. A smaller value indicates a lower dimensional variation and a better performance.

### [Film Thickness (Nm) of Pattern Part]

The cross-sectional profile of each pattern at the optimal exposure dose above was observed by using a scanning electron microscope (S-4800, manufactured by Hitachi, Ltd.). The resist-remaining part in the hole pattern was measured for the pattern height. A larger value indicates a smaller film loss, and this is better.

[Line Width Roughness (LWR, nm)]

The obtained wafer was exposed through a 6% halftone mask having a 1:1 line-and-space pattern with a line width of 45 nm by using an ArF excimer laser immersion scanner (XT1700i, manufactured by ASML, NA: 1.20). As for the immersion liquid, ultrapure water was used. Thereafter, the resist film was heated at 105° C. for 60 seconds, then developed by puddling the developer shown in Table 6 below for 30 seconds, and rinsed by puddling the rinsing solution shown in Table 1 for 30 seconds while spinning the wafer at a rotational speed of 1,000 rpm. In the measurement of the obtained 1:1 line-and-space resist pattern having a line width of 45 nm, at the time of measuring the pattern from above by using a critical dimension scanning electron microscope (SEM: S-9380II, manufactured by Hitachi, Ltd.), the line width was measured at arbitrary points, and the measurement variation was evaluated by 3c. A smaller value indicates a better performance.

### <Hydrophobic Resin>

As the hydrophobic resin, a resin appropriately selected from Resins (HR-1) to (HR-84), (C-1) to (C-28) and (D-1) to (D-16) was used.

### <Surfactant>

As the surfactant, the followings were used.

W-1: Megaface F176 (produced by DIC Corporation; fluorine-containing)
W-2: PolyFox PF-6320 (produced by OMNOVA Solutions Inc.: fluorine-containing)
W-3: Polysiloxane Polymer KP-341 (produced by Shin-Etsu Chemical Co., Ltd.; silicon-containing)
W-4: Troysol S-366 (produced by Troy Chemical)
W-5: KH-20 (produced by Asahi Glass Co., Ltd.)

### <Solvent>

As the solvent, the followings were used.

### (Group a)

SL-1: Propylene glycol monomethyl ether acetate (PGMEA)
SL-2: Propylene glycol monomethyl ether propionate

### SL-3: 2-Heptanone

### (Group b)

SL-4: Ethyl lactate
SL-5: Propylene glycol monomethyl ether (PGME)

### SL-6: Cyclohexanone

### (Group c)

### SL-7: γ-Butyrolactone

SL-8: Propylene carbonate

These evaluation results are shown in Table 7.

TABLE 6

| | | | Compound | | Compound | | Basic | | Hydrophobic Resin | |
|---|---|---|---|---|---|---|---|---|---|---|
| Example | Resin | (g) | (B) | (g) | (C) | (g) | Compound | (g) | (E) | (g) |
| Example 1 | P-1 | 10 | | | C-1 | 1.18 | | | D-12 | 0.06 |
| Example 2 | P-2 | 10 | | | C-2 | 1.34 | | | HR-16 | 0.06 |
| Example 3 | P-3 | 10 | | | C-3 | 1.12 | | | D-4 | 0.06 |
| Example 4 | P-4 | 10 | | | C-4 | 1.24 | | | HR-59 | 0.06 |
| Example 5 | P-5 | 10 | PAG-1 | 1.50 | C-5 | 1.14 | | | C-10 | 0.06 |
| Example 6 | P-6 | 10 | PAG-2 | 1.04 | C-6 | 2.22 | | | C-14 | 0.06 |
| Example 7 | P-7 | 10 | | | C-7 | 1.32 | N-1 | 0.12 | HR-39 | 0.06 |
| Example 8 | P-8 | 10 | | | C-1 | 1.54 | N-2 | 0.42 | HR-83 | 0.06 |
| Example 9 | P-9 | 10 | PAG-3 | 1.28 | C-3 | 1.33 | | | HR-84 | 0.06 |
| Example 10 | P-10 | 10 | PAG-4 | 2.39 | C-4 | 1.28 | | | HR-51 | 0.06 |
| Example 11 | P-11 | 10 | PAG-5 | 1.45 | C-5 | 2.02 | N-3 | 0.08 | D-1 | 0.06 |
| Example 12 | P-12 | 10 | PAG-6 | 1.12 | C-6 | 1.45 | N-1 | 0.11 | D-4/C-10 | 0.04/0.02 |
| Example 13 | P-13 | 10 | PAG-7 | 1.14 | C-4 | 1.12 | N-2 | 0.42 | HR-81 | 0.06 |
| Example 14 | P-14 | 10 | PAG-8 | 1.48 | C-1 | 1.38 | | | HR-24/C-14 | 0.03/0.03 |
| Example 15 | P-1/P-14 | 5/5 | PAG-9 | 1.33 | C-2 | 1.24 | | | C-1 | 0.06 |
| Example 16 | P-2 | 10 | | | C-1/C-5 | 1.46/0.35 | | | HR-26 | 0.06 |
| Example 17 | P-7 | 10 | | | C-1 | 1.32 | | | HR-83 | 0.06 |
| Example 18 | P-1 | 10 | | | C-7 | 1.23 | | | C-10 | 0.06 |
| Comparative | P-1 | 10 | PAG-2 | 0.51 | | | N-2 | 0.42 | D-12 | 0.06 |
| Example 1 | | | | | | | | | | |

| Example | Solvent | Mass Ratio | Surfactant | (g) | Developer | Mass Ratio | Rinsing Solution | Mass Ratio |
|---|---|---|---|---|---|---|---|---|
| Example 1 | SL-1/SL-5 | 60/40 | W-4 | 0.003 | SG-1 | 100 | SR-1 | 100 |
| Example 2 | SL-1 | 100 | W-1 | 0.003 | SG-1 | 100 | SR-1 | 100 |
| Example 3 | SL-1/SL-5 | 60/40 | W-5 | 0.003 | SG-7 | 100 | SR-1 | 100 |
| Example 4 | SL-1/SL-4 | 90/10 | W-1 | 0.003 | SG-1 | 100 | SR-3 | 100 |
| Example 5 | SL-1/SL-5 | 60/40 | W-1 | 0.003 | SG-5 | 100 | SR-1 | 100 |
| Example 6 | SL-1/SL-3 | 60/40 | W-1 | 0.003 | SG-1 | 100 | SR-1 | 100 |
| Example 7 | SL-1/SL-5 | 60/40 | W-4 | 0.003 | SG-1 | 100 | SR-1 | 100 |
| Example 8 | SL-1/SL-8 | 70/30 | W-1 | 0.003 | SG-2 | 100 | | |
| Example 9 | SL-1/SL-5 | 60/40 | W-3 | 0.003 | SG-5 | 100 | SR-1 | 100 |
| Example 10 | SL-5/SL-6 | 30/70 | W-5 | 0.003 | SG-6 | 100 | SR-1/SR-3 | 90/10 |
| Example 11 | SL-1/SL-2 | 60/40 | W-2 | 0.003 | SG-3 | 100 | SR-1 | 100 |
| Example 12 | SL-1/SL-5 | 60/40 | W-2 | 0.003 | SG-1 | 100 | SR-2 | 100 |
| Example 13 | SL-1/SL-5 | 60/40 | W-1 | 0.003 | SG-1/SG-4 | 50/50 | SR-3 | 100 |
| Example 14 | SL-1/SL-5 | 60/40 | none | none | SG-8 | 100 | SR-1 | 100 |
| Example 15 | SL-5/SL-6 | 30/70 | W-1 | 0.003 | SG-1 | 100 | SR-1 | 100 |
| Example 16 | SL-1/SL-7 | 70/30 | W-1 | 0.003 | SG-1 | 100 | SR-1 | 100 |
| Example 17 | SL-1/SL-5 | 60/40 | W-3 | 0.003 | SG-1 | 100 | SR-1 | 100 |
| Example 18 | SL-1/SL-5 | 60/40 | W-2 | 0.003 | SG-1 | 100 | SR-1 | 100 |
| Comparative | SL-1/SL-5 | 60/40 | W-4 | 0.003 | SG-1 | 100 | SR-1 | 100 |
| Example 1 | | | | | | | | |

### <Developer>

As the developer, the followings were used

SG-1: Butyl acetate
SG-2: Diisobutyl ketone
SG-3: Cyclohexyl acetate
SG-4: Isobutyl isobutyrate
SG-5: Isopentyl acetate

### SG-6: Phenetole

SG-7: Dibutyl ether

### SG-8: 2-Nonanone

SG-9: An aqueous tetramethylammonium hydroxide solution at a concentration of 2.38 mass %

### <Rinsing Solution>

As the rinsing solution, the followings were used.

SR-1: 4-Methyl-2-pentanol

### SR-2: 1-Hexanol

SR-3: Butyl acetate
SR-4: Pure water

TABLE 7

| | Local | | Film Thickness of | |
|---|---|---|---|---|
| Example | CDU (nm) | EL (%) | Pattern Part (nm) | LWR (nm) |
| Example 1 | 4.4 | 19.0 | 86 | 4.7 |
| Example 2 | 4.9 | 19.3 | 85 | 5.0 |
| Example 3 | 4.7 | 19.4 | 84 | 4.8 |
| Example 4 | 4.6 | 18.8 | 84 | 4.8 |
| Example 5 | 5.7 | 18.1 | 80 | 5.4 |
| Example 6 | 4.4 | 19.0 | 81 | 5.0 |
| Example 7 | 4.3 | 19.1 | 81 | 4.9 |
| Example 8 | 4.1 | 19.0 | 82 | 4.6 |
| Example 9 | 4.2 | 18.9 | 83 | 4.5 |
| Example 10 | 4.6 | 19.3 | 84 | 4.9 |
| Example 11 | 5.5 | 17.7 | 76 | 5.2 |
| Example 12 | 4.3 | 19.0 | 83 | 4.7 |
| Example 13 | 4.4 | 19.1 | 83 | 4.9 |
| Example 14 | 4.3 | 19.0 | 83 | 4.9 |
| Example 15 | 4.9 | 19.1 | 84 | 4.7 |
| Example 16 | 4.9 | 19.0 | 82 | 4.7 |
| Example 17 | 4.5 | 19.0 | 83 | 4.8 |
| Example 18 | 4.4 | 19.0 | 81 | 4.8 |
| Comparative | 6.3 | 15.2 | 70 | 6.0 |
| Example 1 | | | | |

It is seen from the results in Table 7 that in Examples 1 to 18 using the composition according to the present invention, in the ArF immersion exposure using an organic solvent developer, the roughness performance such as line width roughness, the local pattern dimension uniformity and the exposure latitude were excellent and the reduction in the film thickness of the pattern part formed by development, so-called film loss, was suppressed, as compared with Comparative Example 1 using a composition not containing the compound (C1) or (C2).

Also, when the compound (C−1) is a compound capable of generating, as the first acidic functional group and the second acidic functional group, groups different from each other selected from the group consisting of groups represented by formulae (Ca-1) to (Ca-19) upon irradiation with an actinic ray or radiation, the roughness performance such as line width roughness, the local pattern dimension uniformity and the exposure latitude were more excellent and the reduction in the film thickness of the pattern part formed by development, so-called film loss, was more suppressed.

### <ArF Immersion Exposure, Alkali Development>

The components shown in Table 8 below were dissolved in the solvent shown in the same Table to give a concentration of 3.8 mass % as the solid content, and each of the obtained solutions was filtered through a polyethylene filter having a pore size of 0.03 μm to prepare an actinic ray-sensitive or radiation-sensitive resin composition (resist composition). An organic antireflection film, ARC29SR (produced by Nissan Chemical Industries, Ltd.), was coated on a silicon wafer and baked at 205° C. for 60 seconds to form an antireflection film having a thickness of 95 nm, and the actinic ray-sensitive or radiation-sensitive resin composition was coated thereon and baked (PB: Prebake) at 100° C. over 60 seconds to form a resist film having a thickness of 100 nm.

The obtained wafer was exposed through a 6% halftone mask having a 1:1 line-and-space pattern with a line width of 65 nm by using an ArF excimer laser immersion scanner (XT1250i, manufactured by ASML, NA: 0.85). As for the immersion liquid, ultrapure water was used. Thereafter, the resist film was heated at 130° C. for 60 seconds, then developed with an aqueous tetramethylammonium hydroxide solution (2.38 mass %) for 30 seconds, rinsed with pure water, and spin-dried to obtain a resist pattern.

### [Exposure Latitude (EL, %)]

The optimum exposure dose when resolving a 1:1 line-and-space mask pattern having a line width of 65 nm was taken as the optimum exposure dose, the exposure dose range within a tolerance of 65 nm±10% of the pattern size was determined by varying the exposure dose, and the determined value was divided by the optimum exposure dose and expressed in percentage. A larger value indicates a smaller performance change due to change in the exposure dose and a better exposure latitude.

[Line Width Roughness (LWR, nm)]

In the measurement of the obtained line-and-space resist pattern with 65 nm (1:1), at the time of measuring the pattern from above by using a critical dimension scanning electron microscope (SEM: S-93801, manufactured by Hitachi, Ltd.), the line width was measured at arbitrary points, and the measurement variation was evaluated by 3a. A smaller value indicates a better performance.

TABLE 8

| | | | Com- | | Com- | | Basic | | Hydro- | | | | | | | |
|---|---|---|---|---|---|---|---|---|---|---|---|---|---|---|---|---|
| | | | pound | | pound | | Com- | | phobic | | | Mass | | | EL | LWR |
| Example | Resin | (g) | (B) | (g) | (C) | (g) | pound | (g) | Resin (E) | (g) | Solvent | Ratio | Surfactant | (g) | (%) | (nm) |
| Example 19 | P-15 | 10 | | | C-1 | 1.23 | | | C-14 | 0.06 | SL-1/SL-5 | 60/40 | W-1 | 0.003 | 19.2 | 4.8 |
| Comparative | P-15 | 10 | PAG-2 | 0.51 | | | N-2 | 0.42 | C-14 | 0.06 | SL-1/SL-5 | 60/40 | W-1 | 0.003 | 15.1 | 6.1 |
| Example 2 | | | | | | | | | | | | | | | | |

It is seen from the results in Table 8 that in Example 19 using the composition according to the present invention, the roughness performance such as line width roughness and the exposure latitude were excellent in the ArF immersion exposure using an alkali developer, as compared with Comparative Example 2 using a composition not containing the compound (C1) or (C2).

### <EB Resist Evaluation>

### (1) Preparation and Coating of Coating Solution of Actinic Ray-Sensitive or Radiation-Sensitive Resin Composition

The components shown in Table 9 below were dissolved in the solvent shown in the same Table to give a concentration of 4.0 mass % as the solid content, and the obtained solution was microfiltered through a membrane filter having a pore size of 0.05 μm to obtain an actinic ray-sensitive or radiation-sensitive resin composition (resist composition) solution.

This actinic ray-sensitive or radiation-sensitive resin composition solution was coated on a 6-inch Si wafer previously subjected to a hexamethyidisilazane (HMDS) treatment, by using a spin coater, Mark 8, manufactured by Tokyo Electron Ltd. and heat-dried by using a hot plate at 130° C. over 90 seconds to form a resist film having a thickness of 100 nm.

### (2) EB Exposure and Development

This resist film was irradiated with an electron beam by using an electron beam irradiation apparatus (HL750, manufactured by Hitachi, Ltd., accelerating voltage: 50 KeV). Immediately after the irradiation, the wafer was heated on a hot plate at 120° C. for 90 seconds, then developed at 23° C. for 60 seconds by using an aqueous tetramethylammonium hydroxide solution at a concentration of 2.38 mass %, rinsed with pure water for 30 seconds, and dried to form a 1:1 line-and-space resist pattern having a line width of 50 nm.

[Line Edge Roughness (LER, nm)]

At arbitrary 30 points in the longitudinal 50 μm region of the 1:1 line-and-space resist pattern having a line width of 50 nm formed above, the distance from the reference line where the edge should be present was measured using a scanning electron microscope (S-9220, manufactured by Hitachi, Ltd.), and after determining the standard deviation, 3a was computed. A smaller value indicates a better performance.

TABLE 9

| | | | Compound | | Compound | | Basic | | | mass | | | LER |
|---|---|---|---|---|---|---|---|---|---|---|---|---|---|
| Example | Resin | (g) | (B) | (g) | (C) | (g) | Compound | (g) | Solvent | ratio | Surfactant | (g) | (nm) |
| Example 20 | P-16 | 10 | | | C-1 | 1.50 | | | SL-1/SL-5 | 60/40 | W-1 | 0.003 | 4.0 |
| Comparative | P-16 | 10 | PAG-4 | 1.48 | | | N-4 | 0.02 | SL-1/SL-5 | 60/40 | W-1 | 0.003 | 5.2 |
| Example 3 | | | | | | | | | | | | | |

It is seen from the results in Table 9 that in Example 20 using the composition according to the present invention, the line width roughness performance was excellent in the electron beam exposure using an alkali developer, as compared with Comparative Example 3 using a composition not containing the compound (C1) or (C2).

### <EUV Resist Evaluation>

### (1) Preparation and Coating of Coating Solution of Actinic Ray-Sensitive or Radiation-Sensitive Resin Composition

The components shown in Table 10 below were dissolved in the solvent shown in the same Table to give a concentration of 4.0 mass % as the solid content, and the obtained solution was microfiltered through a membrane filter having a pore size of 0.05 μm to obtain an actinic ray-sensitive or radiation-sensitive resin composition (resist composition) solution.

This actinic ray-sensitive or radiation-sensitive resin composition solution was coated on a 6-inch Si wafer previously subjected to a hexamethyldisilazane (HMDS) treatment, by using a spin coater, Mark 8, manufactured by Tokyo Electron Ltd. and dried on a hot plate at 100° C. for 60 seconds to obtain a resist film having a thickness of 50 nm.

### (2) EUV Exposure and Development

The resist film-coated wafer obtained in (1) above was patternwise exposed through an exposure mask (line/space=1/1) by using an EUV exposure apparatus (Micro Exposure Tool, manufactured by Exitech, NA: 0.3, Quadrupole, outer sigma: 0.68, inner sigma: 0.36). After the irradiation, the resist film was heated on a hot plate at 110° C. for 60 seconds, then developed by puddling the developer shown in the Table below for 30 seconds, rinsed by using the rinsing solution shown in the Table below, spun at a rotational speed of 4,000 rpm for 30 seconds and baked at 90° C. for 60 seconds to obtain a resist pattern of a 1:1 line-and-space pattern having a line width of 50 nm.

[Line Edge Roughness (LER, nm)]

The 1:1 line-and-space pattern having a line width of 50 nm obtained above was observed using a scanning electron microscope (S-9380, manufactured by Hitachi, Ltd.), and with respect to 50 points at regular intervals in the longitudinal 2 μm region of the resist pattern, the distance between the reference line where the edge should be present and the actual edge was measured. Thereafter, the standard deviation of the distance was determined, and 3σ was computed. This 3σ was taken as “LER (nm)”. A smaller value indicates a better performance in terms of line width roughness.

TABLE 10

| | | | Com- | | Com- | | | | | | | | | | |
|---|---|---|---|---|---|---|---|---|---|---|---|---|---|---|---|
| | | | pound | | pound | | Basic | | | mass | | | | Rinsing | LER |
| Example | Resin | (g) | (B) | (g) | (C) | (g) | Compound | (g) | Solvent | ratio | Surfactant | (g) | Developer | Solution | (nm) |
| Example 21 | P-16 | 10 | | | C-1 | 1.50 | | | SL-1/SL-5 | 60/40 | W-1 | 0.003 | SG-9 | SR-4 | 4.8 |
| Example 22 | P-17 | 10 | | | C-1 | 1.50 | | | SL-1/SL-6 | 60/40 | W-1 | 0.003 | SG-1 | SR-1 | 4.7 |
| Comparative | P-16 | 10 | PAG-4 | 1.48 | | | N-4 | 0.02 | SL-1/SL-5 | 60/40 | W-1 | 0.003 | SG-9 | SR-4 | 5.8 |
| Example 4 | | | | | | | | | | | | | | | |
| Comparative | P-17 | 10 | PAG-4 | 1.48 | | | N-4 | 0.02 | SL-1/SL-6 | 60/40 | W-1 | 0.003 | SG-1 | SR-1 | 6.1 |
| Example 5 | | | | | | | | | | | | | | | |

It is seen from the results in Table 10 that in Examples 21 and 22 using the composition according to the present invention, the line width roughness performance was excellent in the extreme-ultraviolet exposure using an organic solvent developer, as compared with Comparative Examples 4 and 5 using a composition not containing the compound (C1) or (C2).

### INDUSTRIAL APPLICABILITY

According to the present invention, a pattern forming method ensuring that the roughness performance such as line width roughness, the local pattern dimension uniformity and the exposure latitude are excellent and reduction in the film thickness of the pattern part formed by development, so-called film loss, can be suppressed, a compound used therein, an actinic ray-sensitive or radiation-sensitive resin composition, a resist film, a manufacturing method of an electronic device, and an electronic device can be provided.

This application is based on a Japanese patent application filed on Jan. 31, 2013 (Japanese Patent Application No. 2013-017949), US provisional application filed on Jan. 31, 2013 (U.S. Provisional Application No. 61/758,973), and the contents thereof are incorporated herein by reference.

1. An actinic ray-sensitive or radiation-sensitive resin composition comprising:
(A) a resin having a group capable of decomposing by an action of an acid to produce a polar group,
(C1) a compound containing a group capable of generating a first acidic functional group upon irradiation with an actinic ray or radiation and a group capable of generating a second acidic functional group different from the first acidic functional group upon irradiation with an actinic ray or radiation, and
(C2) at least one compound containing two or more groups selected from the group consisting of a group capable of generating a structure represented by the following formula (a) upon irradiation with an actinic ray or radiation, a group capable of generating a structure represented by the following formula (b) upon irradiation with an actinic ray or radiation, a group capable of generating a structure represented by the following formula (c) upon irradiation with an actinic ray or radiation, and a group capable of generating a structure represented by the following formula (d) upon irradiation with an actinic ray or radiation: 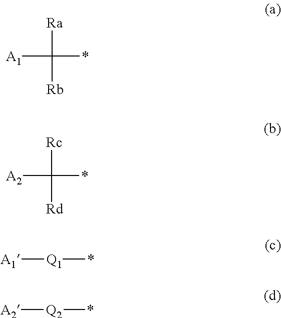
wherein in formulae (a), (b), (c) and (d),
A₁, A₂, A₁′ and A₂′ represent the same acidic functional group,
each of Ra, Rb, Rc and Rd independently represents a hydrogen atom or a substituent,
each of Q₁ and Q₂ represents a cyclic group,
provided that the structure represented by formula (a) is different from the structure represented by formula (b) and the structure represented by formula (c) is different from the structure represented by formula (d), and
* represents a bond.

2. The actinic ray-sensitive or radiation-sensitive resin composition as claimed in claim 1,
wherein the compound (C1) is a compound capable of generating, as the first acidic functional group and the second acidic functional group, different groups from each other selected from the group consisting of groups represented by the following formulae (Ca-1) to (Ca-19), upon irradiation with an actinic ray or radiation: 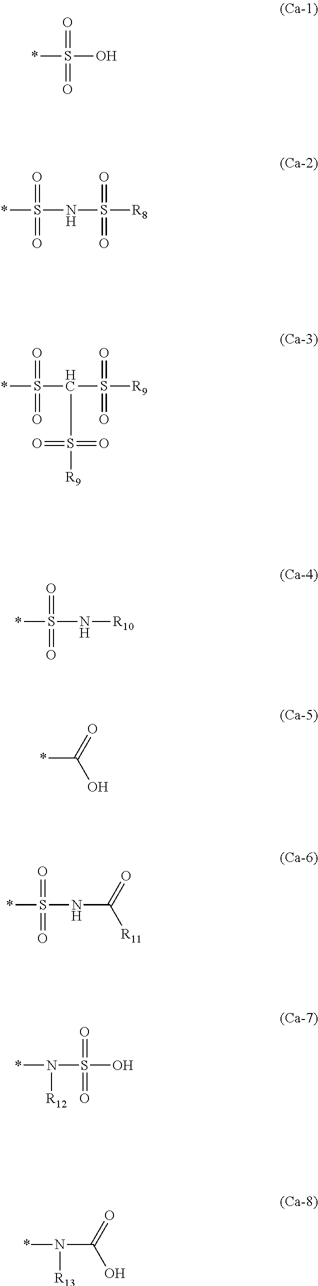 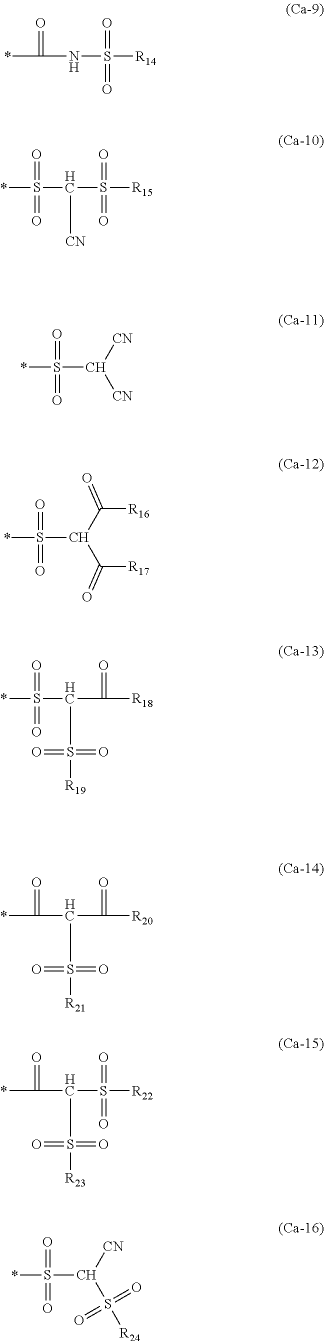 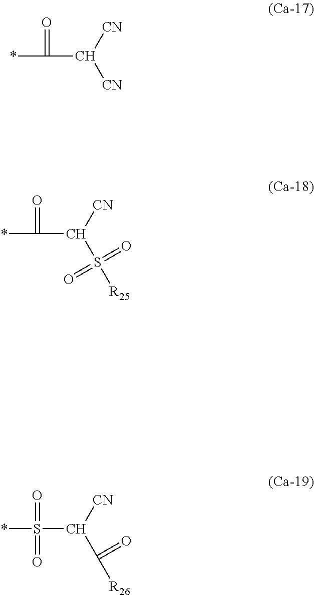
wherein in formulae (Ca-2) to (Ca-4), (Ca-6) to (Ca-10), (Ca-12) to (Ca-16), (Ca-18) and (Ca-19),
each of R₈, R₉, R₁₁ and R₁₄ to R₂₆ independently represents an alkyl group, a cycloalkyl group or an aryl group,
R₁₀ represents a hydrogen atom, an alkyl group, a cycloalkyl group or an aryl group, and
each of R₁₂ and R₁₃ independently represents a hydrogen atom, an alkyl group, an aryl group, or a single bond, alkylene group or arylene group capable of bonding to any one atom in the molecule to form a ring.

3. The actinic ray-sensitive or radiation-sensitive resin composition as claimed in claim 2,
wherein the compound (C1) is a compound capable of generating a group selected from the group consisting of groups represented by the following formulae (Cb-1) to (Cb-4), upon irradiation with an actinic ray or radiation: 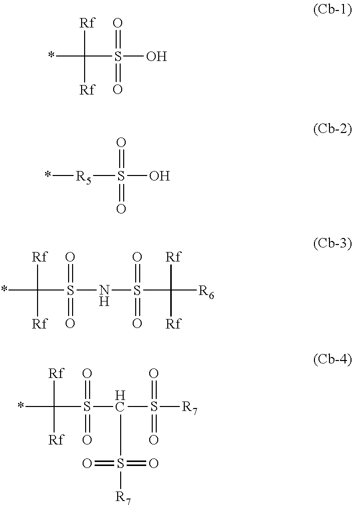
wherein in formulae (Cb-1) to Cb-4),
each Rf independently represents a fluorine atom or an alkyl group substituted with at least one fluorine atom,
R₅ represents an arylene group containing a fluorine atom or an alkyl group substituted with at least one fluorine atom,
R₆ represents a hydrogen atom, a fluorine atom or an alkyl group,
each R₇ independently represents an alkyl group, a cycloalkyl group or an aryl group, and
* represents a bond.

4. The actinic ray-sensitive or radiation-sensitive resin composition as claimed in claim 1,
wherein the compound (C2) is a compound capable of generating, as A₁ in formula (a), A₂ in formula (b), A₁′ in formula (c) and A₂′ in formula (d), the same groups as each other selected from the group consisting of groups represented by the following formulae (Ca-1) to (Ca-19), upon irradiation with an actinic ray or radiation: 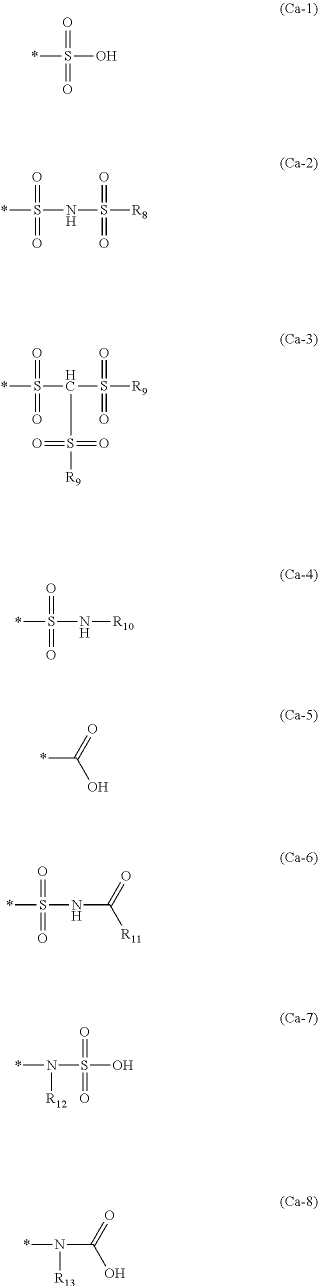 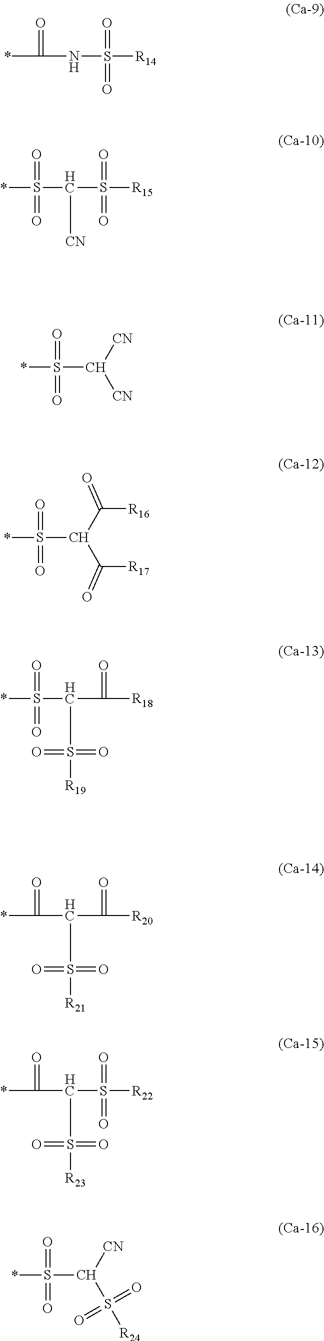 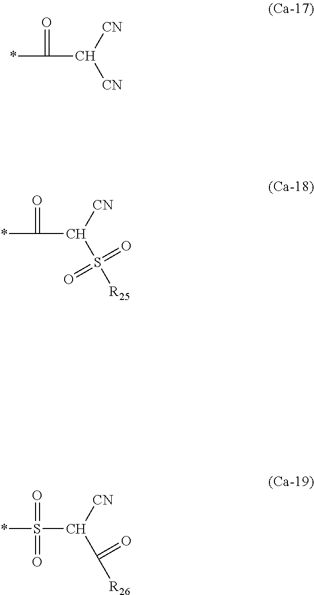
wherein in formulae (Ca-2) to (Ca-4), (Ca-6) to (Ca-10), (Ca-12) to (Ca-16), (Ca-18) and (Ca-19),
each of R₈, R₉, R₁₁ and R₁₄ to R₂₆ independently represents an alkyl group, a cycloalkyl group or an aryl group,
R₁₀ represents a hydrogen atom, an alkyl group, a cycloalkyl group or an aryl group, and
each of R₁₂ and R₁₃ independently represents a hydrogen atom, an alkyl group, an aryl group, or a single bond, alkylene group or arylene group capable of bonding to any one atom in the molecule to form a ring.

5. The actinic ray-sensitive or radiation-sensitive resin composition as claimed in claim 4,
wherein the compound (C2) is a compound capable of generating a group selected from the group consisting of groups represented by the following formulae (Cb-1) to (Cb-4), upon irradiation with an actinic ray or radiation: 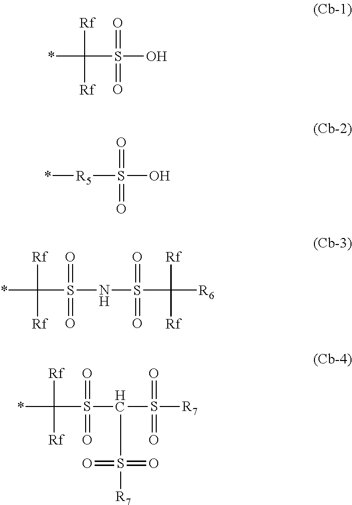
wherein in formulae (Cb-1) to (Cb-4),
each Rf independently represents a fluorine atom or an alkyl group substituted with at least one fluorine atom,
R₅ represents an arylene group containing a fluorine atom or an alkyl group substituted with at least one fluorine atom,
R₆ represents a hydrogen atom, a fluorine atom or an alkyl group,
each R₇ independently represents an alkyl group, a cycloalkyl group or an aryl group, and
* represents a bond.

6. The actinic ray-sensitive or radiation-sensitive resin composition as claimed in claim 1,
wherein the compound (C2) is a compound containing a group capable of generating a structure represented by formula (a) upon irradiation with an actinic ray or radiation and a group capable of generating a structure represented by formula (b) upon irradiation with an actinic ray or radiation,
at least either one of Ra and Rb in formula (a) represents a fluorine atom or an alkyl fluoride group, and
each of Rc and Rd in formula (b) independently represents a hydrogen atom or an alkyl group not substituted with a fluorine atom.

7. The actinic ray-sensitive or radiation-sensitive resin composition as claimed in claim 1, further comprising:
(B) a compound capable of generating an acid upon irradiation with an actinic ray or radiation, which is different from the compounds (C1) and (C2).

8. The actinic ray-sensitive or radiation-sensitive resin composition as claimed in claim 7,
wherein the compound (B) is a compound capable of generating an organic acid represented by the following formula (V) or (VI) upon irradiation with an actinic ray or radiation: 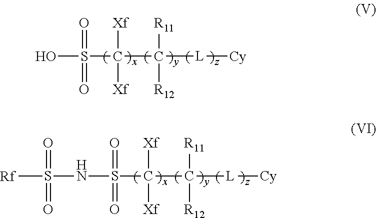
wherein in formulae (V) and (VI),
each Xf independently represents a fluorine atom or an alkyl group substituted with at least one fluorine atom,
each L independently represents a divalent linking group,
each of R₁₁ and R₁₂ independently represents a hydrogen atom, a fluorine atom or an alkyl group,
Cy represents a cyclic organic group,
Rf represents a fluorine atom-containing group,
x represents an integer of 1 to 20,
y represents an integer of 0 to 10, and
z represents an integer of 0 to 10.

9. The actinic ray-sensitive or radiation-sensitive resin composition as claimed in claim 1,
wherein the resin (A) contains (AI) a repeating unit capable of decomposing by an action of an acid to produce a carboxyl group.

10. The actinic ray-sensitive or radiation-sensitive resin composition as claimed in claim 9,
wherein the content of the repeating unit (AI) is 50 mol % or more based on all repeating units in the resin (A).

11. The actinic ray-sensitive or radiation-sensitive resin composition as claimed in claim 1, further comprising:
(D) a hydrophobic resin different from the resin (A).

12. A pattern forming method comprising:
(i) a step of forming a film by using the actinic ray-sensitive or radiation-sensitive resin composition claimed in claim 1,
(ii) a step of exposing the film, and
(iii) a step of developing the exposed film by using a developer to form a pattern.

13. The pattern forming method as claimed in claim 12,
wherein the step (iii) is a step of developing the exposed film by using an organic solvent-containing developer to form a negative pattern.

14. The pattern forming method as claimed in claim 12,
wherein the exposure in the step (ii) is immersion exposure.

15. The pattern forming method as claimed in claim 13,
wherein the developer is a developer containing at least one kind of an organic solvent selected from the group consisting of a ketone-based solvent, an ester-based solvent, an alcohol-based solvent, an amide-based solvent and an ether-based solvent.

16. A resist film formed of the actinic ray-sensitive or radiation-sensitive resin composition claimed in claim 1.

17. A method for manufacturing an electronic device, comprising the pattern forming method claimed in claim 12.

18. An electronic device manufactured by the manufacturing method of an electronic device claimed in claim 17.

19. A compound represented by the following formula (C-1) or (C-2): 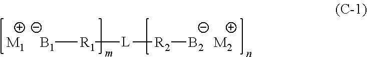
wherein in formula (C-1),
each of M₁ and M₂ represents an organic counter cation structure,
B₁ represents an acid anion moiety of a first acidic functional group,
B₂ represents an acid anion moiety of a second acidic functional group different from the first acidic functional group,
each of R₁ and R₂ independently represents a single bond, an alkylene group, a cycloalkylene group or an arylene group,
L represents an (m+n)-valent linking group,
each of m and n represents an integer, and m≧n; 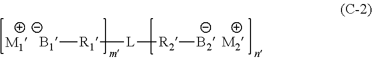
wherein in formula (C-2),
M₁′, M₂′, R₁′, R₂′, L′, m′ and n′ have the same meanings as M₁, M₂, R₁, R₂, L, m and n, respectively, in formula (C-1), m′≧n′, and
B₁′ and B₂′ represent different kinds of acid anion structures selected from the group consisting of an acid anion structure of a structure represented by the following formula (a), an acid anion structure of a structure represented by the following formula (b), an acid anion structure of a structure represented by the following formula (c), and an acid anion structure of a structure represented by the following formula (d): 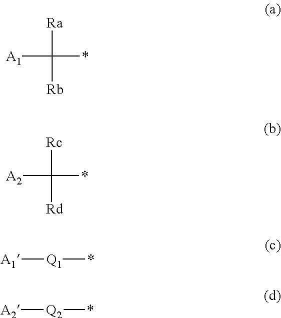
wherein in formulae (a), (b), (c) and (d),
A₁, A₂, A₁′ and A₂′ represent the same acidic functional group,
each of Ra, Rb, Rc and Rd independently represents a hydrogen atom or a substituent,
each of Qt and Q₂ represents a cyclic group,
provided that the structure represented by formula (a) is different from the structure represented by formula (b) and the structure represented by formula (c) is different from the structure represented by formula (d), and
* represents a bond.
